(19) United States
(12) Patent Application Publication
(10) Pub. No.: US 2023/0009626 A1
(43) Pub. Date: Jan. 12, 2023

(54) INDOLINONE COMPOUNDS FOR USE AS MAP4K1 INHIBITORS

(71) Applicant: ICHNOS SCIENCES S.A., La Chaux-de-Fonds (CH)

(72) Inventors: Sachin Sundarlal CHAUDHARI, La Chaux-de-Fonds (CH); Laxmikant Atmaram GHARAT, La Chaux-de-Fonds (CH); Pravin IYER, La Chaux-de-Fonds (CH); Sachin Vasantrao DHONE, La Chaux-de-Fonds (CH); Bharat Gangadhar ADIK, La Chaux-de-Fonds (CH); Prashant Dilip WADEKAR, La Chaux-de-Fonds (CH); Nagaraj GOWDA, La Chaux-de-Fonds (CH); Malini BAJPAI, La Chaux-de-Fonds (CH)

(21) Appl. No.: 17/282,432

(22) PCT Filed: Oct. 7, 2019
(86) PCT No.: PCT/EP2019/077086

§ 371 Date: Apr. 2, 2021

(30) Foreign Application Priority Data
Oct. 5, 2018 (IN) 201821037777
Mar. 8, 2019 (IN) 201921009045
Jun. 21, 2019 (IN) 201921024673

(51) Int. Cl. C07D 401/12; C07D 209/34; C07D 405/14; C07D 403/12; C07D 471/04; C07F 7/08; C07D 401/14

(52) U.S. Cl. CPC C07D 401/12; C07D 209/34; C07D 405/14; C07D 401/14; C07D 471/04; C07F 7/0816; C07D 403/12

(57) ABSTRACT

The present disclosure is directed to compounds of formula (I) and pharmaceutically acceptable salts thereof, wherein ring A, ring C, X¹, X², L¹, R¹, R², R³, R⁴, R⁵, R⁶, R⁷, m and n are as defined herein, which are useful as MAP4K1 inhibitors, processes for their preparation, pharmaceutical compositions comprising the compounds, and the use of the compounds or the compositions in the treatment or prevention of various diseases, conditions and/or disorders mediated by MAP4K1.

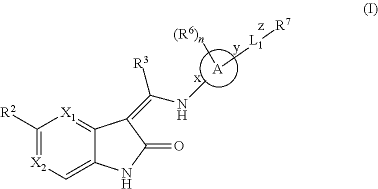

### RELATED APPLICATIONS

This application claims the benefit of Indian Provisional Application No. 201821037777 filed on Oct. 5, 2018, 201921009045 filed on Mar. 8, 2019 and 201921024673 filed on Jun. 21, 2019; which is hereby incorporated by reference in its entirety.

### TECHNICAL FIELD

The present patent application is directed to novel inhibitors of the mitogen-activated protein kinase kinase kinase kinase 1, also known as MAP4K1 or HPK1 (hematopoietic progenitor kinase 1).

### BACKGROUND OF THE INVENTION

Protein kinases represent a large family of proteins which play a variety of crucial roles in the regulation of a wide range of cellular processes. Such kinases include Akt, Axl, Aurora A, Aurora B, DYRK2, EPHAa2, FGFR3, FLT-3, VEGFr3, IGFLr, IKK2, JNK3, VEGFr2, MEK1, MET, P70s6K, Plk1, RSK1, Src, TrkA, Zap70, cKit, bRaf, EGFR, Jak2, PI3K, NPM-Alk, c-Abl, BTK, FAK, PDGFR, TAK1, LimK, Flt1, PDK1, Erk and RON Inhibition of various protein kinases, especially selective inhibition, has become an important strategy in treating many diseases and disorders.

MAP4K1 is a serine/threonine kinase of the Ste20 family. MAP4K enzymes (MAP kinase kinases) are generally involved at the highest level of a largely linear kinase activation pathway. A MAP4K will phosphorylate and activate a particular substrate which is a MAP3K (a MAP kinase kinase). A MAP3K in turn phosphorylates and activates a MAP2K (a MAP kinase kinase). A MAP2K in turn phosphorylates and activates a MAPK (MAP kinase). The MAP kinase is the final effector of the pathway and it in turn phosphorylates a substrate to control key cellular processes such as cell proliferation, cell differentiation, gene expression, transcription regulation, and apoptosis. The substrate of MAPK is generally a nuclear protein, such as nuclear factor kappa-B (NF-κB). Activation of the MAPK by its phosphorylation by an MAP2K results in translocation of this final enzyme in the cascade into the nucleus.

MAP4K1, also known as HPK1, is primarily expressed in the immune system's Tcells and B cells, which are critical in regulation of the immune system. Overstimulation of T cell and B cell activation pathways can result in auto-immune diseases, while understimulation of these pathways can result in immune dysfunction, susceptibility to viral and bacterial infection and increased susceptibility to cancer. MAP4K1 is activated by its interaction with activated T cell receptors (TCRs) and B cell receptors (BCRs), so MAP4K1 activation serves to convey the cellular activation signal from the surface of a T or B cell to the effector proteins in the nucleus. There is also evidence that MAP4K1 can be activated via the TGF-β receptor, the erythropoietin receptor and the FAS protein (which is involved in apoptosis signaling). MAP4K1 activation ultimately results in activation of several identified nuclear effector proteins, including those involved in the NF-κ1, AP-1, ERK2, and Fos signaling pathways.

MAP4K1 is considered a negative regulator of T cell receptor (TCR) activation signals, and it is one of the effector molecules that mediates immunosuppression of T cell responses upon exposure to prostaglandin E2 (PGE2). Studies have shown that MAPK1 activity dampens the strength of the T cell receptor signal transduction cascade, and thus, targeted genetic disruption of MAP4K1 results in strengthened TCR activation signals.

One particularly important pathway that MAP4K1 appears to be involved with is the JNK pathway. MAP4K1 regulates the MAP3K's MEKK1, TAK1 and MLK3. These in turn regulate the MAP2K's MKK4 and MKK7. These in turn regulate the MAPK JNK. JNK then regulates important transcription factors and other proteins, including p53, SMAD4, NFAT-2, NFAT-4, ELK1, ATF2, HSF1, c-Jun, and JunD. JNK has been implicated in apoptosis, neurodegeneration, cell differentiation and proliferation, inflammatory conditions and cytokine production.

The JNK signal transduction pathway is activated in response to environmental stress and by the engagement of several classes of cell surface receptors, including cytokine receptors, serpentine receptors and receptor tyrosine kinases. In mammalian cells, the JNK pathway has been implicated in biological processes such as oncogenic transformation and mediating adaptive responses to environmental stress. JNK has also been associated with modulating immune responses, including maturation and differentiation of immune cells, as well as effecting programmed cell death in cells identified for destruction by the immune system. Among several neurological disorders, JNK signaling is particularly implicated in ischemic stroke and Parkinson's disease, but also in other diseases as mentioned further below.

It is noteworthy that the MAPK p3 8alpha was shown to inhibit cell proliferation by antagonizing the JNK-c-Jun-pathway. p38a1pha appears to be active in suppression of proliferation in both normal cells and cancer cells, and this strongly suggests the involvement of JNK in hyperproliferative diseases (see, e.g., Hui et al., Nature Genetics, Vol. 39, No. 6, June 2007). JNK signaling has also been implicated in diseases such as excitotoxicity of hippocampal neurons, liver ischemia, reperfusion, neurodegenerative diseases, hearing loss, deafness, neural tube birth defects, cancer, chronic inflammatory diseases, obesity, diabetes, in particular, insulin-resistant diabetes, and it has been proposed that selective JNK inhibitors are needed for treatment of various diseases with a high degree of specificity and lack of toxicity.

Because MAP4K1 is an upstream regulator of JNK, effective inhibitors of MAP4K1 would be useful in treating the same diseases which have been suggested or implicated for JNK inhibitors, especially where such disease or dysfunction is manifested in hematopoietic cells such as T cells and B cells.

Targeted disruption of MAP4K1 (HPK1) alleles has been shown to confer T cells with an elevated Th1 cytokine production in response to TCR engagement. Burakoff et al., Immunologic Research, 54(1): 262-265 (2012). HPK1−/− T cells were found to proliferate more rapidly than the haplotype-matched wild-type counterpart and were resistant to prostaglandin E2 (PGE2)-mediated suppression. Most strikingly, mice that received adoptive transfer of HPK1−/− T cells became resistant to lung tumor growth. Also, the loss of HPK1 from dendritic cells (DCs) endowed them with superior antigen presentation ability, enabling HPK1−/− DCs to elicit a more potent anti-tumor immune response when used as cancer vaccine. It was considered probable that blocking the MAP4K1 kinase activity with a small molecule inhibitor may activate the superior antitumor activity of both cell types, resulting in a synergistic amplification of anti-tumor potential. Given that MAP4K1 is not expressed in any major organs, it is less likely that a selective inhibitor of MAP4K1 would cause any serious side effects. The relationship between MAP4K1 and PGE2 is particularly noteworthy because PGE2 is the predominant eicosanoid product released by cancer cells, including lung, colon and breast cancer cells. Tumor-produced PGE2 is known to contribute significantly to tumor-mediated immune suppression.

Zhang et al., J. Autoimmunity, 37:180-189 (2011), described diminished HPK1 expression in CD4 T cells of lupus patients due to the selective loss of JMJD3 histone demethylase binding to the HPK1 locus. This suggests that HPK1 is one of the key molecules involved in the maintenance of peripheral tolerance. Peripheral tolerance is one of the major obstacles to the development of effective anti-tumor immunity.

Several small molecule inhibitors of MAP4K1 have been reported, but they do not inhibit MAP4K1 selectively, or even preferentially. Such inhibitors include staurosporine, bosutinib, sunitinib, lestaurtinib, crizotinib, foretinib, dovitinib and KW-2449. Staurosporine, for example, broadly inhibits a wide range of protein kinases across both the serine/threonine and tyrosine kinase families. Bosutinib is primarily an inhibitor of the tyrosine kinase BCR-Abl, with additional activity against the Src family tyrosine kinases. Sunitinib is a broad inhibitor of tyrosine kinases. Lestaurtinib is primarily an inhibitor of the FLT, JAK and TRK family tyrosine kinases. Crizotinib is primarily an inhibitor of the c-met and ALK tyrosine kinases. Foretinib was under study as an inhibitor of the c-Met and VEGFR tyrosine kinases. Dovitinib is primarily an inhibitor of the FGFR receptor tyrosine kinase. KW-2449 is an experimental inhibitor primarily of the FLT3 tyrosine kinase.

Sunitinib inhibits MAP4K1 at nanomolar concentrations, but it is a broad-spectrum receptor tyrosine kinase inhibitor. Treating T-cells with sunitinib results in enhanced cytokine product similar to that observed with HPK1 −/− Tcells, which suggests that in T cells a selective MAP4K1 inhibitor could produce the same enhanced immune response phenotype.

Currently, there is a largely unmet need for an effective way of treating disease and disorders associated disrupted protein kinase signaling. Autoimmune diseases, inflammatory diseases, neurological and neurodegenerative diseases, cancer, cardiovascular diseases, allergies and asthma, are all diseases and disorder which can be affected by dysfunctional protein kinase signaling. Improved therapeutic compounds, compositions and methods for the treatment for these disease and disorders are urgently required. MAP4K1 inhibition is an especially attractive target for cancer immunotherapy.

The major challenge currently faced in the field is the lack of MAP4K1 specific inhibitors. The present disclosure provides novel, highly effective small-molecule inhibitors of MAP4K1.

### SUMMARY OF THE INVENTION

The invention provides a compound of formula (I)

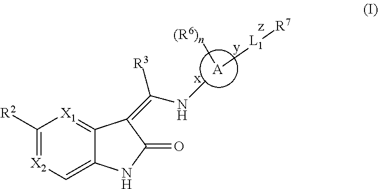

stereoisomer, diastereoisomer, enantiomer or a pharmaceutically acceptable salt thereof,
wherein,

X¹ is selected from CH and N;

X² is selected from CH, CR¹ and N;

R¹ is selected from halogen, cyano and C₁₋₈alkyl;

R² is

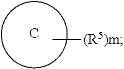

Ring C is selected from

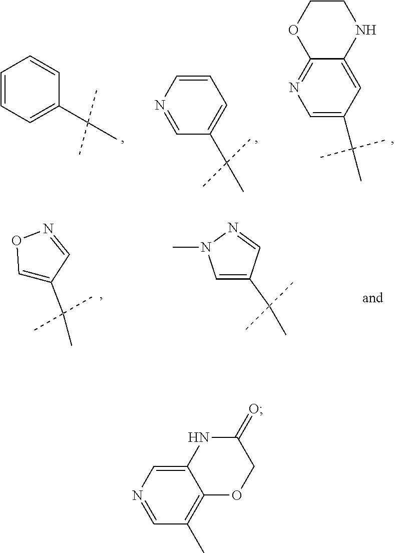

each occurrence of R⁵ is selected from cyano, halogen, C₁₋₈alkyl, C₁₋₈alkoxy, haloC₁₋₈alkoxy, C₃₋₁₂cycloalkyl, C₁₋₈alkoxy C₃₋₁₂cycloalkyl, hydroxyC₁₋₈alkyl and amino;

R³ is C₁₋₈alkyl;

Ring A is selected from

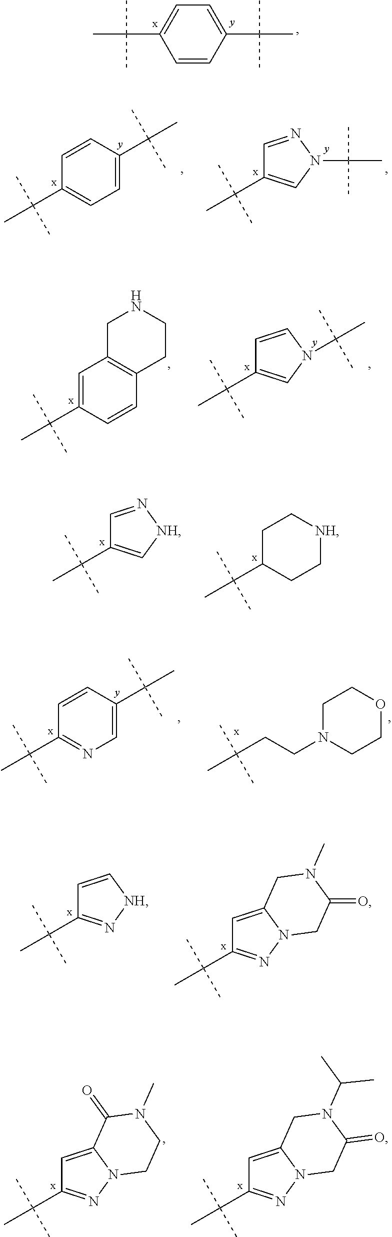

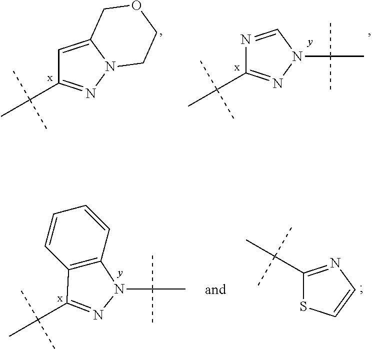

L¹ is absent or selected from

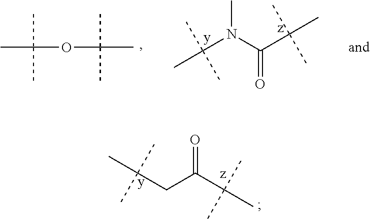

x, y and z are point of attachments;

R⁷ is selected from

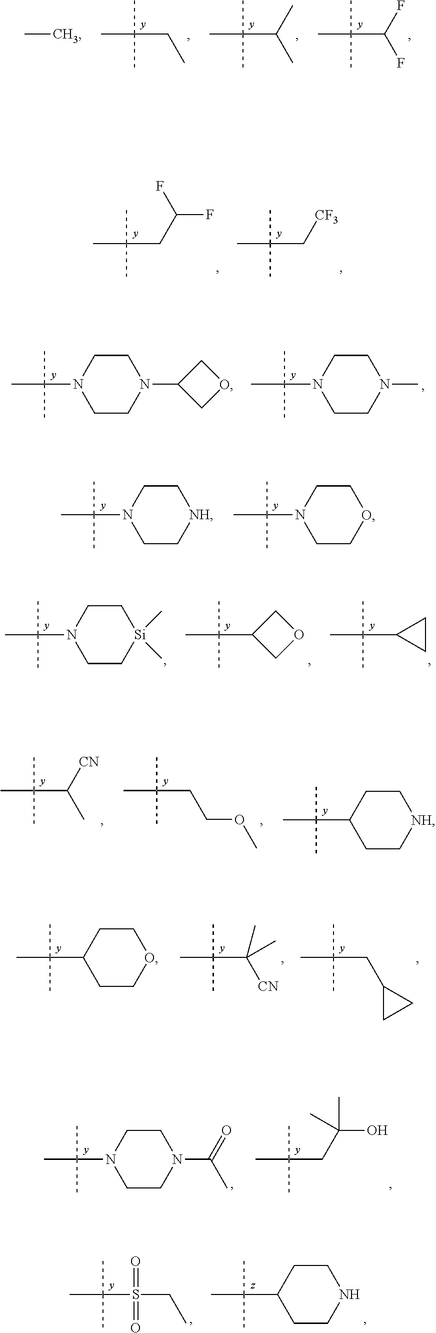

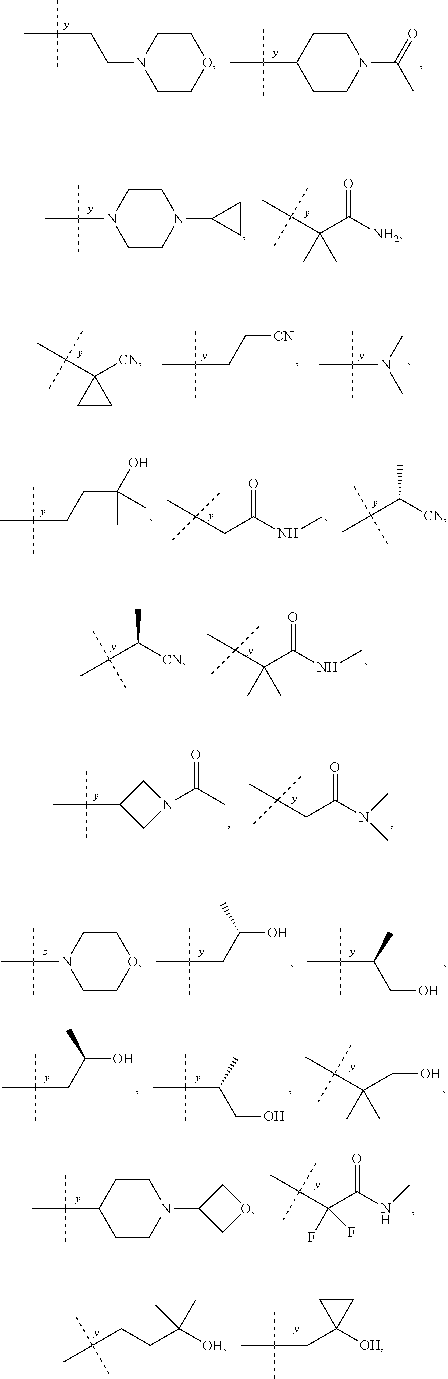

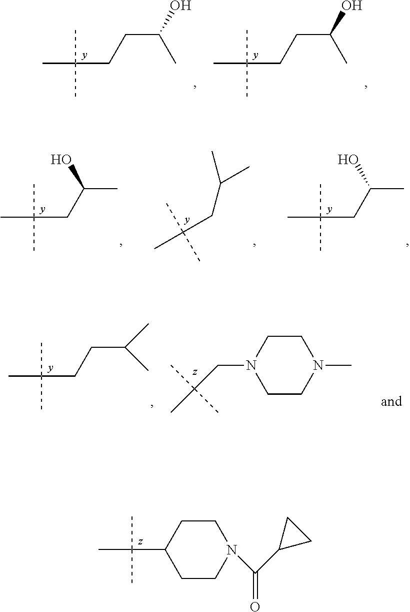

each occurrence of R⁶ is selected from C₁₋₈alkyl, C₁₋₈alkoxy, haloC₁₋₈alkyl, hydroxyC₁₋₈alkyl and C₃₋₁₂cycloalkyl;

‘m’ is 0, 1 or 2; and

‘n’ is 0, 1 or 2.

The compounds of formula (I) may involve one or more embodiments. It is to be understood that the embodiments below are illustrative of the present invention and are not intended to limit the claims to the specific embodiments exemplified. It is also to be understood that the embodiments defined herein may be used independently or in conjunction with any definition and any other embodiment defined herein. Thus the invention contemplates all possible combinations and permutations of the various independently described embodiments. For example, the invention provides compounds of formula (I) as defined above wherein R³ is hydrogen, methyl, ethyl, isopropyl or phenyl (according to an embodiment defined below), ‘n’ is 0, 1 or 2 (according to another embodiment defined below).

According to yet another embodiment, specifically provided are compounds of formula (I), in which X¹ is CH.

According to yet another embodiment, specifically provided are compounds of formula (I), in which X¹ is N.

According to yet another embodiment, specifically provided are compounds of formula (I), in which X¹ and X² are CH.

According to yet another embodiment, specifically provided are compounds of formula (I), in which X¹ and X² are N.

According to yet another embodiment, specifically provided are compounds of formula (I), in which X¹ is CH and X² is N.

According to yet another embodiment, specifically provided are compounds of formula (I), in which X¹ is N and X² is CH.

According to yet another embodiment, specifically provided are compounds of formula (I), in which X¹ is CH and X² is CR¹.

According to yet another embodiment, specifically provided are compounds of formula (I), in which R¹ is a halogen (e.g. fluoro or chloro) C₁₋₈alkyl (e.g. methyl) or cyano.

According to yet another embodiment, specifically provided are compounds of formula (I), in which R¹ is fluoro, chloro, methyl or cyano.

According to yet another embodiment, specifically provided are compounds of formula (I), in which R⁵ is halogen (e.g. fluoro), C₁₋₈alkyl (e.g. methyl), C₁₋₈alkoxy (e.g. methoxy or ethoxy), haloC₁₋₈alkoxy (e.g. difluoromethoxy), C₃₋₁₂cycloalkyl C₁₋₈alkoxy (e.g. cyclopropylmethoxy) or amino.

According to yet another embodiment, specifically provided are compounds of formula (I), in which R⁵ is fluoro, methyl, methoxy, ethoxy, difluoromethoxy or amino.

According to yet another embodiment, specifically provided are compounds of formula (I), in which

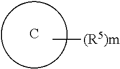

is

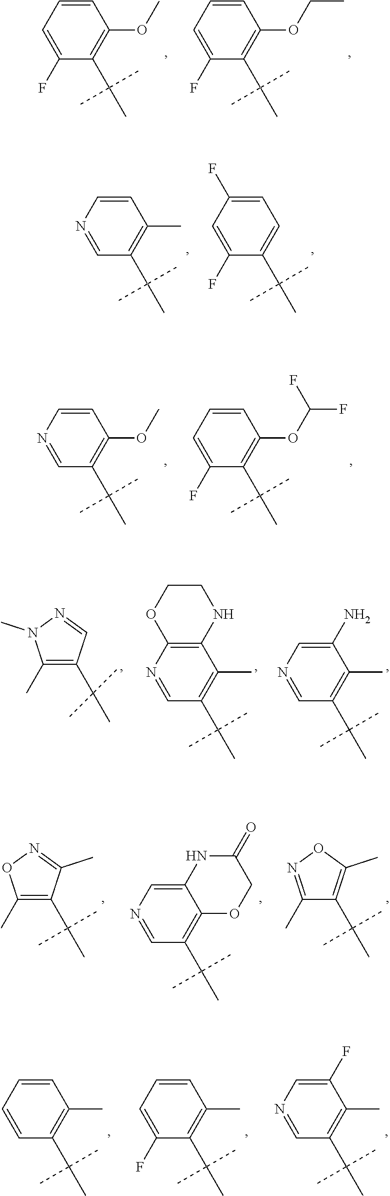

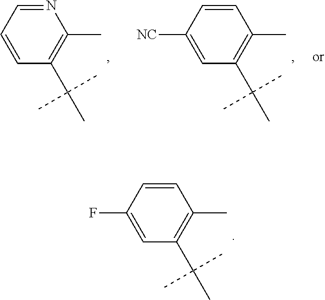

According to yet another embodiment, specifically provided are compounds of formula (I), in which R³ is methyl, ethyl or isopropyl.

According to yet another embodiment, specifically provided are compounds of formula (I), in which R⁶ is C₁₋₈alkyl (e.g. methyl or ethyl), C₁₋₈alkoxy (e.g. methoxy), hydroxyC₁₋₈alkyl (e.g.

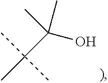

C₃₋₁₂cycloalkyl (e.g. cyclopropyl) or

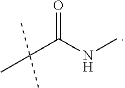

According to yet another embodiment, specifically provided are compounds of formula (I), in which R⁶ is methyl, ethyl, methoxy,

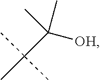

cyclopropyl or

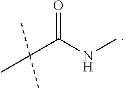

According to yet another embodiment, specifically provided are compounds of formula (I), in which L¹ is absent.

According to yet another embodiment, specifically provided are compounds of formula (I), in which L¹ is

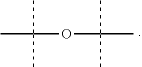

According to yet another embodiment, specifically provided are compounds of formula (I), in which L¹ is

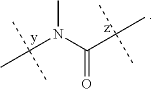

According to yet another embodiment, specifically provided are compounds of formula (I), in which L¹ is

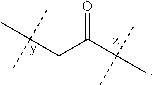

According to yet another embodiment, specifically provided are compounds of formula (I), in which is

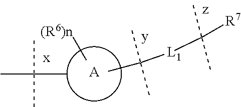

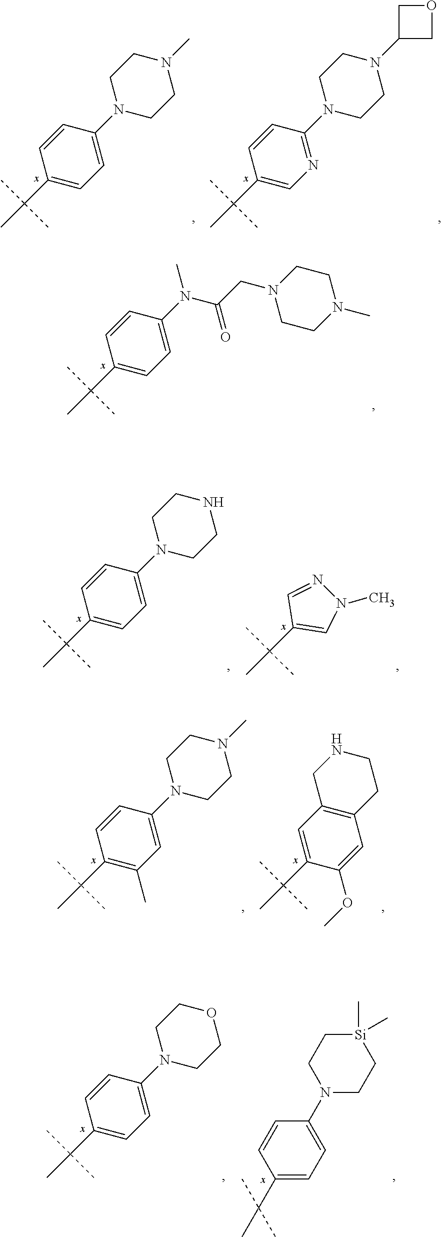

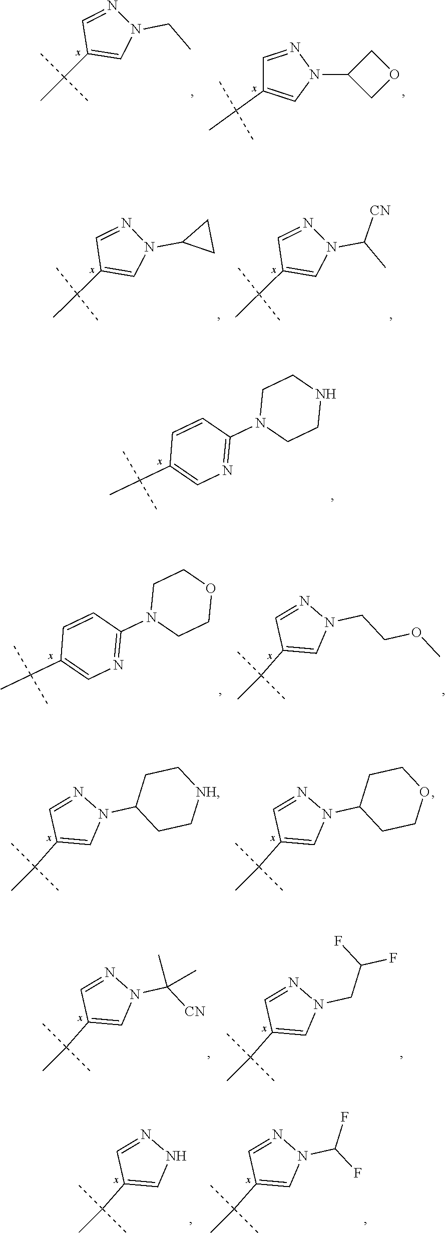

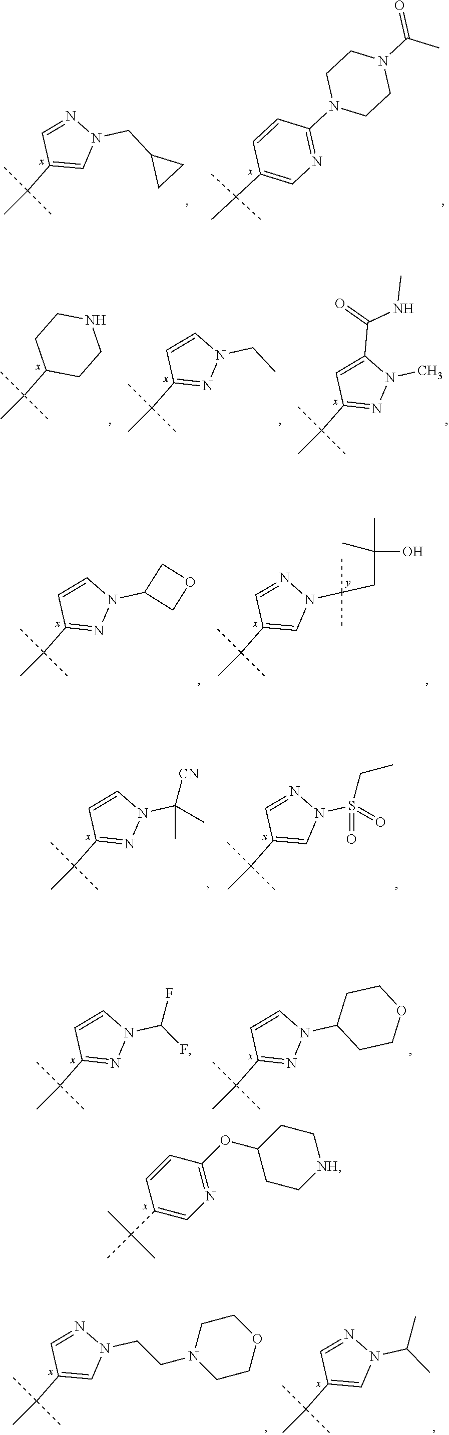

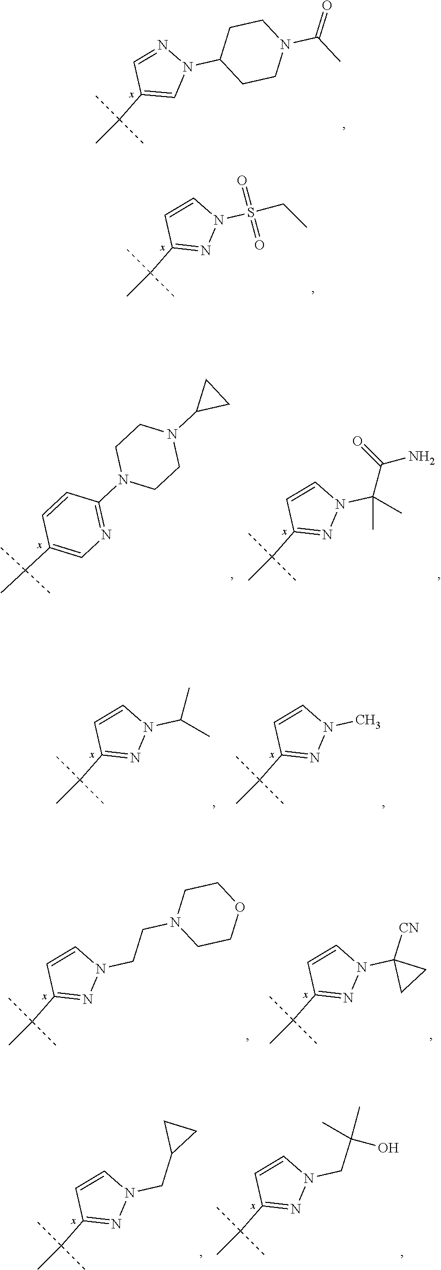

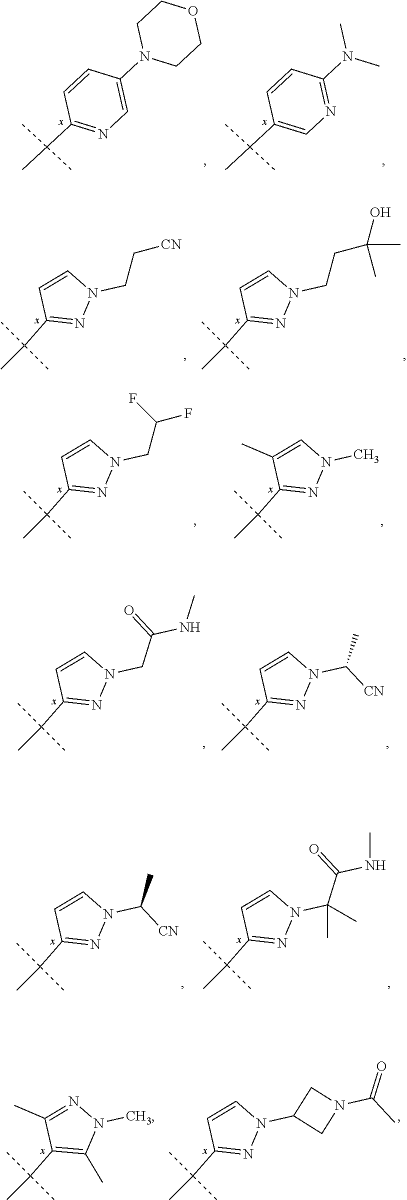

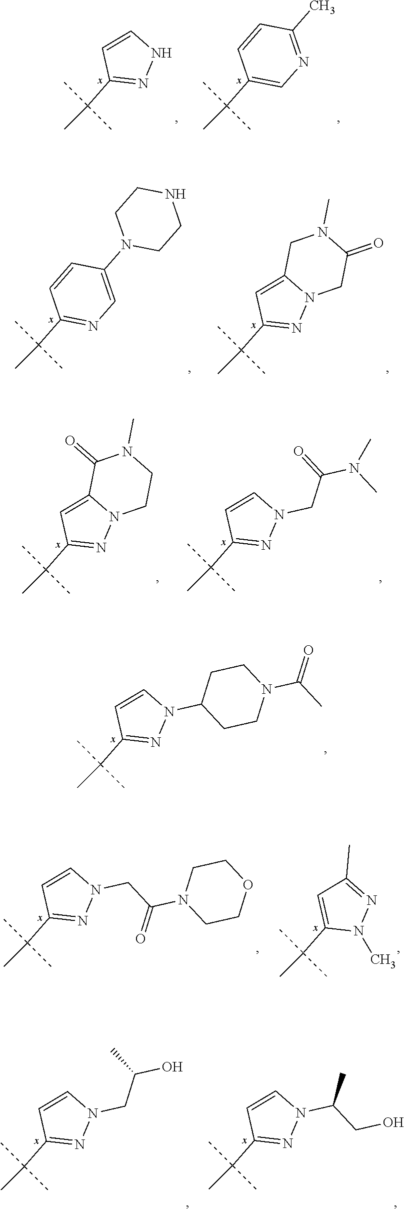

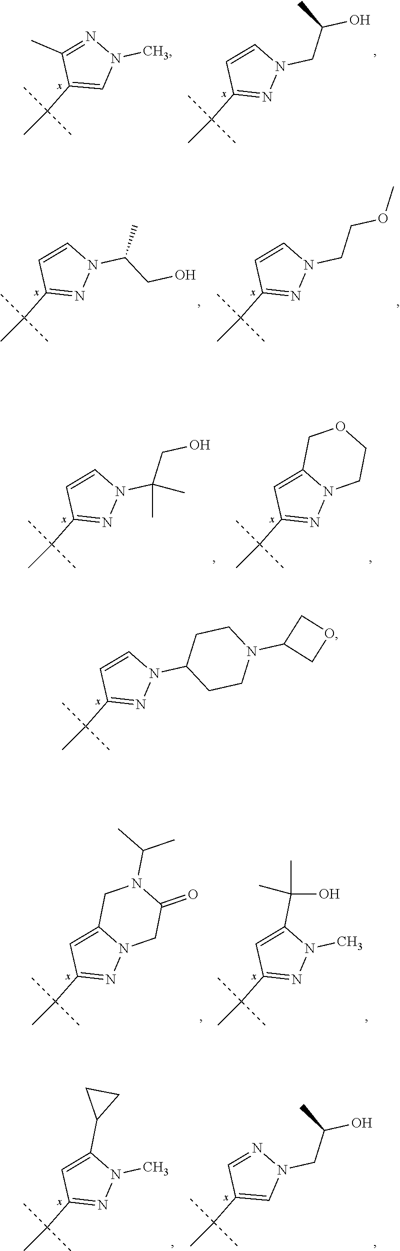

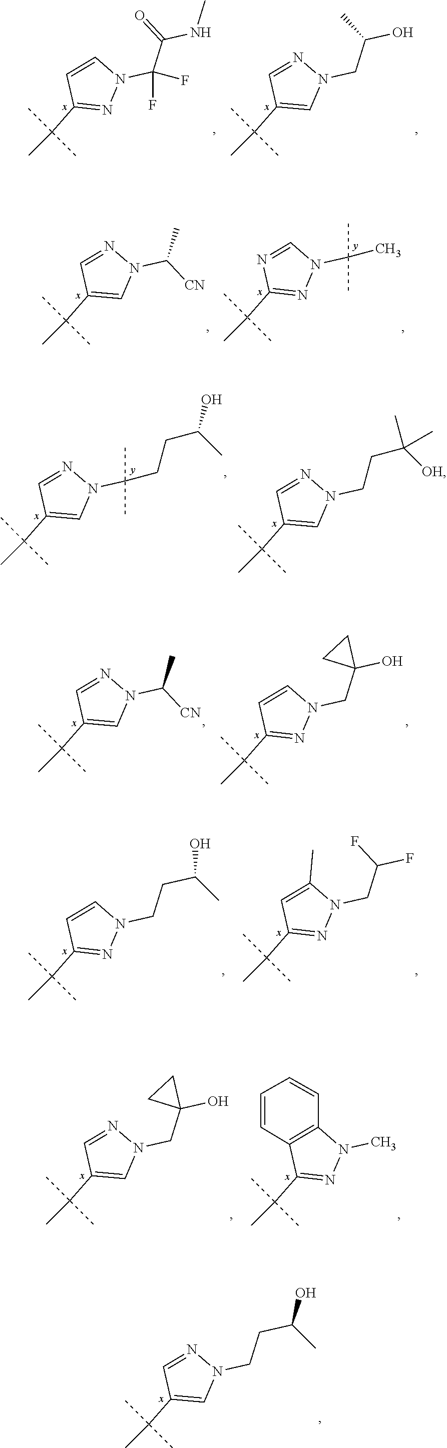

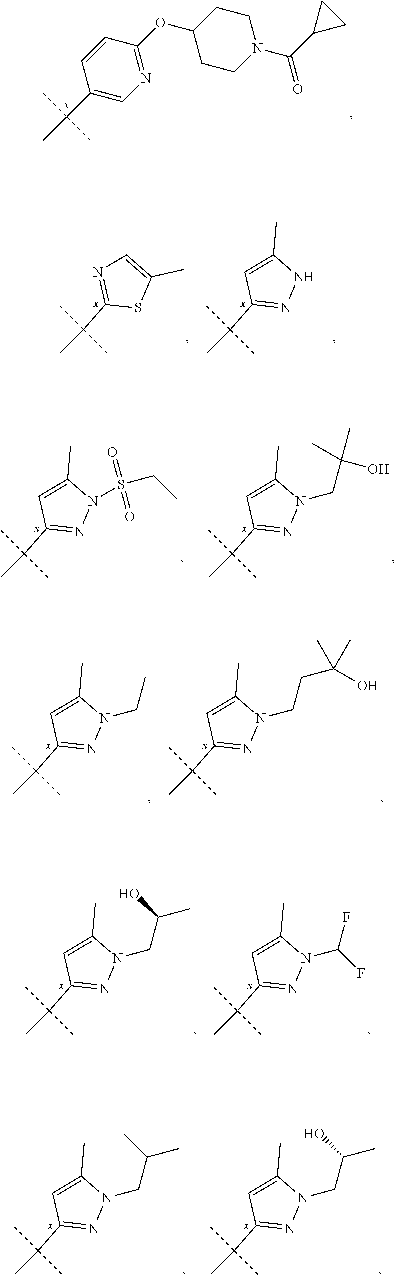

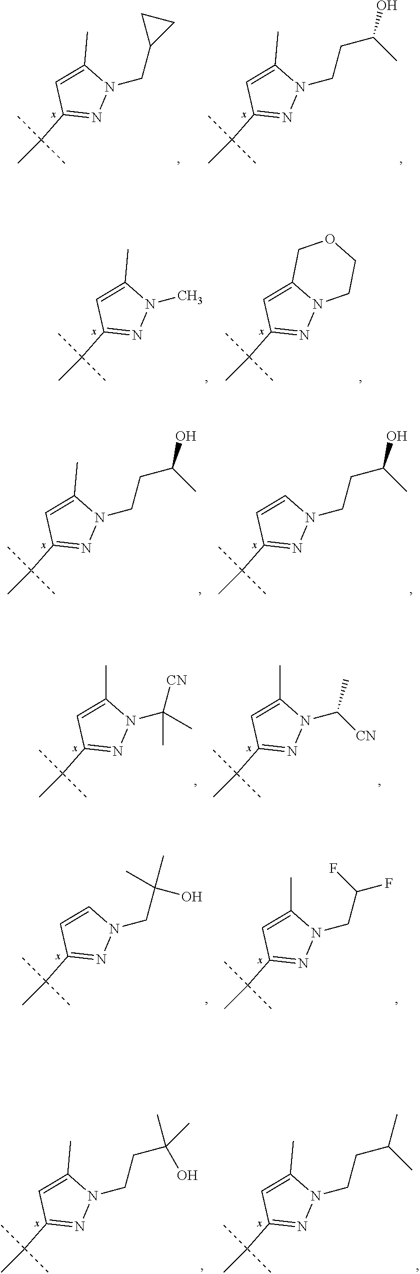

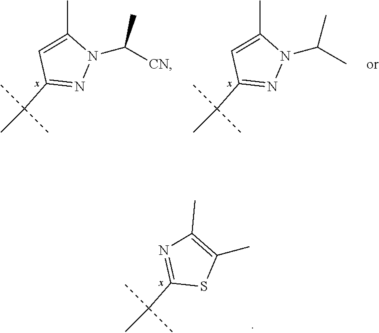

According to yet another embodiment, specifically provided are compounds of formula (I), in which

X¹ is CH or N;

X² is CH, CR¹ or N;

R¹ is fluoro, chloro, methyl or cyano;

R² is

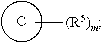

ring C is

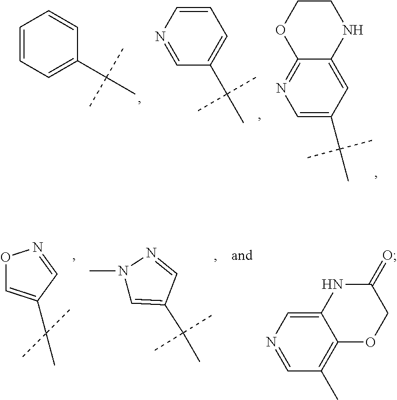

R⁵ is fluoro, methyl, methoxy, ethoxy, difluoromethoxy or amino;

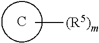

is

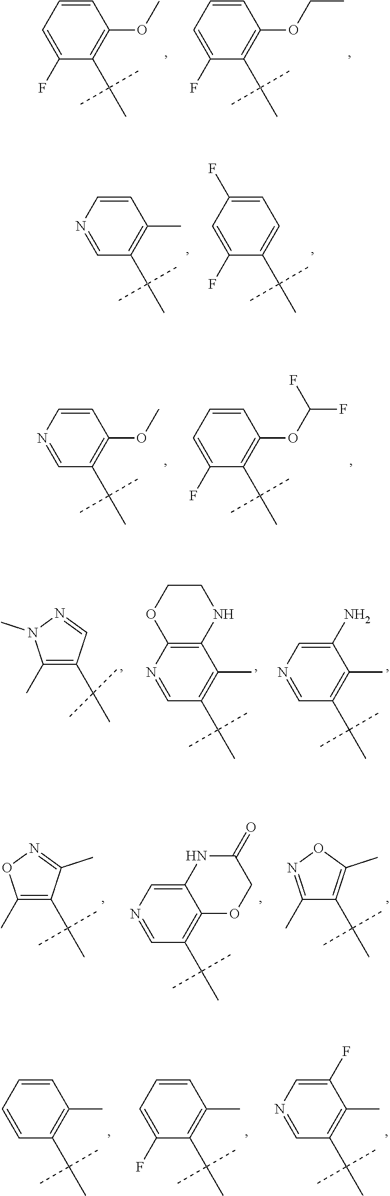

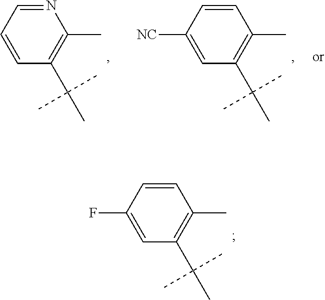

R³ is hydrogen, methyl, ethyl or isopropyl;

ring A is

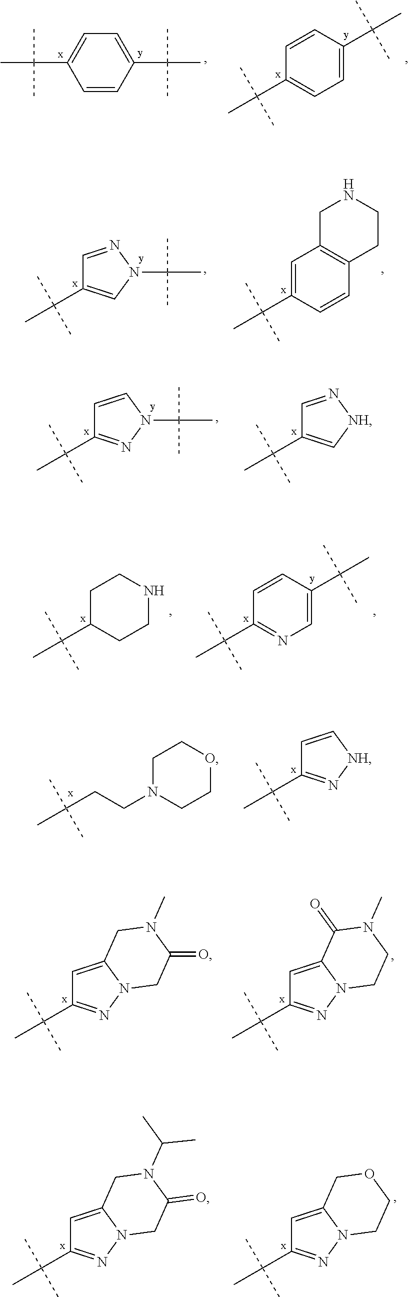

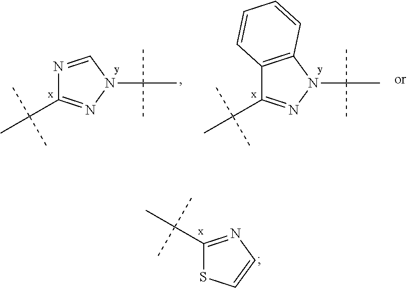

R⁶ is methyl, ethyl, methoxy,

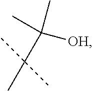

cyclopropyl or

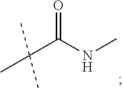

L¹ is absent; or

L¹ is

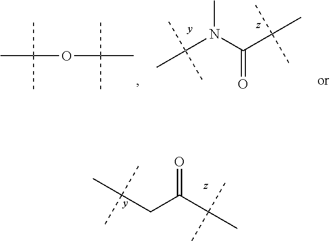

R⁷ is —CH₃,

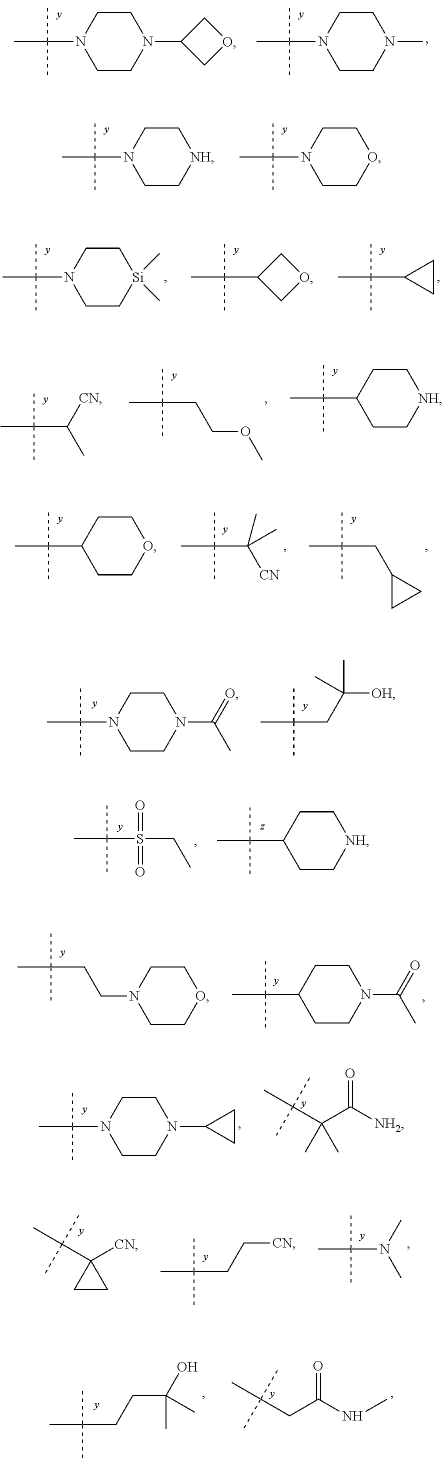

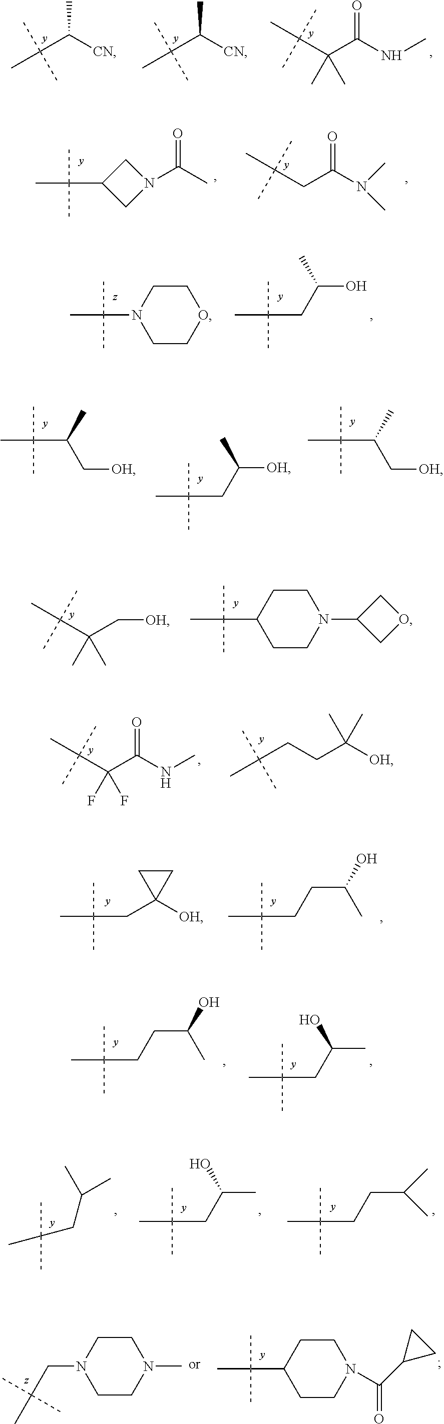

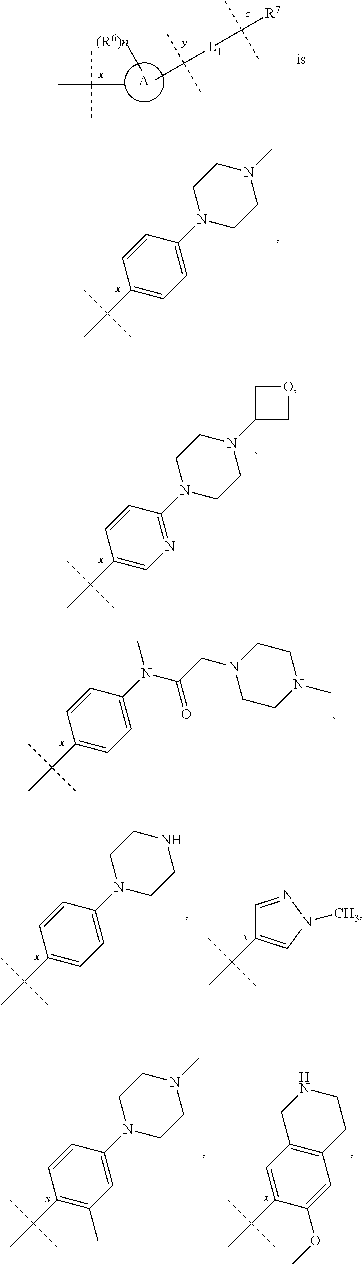

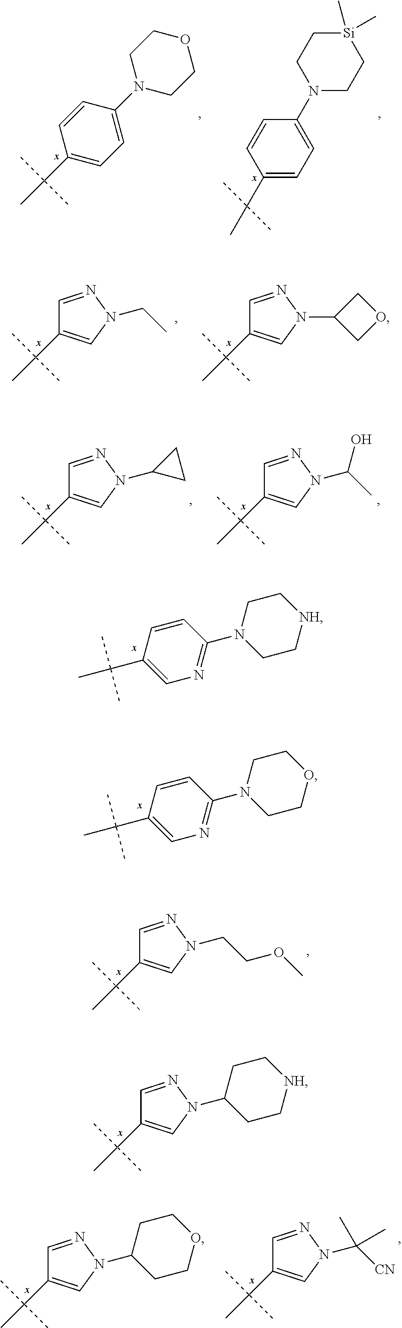

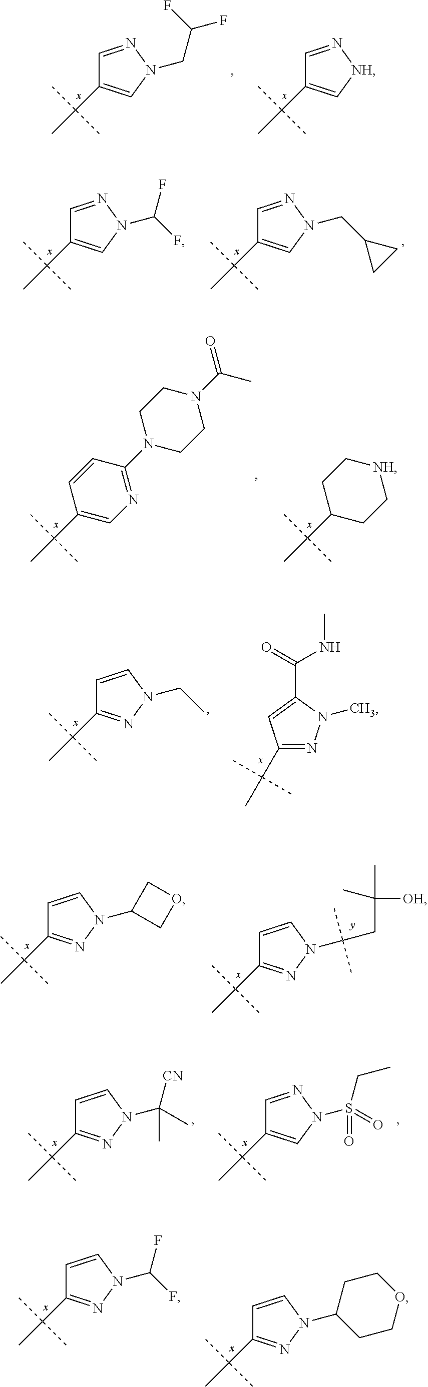

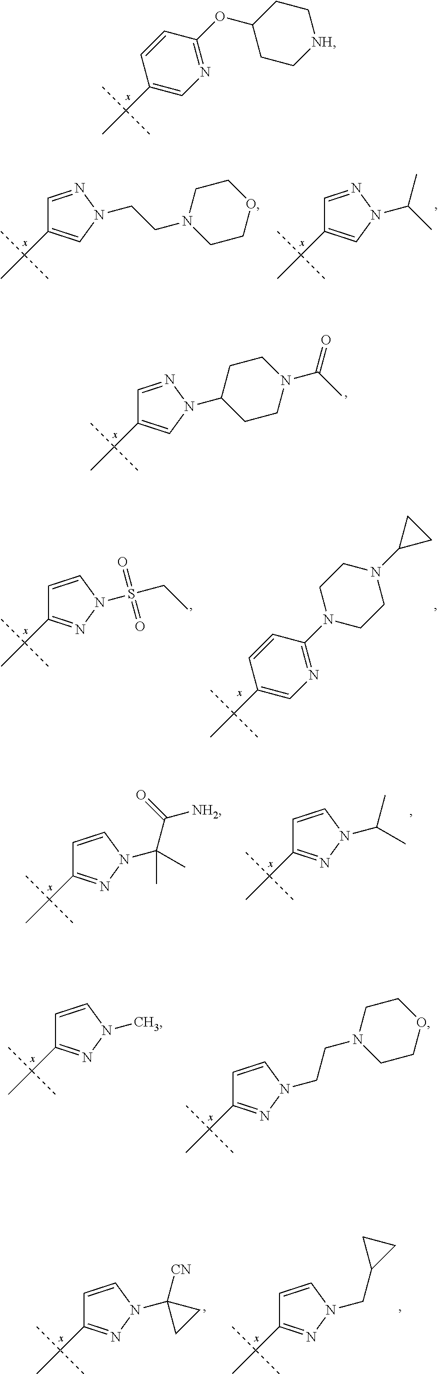

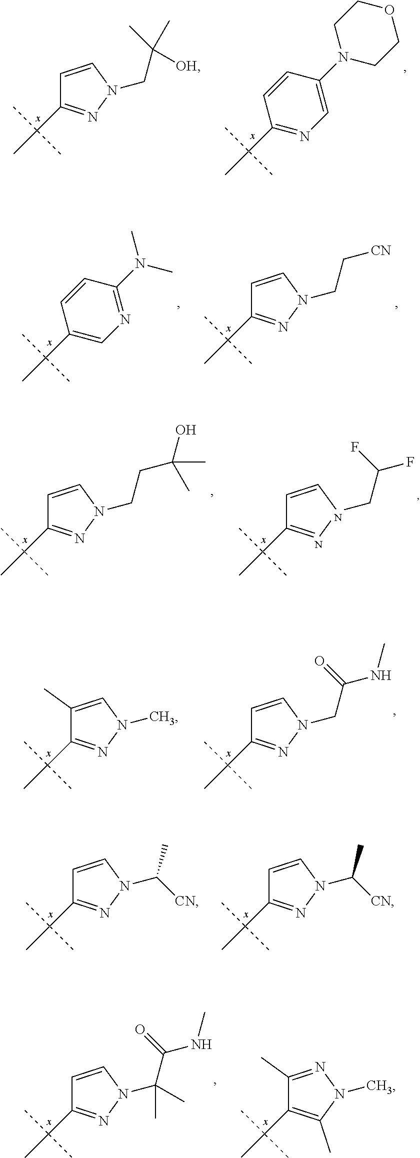

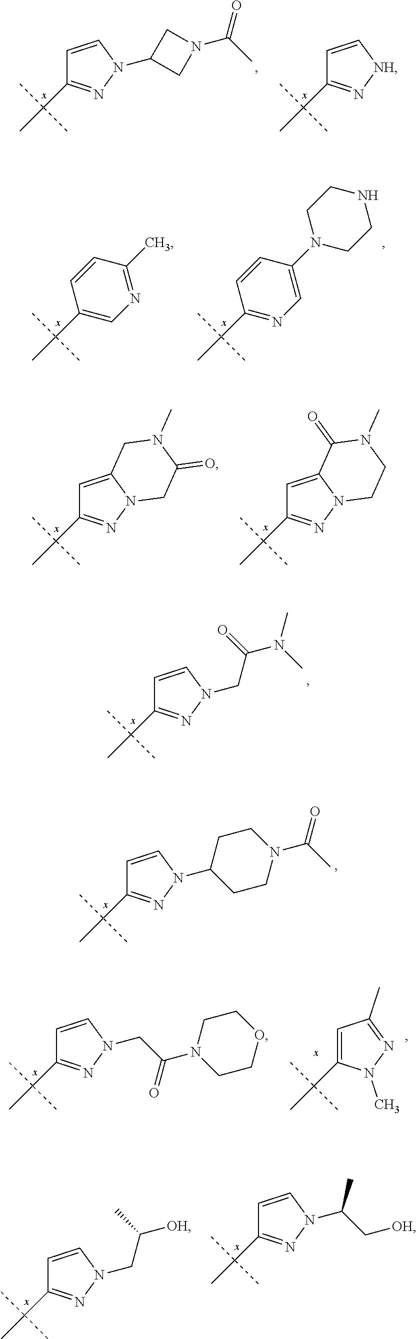

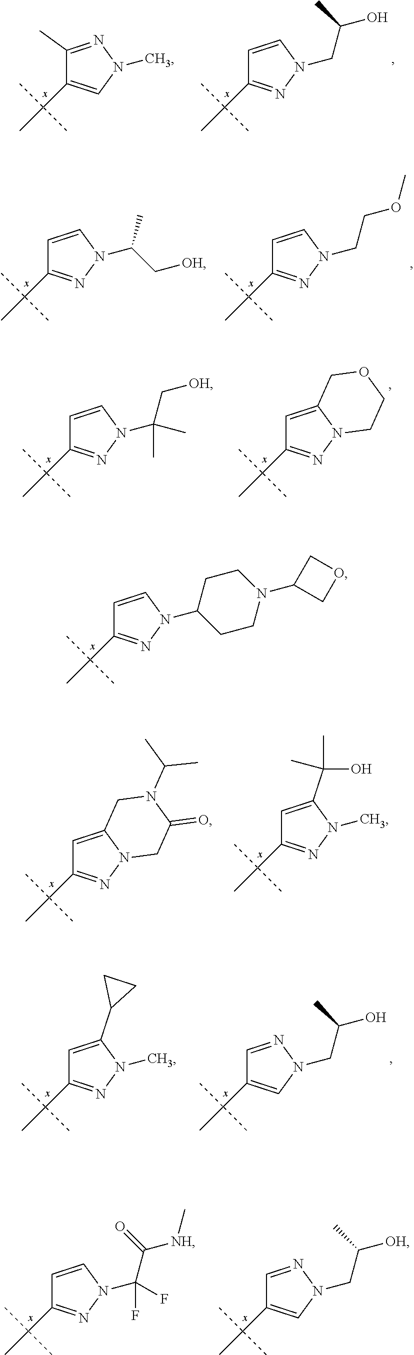

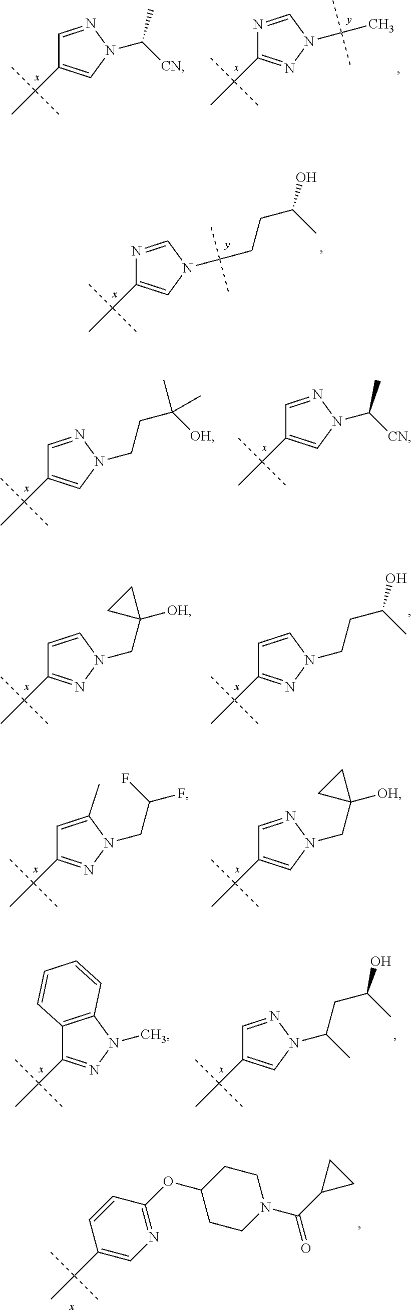

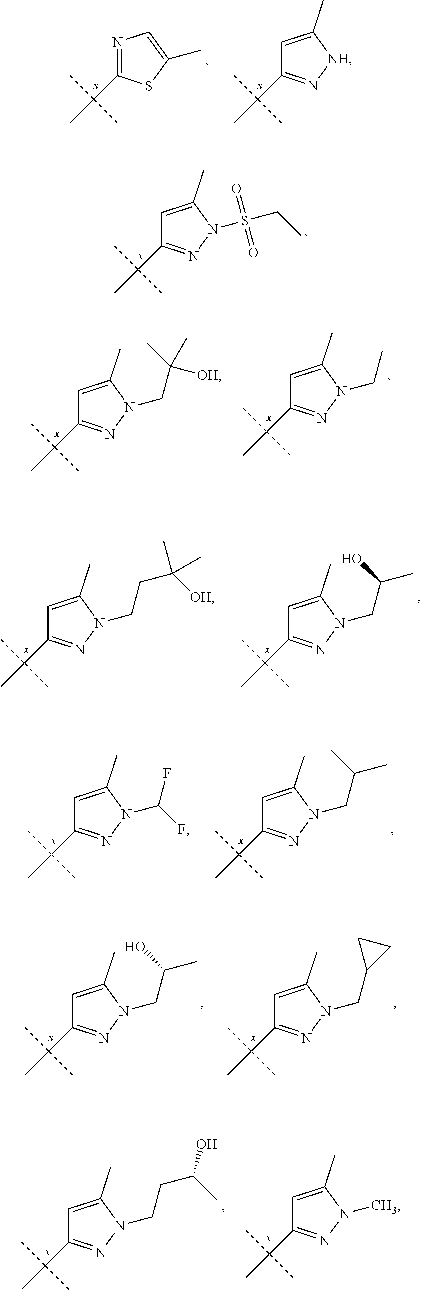

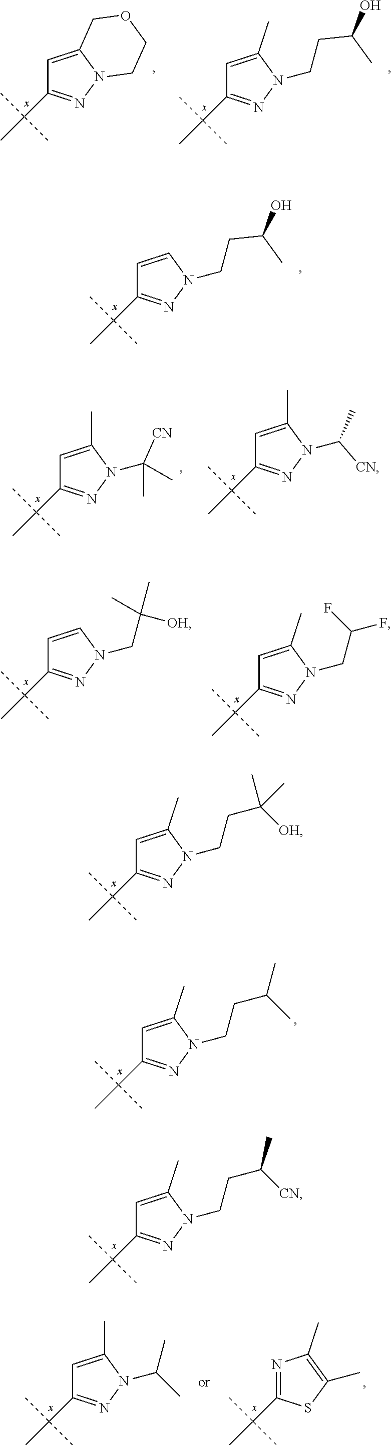

‘m’ is 0, 1 or 2; and

‘n’ is 0, 1 or 2;

According to yet another embodiment, specifically provided are compounds of formula (I), in which

X¹ is CH or N;

X² is CH, CR¹ or N;

R¹ is fluoro, chloro, methyl or cyano;

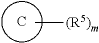

is

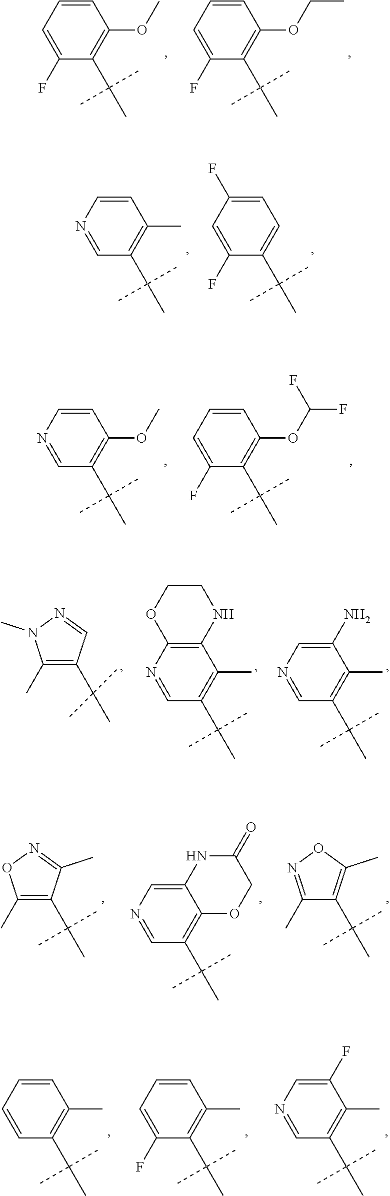

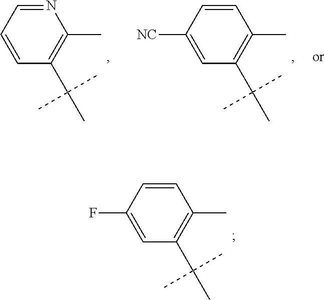

R³ is methyl, ethyl or isopropyl;

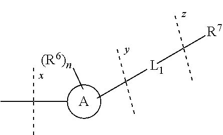

is

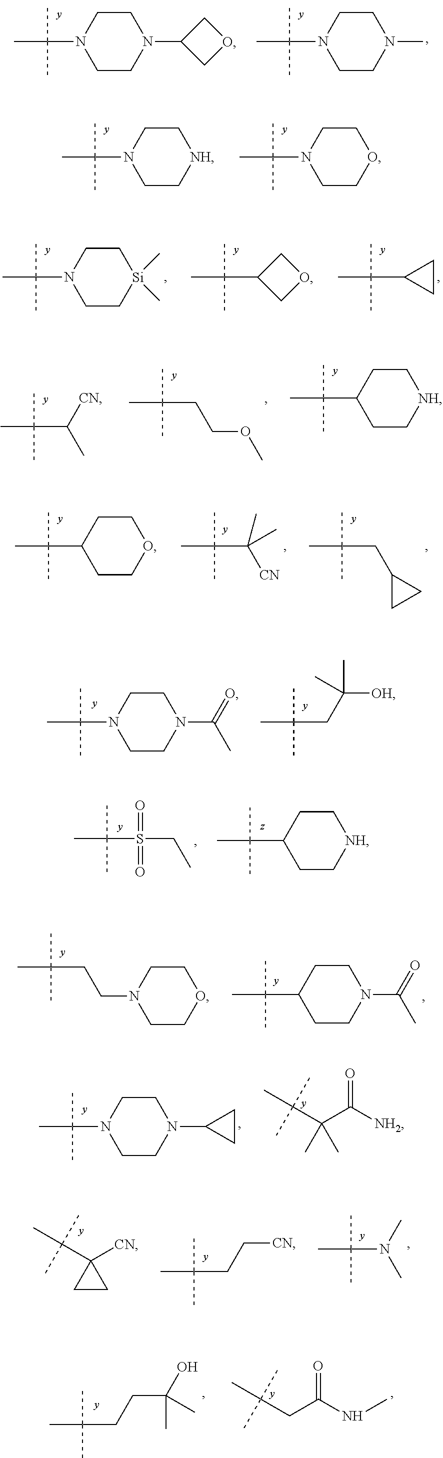

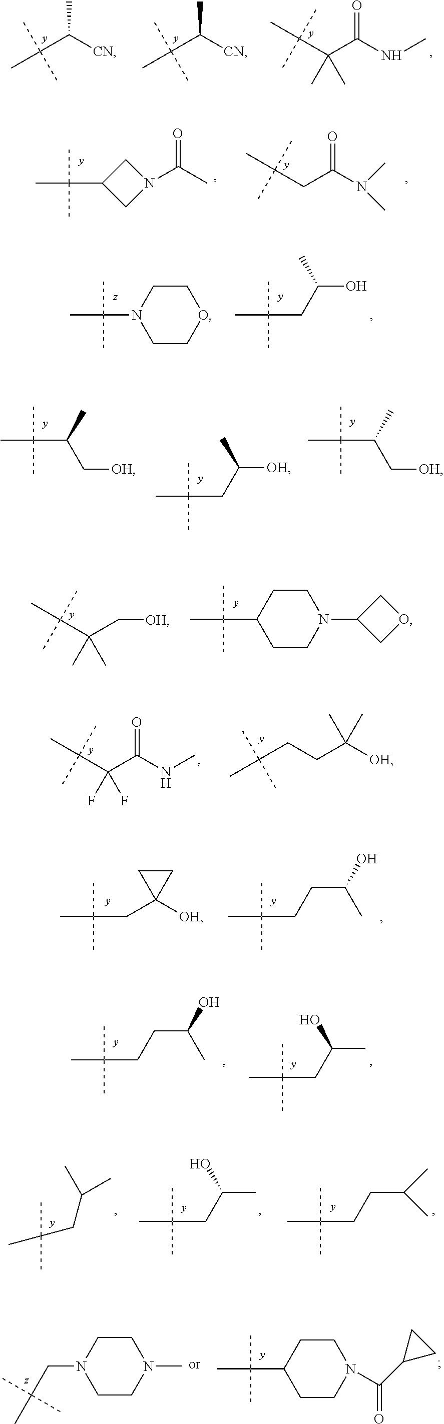

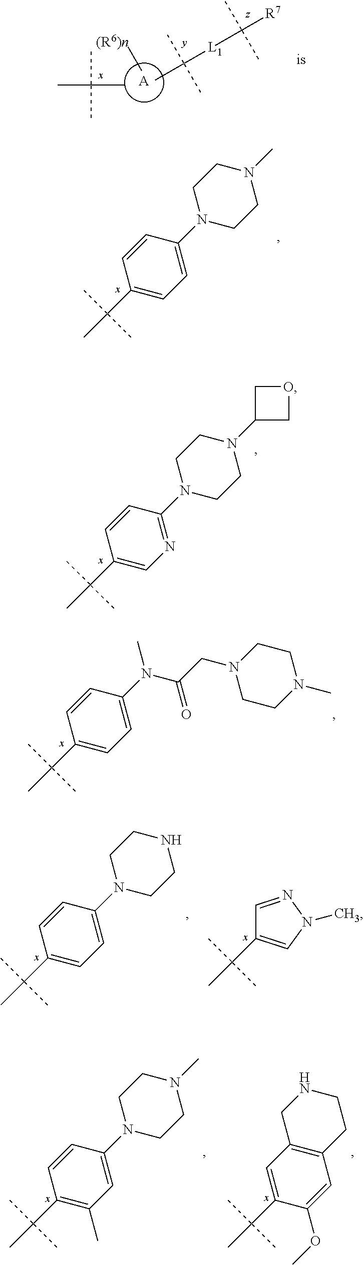

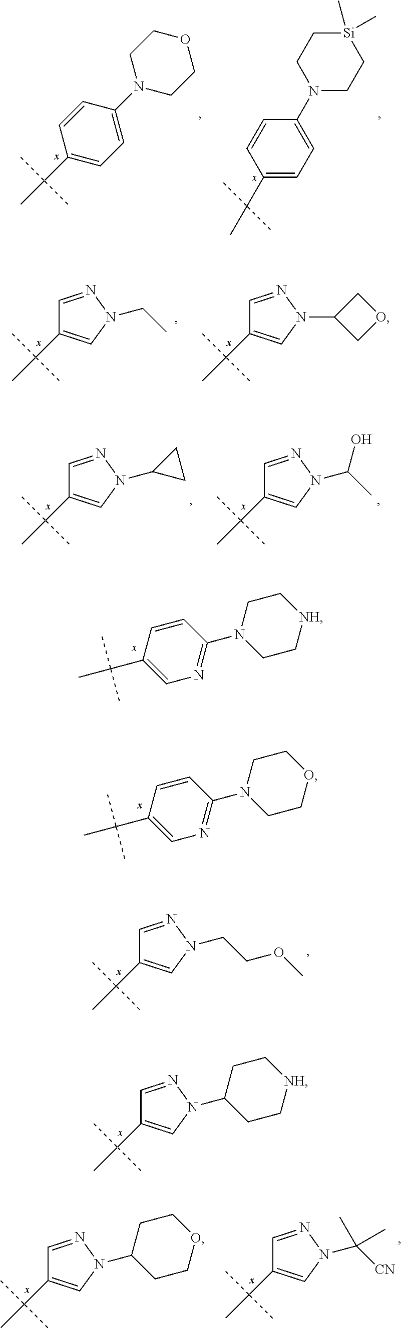

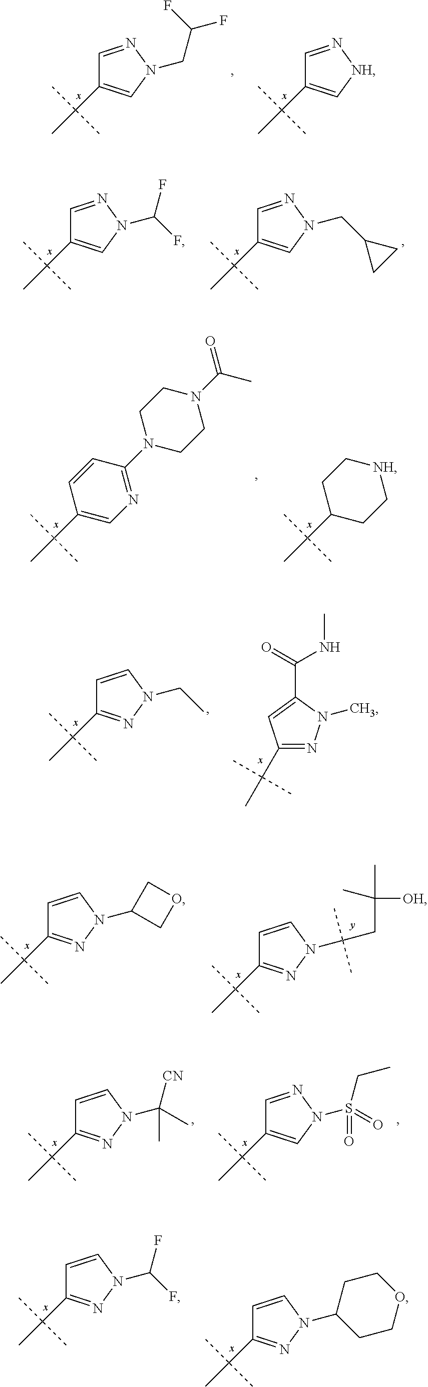

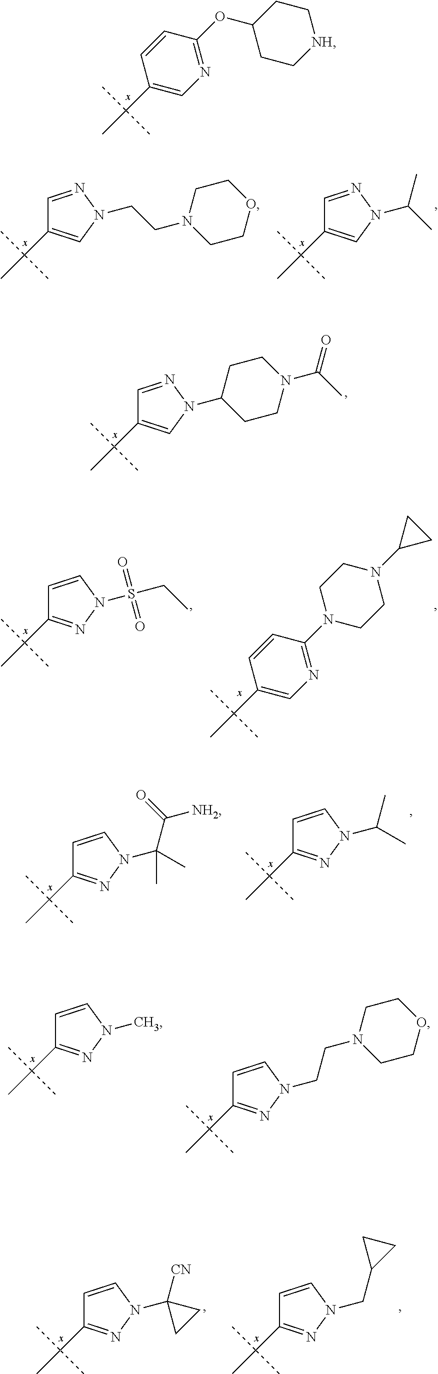

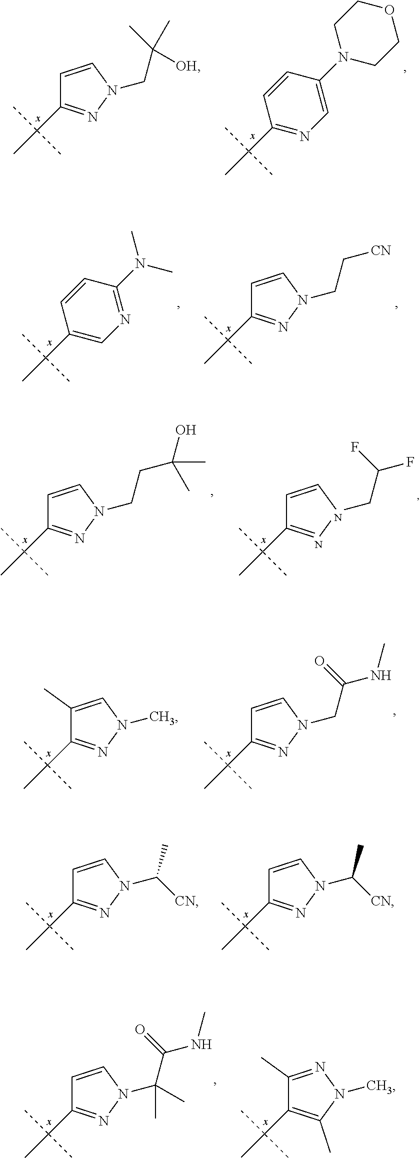

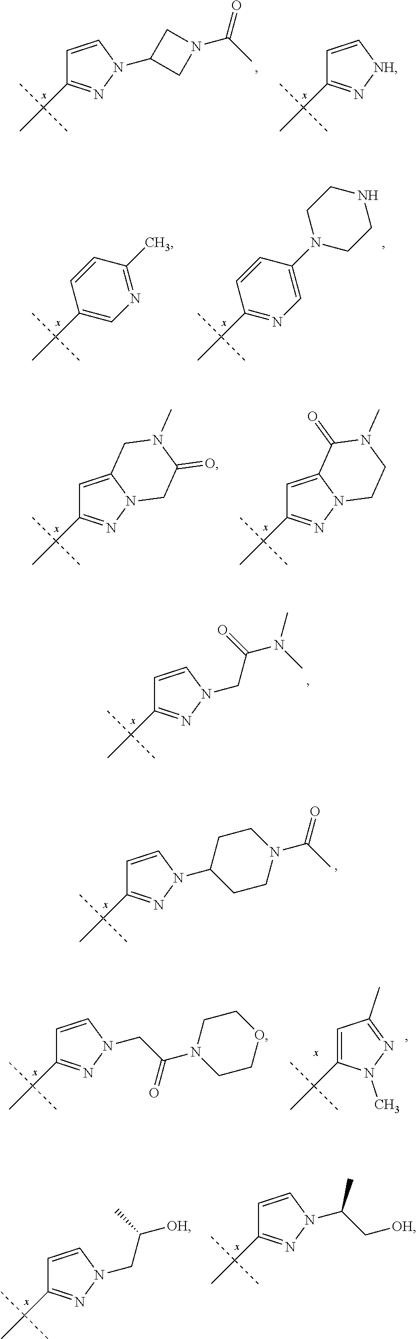

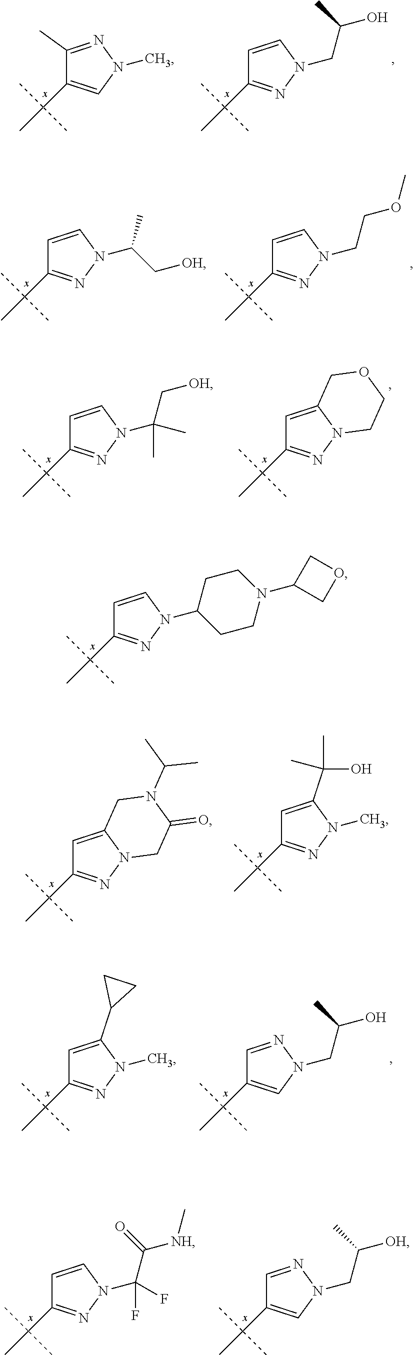

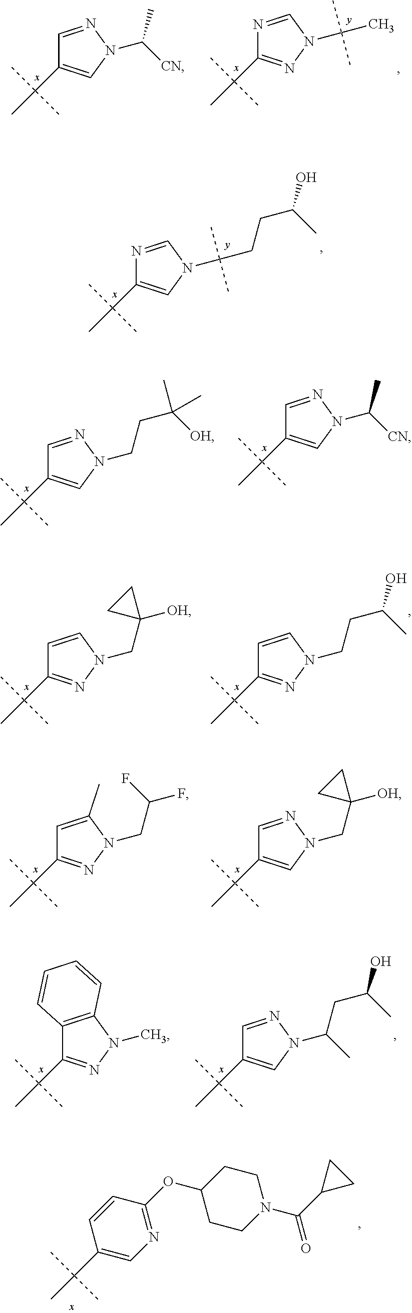

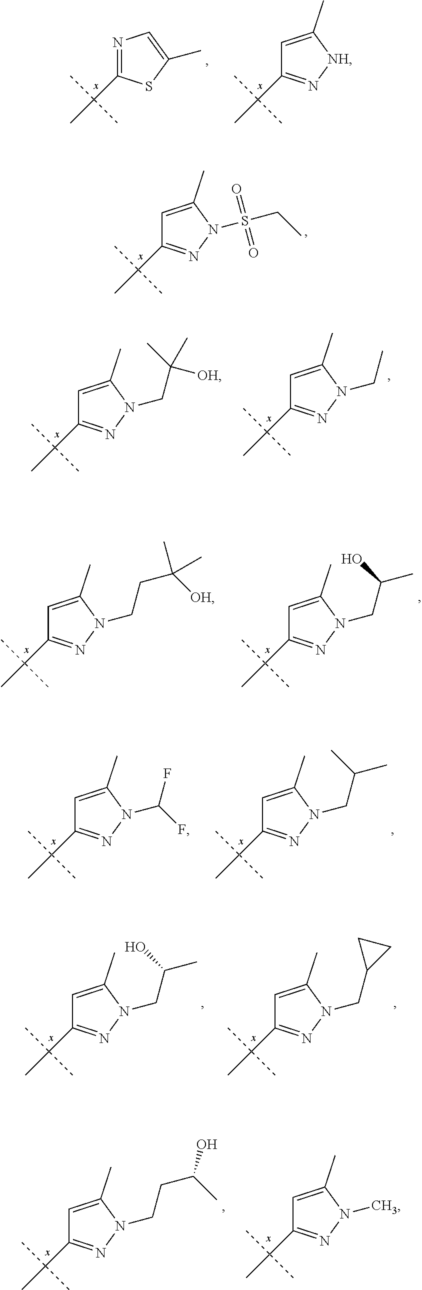

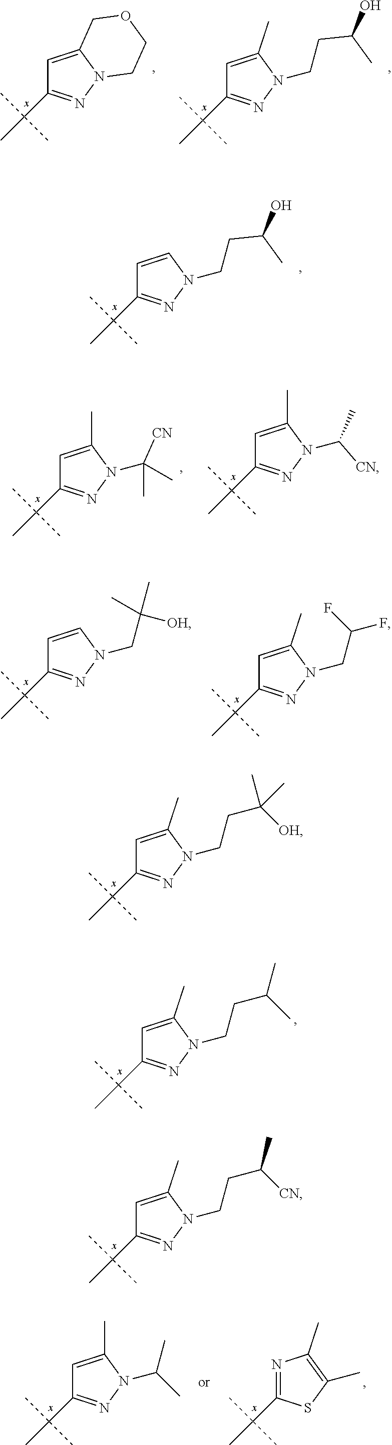

According to an embodiment, specifically provided are compounds of formula (I) with an IC50 value of less than 1000 nM, preferably less than 500 nM, more preferably less than 50 nM, with respect to MAP4K1 inhibition.

Compounds of the present invention include the compounds in Examples 1-219. It should be understood that the formula (I) structurally encompasses all geometrical isomers, stereoisomers, enantiomers and diastereomers, N-oxides, and pharmaceutically acceptable salts that may be contemplated from the chemical structure of the genera described herein.

The present application also provides a pharmaceutical composition that includes at least one compound described herein and at least one pharmaceutically acceptable excipient (such as a pharmaceutically acceptable carrier or diluent). Preferably, the pharmaceutical composition comprises a therapeutically effective amount of at least one compound described herein. The compounds described herein may be associated with a pharmaceutically acceptable excipient (such as a carrier or a diluent) or be diluted by a carrier, or enclosed within a carrier which can be in the form of a tablet, capsule, sachet, paper or other container.

Dosages employed in practicing the present invention will of course vary depending, e.g. on the particular disease or condition to be treated, the particular compound used, the mode of administration, and the therapy desired. The compound may be administered by any suitable route, including orally, parenterally, transdermally, or by inhalation. In general, satisfactory results, e.g. for the treatment of diseases as hereinbefore set forth are indicated to be obtained on oral administration at dosages of the order from about 0.01 to 2.0 mg/kg. In larger mammals, for example humans, an indicated daily dosage for oral administration will accordingly be in the range of from about 0.75 to 300 mg, conveniently administered once, or in divided doses 2 to 4 times, daily or in sustained release form. Unit dosage forms for oral administration thus for example may comprise from about 0.2 to 75 or 150 mg or 300 mg, e.g. from about 0.2 or 2.0 to 10, 25, 50, 75, 100, 150, 200 or 300 mg of the compound disclosed herein, together with a pharmaceutically acceptable diluent or carrier therefor.

Pharmaceutical compositions comprising Compounds of the Invention may be prepared using conventional diluents or excipients and techniques known in the galenic art. Thus oral dosage forms may include tablets, capsules, solutions, suspensions and the like.

### DETAILED DESCRIPTION OF THE INVENTION

### Definitions

The terms “halogen” or “halo” means fluorine (fluoro), chlorine (chloro), bromine (bromo), or iodine (iodo).

The term “alkyl” refers to a hydrocarbon chain radical that includes solely carbon and hydrogen atoms in the backbone, containing no unsaturation, having from one to eight carbon atoms (i.e. C₁₋₈alkyl), and which is attached to the rest of the molecule by a single bond, such as, but not limited to, methyl, ethyl, n-propyl, 1-methylethyl (isopropyl), n-butyl, n-pentyl, and 1,1-dimethylethyl (t-butyl). The term “C₁₋₆alkyl” refers to an alkyl chain having 1 to 6 carbon atoms. The term “C₁₋₄alkyl” refers to an alkyl chain having 1 to 4 carbon atoms. Unless set forth or recited to the contrary, all alkyl groups described or claimed herein may be straight chain or branched.

The term “haloalkyl” refers to at least one halo group (selected from F, Cl, Br or I), linked to an alkyl group as defined above (i.e. haloC₁₋₈alkyl). Examples of such haloalkyl moiety include, but are not limited to, trifluoromethyl, difluoromethyl and fluoromethyl groups. The term “haloC₁₋₄alkyl” refers to at least one halo group linked an alkyl chain having 1 to 4 carbon atoms. Unless set forth or recited to the contrary, all haloalkyl groups described herein may be straight chain or branched.

The term “alkoxy” denotes an alkyl group attached via an oxygen linkage to the rest of the molecule (i.e. C1-8 alkoxy). Representative examples of such groups are —OCH₃ and —OC₂H₅. Unless set forth or recited to the contrary, all alkoxy groups described or claimed herein may be straight chain or branched.

The term “alkoxyalkyl” or “alkyloxyalkyl” refers to an alkoxy or alkyloxy group as defined above directly bonded to an alkyl group as defined above (i.e. C₁₋₈alkoxyC₁₋₈alkyl or C₁₋₈alkyloxyC₁₋₈alkyl). Example of such alkoxyalkyl moiety includes, but are not limited to, —CH₂OCH₃ (methoxymethyl) and —CH₂OC₂H₅ (ethoxymethyl). Unless set forth or recited to the contrary, all alkoxyalkyl groups described herein may be straight chain or branched.

The term “hydroxyC₁₋₈alkyl” refers to a C₁₋₈alkyl group as defined above wherein one to three hydrogen atoms on different carbon atoms is/are replaced by hydroxyl groups (i.e. hydroxyC₁₋₄alkyl). Examples of hydroxyC₁₋₄alkyl moieties include, but are not limited to —CH₂OH and —C₂H₄OH.

The term “cyanoalkyl” refers to a alkyl group as defined above directly bonded to cyano group (i.e. cyanoC₁₋₈alkyl). Examples of such cyanoC₁₋₈alkyl moiety include, but are not limited to, cyanomethyl, cyanoethyl and cyanoisopropyl. Unless set forth or recited to the contrary, all cyanoalkyl groups described herein may be straight chain or branched.

The term “cyanocycloalkyl” refers to a cycloalkyl group as defined above directly bonded to cyano group (i.e. cyanoC₃₋₁₂cycloalkyl). Examples of such cyanoC₃₋₁₂ cycloalkyl moiety include, but are not limited to, cyanocyclopropyl and cyanocyclobutyl.

The term “cycloalkyl” denotes a non-aromatic mono or multicyclic ring system of 3 to about 12 carbon atoms, (i.e.C₃₋₁₂cycloalkyl). Examples of monocyclic cycloalkyl include but are not limited to cyclopropyl, cyclobutyl, cyclopentyl, and cyclohexyl. Examples of multicyclic cycloalkyl groups include, but are not limited to, perhydronapthyl, adamantyl and norbornyl groups, bridged cyclic groups or spirobicyclic groups, e.g., spiro(4,4)non-2-yl. The term “C₃₋₆cycloalkyl” refers to the cyclic ring having 3 to 6 carbon atoms. Examples of “C₃₋₆cycloalkyl” include but are not limited to cyclopropyl, cyclobutyl, cyclopentyl, or cyclohexyl.

The term “cycloalkylalkyl” refers to a cyclic ring-containing radical having 3 to about 6 carbon atoms directly attached to an alkyl group (i.e. C₃₋₆cycloalkylC₁₋₈alkyl). The cycloalkylalkyl group may be attached to the main structure at any carbon atom in the alkyl group that results in the creation of a stable structure. Non-limiting examples of such groups include cyclopropylmethyl, cyclobutylethyl, and cyclopentylethyl.

The term “aryl” refers to an aromatic radical having 6 to 14 carbon atoms (i.e. C₆₋₁₄aryl), including monocyclic, bicyclic and tricyclic aromatic systems, such as phenyl, naphthyl, tetrahydronapthyl, indanyl, and biphenyl.

The term “heterocyclic ring” or “heterocyclyl” unless otherwise specified refers to substituted or unsubstituted non-aromatic 3 to 15 membered ring radical (i.e. 3 to 15 membered heterocyclyl) which consists of carbon atoms and from one to five hetero atoms selected from nitrogen, phosphorus, oxygen and sulfur. The heterocyclic ring radical may be a mono-, bi- or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; also, unless otherwise constrained by the definition the heterocyclic ring or heterocyclyl may optionally contain one or more olefinic bond(s). Examples of such heterocyclic ring radicals include, but are not limited to azepinyl, azetidinyl, benzodioxolyl, benzodioxanyl, chromanyl, dioxolanyl, dioxaphospholanyl, decahydroisoquinolyl, indanyl, indolinyl, isoindolinyl, isochromanyl, isothiazolidinyl, isoxazolidinyl, morpholinyl, oxazolinyl, oxazolidinyl, 2-oxopiperazinyl, 2-oxopiperidinyl, 2-oxopyrrolidinyl, 2-oxoazepinyl, octahydroindolyl, octahydroisoindolyl, perhydroazepinyl, piperazinyl, 4-piperidonyl, pyrrolidinyl, piperidinyl, phenothiazinyl, phenoxazinyl, quinuclidinyl, tetrahydroisquinolyl, tetrahydrofuryl or tetrahydrofuranyl, tetrahydropyranyl, thiazolinyl, thiazolidinyl, thiamorpholinyl, thiamorpholinyl sulfoxide and thiamorpholinyl sulfone. The heterocyclic ring radical may be attached to the main structure at any heteroatom or carbon atom that results in the creation of a stable structure.

The term “heterocyclylalkyl” refers to a heterocyclic ring radical directly bonded to an alkyl group (i.e. 3 to 15 membered heterocyclylC₁₋₈alkyl). The 20 heterocyclylalkyl radical may be attached to the main structure at any carbon atom in the alkyl group that results in the creation of a stable structure.

The term “heteroaryl” unless otherwise specified refers to 5 to 14 membered aromatic heterocyclic ring radical with one or more heteroatom(s) independently selected from N, O or S (i.e. 5 to 14 membered heteroaryl). The heteroaryl may be a mono-, bi- or tricyclic ring system. The heteroaryl ring radical may be attached to the main structure at any heteroatom or carbon atom that results in the creation of a stable structure. Examples of such heteroaryl ring radicals include, but are not limited to oxazolyl, isoxazolyl, imidazolyl, furyl, indolyl, isoindolyl, pyrrolyl, triazolyl, triazinyl, tetrazoyl, thienyl, oxadiazolyl, thiazolyl, isothiazolyl, pyridyl, pyrimidinyl, pyrazinyl, pyridazinyl, pyrazolyl, benzofuranyl, benzothiazolyl, benzoxazolyl, benzimidazolyl, benzothienyl, benzopyranyl, carbazolyl, quinolinyl, isoquinolinyl, quinazolinyl, cinnolinyl, naphthyridinyl, pteridinyl, purinyl, quinoxalinyl, quinolyl, isoquinolyl, thiadiazolyl, indolizinyl, acridinyl, phenazinyl and phthalazinyl.

The term “pharmaceutically acceptable salt” includes salts prepared from pharmaceutically acceptable bases or acids including inorganic or organic bases and inorganic or organic acids. Examples of such salts include, but are not limited to, acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate, diphosphate, polygalacturonate, salicylate, stearate, sulfate, subacetate, succinate, tannate, tartrate, teoclate, tosylate, triethiodide and valerate. Examples of salts derived from inorganic bases include, but are not limited to, aluminum, ammonium, calcium, copper, ferric, ferrous, lithium, magnesium, manganic, mangamous, potassium, sodium, and zinc.

The term “treating” or “treatment” of a state, disorder or condition includes: (a) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (b) inhibiting the state, disorder or condition, i.e., arresting or reducing the development of the disease or at least one clinical or subclinical symptom thereof; or (c) relieving the disease, i.e., causing regression of the state, disorder or condition or at least one of its clinical or subclinical symptoms.

The term “subject” includes mammals (especially humans) and other animals, such as domestic animals (e.g., household pets including cats and dogs) and non-domestic animals (such as wildlife).

A “therapeutically effective amount” means the amount of a compound that, when administered to a subject for treating a state, disorder or condition, is sufficient to effect such treatment. The “therapeutically effective amount” will vary depending on the compound, the disease and its severity and the age, weight, physical condition and responsiveness of the subject to be treated.

The compounds of formula (I) may contain asymmetric or chiral centers, and, therefore, exist in different stereoisomeric forms. It is intended that all stereoisomeric forms of the compounds of formula (I) as well as mixtures thereof, including racemic mixtures, form part of the present invention. In addition, the present invention embraces all geometric and positional isomers. Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolysing) the individual diastereomers to the corresponding pure enantiomers. Enantiomers can also be separated by use of chiral HPLC column. The chiral centres of the present invention can have the S or R configuration as defined by the IUPAC 1974.

The terms “salt” or “solvate”, and the like, is intended to equally apply to the salt, solvate and prodrug of enantiomers, stereoisomers, rotamers, tautomers, positional isomers or racemates of the inventive compounds.

### Pharmaceutical Compositions

The compounds of the invention are typically administered in the form of a pharmaceutical composition. Such compositions can be prepared using procedures well known in the pharmaceutical art and comprise at least one compound of the invention. The pharmaceutical compositions described herein comprise one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents, solvents and the like.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters, and polyoxyethylene.

The pharmaceutical compositions described herein may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, capsules, tablets, solutions, suspensions, injectables or products for topical application. Further, the pharmaceutical composition of the present invention may be formulated so as to provide desired release profile.

Administration of the compounds of the invention, in pure form or in an appropriate pharmaceutical composition, can be carried out using any of the accepted routes of administration of such compounds or pharmaceutical compositions. The route of administration may be any route which effectively transports the active compound of the patent application to the appropriate or desired site of action. Suitable routes of administration include, but are not limited to, oral, nasal, buccal, dermal, intradermal, transdermal, parenteral, rectal, subcutaneous, intravenous, intraurethral, intramuscular, and topical.

Solid oral formulations include, but are not limited to, tablets, capsules (soft or hard gelatin), dragees (containing the active ingredient in powder or pellet form), troches and lozenges.

Liquid formulations include, but are not limited to, syrups, emulsions, and sterile injectable liquids, such as suspensions or solutions.

Topical dosage forms of the compounds include, but are not limited to, ointments, pastes, creams, lotions, powders, solutions, eye or ear drops, impregnated dressings, and may contain appropriate conventional additives such as preservatives, solvents to assist drug penetration.

Suitable doses of the compounds for use in treating the diseases and disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms, and suitable pharmaceutical excipients can also be well used and adjusted by those skilled in the art.

### Methods of Treatment

The compounds of Formula (I) as described herein are highly effective inhibitors of the MAP4K1 kinase, producing inhibition at nanomolar concentrations. MAP4K1 inhibitors according to the invention are therefore useful for treatment and prophylaxis of diseases associated with protein kinase signaling dysfunction. Accordingly, without being bound by any theory, it is believed that inhibition of MAP4K1 could, for example, reverse or prevent the cellular dysfunction associated with perturbations of the JNK signaling pathway, especially in T and B cells. Therefore, administration of a MAP4K1 inhibitor as described herein could provide a potential means to regulate MAPK signal transduction pathways, especially the JNK pathway, and by extension provide a treatment for a variety of diseases and disorders including autoimmune, neurodegenerative, neurological, inflammatory, hyperproliferative, and cardiovascular diseases and disorders.

In addition, without being bound by theory, selective MAP4K1 inhibition, as provided by the Compounds of the Invention, may provide a novel means of cancer treatment. Traditional signal transduction strategies relate to interference with the pathways that promote tumor cell proliferation or metastasis. The present invention provides instead a means of enhancing the activity and effectiveness of the body's T cells, for example, to overcome the immunosuppressive strategies used by many cancers. The U.S. Food and Drug Administration (FDA) has recently approved some monoclonal antibody-based treatments that achieve the same result by interfering with T-cell surface receptors which promote inhibition of TCR activity (e.g., anti-CTLA-4 and anti-PD-1 antibodies, marketed as Ipilimumab and Pembrolizumab, respectively). The success of the treatments demonstrate proof of the concept that cancer can be effectively treated by interfering with pathways which inhibit TCR signaling, Targeting these pathways using a small molecule inhibitor of MAP4K1 should produce improved results using more patient-friendly administration techniques.

Therefore, in the third aspect, the invention provides a method for the treatment or prophylaxis of a disease or disorder which may be ameliorated by modulating (e.g., inhibiting) MAP4K1-dependent signaling pathways, including the JNK pathway, e.g., autoimmune, neurodegenerative, neurological, inflammatory, hyperproliferative, and cardiovascular diseases and disorders, comprising administering to a patient in need thereof an effective amount of the compound of Formula I as described herein, in free or pharmaceutically acceptable salt form.

In particular embodiments, administration of the compounds of the present invention results in enhanced T cell receptor (TCR) signaling, such as resulting in an enhanced T cell-mediated immune response (e.g., increased T cell cytokine production).

In other particular embodiments, administration of the compounds of the present invention results in increased T cell resistance to PGE2-mediated T cell suppression.

The disease or disorder may be selected from the group consisting of: neurodegenerative diseases, such as Parkinson's disease or Alzheimer's disease; stroke and associated memory loss; autoimmune diseases such as arthritis; allergies and asthma; diabetes, especially insulin-resistant diabetes; other conditions characterized by inflammation, including chronic inflammatory diseases; liver ischemia; reperfusion injury; hearing loss or deafness; neural tube birth defects; obesity; hyperproliferative disorders including malignancies, such as leukemias, e.g. chronic myelogenous leukemia (CML); oxidative damage to organs such as the liver and kidney; heart diseases; and transplant rejections. In certain embodiments, the disease or disorder to be treated may also relate to impaired MAP4K1-dependent signaling. Impaired MAP4K1 signaling can lead to reduced immune cell, e.g. T and B cell, function which can permit or enhance the escape of nascent cancer cells from immune surveillance. Restoration of T and B cell function via treatment with a MAP4K1-inhibitor can therefore promote the clearance of carcinogenic and pre-carcinogenic cells from the body. Thus, in a particular embodiment, the invention provides a method for the treatment or prevention of cancer using the compounds of the present invention. In a particular embodiment, the invention provides a method for the treatment of cancer using the compounds of the present invention. In a particular embodiment, the invention provides a method for the treatment or prevention of hyperproliferative diseases, such as cancer, including melanomas, thyroid cancers, adenocarcinoma, breast cancer, central nervous system cancers such as glioblastomas, astrocytomas and ependymomas, colorectal cancer, squamous cell carcinomas, small and non-small cell lung cancers, ovarian cancer, endometrial cancer, pancreatic cancer, prostate cancer, sarcoma and skin cancers. In particular embodiments, owing to the unique role of immune cell dysfunction in hematologic cancers, the invention provides a method of treatment or prevention of hematologic cancers such as leukemias, acute myelogenous leukemia (AML), myelodysplastic syndromes, chronic myelogenous leukemia (CML), Hodgkin's lymphoma, non-Hodgkin's lymphoma, megakaryoblastic leukemia, and multiple myeloma.

The MAP4K1 inhibitor compounds described herein for the treatment or prophylaxis of disease or disorder according to the foregoing methods may be used as a sole therapeutic agent or may be used in combination with one or more other therapeutic agents useful for the treatment of said diseases or disorders. Such other agents include inhibitors of other protein kinases in the JNK pathway, including, for example, inhibitors of JNK (e.g., JNK1 or JNK2), MKK4, MKK7, p38, MEKK (e.g., MEKK1, MEKK2, MEKK5), and GCK,

Therefore, in a particular embodiment, the MAP4K1 inhibitor of the invention may be administered in combination with inhibitors of JNK (e.g., JNK1 or JNK2), MKK4, MKK7, p38, MEKK (e.g., MEKK1, MEKK2, MEKK5), and GCK.

In another aspect, the invention provides the following:
(i) the compound of Formula (I) as described herein, in free or pharmaceutically acceptable salt form, for use in any of the methods or in the treatment or prophylaxis of any disease or disorder as set forth herein,
(ii) a combination as described hereinbefore, comprising a MAP4K1 inhibitor of the invention, e.g., the compound of Formula (I) as described herein, in free or pharmaceutically acceptable salt form and a second therapeutic agent useful for the treatment or prophylaxis of any disease or disorder set forth herein;
(iii) use of the compound of Formula (I) in free or pharmaceutically acceptable salt form, or the combination described herein, (in the manufacture of a medicament) for the treatment or prophylaxis of any disease or condition as set forth herein,
(iv) the compound of Formula (I) in free or pharmaceutically acceptable salt form, the combination described herein or the pharmaceutical composition of the invention as hereinbefore described for use in the treatment or prophylaxis of any disease or condition as set forth herein.

### General Methods of Preparation

The compounds, described herein, including those of general formula (I), intermediates and specific examples are prepared through the synthetic methods as depicted in Schemes 1 to 3. Furthermore, in the following schemes, where specific acids, bases, reagents, coupling reagents, solvents, etc. are mentioned, it is understood that other suitable acids, bases, reagents, coupling reagents, solvents etc. may be used and are included within the scope of the present invention. The modifications to reaction conditions, for example, temperature, duration of the reaction or combinations thereof, are envisioned as part of the present invention. The compounds obtained using the general reaction sequences may be of insufficient purity. These compounds can be purified using any of the methods for purification of organic compounds known to persons skilled in the art, for example, crystallization or silica gel or alumina column chromatography using different solvents in suitable ratios. All possible geometrical isomers and stereoisomers are envisioned within the scope of this invention.

### General Schemes

A general approach for the preparation of compounds of the formulae (IIIb) (wherein R⁵, R⁶ and n are as defined in the general description) is depicted in synthetic scheme 1.

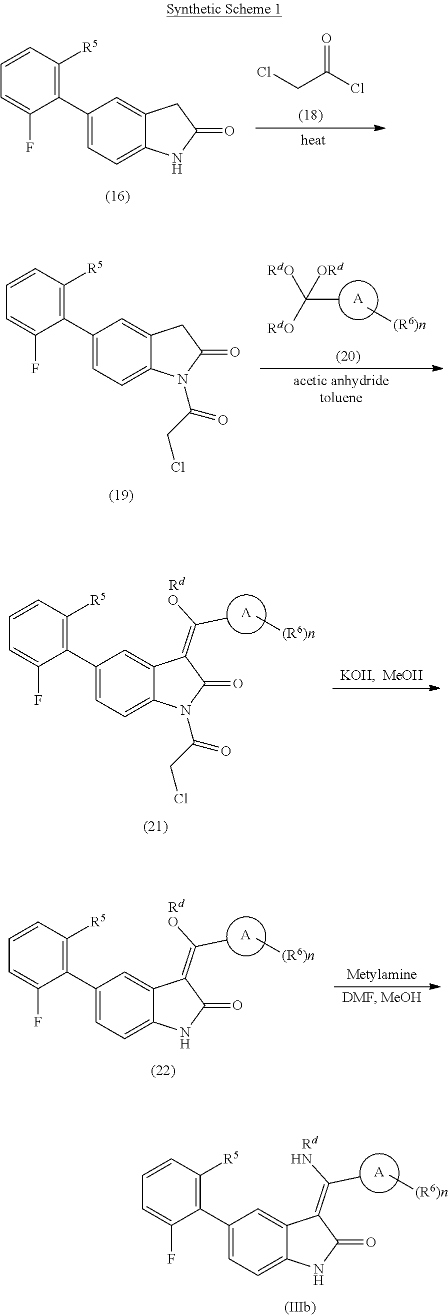

The reaction of compound of formula (16) with chloroacetyl chloride ((18) under heating yields the N-protected compound of formula (19). The reaction of compound of formula (19) with appropriate ortho-arylate of formula (20) (wherein R^{d}=C₁₋₈alkyl) in the presence of acetic anhydride in the presence of suitable solvent such as toluene affords the compound of formula (21). The compound of formula (21) on base mediated de-protection reaction in a suitable solvent such as methanol yields the compound of formula (22). The suitable base for the reaction may be potassium or sodium hydroxide. The reaction of compound of formula (22) with methylamine in a mixture of DMF and methanol as solvent affords the desired compound of general formula (IIIb).

A general approach for the preparation of compounds of the formulae (IIIc) (wherein R⁵, R⁶, m and n are as defined in the general description) is depicted in synthetic scheme 2.

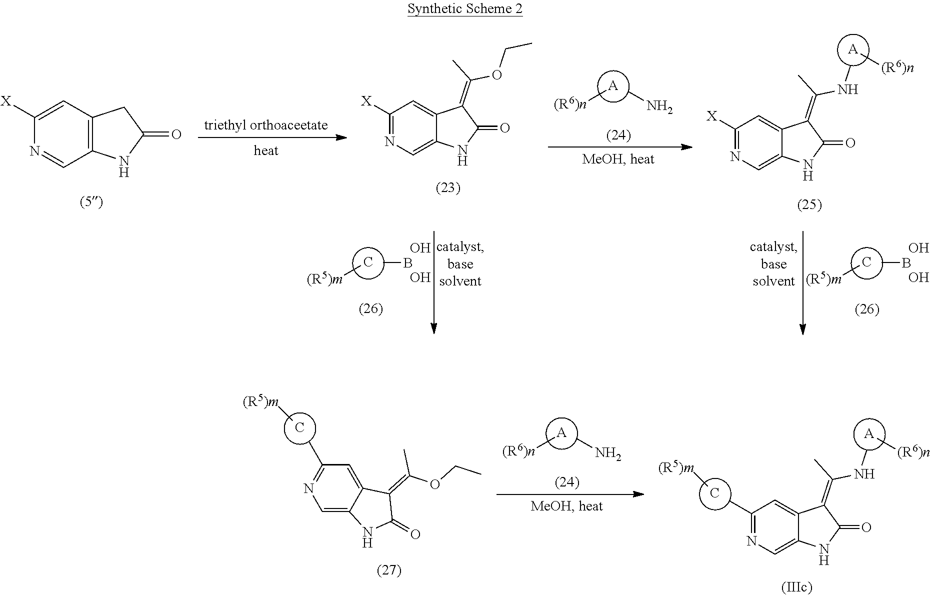

The reaction of compound of formula (5″) (wherein X=Cl, Br, I) with triethyl orthoacetate under heating yields the compound of formula (23). The reaction of compound of formula (23) with amine of formula (24) in methanol at elevated temperature (more than 80° C.) affords the compound of formula (25). The Suzuki coupling reaction of compound of formula (25) with the suitable boronic acid (or pinacol ester of the boronic acid) of formula (26) in the presence of suitable base, catalyst and solvent yields the compound of formula (IIIc). The suitable base used in the reaction may be potassium acetate, sodium or potassium tert-butoxide, sodium carbonate, cesium carbonate, etc. The suitable palladium catalyst used in the reaction may be tetrakis(triphenylphosphine)palladium(0), 1,1′-bis(diphenylphosphino)ferrocene]dichloropalladium(II) complex with dichloromethane, bis(dibenzylideneacetone)palladium(0), palladium acetate along with a suitable phosphine ligand, etc. The coupling reaction may be carried out in a suitable polar solvent or mixture thereof. The suitable solvent may be selected from ethanol, toluene, 1,4-dioxane, DMSO, water or a combination thereof. In an alternative sequence, the Suzuki reaction can be performed first followed by the amine coupling as shown in the scheme, keeping all the reaction conditions same as mentioned above.

A general approach for the preparation of compounds of the formulae (IIId) (wherein R⁵, R⁶, m and n are as defined in the general description) is depicted in synthetic scheme 3.

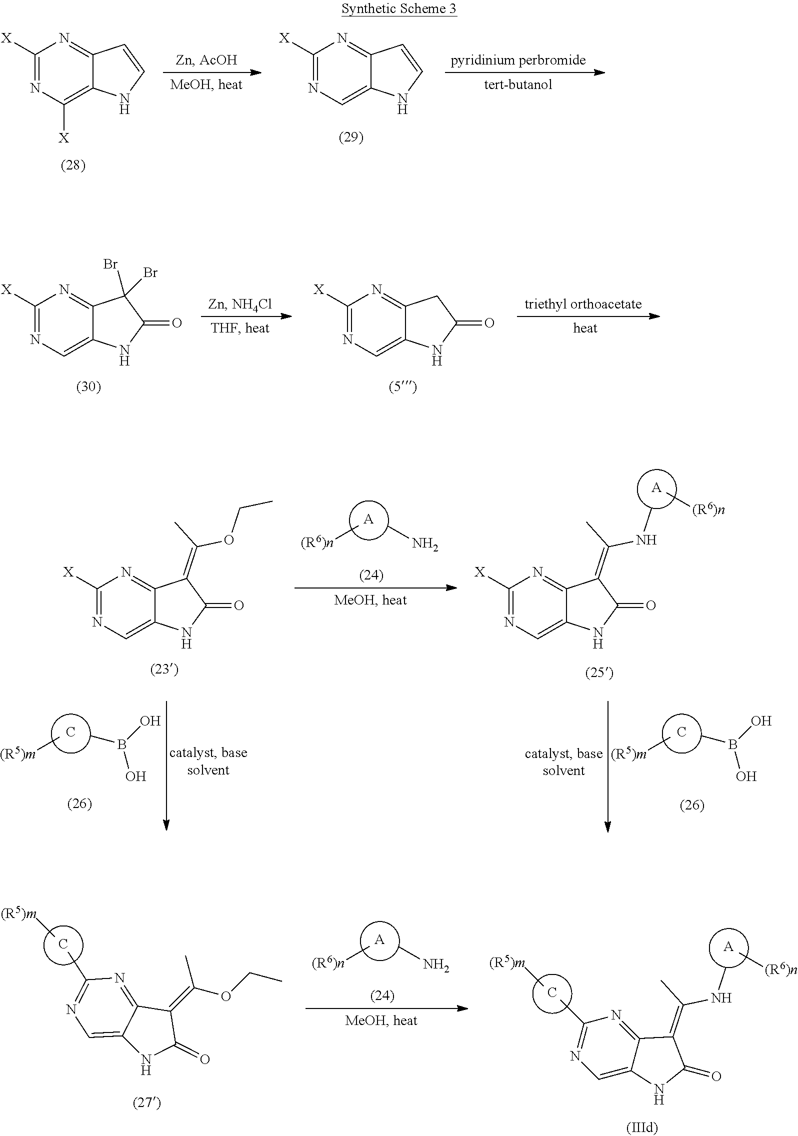

The reaction of compound of formula (28) (wherein X=Cl, Br, I) with zinc powder and acetic acid in methanol as solvent under heating conditions yields compound of formula (29). The compound of formula (29) on reaction with pyridinium perbromide in tert-butanol as solvent, furnishes compound of formula (30). Compound (30) reacts with zinc and ammonium chloride to yield compound of formula (5′″). Suitable solvent for the reaction may be THF, dichloroethane, etc. The reaction of compound of formula (5′″) (wherein X=Cl, Br, I) with triethyl orthoacetate under heating yields the compound of formula (23′). The reaction of compound of formula (23′) with amine of formula (24) in methanol at elevated temperature (more than 80° C.) affords the compound of formula (25′). The Suzuki coupling of compound of formula (25′) with the suitable boronic acid (or pinacol ester of the boronic acid) of formula (26) in the presence of suitable base, catalyst and solvent yields the compound of formula (IIId). The suitable base used in the reaction may be potassium acetate, sodium or potassium tert-butoxide, sodium carbonate, cesium carbonate, etc. The suitable palladium catalyst used in the reaction may be tetrakis(triphenylphosphine)palladium(0), 1,1′-bis(diphenylphosphino)ferrocene] dichloropalladium(II) complex with dichloromethane, bis(dibenzylideneacetone)palladium(0), palladium acetate along with a suitable phosphine ligand, etc. The coupling reaction may be carried out in a suitable polar solvent or mixture thereof. The suitable solvent may be selected from ethanol, toluene, 1,4-dioxane, DMSO, water or a combination thereof. In an alternative sequence, the Suzuki reaction can be performed first followed by the amine coupling as shown in the scheme, keeping all the reaction conditions same as mentioned above.

### Intermediates

### Boronic Acid/Boronate Ester Intermediates (A)

### Intermediate A1

(4-((4-methylpiperazin-1-yl)methyl)phenyl)boronic acid

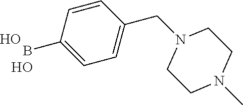

1-Methylpiperazine (5.5 mL, 50.0 mmol) and 4-formylphenylboronic acid (5.0 g, 33.3 mmol) were dissolved in THF (25 mL). Methanol (25 mL) and acetic acid (5 mL) was added to the mixture and stirred for 1.5 h at RT. To that mixture was added sodium triacetoxyborohydride (17.6 g, 83.3 mmol) and the resultant mixture was heated to 60° C. for 18 h. The solvents were removed under reduced pressure and the residue was purified by silica gel column chromatography to yield 6.0 g of the desired compound; ESI-MS (m/z) 235 (M+H)⁺.

### Intermediate A2

(4-(Morpholine-4-carbonyl)phenyl)boronic acid

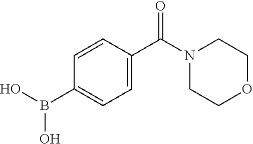 [SMILES: O=C(c1ccc(B(O)O)cc1)N1CCOCC1]

To a suspension of 4-carboxyphenylboronic acid (1.0 g, 6.03 mmol) in dichloromethane (10 mL) were added oxalyl chloride followed by catalytic amount of DMF at 0° C. and the mixture was stirred overnight at RT. The solvent was removed under reduced pressure and the acid chloride residue was dissolved in dichloromethane (10 mL). Morpholine (0.53 mL, 5.96 mmol) and triethylamine (1.68 mL, 12 mmol) were added to the above solution at 0° C. The resulting mixture was stirred for 3 h at RT. The solvent was removed under reduced pressure and the crude compound was purified by silica gel column chromatography to afford 1.5 g of the desired product. ¹H NMR (400 MHz, DMSO-d₆) δ 2.87-3.09 (m, 4H), 3.49-3.66 (m, 4H), 7.35 (d, J=8.0 Hz, 2H), 7.84 (d, J=8.0 Hz, 2H), 8.19 (s, 2H); ESI-MS (m/z) 236 (M+H)⁺.

The analytical data for the boronic acid Intermediate A3 prepared by following the procedure described above is given in Table 1.

TABLE 1
Analytical data of the Boronic acid Intermediate A3

| Intermediate No. | Structure | Name and Analytical data |
|---|---|---|
| A3 | 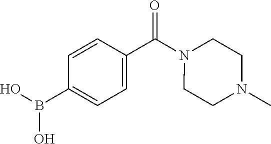 [SMILES: CN1CCN(C(=O)c2ccc(B(O)O)cc2)CC1] | (4-(4-Methylpiperazine-1- carbonyl)phenyl)boronic acid; ESI-MS (m/z) 249 (M + H)⁺. |

### Intermediate A4

tert-Butyl 4-(4-(4,4,5,5-tetramethyl-1,3,2-dioxaborolan-2-yl)-1H-pyrazol-1-yl)piperidine-1-carboxylate

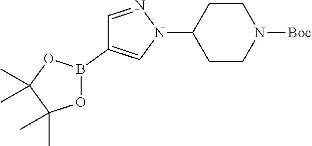 [SMILES: CC(C)(C)OC(=O)N1CCC(n2cc(B3OC(C)(C)C(C)(C)O3)cn2)CC1]

Step 1: tert-Butyl 4-(4-iodo-1H-pyrazol-1-yl)piperidine-1-carboxylate

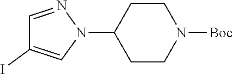 [SMILES: CC(C)(C)OC(=O)N1CCC(n2cc(I)cn2)CC1]

To a mixture of 4-iodo-1H-pyrazole (750 mg, 3.86 mmol) and tert-butyl 4-((methylsulfonyl)oxy)piperidine-1-carboxylate (1.2 g, 4.30 mmol) in NMP (10 mL) was added cesium carbonate (1.51 g, 4.64 mmol) at RT and the mixture was heated at 80° C. for 16 h. The reaction mixture was cooled to RT and diluted with water. The aqueous mixture was extracted twice with ethyl acetate and the combined organic extracts were washed with water followed by brine. The organic layer was dried over anhydrous sodium sulfate, filtered and concentrated under reduced pressure. The residue obtained was purified by silica gel column chromatography to yield 900 mg of the desired product. ¹H NMR (400 MHz, CDCl₃) δ 1.48 (s, 9H), 1.84-1.95 (m, 2H), 2.09-2.15 (m, 2H), 2.85-2.93 (m, 2H), 4.23-4.34 (m, 3H), 7.47 (s, 1H), 7.53 (s, 1H).

Step 2: tert-Butyl 4-(4-(4,4,5,5-tetramethyl-1,3,2-dioxaborolan-2-yl)-1H-pyrazol-1-yl)piperidine-1-carboxylate

In a sealed tube, to a degassed and stirred solution of tert-butyl 4-(4-iodo-1H-pyrazol-1-yl)piperidine-1-carboxylate (step 1 intermediate) (500 mg, 1.32 mmol) in DMSO (10 mL) were added bis(pinacolato)diboron (503 mg, 1.98 mmol), dichlorobis(triphenylphosphine)palladium(II) (46 mg, 0.07 mmol) and potassium acetate (519 mg, 5.29 mmol) at RT. The mixture was purged with nitrogen for 10 min and heated at 80° C. for 30 min. The reaction mixture was cooled to RT and diluted with water. The aqueous mixture was extracted twice with ethyl acetate and the combined organic extracts were washed with water followed by brine. The organic layer was dried over anhydrous sodium sulfate, filtered and concentrated under reduced pressure to yield 155 mg of the desired product. ¹H NMR (400 MHz, DMSO-d₆) δ 1.16 (s, 12H), 1.41 (s, 9H), 1.74-1.80 (m, 2H), 1.96-2.01 (m, 2H), 2.86-2.92 (m, 2H), 3.98-4.05 (m, 2H), 4.35-4.39 (m, 1H), 7.59 (s, 1H), 7.95 (s, 1H); ESI-MS (m/z) 378 (M+H)⁺.

The analytical data of the Boronate ester Intermediate A5 prepared by following the procedure described in step 2 of Intermediate A4 is given in Table 2.

TABLE 2
Analytical data of the Boronic acid Intermediate A5

| Intermediate No. | Structure | Name and Analytical data |
|---|---|---|
| A5 | 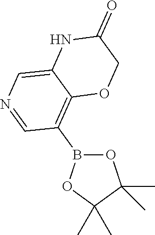 [SMILES: CC1(C)OB(c2cncc3c2OCC(=O)N3)OC1(C)C] | 8-(4,4,5,5-Tetramethyl-1,3,2- dioxaborolan-2-yl)-2H- pyrido[4,3-b][1,4]oxazin-3(4H)- one; ESI-MS (m/z) 277 (M + H)⁺. |

### Oxindole Intermediates (B)

### Intermediate B1

### 5-(2-Fluorophenyl)indolin-2-one

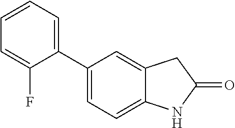 [SMILES: O=C1Cc2cc(-c3ccccc3F)ccc2N1]

To a degassed and stirred solution of 5-bromoindolin-2-one (500 mg, 2.35 mmol) and 2-fluorophenylboronic acid (395 mg, 2.83 mmol) in a mixture of toluene (10 mL) and ethanol (10 mL) were added sodium carbonate (750 mg, 7.06 mmol), tetrakis(triphenylphosphine)palladium(0) (163 mg, 0.14 mmol) and water (5 mL) at RT. The mixture was refluxed for 18 h. The reaction mixture was cooled to RT and diluted with water. The aqueous mixture was extracted twice with ethyl acetate and the combined organic extracts were washed with water followed by brine. The organic layer was dried over anhydrous sodium sulfate, filtered and concentrated under reduced pressure. The residue obtained was purified by silica gel column chromatography to yield 380 mg of the desired product. ¹H NMR (400 MHz, DMSO-d₆) δ 3.54 (s, 2H), 6.91 (d, J=8.0 Hz, 1H), 7.24-7.31 (m, 2H), 7.33-7.40 (m, 3H), 7.44-7.50 (m, 1H), 10.49 (s, 1H); ESI-MS (m/z) 228 (M+H)⁺.

The analytical data of the oxindole intermediates B2 and B3 prepared by following the procedure described above are given in Table 3. (Catalyst used for the reaction was 1,1′-bis(diphenylphosphino)ferrocene-palladium(II)dichloride)

TABLE 3
Analytical data of Oxindole Intermediate B2-B3

| Intermediate No. | Structure | Name and Analytical data |
|---|---|---|
| B2 | 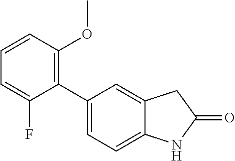 [SMILES: COc1cccc(F)c1-c1ccc2c(c1)CC(=O)N2] | 5-(2-Fluoro-6- methoxyphenyl)indolin-2-one; ¹H NMR (400 MHz, DMSO-d₆) δ 3.51 (s, 2H), 3.73 (s, 3H), 6.84- 6.91 (m, 2H), 6.94 (d, J = 8.4 Hz, 1H), 7.11 (d, J = 8.0 Hz, 1H), 7.14 (s, 1H), 7.30-7.38 (s, 1H), 10.44 (s, 1H). |
| B3 | 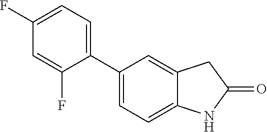 [SMILES: O=C1Cc2cc(-c3ccc(F)cc3F)ccc2N1] | 5-(2,4-Difluorophenyl)indolin-2- one; ¹H NMR (400 MHz, DMSO- d₆) δ 3.54 (s, 2H), 6.91 (d, J = 8.0 Hz, 1H), 7.13-7.20 (m, 1H), 7.29- 7.37 (m, 3H), 7.47-7.55 (m, 1H), 10.50 (s, 1H). |

### Intermediate B4

### 5-(2,6-Difluorophenyl)indolin-2-one

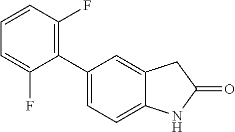 [SMILES: O=C1Cc2cc(-c3c(F)cccc3F)ccc2N1]

To a degassed and stirred solution of 5-bromoindolin-2-one (300 mg, 1.41 mmol), and 2,6-difluorophenylboronic acid (268 mg, 1.69 mmol) in a mixture of 1,4-dioxane (2.0 mL), water (1.0 mL) and ethanol (2.0 mL) were added sodium carbonate (449 mg, 4.24 mmol), tetrakis(triphenylphosphine)palladium(0) (163 mg, 0.14 mmol) at RT. The mixture was degassed and irradiated in microwave for 2 h at 170° C. The residue obtained was purified by silica gel column chromatography to yield 84 mg of the desired product. ¹H NMR (400 MHz, DMSO-d₆) δ 3.54 (s, 2H), 6.93 (d, J=8.0 Hz, 1H), 7.16-7.28 (m, 4H), 7.40-7.45 (m, 1H), 10.53 (s, 1H).

### Intermediate B5

6-Chloro-5-(2-fluoro-6-methoxyphenyl)indolin-2-one

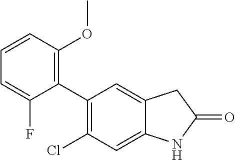 [SMILES: COc1cccc(F)c1-c1cc2c(cc1Cl)NC(=O)C2]

Step 1: 5-Bromo-6-chloroindolin-2-one

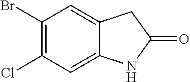 [SMILES: O=C1Cc2cc(Br)c(Cl)cc2N1]

To a stirred solution of 6-chloroindolin-2-one (3.5 g, 20.9 mmol) in acetonitrile (35 mL) was added N-bromosuccinimide (4.4 g, 25.1 mmol) at −10° C. and stirred for 1 h at the same temperature. The mixture was gradually warmed up to RT and stirred for 4 h. The mixture was partitioned between ethyl acetate and water. The layers were separated. The organic layer was concentrated under reduced pressure and the crude material was purified by silica gel column chromatography to yield 4.5 g of the desired compound. ¹H NMR (400 MHz, DMSO-d₆) δ 3.51 (s, 2H), 6.97 (s, 1H), 7.56 (s, 1H), 10.60 (s, 1H).

Step 2: 6-Chloro-5-(2-fluoro-6-methoxyphenyl)indolin-2-one

To a degassed mixture of 1,4-dioxane (20 mL) and water (3.0 mL) were added 5-bromo-6-chloroindolin-2-one (step 1 intermediate) (250 mg, 1.01 mmol) and (2-fluoro-6-methoxyphenyl)boronic acid (344 mg, 2.03 mmol) and the mixture was evacuated for 15 min. XPhos Pd G2 (80 mg, 0.10 mmol) and tribasic potassium phosphate (430 mg, 2.03 mmol) were added to the mixture. The resulting reaction mixture was heated on a pre-heated oil bath at 100° C. for 2 h. The mixture was cooled to RT and partitioned between ethyl acetate and water. The layers were separated. The organic layer was concentrated under reduced pressure and the crude material was purified by silica gel column chromatography to yield 90 mg of the desired compound. ¹H NMR (400 MHz, DMSO-d₆) δ 3.34 (s, 2H), 3.72 (s, 3H), 6.85-6.98 (m, 3H), 7.11 (s, 1H), 7.37-7.45 (m, 1H), 10.56 (s, 1H).

The analytical data of the oxindole intermediates B6 and B11 prepared by following the procedure described above are given in Table 4.

TABLE 4
Analytical data of Oxindole Intermediate B6 and B11

| Intermediate No. | Structure | Name and Analytical data |
|---|---|---|
| B6 | 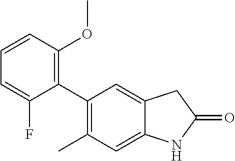 [SMILES: COc1cccc(F)c1-c1cc2c(cc1C)NC(=O)C2] | 5-(2-Fluoro-6-methoxyphenyl)-6- methylindolin-2-one; ¹H NMR (400 MHz, DMSO-d₆) δ 1.97 (s, 3H), 3.34 (s, 2H), 3.70 (s, 3H), 6.74 (s, 1H), 6.85-6.95 (m, 3H), 7.37 (q, J = 2.8 Hz, 1H), 10.39 (s, 1H); ESI- MS (m/z) 272 (M + H)⁺. |
| B11 | 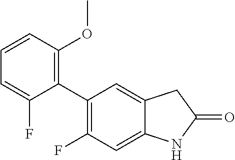 [SMILES: COc1cccc(F)c1-c1cc2c(cc1F)NC(=O)C2] | 6-Fluoro-5-(2-fluoro-6- methoxyphenyl)indolin-2-one; ¹H NMR (400 MHz, DMSO-d₆) δ 3.49 (s, 2H), 3.74 (s, 3H), 6.71 (d, J = 9.6 Hz, 1H), 6.90 (d, J = 8.8 Hz, 1H), 6.96 (d, J = 8.4 Hz, 1H), 7.11 (d, J = 7.2 Hz, 1H), 7.36-7.44 (m, 1H), 10.58 (s, 1H). |

### Intermediate B7

5-(2-Fluoro-6-methoxyphenyl)-1H-pyrrolo[2,3-c]pyridin-2(3H)-one

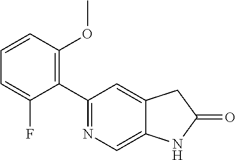 [SMILES: COc1cccc(F)c1-c1cc2c(cn1)NC(=O)C2]

Step 1: Diethyl 2-(2-chloro-5-nitropyridin-4-yl)malonate

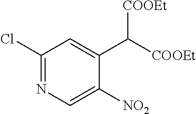 [SMILES: CCOC(=O)C(C(=O)OCC)c1cc(Cl)ncc1[N+](=O)[O-]]

To a stirred solution of diethyl malonate (4.74 mL, 31.1 mmol) in THF (80 mL) was added sodium hydride (60% w/w, 1.24 g, 31.1 mmol) at 0° C. and the mixture was stirred at the same temperature for 1 h. 2,4-Dichloro-5-nitropyridine (5.0 g, 25.9 mmol) was added to the mixture in small portions and refluxed overnight at RT. The mixture was cooled to RT and quenched with cold water. The aqueous mixture was extracted twice with ethyl acetate. The combined organic extracts were washed with water followed by brine. The organic layer was dried over anhydrous sodium sulfate, filtered and concentrated under reduced pressure. The residue obtained was purified by silica gel column chromatography to yield 4.6 g of the desired product. ¹H NMR (400 MHz, DMSO-d₆) δ 1.18 (d, J=7.2 Hz, 6H), 4.19 (q, J=7.2 Hz, 4H), 5.62 (s, 1H), 7.84 (s, 1H), 9.18 (s, 1H).

Step 2: Ethyl 2-(2-chloro-5-nitropyridin-4-yl)acetate

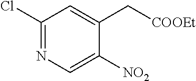 [SMILES: CCOC(=O)Cc1cc(Cl)ncc1[N+](=O)[O-]]

To a stirred solution of diethyl 2-(2-chloro-5-nitropyridin-4-yl)malonate (step 1 intermediate) (1.5 g, 4.73 mmol) in DMSO (4.0 mL) and were added a lithium chloride (401 mg, 9.47 mmol) and water (1.0 mL). The mixture was stirred at 100° C. for 5 h. The mixture was cooled to RT, diluted with ethyl acetate and water. The organic layer was separated, washed with water and brine. The solution was dried over anhydrous sodium sulfate, filtered and concentrated under reduced pressure. The residue was purified by column chromatography to yield 800 mg of the desired compound. ¹H NMR (400 MHz, DMSO-d₆) δ 1.17 (t, J=6.8 Hz, 3H), 4.11 (q, J=6.8 Hz, 2H), 4.17 (s, 2H), 7.89 (s, 1H), 9.14 (s, 1H).

### Step 3: 5-Chloro-1H-pyrrolo[2,3-c]pyridin-2(3H)-one

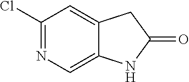 [SMILES: O=C1Cc2cc(Cl)ncc2N1]

To a stirred solution of ethyl 2-(2-chloro-5-nitropyridin-4-yl)acetate (1.5 g, 6.13 mmol) in a mixture of ethanol (20 mL) and water (5.0 mL) was added zinc powder (2.0 g, 30.6 mmol) followed by ammonium chloride (2.6 g, 49.0 mmol) and the mixture was stirred at 100° C. for 48 h. The mixture was filtered and concentrated. The residue was diluted with ethyl acetate and water. The organic layer was separated, washed with water and brine. The solution was dried over anhydrous sodium sulfate, filtered and concentrated under reduced pressure. The residue was purified by column chromatography to yield 300 mg of the desired compound. ¹H NMR (400 MHz, DMSO-d₆) δ 3.61 (s, 2H), 7.39 (s, 1H), 7.86 (s, 1H), 10.69 (s, 1H); ESI-MS (m/z) 169 (M+H)⁺.

Step 4: 5-(2-Fluoro-6-methoxyphenyl)-1H-pyrrolo[2,3-c]pyridin-2(3H)-one

To a degassed mixture of 1,4-dioxane (20 mL) and water (3.0 mL) were added 5-chloro-1H-pyrrolo[2,3-c]pyridin-2(3H)-one (step 1 intermediate) (300 mg, 1.78 mmol) and (2-fluoro-6-methoxyphenyl)boronic acid (453 mg, 2.67 mmol) and the mixture was evacuated for 15 min. XPhos Pd G2 (140 mg, 0.18 mmol) and tribasic potassium phosphate (756 mg, 3.56 mmol) were added to the mixture. The resulting reaction mixture was heated on a pre-heated oil bath at 90° C. for 2 h. The mixture was cooled to RT and concentrated under reduced pressure. The crude material was purified by silica gel column chromatography to yield 140 mg of the desired compound. ¹H NMR (400 MHz, DMSO-d₆) δ 3.61 (s, 2H), 3.78 (s, 3H), 6.88 (t, J=9.2 Hz, 1H), 6.96 (d, J=8.4 Hz, 1H), 7.27 (s, 1H), 7.36-7.44 (m, 1H), 8.15 (s, 1H), 10.62 (s, 1H).

The analytical data of the oxindole intermediates B8 to B10, B13 and B14 prepared by following the procedure described above are given in Table 5.

TABLE 5
Analytical data of Oxindole Intermediate B8-B10, B13, B14

| Intermediate No. | Structure | Name and Analytical data |
|---|---|---|
| B8 | 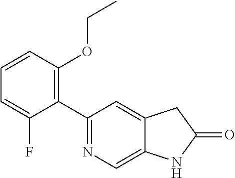 [SMILES: CCOc1cccc(F)c1-c1cc2c(cn1)NC(=O)C2] | 5-(2-Ethoxy-6-fluorophenyl)-1H- pyrrolo[2,3-c]pyridin-2(3H)-one; ¹H NMR (400 MHz, DMSO-d₆) δ 1.16 (t, J = 6.8 Hz, 3H), 3.61 (s, 2H), 4.04 (q, J = 6.8 Hz, 2H), 6.85 (t, J = 8.4 Hz, 1H), 6.91 (d, J = 12.4 Hz, 1H), 7.35 (q, J = 1.2 Hz, 1H), 8.15 (s, 1H), 10.61 (s, 1H). |
| B9 | 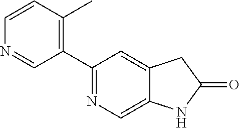 [SMILES: Cc1ccncc1-c1cc2c(cn1)NC(=O)C2] | 5-(4-Methylpyridin-3-yl)-1H- pyrrolo[2,3-c]pyridin-2(3H)-one; ¹H NMR (400 MHz, DMSO-d₆) δ 2.36 (s, 3H), 3.64 (s, 2H), 7.32 (d, J = 5.2 Hz, 1H), 7.52 (s, 1H), 8.43 (d, J = 5.2 Hz, 1H), 8.22 (s, 1H), 8.50 (s, 1H), 10.66 (s, 1H); ESI-MS (m/z) 227 (M + H)⁺. |
| B10 | 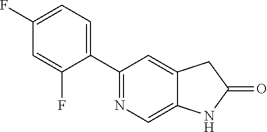 [SMILES: O=C1Cc2cc(-c3ccc(F)cc3F)ncc2N1] | 5-(2,4-Difluorophenyl)-1H- pyrrolo[2,3-c]pyridin-2(3H)-one; ¹H NMR (400 MHz, DMSO-d₆) δ 3.65 (s, 2H), 7.16-7.23 (m, 1H), 7.32-7.40 (m, 1H), 7.64 (s, 1H), 7.88-7.96 (m, 1H), 8.22 (s, 1H), 10.68 (s, 1H); ESI-MS (m/z) 248 (M + H)⁺. |
| B13 | 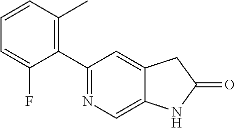 [SMILES: Cc1cccc(F)c1-c1cc2c(cn1)NC(=O)C2] | 5-(2-Fluoro-6-methylphenyl)-1H- pyrrolo[2,3-c]pyridin-2(3H)-one; ¹H NMR (400 MHz, DMSO-d₆) δ 2.13 (s, 3H), 3.64 (s, 2H), 7.10-7.15 (m, 2H), 7.29-7.33 (m, 2H), 8.20 (s, 1H), 10.65 (s, 1H). |
| B14 | 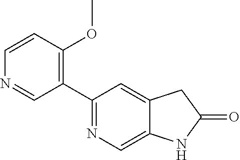 [SMILES: COc1ccncc1-c1cc2c(cn1)NC(=O)C2] | 5-(4-methoxypyridin-3-yl)-1H- pyrrolo[2,3-c]pyridin-2(3H)-one; ¹H NMR (400 MHz, DMSO-d₆) δ 3.63 (s, 2H), 3.91 (s, 3H), 7.19 (d, J = 6.0 Hz, 1H), 7.72 (s, 1H), 8.21 (s, 1H), 8.45 (d, J = 5.6 Hz, 1H), 8.69 (s, 1H), 10.65 (s, 1H). |

### Intermediate B12

### 5-Chloro-1H-pyrrolo[3,2-b]pyridin-2(3H)-one

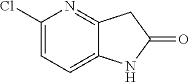 [SMILES: O=C1Cc2nc(Cl)ccc2N1]

Step 1: Diethyl 2-(6-chloro-3-nitropyridin-2-yl)malonate

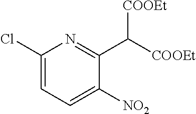 [SMILES: CCOC(=O)C(C(=O)OCC)c1nc(Cl)ccc1[N+](=O)[O-]]

The titled compound was prepared by the reaction of 2,6-dichloro-3-nitropyridine (10 g, 51.8 mmol) with diethylmalonate (19.7 mL, 129 mmol) in the presence of sodium hydride (60% w/w, 5.18 g, 129 mmol) in DME (50 mL) as per the procedure described in step 1 of Intermediate B7 to yield 6.0 g of the desired compound. (Crude) ¹H NMR (400 MHz, CDCl₃) δ 1.30-1.35 (m, 6H), 4.26-4.37 (m, 4H), 7.53-7.55 (m, 1H), 8.46-8.48 (m, 1H).

Step 2: Diethyl 2-(3-amino-6-chloropyridin-2-yl)malonate

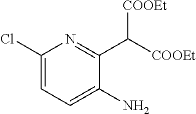 [SMILES: CCOC(=O)C(C(=O)OCC)c1nc(Cl)ccc1N]

A mixture of diethyl 2-(6-chloro-3-nitropyridin-2-yl)malonate (step 1 intermediate) (1.0 g, 3.16 mmol) and Raney nickel (300 mg) in ethanol (30 mL) was hydrogenated at 45 psi of hydrogen pressure for 2 h. The mixture was filtered through celite and the filtrate was concentrated to yield 800 mg of the desired compound. The crude compound was as such taken forward for next step.

### Step 3: 5-Chloro-1H-pyrrolo[3,2-b]pyridin-2(3H)-one

A mixture of diethyl 2-(3-amino-6-chloropyridin-2-yl)malonate (800 mg, 0.35 mmol) and 6N aqueous hydrochloric acid (17 mL) was refluxed for 5 h. The product was isolated to get 250 mg of the desired compound. ¹H NMR (400 MHz, DMSO-d₆) δ 3.63 (s, 2H), 7.19 (d, J=8.0 Hz, 1H), 7.27 (dd, J=8.4, 4.8 Hz, 1H), 10.65 (s, 1H).

### Amine Intermediates (D)

### Intermediate D1

tert-Butyl 7-amino-6-methoxy-3,4-dihydroisoquinoline-2(1H)-carboxylate

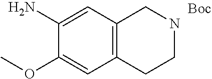

Step 1: Ethyl 3-methoxyphenethylcarbamate

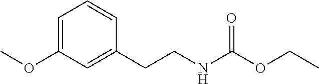 [SMILES: CCOC(=O)NCCc1cccc(OC)c1]

To a solution of ethyl chloroformate (10.4 mL, 109 mmol) in dichloromethane (100 mL) at 0° C. was added 2-(3-methoxyphenyl)ethylamine (14.7 mL, 99.2 mmol). The mixture was gradually warmed up to RT and quenched with water. The layers were separated and the aqueous layer was extracted with chloroform. The combined organic layers were dried over anhydrous sodium sulfate and the solvents were removed under reduced pressure to yield 12.5 g of the desired compound. ¹H NMR (400 MHz, CDCl₃) δ 1.24 (t, J=6.8 Hz, 3H), 2.80 (t, J=6.8 Hz, 2H), 3.46 (q, J=6.8 Hz, 2H), 3.82 (s, 3H), 4.12 (q, J=6.8 Hz, 2H), 4.70 (br s, 1H), 6.75 (s, 1H), 6.77-6.82 (m, 2H), 7.21-7.29 (m, 1H).

Step 2: 6-Methoxy-3,4-dihydroisoquinolin-1(2H)-one

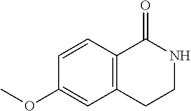

A solution of ethyl 3-methoxyphenethylcarbamate (step 1 intermediate) (12.5 g, 55.8 mmol) in polyphosphonic acid (40 mL) was stirred at 120° C. for 2 h. The mixture was cooled to 0° C. and basified aqueous with ammonia solution. The aqueous solution was extracted twice with chloroform. The combined organic layers were dried over anhydrous sodium sulfate and the solvents were removed under reduced pressure. The crude was purified by silica gel column chromatography to yield 5.5 g of the desired compound. ¹H NMR (400 MHz, CDCl₃) δ 2.98 (t, J=6.8 Hz, 2H), 3.57 (t, J=6.8 Hz, 2H), 3.87 (s, 3H), 6.35 (br s, 1H), 6.73 (d, J=2.4 Hz, 1H), 6.88 (dd, J=8.4, 2.4 Hz, 1H), 8.03 (d, J=8.8 Hz, 1H).

Step 3: 6-Methoxy-1,2,3,4-tetrahydroisoquinoline

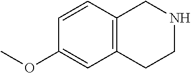

To a stirred solution of lithium aluminum hydride (2.94 g, 77.7 mmol) in dry THF (40 mL) was dropwise added a solution of 6-methoxy-3,4-dihydroisoquinolin-1(2H)-one (step 2 intermediate) (5.5 g, 31.1 mmol) in THF (40 mL) at 0° C. and the mixture was stirred at 70° C. for 2 h. The mixture was cooled to 0° C. and quenched with ice-cooled water and 15% aq. sodium hydroxide solution. The mixture was diluted with ethyl acetate and filtered through celite. The filtrate was dried over anhydrous sodium sulfate, filtered and concentrated under reduced pressure to yield 5.5 g of the desired compound. ¹H NMR (400 MHz, CDCl₃) δ 2.80 (t, J=6.0 Hz, 2H), 3.14 (t, J=6.4 Hz, 2H), 3.81 (s, 3H), 3.97 (s, 2H), 6.65 (d, J=2.8 Hz, 1H), 6.73 (dd, J=8.4, 2.8 Hz, 1H), 6.94 (d, J=8.4 Hz, 1H).

Step 4: 6-Methoxy-7-nitro-1,2,3,4-tetrahydroisoquinoline

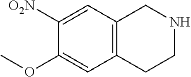

To a solution of 6-methoxy-1,2,3,4-tetrahydroisoquinoline (2.0 g, 12.3 mmol)in sulfuric acid (10 mL) at −5° C. was slowly added guanidine nitrate (750 mg, 6.15 mmol) and the mixture was stirred for 15 min at the same temperature. The reaction was quenched with ice-cold water and basified using potassium carbonate. The aqueous mixture was extracted with ethyl acetate. The organic layer was dried over anhydrous sodium sulfate, filtered and concentrated under reduced pressure to yield 1.7 g of the desired compound. ¹H NMR (400 MHz, DMSO-d₆) δ 2.76 (t, J=6.0 Hz, 2H), 2.94 (t, J=6.0 Hz, 2H), 3.83 (d, J=6.4 Hz, 2H), 3.87 (s, 3H), 7.07 (s, 1H), 7.60 (s, 1H), 8.32 (s, 1H).

Step 5: tert-Butyl 6-methoxy-7-nitro-3,4-dihydroisoquinoline-2(1H)-carboxylate

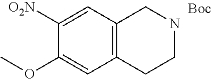

To a stirred solution of 6-methoxy-7-nitro-1,2,3,4-tetrahydroisoquinoline (step 4 of Example 1) (1.7 g, 8.17 mmol) in dichloromethane (50 mL) were added triethylamine (1.7 mL, 8.98 mmol) followed by di-tert-butyl dicarbonate (1.95 mg, 12.3 mmol) and the mixture was stirred at RT for 4 h. The reaction mixture was diluted with water. The aqueous mixture was extracted twice with ethyl acetate and the combined organic extracts were washed with brine. The organic layer was dried over anhydrous sodium sulfate, filtered and concentrated under reduced pressure. The residue thus obtained was purified by silica gel column chromatography to yield 700 mg of the desired product. ¹H NMR (400 MHz, CDCl₃) δ 1.51 (s, 9H), 2.89 (t, J=5.6 Hz, 2H), 3.68 (t, J=5.6 Hz, 2H), 3.96 (s, 3H), 4.56 (s, 2H), 6.85 (s, 1H), 7.69 (s, 1H).

Step 6: tert-Butyl 7-amino-6-methoxy-3,4-dihydroisoquinoline-2(1H)-carboxylate

A solution of tert-butyl 6-methoxy-7-nitro-3,4-dihydroisoquinoline-2(1H)-carboxylate (step 5 intermediate) (700 mg, 2.27 mmol) in methanol (10 mL) was subjected to hydrogenation in the presence of palladium on carbon as catalyst under 35 psi of hydrogen pressure at RT for 3 h. The mixture was filtered and the filtrate was concentrated under reduced pressure. The solid was triturated with n-pentane and dried well to yield 500 mg of the desired compound. ¹H NMR (400 MHz, DMSO-d₆) δ 1.43 (s, 9H), 2.85 (t, J=6.0 Hz, 2H), 3.55 (t, J=5.6 Hz, 2H), 3.89 (s, 3H), 4.48 (br s, 2H), 7.18 (s, 1H), 7.79 (s, 1H).

### Intermediate D2

N-(4-Aminophenyl)-N-methyl-2-(4-methylpiperazin-1-yl)acetamide

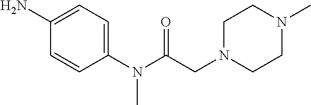 [SMILES: CN1CCN(CC(=O)N(C)c2ccc(N)cc2)CC1]

Step 1: N-Methyl-4-nitroaniline

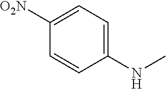

A mixture of 1-bromo-4-nitrobenzene (5.0 g, 24.7 mmol) and 40% aqueous methylamine solution (40 mL) was heated at 90° C. in a sealed tube for 16 h. The reaction mixture was cooled to RT. The precipitated solid was filtered and washed with pentane to give 2.4 g of the desired compound. ¹H NMR (300 MHz, CDCl₃) δ 2.94 (s, 3H), 6.53 (d, J=9.0 Hz, 2H), 6.10 (d, J=9.0 Hz, 2H).

Step 2: 2-Chloro-N-methyl-N-(4-nitrophenyl)acetamide

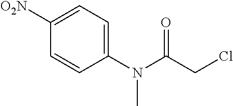

A suspension of N-methyl-4-nitroaniline (3.3 g, 21.6 mmol) in ethyl acetate (20 mL) was heated to 70° C. for 1 h and added chloroacetyl chloride (2.1 mL, 26.0 mmol) at the same temperature. The mixture was refluxed for 2 h. The reaction mixture was cooled to RT and quenched with hexane. The solution was cooled to 0° C. and stirred for 1 h. The precipitated solid was filtered, washed with hexane and dried to give 3.7 g of the desired compound. ¹H NMR (300 MHz, DMSO-d₆) δ 3.65 (s, 3H), 4.34 (s, 2H), 7.67 (d, J=9.0 Hz, 2H), 8.26 (d, J=9.0 Hz, 2H).

Step 3: N-(4-Aminophenyl)-N-methyl-2-(4-methylpiperazin-1-yl)acetamide

A suspension of 2-chloro-N-methyl-N-(4-nitrophenyl)acetamide (1.0 g, 4.37 mmol) in ethyl acetate (10 mL) was heated to 40° C. for 30 min and added 1-methylpiperazine (1.2 mL, 10.9 mmol) at the same temperature. The mixture was stirred at 50° C. for 2 h. The reaction mixture was cooled to RT and diluted with ethyl acetate. The solution was washed with water and dried over anhydrous sodium sulfate. The solution was filtered, concentrated and diluted with methanol. The solution was subjected to hydrogenation in the presence of palladium on carbon as catalyst under 25 bar of hydrogen pressure at 25° C. for 2 h. The catalyst was removed by filtration and the solvent was evaporated at 60° C. to yield 400 mg of the desired compound. ¹H NMR (400 MHz, DMSO-d₆) δ 1.88 (s, 3H), 2.14-2.19 (m, 4H), 2.63-2.68 (m, 4H), 2.80 (s, 2H), 3.01 (s, 3H), 5.20 (br s, 2H), 6.53 (d, J=8.1 Hz, 2H), 6.88 (d, J=8.7 Hz, 2H).

### Intermediate D3

4-(4,4-Dimethyl-1,4-azasilinan-1-yl)aniline

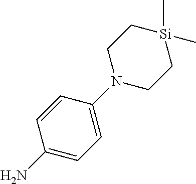

Step 1: 4,4-Dimethyl-1-(4-nitrophenyl)-1,4-azasilinane

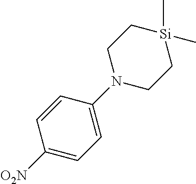 [SMILES: C[Si]1(C)CCN(c2ccc([N+](=O)[O-])cc2)CC1]

To a stirred solution of 1-fluoro-4-nitrobenzene (102 mg, 0.72 mmol) in DMF (3.0 mL) were added 4,4-dimethyl-1,4-azasilinane hydrochloride (128 mg, 0.723 mmol) and potassium carbonate (300 mg, 2.17 mmol) at RT. The mixture was heated to 90° C. for 3 h. The mixture was cooled to RT and diluted with ethyl acetate. The organic solution was washed with water followed by brine and dried over anhydrous sodium sulfate. The solution was filtered and concentrated under reduced pressure and the residue obtained was purified by silica gel column chromatography to yield 125 mg of the desired product. ¹H NMR (400 MHz, DMSO-d₆) δ 0.09 (s, 6H), 0.75-0.80 (m, 4H), 3.79 (t, J=6.4 Hz, 4H), 6.94 (d, J=9.2 Hz, 2H), 8.04 (d, J=9.6 Hz, 2H); ESI-MS (m/z) 251 (M+H)⁺.

Step 2: 4-(4,4-Dimethyl-1,4-azasilinan-1-yl)aniline

A solution of 4,4-dimethyl-1-(4-nitrophenyl)-1,4-azasilinane (step 1 intermediate) (120 mg, 0.48 mmol) in THF (10 mL) was subjected to hydrogenation in the presence of palladium on carbon as catalyst under 35 psi of hydrogen pressure at RT. The mixture was filtered and the filtrate was concentrated under reduced pressure to yield 50 mg of the desired compound. ¹H NMR (400 MHz, DMSO-d₆) δ 0.05 (s, 6H), 0.66-0.71 (m, 4H), 3.40 (t, J=6.0 Hz, 4H), 4.40 (br s, 2H), 6.45 (d, J=8.8 Hz, 2H), 6.62 (d, J=8.8 Hz, 2H); ESI-MS (m/z) 221 (M+H)⁺.

The analytical data of the intermediates prepared by following the procedure described above are given in below Table 6.

TABLE 6
Analytical data of Intermediate D9, D19, D37, D39, D40, D43, D44, D46,
D48 and D51

| Intermediate No. | Structure | Name and Analytical data |
|---|---|---|
| D9 | 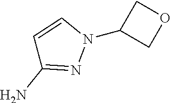 | 1-(Oxetan-3-yl)-1H-pyrazol-3- amine; ¹H NMR (400 MHz, DMSO- d₆) δ 4.75-4.85 (m, 6H), 5.25-5.32 (m, 1H), 5.42 (d, J = 2.0 Hz, 1H), 7.41 (d, J = 2.0 Hz, 1H). |
| D19 | 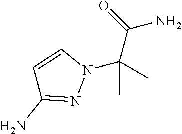 | 2-(3-amino-1H-pyrazol-1-yl)-2- methylpropanamide; ¹H NMR (400 MHz, DMSO-d₆) δ 1.61 (s, 6H), 3.35 (br s, 2H), 5.65 (d, J = 2.4 Hz, 1H), 6.63 (s, 1H), 7.16 (s, 1H), 7.58 (d, J = 2.4 Hz, 1H); ESI-MS (m/z) 169 (M + H)⁺. |
| D37 | 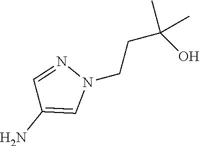 | 4-(4-Amino-1H-pyrazol-1-yl)-2- methylbutan-2-ol; ¹H NMR (400 MHz, DMSO-d₆) δ 1.09 (s, 6H), 1.77-1.83 (m, 2H), 3.34 (br s, 1H), 3.97-4.03 (m, 2H), 4.40 (br s, 2H), 6.93 (s, 1H), 7.15 (s, 1H). |
| D39 | 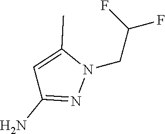 | 1-(2,2-Difluoroethyl)-5-methyl-1H- pyrazol-3-amine; ¹H NMR (400 MHz, DMSO-d₆) δ 2.12 (s, 3H), 4.15-4.25 (m, 2H), 4.54 (br s, 2H), 5.28 (s, 1H), 6.04-6.34 (m, 1H). |
| D40 | 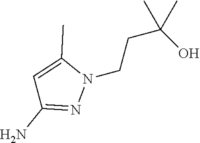 | 4-(3-Amino-5-methyl-1H-pyrazol-1- yl)-2-methylbutan-2-ol; ¹H NMR (400 MHz, DMSO-d₆) δ 1.11 (s, 6H), 1.71-1.77 (m, 2H), 2.23 (s, 3H), 3.63 (br s, 1H), 3.98-4.02 (m, 4H), 5.73 (s, 1H). |
| D43 | 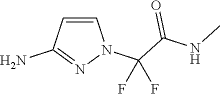 | 2-(3-Amino-1H-pyrazol-1-yl)-2,2- difluoro-N-methylacetamide; ¹H NMR (400 MHz, DMSO-d₆) δ 2.73 (d, J = 4.8 Hz, 3H), 5.28 (s, 2H), 5.74 (d, J = 2.8 Hz, 1H); 7.76 (d, J = 2.4 Hz, 1H), 9.02 (d, J = 4.0 Hz, 1H), ESI-MS (m/z) 191 (M + H)⁺. |
| D44 | 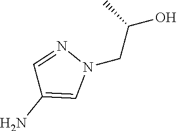 | (S)-1-(4-Amino-1H-pyrazol-1- yl)propan-2-ol; ¹H NMR (400 MHz, DMSO-d₆) δ 0.98 (d, J = 4.8 Hz, 3H), 3.76-3.89 (m, 4H), 4.82 (d, J = 4.8 Hz, 1H), 5.76 (br s, 1H), 6.88 (s, 1H), 7.01 (s, 1H). |
| D46 | 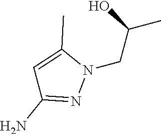 | (S)-1-(3-amino-5-methyl-1H- pyrazol-1-yl)propan-2-ol; ¹H NMR (400 MHz, DMSO-d₆) δ 1.04 (d, J = 6.4 Hz, 3H), 2.18 (s, 3H), 3.75-3.77 (m, 2H), 3.91 (q, J = 6.4 Hz, 1H), 5.00 (br s, 1H), 5.53 (s, 1H). |
| D48 | 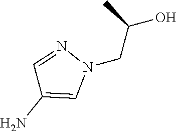 | (R)-1-(4-Amino-1H-pyrazol-1- yl)propan-2-ol; ¹H NMR (400 MHz, DMSO-d₆) δ 0.97 (d, J = 6.0 Hz, 3H), 3.74-3.86 (m, 5H), 4.80-4.84 (m, 1H), 6.89 (s, 1H), 7.01 (s, 1H). |
| D51 | 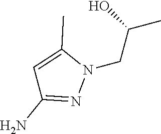 | (R)-1-(3-Amino-5-methyl-1H- pyrazol-1-yl)propan-2-ol; ¹H NMR (400 MHz, DMSO-d₆) δ 1.01 (d, J = 6.0 Hz, 3H), 2.12 (s, 3H), 3.58-3.72 (m, 3H), 3.85-3.90 (m, 1H), 4.88 (br s, 2H), 5.26 (s, 1H). |

### Intermediate D4

2-Methyl-4-(4-methylpiperazin-1-yl)aniline

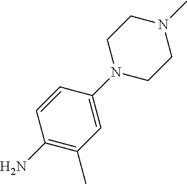

Step 1: 1-Methyl-4-(3-methyl-4-nitrophenyl)piperazine

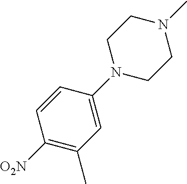 [SMILES: Cc1cc(N2CCN(C)CC2)ccc1[N+](=O)[O-]]

The titled compound was prepared by the reaction of 5-fluoro-2-nitrotoluene (2.0 g, 12.8 mmol) with N-methylpiperazine (1.7 mL, 15.4 mmol) in the presence of potassium carbonate (3.5 g, 25.7 mmol) in DMF (10 mL) as per the procedure described in step 1 of amine Intermediate 3 to yield 2.4 g of the compound. ¹H NMR (400 MHz, DMSO-d₆) δ 2.21 (s, 3H), 2.40-2.56 (m, 4H), 2.55 (s, 3H), 3.39-3.43 (m, 4H), 6.89 (d, J=7.2 Hz, 1H), 7.98 (d, J=10.0 Hz, 1H).

Step 2: 2-Methyl-4-(4-methylpiperazin-1-yl)aniline

To a solution of 1-methyl-4-(3-methyl-4-nitrophenyl)piperazine (1.0 g, 4.25 mmol) in ethanol (20 mL) was added catalytic amount of 10% palladium on carbon and the mixture was stirred at RT for 15 min under nitrogen atmosphere. Ammonium formate (2.6 g, 42.5 mmol) was added to the mixture and stirred for 2 min. The mixture was cooled to RT and filtered through celite. The filtrate was concentrated, dissolved in ethyl acetate and the organic solution was washed with saturated sodium bicarbonate solution followed by brine and dried over anhydrous sodium sulfate. The solution was filtered and concentrated under reduced pressure to yield 800 mg of the desired product. ¹H NMR (400 MHz, DMSO-d₆) δ 2.02 (s, 3H), 2.20 (s, 3H), 2.67-2.72 (m, 4H), 2.87-2.91 (m, 4H), 4.33 (br s, 2H), 6.48-6.56 (m, 2H), 6.59 (d, J=2.4 Hz, 1H); ESI-MS (m/z) 206 (M+H)⁺.

### Intermediate D5

6-(4-(Oxetan-3-yl)piperazin-1-yl)pyridin-3-amine

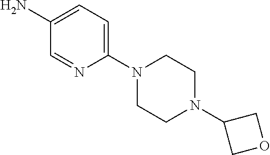 [SMILES: Nc1ccc(N2CCN(C3COC3)CC2)nc1]

### Step 1: 1-(5-Nitropyridin-2-yl)-4-(oxetan-3-yl)piperazine

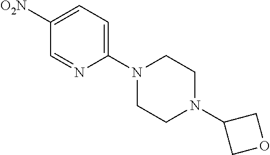 [SMILES: O=[N+]([O-])c1ccc(N2CCN(C3COC3)CC2)nc1]

To a stirred solution of 2-chloro-5-nitropyridine (300 mg, 1.89 mmol) in THF (5.0 mL) were added 1-(oxetan-3-yl)piperazine (296 mg, 2.08 mmol) and triethylamine (0.4 mL, 2.85 mmol) and the mixture was stirred at RT for 16 h. The mixture was filtered and the solid was washed with pet ether to yield 709 mg of the desired product. ¹H NMR (400 MHz, DMSO-d₆) δ 2.36 (t, J=4.8 Hz, 4H), 3.40-3.48 (m, 1H), 3.79 (t, J=4.8 Hz, 4H), 4.47 (t, J=6.0 Hz, 2H), 4.56 (t, J=6.4 Hz, 2H), 6.97 (d, J=9.6 Hz, 1H), 8.22 (dd, J=9.2, 2.8 Hz, 1H), 8.96 (d, J=2.8 Hz, 1H).

Step 2: 6-(4-(Oxetan-3-yl)piperazin-1-yl)pyridin-3-amine

A solution of 1-(5-nitropyridin-2-yl)-4-(oxetan-3-yl)piperazine (step 1 intermediate) (700 mg, 2.65 mmol) in a mixture of THF (18 mL), methanol (18 mL) and ethyl acetate (18 mL) was subjected to hydrogenation in the presence of palladium on carbon as catalyst under 35 psi of hydrogen pressure at RT. The mixture was filtered and the filtrate was concentrated under reduced pressure to yield 250 mg of the desired compound. ¹H NMR (400 MHz, DMSO-d₆) δ 2.33 (t, J=5.2 Hz, 4H), 3.24 (t, J=5.2 Hz, 4H), 3.40-3.44 (m, 1H), 4.46 (t, J=6.4 Hz, 2H), 4.55 (t, J=6.4 Hz, 2H), 4.60 (br s, 2H), 6.63 (d, J=8.8 Hz, 1H), 6.91 (dd, J=8.8, 3.2 Hz, 1H), 7.60 (d, J=2.4 Hz, 1H).

### Intermediate D6

1-cyclopropyl-1H-pyrazol-4-amine

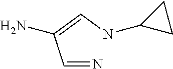

Step 1: 1-Cyclopropyl-4-nitro-1H-pyrazole

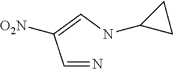 [SMILES: N=C/C(=C\NC1CC1)[N+](=O)[O-]]

To a mixture of 4-nitro-1H-pyrazole (5.0 g, 44.2 mmol), cyclopropylboronic acid (11.3 g, 132 mmol), copper (II) acetate (12.0 g, 66.3 mmol) and DMAP (16.2 g, 132 mmol) in 1,4-dioxane (100 mL) was added pyridine (5.3 mL, 65.7 mmol) at RT. The mixture was heated at 100° C. for 16 h. The mixture was cooled to RT and diluted with ethyl acetate. The organic solution was washed with water followed by brine and dried over anhydrous sodium sulfate. The solution was filtered and concentrated under reduced pressure and the residue obtained was purified by silica gel column chromatography to yield 1.0 g of the desired product. ¹H NMR (400 MHz, DMSO-d₆) δ 1.00-1.07 (m, 2H), 1.13-1.18 (m, 2H), 3.84-3.92 (m, 1H), 8.23 (s, 1H), 8.96 (s, 1H).

Step 2: 1-Cyclopropyl-1H-pyrazol-4-amine

A solution of 1-cyclopropyl-4-nitro-1H-pyrazole (step 1 intermediate) (1.0 g, 6.52 mmol) in methanol (20 mL) was subjected to hydrogenation in the presence of palladium on carbon as catalyst under 35 psi of hydrogen pressure at RT for 4 h. The mixture was filtered and the filtrate was concentrated under reduced pressure to yield 600 mg of the desired compound. ¹H NMR (400 MHz, DMSO-d₆) δ 0.82-0.93 (m, 4H), 3.46-3.52 (m, 1H), 3.85 (br s, 2H), 6.88 (s, 1H), 7.03 (s, 1H); ESI-MS (m/z) 124 (M+H)⁺.

### Intermediate D7

### 2-(4-Amino-1H-pyrazol-1-yl)propanenitrile

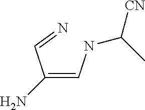

### Step 1: 2-(4-Nitro-1H-pyrazol-1-yl)propanenitrile

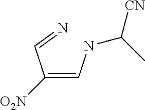

To a mixture of 4-nitro-1H-pyrazole (1.0 g, 8.05 mmol), DL-lactonitrile (628 mg, 8.05 mmol) and triphenylphosphine (2.78 g, 10.6 mmol) in THF (20 mL) was dropwise added DIAD (2.1 g, 10.6 mmol) and the resulting mixture was stirred at RT for 18 h. The mixture was diluted with ethyl acetate and washed with water followed by brine. The organic layer was dried over anhydrous sodium sulfate. The solution was filtered, concentrated under reduced pressure and the residue obtained was purified by silica gel column chromatography to yield 850 mg of the desired product. ¹H NMR (400 MHz, DMSO-d₆) δ 1.84 (d, J=7.2 Hz, 3H), 5.94 (q, J=7.2 Hz, 1H), 8.88 (s, 2H).

### Step 2: 2-(4-Amino-1H-pyrazol-1-yl)propanenitrile

To a stirred solution of 2-(4-nitro-1H-pyrazol-1-yl)propanenitrile (step 1 intermediate) (840 mg, 5.06 mmol) and ammonium chloride (2.8 g, 50.55 mmol) in a mixture of methanol (10 mL) and water (10 mL) at 80° C. was added iron powder (1.3 g, 25.3 mmol) in small portions. The mixture was stirred at 80° C. for 5 h. The mixture was cooled and methanol was removed under reduced pressure. The residue was partitioned between ethyl acetate and water. The suspension was filtered through celite. The layers were separated and the organic layer was washed with washed with water followed by brine. The organic layer was dried over anhydrous sodium sulfate. The solution was filtered, concentrated under reduced pressure and the residue obtained was purified by silica gel column chromatography to yield 386 mg of the desired product. ¹H NMR (400 MHz, DMSO-d₆) δ 1.70 (d, J=7.2 Hz, 3H), 4.03 (br s, 2H), 5.61 (q, J=7.2 Hz, 1H), 7.07 (s, 1H), 7.16 (s, 1H).

### Intermediate D8

3-Amino-N,1-dimethyl-1H-pyrazole-5-carboxamide

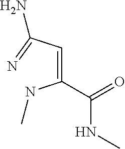

Step 1: N,1-Dimethyl-3-nitro-1H-pyrazole-5-carboxamide

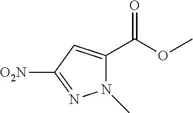

To a stirred solution of 3-nitro-1H-pyrazole-5-carboxylic acid (500 mg, 3.18 mmol) in DMF (5.0 mL) were added potassium carbonate (1.31 g, 9.54 mmol) followed by methyl iodide (0.616 mL, 9.54 mmol) and the mixture was heated at 80° C. for 7 h. The mixture was cooled to RT, diluted with water and extracted with ethyl acetate. The organic layer was washed with brine and dried over anhydrous sodium sulfate. The solvent was distilled of under reduced pressure to yield 300 mg of the desired compound (Isomeric mixture). ¹H NMR (400 MHz, DMSO-d₆) δ 3.89 (s, 3H), 4.20 (s, 3H), 7.56 (s, 1H).

Step 2: N,1-Dimethyl-3-nitro-1H-pyrazole-5-carboxamide

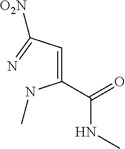

To a solution of N,1-dimethyl-3-nitro-1H-pyrazole-5-carboxamide (step 1 intermediate) (300 mg, 1.62 mmol) in THF (4.0 mL) was added methylamine (2M in THF, 1.0 mL) at RT and the mixture was heated at 90° C. for 18 h. The mixture was concentrated under vacuum and purified by silica gel column chromatography to yield 151 mg of the desired compound. ¹H NMR (400 MHz, DMSO-d₆) δ 2.77 (s, 3H), 4.17 (s, 3H), 7.57 (s, 1H), 8.77 (s, 1H).

Step 3: 3-Amino-N,1-dimethyl-1H-pyrazole-5-carboxamide

A solution of 1 N,1-dimethyl-3-nitro-1H-pyrazole-5-carboxamide (step 2 intermediate) (140 mg, 0.76 mmol) in methanol (15 mL) was subjected to hydrogenation in the presence of palladium on carbon (10% w/w, wet) as catalyst under 35 psi of hydrogen pressure at RT for 4 h. The mixture was filtered and the filtrate was concentrated under reduced pressure to yield 115 mg of the desired compound. ¹H NMR (400 MHz, DMSO-d₆) δ 2.69 (d, J=4.8 Hz, 3H), 3.83 (s, 3H), 4.94 (br s, 2H), 5.93 (s, 1H), 8.21 (s, 1H); ESI-MS (m/z) 155 (M+H)⁺.

### Intermediate D 10

1-(Tetrahydro-2H-pyran-4-yl)-1H-pyrazol-3-amine

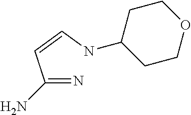 [SMILES: N=C(N)/C=C\NC1CCOCC1]

### Step 1: 3-Nitro-1-(tetrahydro-2H-pyran-4-yl)-1H-pyrazole

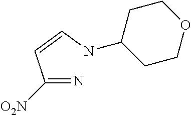 [SMILES: N=C(/C=C\NC1CCOCC1)[N+](=O)[O-]]

To a mixture of 3-nitro-1H-pyrazole (1.0 g, 8.84 mmol), tetrahydro-2H-pyran-4-ol (1.35 g, 13.2 mmol) and triphenylphosphine (3.47 g, 13.2 mmol) in THF (20 mL) was dropwise added DIAD (2.68 g, 13.2 mmol) and the resulting mixture was stirred at RT for 18 h. The mixture was diluted with ethyl acetate and washed with water followed by brine. The organic layer was dried over anhydrous sodium sulfate. The solution was filtered, concentrated under reduced pressure and the residue obtained was purified by silica gel column chromatography to yield 2.52 g of the desired product. ¹H NMR (400 MHz, DMSO-d₆) δ 1.95-2.09 (m, 4H), 3.45-3.51 (m, 2H), 3.96-4.00 (m, 2H), 5.17-5.23 (m, 1H), 7.26 9s, 1H), 7.74 (s, 1H).

Step 2: 1-(Tetrahydro-2H-pyran-4-yl)-1H-pyrazol-3-amine

A solution of 3-Nitro-1-(tetrahydro-2H-pyran-4-yl)-1H-pyrazole (step 1 intermediate) (2.5 g, 12.6 mmol) in methanol (25 mL) was subjected to hydrogenation in the presence of palladium on carbon (10% w/w, wet) as catalyst under 35 psi of hydrogen pressure at RT for 4 h. The mixture was filtered and the filtrate was concentrated under reduced pressure to yield 115 mg of the desired compound. ¹H NMR (400 MHz, DMSO-d₆) δ 1.67-1.72 (m, 2H), 1.88-1.97 (m, 2H), 3.34-3.43 (m, 2H), 3.92-3.96 (m, 2H), 4.17-4.28 (m, 1H), 5.15 (s, 2H), 5.25 (s, 1H), 7.04 (s, 1H).

The analytical data of the intermediates prepared by following the procedure described above are given in below Table 7.

TABLE 7
Analytical data of Intermediate D17, D26, D33, D36, D38, D42 and D57-D61

| Intermediate No. | Structure | Name and Analytical data |
|---|---|---|
| D17 | 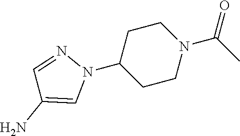 | 1-(4-(4-Amino-1H-pyrazol-1- yl)piperidin-1-yl)ethanone; ¹H NMR (400 MHz, DMSO-d₆) δ 1.59-1.68 (m, 2H), 1.73-1.94 (s, 2H), 2.02 (s, 3H), 2.66 (t, J = 9.3 Hz, 1H), 3.10- 3.18 (m, 1H), 3.84-3.89 (m, 3H), 4.15-4.23 (m, 1H), 4.38-4.43 (m, 1H), 6.11 (s, 1H), 7.06 (s, 1H). |
| D26 | 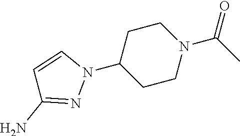 | 1-(4-(3-Amino-1H-pyrazol-1- yl)piperidin-1-yl)ethanone; ¹H NMR (400 MHz, DMSO-d₆) δ 1.61-1.83 (m, 2H), 1.90-1.99 (m, 2H), 2.02 (s, 3H), 2.62-2.69 (m, 1H), 3.11-3.19 (m, 2H), 3.85-3.93 (m, 1H), 4.16- 4.25 (m, 1H), 4.40-4.45 (m, 1H), 5.69 (d, J = 2.4 Hz, 1H), 7.55 (d, J = 2.4 Hz, 1H) |
| D33 | 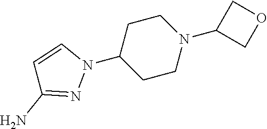 [SMILES: Nc1ccn(C2CCN(C3COC3)CC2)n1] | 1-(1-(oxetan-3-yl)piperidin-4-yl)- 1H-pyrazol-3-amine; ¹H NMR (400 MHz, DMSO-d₆) δ 1.50-1.61 (m, 1H), 1.80-1.96 (m, 3H), 2.02 1.99- 2.06 (m, 3H), 2.46-2.49 (m, 2H), 3.34-3.44 (m, 2H), 4.30-4.45 (m, 2H), 4.49-5.6 (m, 2H), 4.57-4.70 (m, 1H), 5.37 (d, J = 2.4 Hz, 1H), 7.34 (d, J = 2.4 Hz, 1H). |
| D36 | 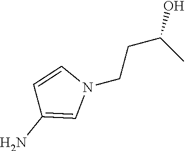 | (R)-4-(4-Amino-1H-pyrazol-1- yl)butan-2-ol; ¹H NMR (400 MHz, DMSO-d₆) δ 1.24 (d, J = 6.4 Hz, 3H), 1.63-1.81 (m, 2H), 3.48-3.56 (m, 2H), 3.85-4.13 (m, 2H), 4.57 (br s, 1H), 6.91 (d, J = 2.4 Hz, 1H), 7.03 (d, J = 2.4 Hz, 1H). |
| D38 | 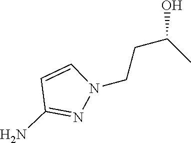 | (R)-4-(3-Amino-1H-pyrazol-1- yl)butan-2-ol; ¹H NMR (400 MHz, DMSO-d₆) δ 1.18 (d, J = 6.4 Hz, 3H), 1.60-1.81 (m, 2H), 3.47-3.57 (m, 1H), 3.87 (t, J = 7.2 Hz, 2H), 4.64 (br s, 2H), 5.37 (d, J = 2.0 Hz, 1H), 7.29 (d, J = 2.0 Hz, 1H). |
| D42 | 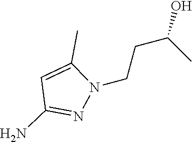 | (R)-4-(3-Amino-5-methyl-1H- pyrazol-1-yl)butan-2-ol; ¹H NMR (400 MHz, DMSO-d₆) δ 1.06 (d, J = 2.8 Hz, 3H), 1.57-1.72 (m, 2H), 1.95 (s, 3H), 3.51-3.58 (m, 1H), 3.76-3.82 (m, 2H), 4.73-4.81 (m, 2H), 5.00 (br s, 2H), 5.76 (s, 1H). |
| D57 | 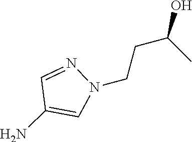 | (S)-4-(4-Amino-1H-pyrazol-1- yl)butan-2-ol; ¹H NMR (400 MHz, DMSO-d₆) δ 1.08 (d, J = 2.8 Hz, 3H), 1.63-1.80 (m, 2H), 3.17 (s, 1H), 3.52- 3.54 (m, 2H), 3.85-4.02 (m, 2H), 4.57 (br s, 1H), 6.95 (s, 1H), 7.06 (s, 1H). |
| D58 | 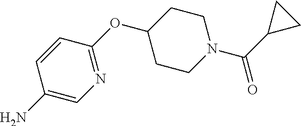 [SMILES: Nc1ccc(OC2CCN(C(=O)C3CC3)CC2)nc1] | (4-((5-Aminopyridin-2- yl)oxy)piperidin-1- yl)(cyclopropyl)methanone; ¹H NMR (400 MHz, CDCl₃) δ 0.75-0.78 (m, 2H), 0.97-1.00 (m, 2H), 1.25- 1.28 (m, 1H), 1.75-1.82 (m, 2H), 2.04-2.07 (m, 2H), 3.19 (br s, 2H), 3.48-3.58 (m, 2H), 3.92-3.98 (m, 2H), 5.13-5.17 (m, 1H), 6.59 (d, J = 8.8 Hz, 1H), 7.06 (dd, J = 8.8, 3.2 Hz, 1H), 7.66 (d, J = 2.8 Hz, 1H). |
| D59 | 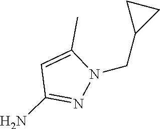 | 1-(Cyclopropylmethyl)-5-methyl- 1H-pyrazol-3-amine; ¹H NMR (400 MHz, DMSO-d₆) δ 0.25-0.26 (m, 2H), 0.42-0.45 (m, 2H), 1.16-1.20 (m, 1H), 2.10 (s, 3H), 3.61 (d, J = 6.8 Hz, 2H), 4.76-4.81 (m, 2H), 5.20 (s, 1H). |
| D60 | 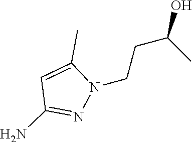 | (S)-4-(3-Amino-5-methyl-1H- pyrazol-1-yl)butan-2-ol; ¹H NMR (400 MHz, DMSO-d₆) δ 1.08 (d, J = 2.8 Hz, 3H), 1.58-1.83 (m, 2H), 1.95 (s, 4H), 3.52-3.57 (m, 2H), 3.79 (t, J = 6.8 Hz, 2H), 4.03 (q, J = 6.8 Hz, 1H), 8.88 (s, 1H). |
| D61 | 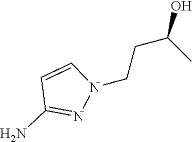 | (S)-4-(3-Amino-1H-pyrazol-1- yl)butan-2-ol; ¹H NMR (400 MHz, DMSO-d₆) δ 1.05 (d, J = 6.0 Hz, 3H), 1.61-1.81 (m, 2H), 3.51-3.54 (m, 1H), 3.86 (t, J = 6.0 Hz, 2H), 4.41- 4.43 (m, 3H), 5.34 (s, 1H), 7.27 (s, 1H). |

### Intermediate D11

2-(3-Amino-1H-pyrazol-1-yl)-2-methylpropanenitrile

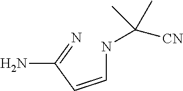

Step 1: 2-Methyl-2-(3-nitro-1H-pyrazol-1-yl)propanamide

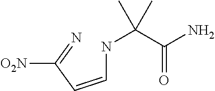

To a mixture of 3-nitro-1H-pyrazole (2.0 g, 17.6 mmol) in DMF (20 mL) were added 2-bromoisobutyramide (4.40 g, 26.5 mmol) and potassium carbonate (4.8 g, 35.3 mmol) and the mixture was heated at 50° C. for 2 h. The mixture was cooled to RT and quenched with water. The precipitated solid was filtered, washed with water and dried to obtain 2.75 g of the desired compound. ¹H NMR (400 MHz, DMSO-d₆) δ 1.77 (s, 6H), 7.09 (d, J=2.4 Hz, 1H), 7.27 (s, 1H), 7.37 (s, 1H), 8.15 (d, J=2.4 Hz, 1H).

Step 2: 2-Methyl-2-(3-nitro-1H-pyrazol-1-yl)propanenitrile

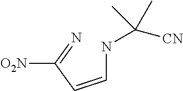

A solution of 2-methyl-2-(3-nitro-1H-pyrazol-1-yl)propanamide (step 1 intermediate) (2.7 g, 13.6 mmol) in phosphorous oxychloride (15 mL) was heated at 90° C. for 1 h. The mixture was poured over ice-water mixture and the solution was neutralized using sodium bicarbonate solution. The aqueous mixture was extracted with ethyl acetate and the organic layer was washed with brine. The solvent was removed under reduced pressure and the residue was purified by silica gel column chromatography to yield 1.25 g of the desired compound. ¹H NMR (400 MHz, DMSO-d₆) δ 2.04 (s, 6H), 7.24 (d, J=2.8 Hz, 1H), 8.39 (d, J=2.8 Hz, 1H).

Step 3: 2-(3-Amino-1H-pyrazol-1-yl)-2-methylpropanenitrile

The titled compound was prepared by the reaction of 2-methyl-2-(3-nitro-1H-pyrazol-1-yl)propanenitrile (step 2 intermediate) (1.2 g, 6.60 mmol) with iron powder (1.85 g, 33.3 mmol) and ammonium chloride (1.77 g, 33.3 mmol) in a mixture of ethanol (40 mL) and water (10 mL) as per the procedure described in step 2 of Intermediate D7 to yield 500 mg of the compound. ¹H NMR (400 MHz, DMSO-d₆) δ 1.85 (s, 6H), 4.91 (s, 2H), 5.53 (d, J=2.4 Hz, 1H), 7.56 (d, J=2.4 Hz, 1H).

The analytical data of the intermediates prepared by following the procedure described above are given in below Table 8.

TABLE 8
Analytical data of amine Intermediate D16

| Intermediate No. | Structure | Name and Analytical data |
|---|---|---|
| D16 | 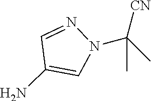 | 2-(4-Amino-1H-pyrazol-1-yl)-2- methylpropanenitrile; ¹H NMR (400 MHz, DMSO-d₆) δ 1.87 (s, 6H), 4.04 (s, 2H), 7.10 (d, J = 2.4 Hz, 1H), 7.24 (d, J = 2.4 Hz, 1H). |

### Intermediate D12

### (S)-2-(3-Amino-1H-pyrazol-1-yl)propanenitrile

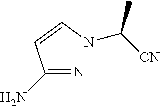

Step 1: (S)-Methyl 2-(3-nitro-1H-pyrazol-1-yl)propanoate

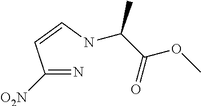

The titled compound was prepared by the reaction of 3-nitro-1H-pyrazole (2.0 g, 17.68 mmol) with methyl (R)-(+)-lactate (2.02 g, 19.45 mmol) in the presence of triphenylphosphine (5.56 g, 21.21 mmol) and DIAD (4.28 g, 21.21 mmol) in THF (30 mL) as per the procedure described in step 1 of Intermediate D10 to yield 1.8 g of the compound. ¹H NMR (400 MHz, DMSO-d₆) δ 1.73 (d, J=7.2 Hz, 3H), 3.69 (s, 3H), 2.53 (q, J=7.2 Hz, 1H), 7.12 (s, 1H), 8.17 (s, 1H).

Step 2: (S)-2-(3-Nitro-1H-pyrazol-1-yl)propanoic acid

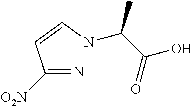

To a solution of (S)-methyl 2-(3-nitro-1H-pyrazol-1-yl)propanoate (step 1 intermediate) (1.8 g, 9.03 mmol) in a mixture of methanol (20 mL) and water (10 mL) was added lithium hydroxide monohydrate (1.68 g, 36.2 mmol) and the mixture was stirred at RT for 18 h. The mixture was concentrated and the residue was diluted with water. The aqueous mixture was acidified with 1N hydrochloric and extracted with ethyl acetate. The organic layer was washed with brine and dried over anhydrous sodium sulfate. The solution was concentrated under reduced pressure to yield 1.51 g of the desired compound. ¹H NMR (400 MHz, DMSO-d₆) δ 1.72 (d, J=7.2 Hz, 3H), 5.36 (s, 3H), 7.10 (s, 1H), 8.15 (s, 1H), 13.37 (br hump, 1H); ESI-MS (m/z) 184 (M+H)⁺.

### Step 3: (S)-2-(3-Nitro-1H-pyrazol-1-yl)propanamide

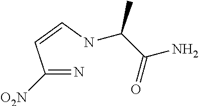

To a solution of (S)-2-(3-nitro-1H-pyrazol-1-yl)propanoic acid (step 2 intermediate) (2.5 g, 13.5 mmol) in THF (20 mL) were added ethyl chloroformate (2.05 g, 18.9 mmol), triethylamine (2.84 mL, 20.25 mmol) and aqueous ammonia (10 mL) at 0° C. The resultant mixture was stirred at RT for 1 h. The mixture was diluted with ethyl acetate and washed with water followed by brine. The organic layer was dried over anhydrous sodium sulfate. The solution was filtered, concentrated under reduced pressure and the residue obtained was purified by silica gel column chromatography to yield 310 mg of the desired product. ¹H NMR (400 MHz, DMSO-d₆) δ 1.67 (d, J=7.2 Hz, 3H), 5.12 (q, J=7.2 Hz, 1H), 7.07 (s, 1H), 7.41 (s, 1H), 7.69 (s, 1H), 8.10 (s, 1H).

### Step 4: (S)-2-(3-Nitro-1H-pyrazol-1-yl)propanenitrile

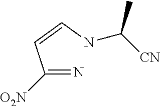

A mixture was (S)-2-(3-nitro-1H-pyrazol-1-yl)propanamide (step 3 intermediate) (800 mg, 4.34 mmol) was heated at 90° C. for 2 h. The mixture was cooled to RT and quenched on crushed ice. The mixture was extracted with ethyl acetate. The organic extract was washed with sat. sodium bicarbonate solution followed by brine and dried over anhydrous sodium sulfate. The solvent was removed under reduced pressure to yield 310 mg of the desired compound. ¹H NMR (400 MHz, DMSO-d₆) δ 1.85 (d, J=7.2 Hz, 3H), 6.04 (q, J=7.2 Hz, 1H), 7.18 (s, 2H), 8.25 (s, 1H).

### Step 5: (S)-2-(3-Amino-1H-pyrazol-1-yl)propanenitrile

The titled compound was prepared by the reaction of (S)-2-(3-nitro-1H-pyrazol-1-yl)propanenitrile (step 4 intermediate) (300 mg, 1.80 mmol) with iron powder (480 mg, 9.0 mmol) and ammonium chloride (480 mg, 9.0 mmol) in a mixture of ethanol (20 mL) and water (10 mL) as per the procedure described in step 2 of Intermediate D7 to yield 187 mg of the compound. ¹H NMR (400 MHz, DMSO-d₆) δ 1.68 (d, J=7.2 Hz, 3H), 4.88 (s, 2H), 5.49 (t, J=4.8 Hz, 2H), 7.46 (s, 1H).

The analytical data of the intermediates prepared by following the procedure described above are given in below Table 9.

TABLE 9
Analytical data of Intermediate D21, D45, D49, D52, D53 and D63

| Intermediate No. | Structure | Name and Analytical data |
|---|---|---|
| D21 | 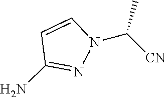 | (R)-2-(3-Amino-1H-pyrazol-1- yl)propanenitrile; ¹H NMR (400 MHz, DMSO-d₆) δ 1.68 (d, J = 7.2 Hz, 3H), 4.88 (s, 2H), 5.48-5.52 (m, 2H), 7.46 (d, J = 2.4 Hz, 1H). |
| D45 | 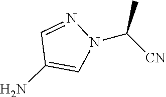 | (S)-2-(4-Amino-1H-pyrazol-1- yl)propanenitrile; ¹H NMR (400 MHz, DMSO-d₆) δ 1.70 (d, J = 7.2 Hz, 3H), 4.08 (br s, 2H), 5.61 (q, J = Hz, 1H), 7.07 (s, 1H), 7.16 (s, 1H). |
| D49 | 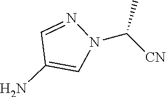 | (R)-2-(4-Amino-1H-pyrazol-1- yl)propanenitrile; ¹H NMR (400 MHz, DMSO-d₆) δ 1.71 (d, J = 7.2 Hz, 3H), 4.05 (s, 2H), 5.58-5.64 (m, 1H), 7.07 (s, 1H), 7.16 (s, 1H). |
| D52 | 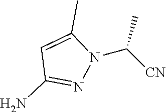 | (R)-2-(3-Amino-5-methyl-1H- pyrazol-1-yl)propanenitrile; ¹H NMR (400 MHz, DMSO-d₆) δ 1.62 (d, J = 7.2 Hz, 3H), 2.14 (s, 3H), 4.78 (s, 2H), 5.31 (s, 1H), 5.47-5.52 (m, 1H). |
| D53 | 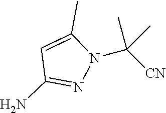 | 2-(3-Amino-5-methyl-1H-pyrazol-1- yl)-2-methylpropanenitrile; ¹H NMR (400 MHz, DMSO-d₆) δ 1.85 (s, 6H), 2.37 (s, 3H), 4.77 (br s, 2H), 5.41 (s, 1H). |
| D63 | 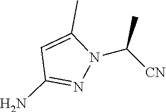 | (S)-2-(3-Amino-5-methyl-1H- pyrazol-1-yl)propanenitrile; ¹H NMR (400 MHz, DMSO-d₆) δ 1.62 (d, J = 7.2 Hz, 3H), 2.14 (s, 3H), 4.77 (s, 2H), 5.31 (s, 1H), 5.50 (q, J = 7.2 Hz, 1H). |

### Intermediate D13

### 1-(3-(3-Amino-1H-pyrazol-1-yl)azetidin-1-yl)ethanone

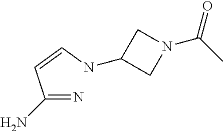

Step 1: tert-Butyl 3-(3-nitro-1H-pyrazol-1-yl)azetidine-1-carboxylate

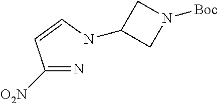 [SMILES: CC(C)(C)OC(=O)N1CC(N/C=C\C(=N)[N+](=O)[O-])C1]

The titled compound was prepared by the reaction of 3-nitro-1H-pyrazole (2.0 g, 17.68 mmol) with 1-Boc-3-hydroxyazetidine (3.36 g, 19.4 mmol) in the presence of triphenylphosphine (5.56 g, 21.2 mmol) and DIAD (4.28 g, 21.21 mmol) in THF (30 mL) as per the procedure described in step 1 of Intermediate D10 to yield 1.2 g of the desired compound. ¹H NMR (400 MHz, DMSO-d₆) δ 1.40 (s, 9H), 4.30-4.32 (m, 4H), 5.69-5.74 (m, 1H), 7.32 (s, 1H), 7.83 (s, 1H).

Step 2: 1-(Azetidin-3-yl)-3-nitro-1H-pyrazole hydrochloride

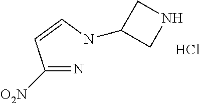 [SMILES: N=C(/C=C\NC1CNC1)[N+](=O)[O-] ; Cl]

To a solution of tert-butyl 3-(3-nitro-1H-pyrazol-1-yl)azetidine-1-carboxylate (step 1 intermediate) (1.2 g, 4.47 mmol) in ethyl acetate (10 mL) was added hydrochloric acid in ethyl acetate (20 mL) at 0° C. and stirred RT for 3 h. The solvent was removed under reduced pressure and the residue was stirred with diethyl ether. The precipitated solid was filtered and dried well to yield 1.0 g of the desired product'H NMR (400 MHz, DMSO-d₆) δ 4.32 (t, J=8.4 Hz, 2H), 4.49 (t, J=11.2 Hz, 2H), 5.85-5.92 (m, 1H), 7.36-7.38 (m, 1H), 7.88-7.90 (m, 1H), 9.74 (s, 2H); ESI-MS (m/z) 168 (M+H)⁺.

### Step 3: 1-(3-(3-Nitro-1H-pyrazol-1-yl)azetidin-1-yl)ethanone

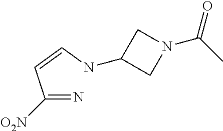

To a stirred solution of 1-(azetidin-3-yl)-3-nitro-1H-pyrazole hydrochloride (1.0 g, 4.90 mmol) in dichloromethane (20 mL) were added triethylamine (1.4 mL, 9.26 mmol) followed by acetyl chloride (0.52 mL, 7.32 mmol) and the mixture was stirred at RT for 7 h. The mixture was diluted with water and extracted with dichloromethane. The organic extract was washed with brine and dried over anhydrous sodium sulfate. The solution was filtered and concentrated to yield 450 mg of the desired compound. ¹H NMR (400 MHz, DMSO-d₆) δ 2.16 (s, 3H), 4.30 (d, J=6.4 Hz, 2H), 4.50-4.54 (m, 1H), 4.59-4.63 (m, 1H), 5.70-5.78 (m, 1H), 7.34 (s, 1H), 7.83 (s, 1H).

### Step 4: 1-(3-(3-Amino-1H-pyrazol-1-yl)azetidin-1-yl)ethanone

A solution of 1-(3-(3-nitro-1H-pyrazol-1-yl)azetidin-1-yl)ethanone (step 3 intermediate) (450 mg, 0.46 mmol) in methanol (15 mL) was subjected to hydrogenation in the presence of palladium on carbon (10% w/w, wet) as catalyst under 35 psi of hydrogen pressure at RT for 4 h. The mixture was filtered and the filtrate was concentrated under reduced pressure to yield 115 mg of the desired compound. ¹H NMR (400 MHz, DMSO-d₆) δ 1.80 (s, 3H), 4.04-4.07 (m, 1H), 4.08-4.20 (m, 1H), 4.33-4.36 (m, 1H), 4.43-4.47 (m, 1H), 5.04-5.08 (m, 1H),5.27-5.76 (m, 3H), 7.18 (s, 1H); ESI-MS (m/z) 181 (M+H)⁺.

### Intermediate D14

6-(4-Cyclopropylpiperazin-1-yl)pyridin-3-amine

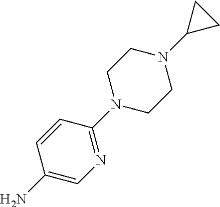 [SMILES: Nc1ccc(N2CCN(C3CC3)CC2)nc1]

Step 1: 1-Cyclopropyl-4-(5-nitropyridin-2-yl)piperazine

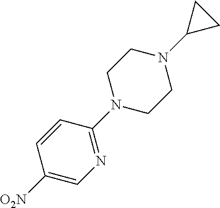 [SMILES: O=[N+]([O-])c1ccc(N2CCN(C3CC3)CC2)nc1]

To a stirred solution of 2-chloro-5-nitropyridine (200 mg, 1.26 mmol) in DMFF (5.0 mL) were added 1-cyclopropylpiperazine (191 mg, 1.57 mmol) and DIPEA (0.54 mL, 3.09 mmol) and the mixture was stirred at 80° C. for 5 h. The mixture was cooled to RT and diluted with water. The solid was filtered and washed with pet ether to yield 250 mg of the desired product. ¹H NMR (400 MHz, DMSO-d₆) δ 0.36-0.47 (m, 4H), 1.64-1.67 (m, 1H), 2.60 (s, 4H), 3.72 (s, 4H), 6.95(d, J=9.6 Hz, 1H), 8.20 (dd, J₁=2.8 Hz, J₂=9.6 Hz, 1H), 4.95 (s, 1H) ESI-MS (m/z) 249 (M+H)⁺.

Step 2: 6-(4-Cyclopropylpiperazin-1-yl)pyridin-3-amine

A solution of 1-cyclopropyl-4-(5-nitropyridin-2-yl)piperazine (step 1 intermediate) (250 mg, 1.01 mmol) in a mixture of THF (20 mL) and methanol (20 mL) was subjected to hydrogenation in the presence of palladium on carbon as catalyst under 35 psi of hydrogen pressure at RT for 3 h. The mixture was filtered and the filtrate was concentrated under reduced pressure to yield 175 mg of the desired compound. ¹H NMR (400 MHz, DMSO-d₆) δ 0.31-0.34 (m, 2H), 0.40-0.46 (m, 2H), 1.60-1.63 (m, 1H), 2.60 (t, J=5.2 Hz, 4H), 3.17 (t, J=4.8 Hz, 4H), 4.56 (s, 2H), 6.60 (d, J=8.4 Hz, 1H), 6.90 (dd, J₁=2.8 Hz, J₂=8.8 Hz, 1H), 7.59 (s, 1H); ESI-MS (m/z) 219 (M+H)⁺.

### Intermediate D15

### 1-(3-Amino-1H-pyrazol-1-yl)cyclopropanecarbonitrile

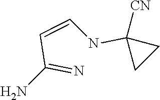 [SMILES: N#CC1(N/C=C\C(=N)N)CC1]

### Step 1: 2-(3-Nitro-1H-pyrazol-1-yl)acetonitrile

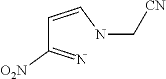 [SMILES: N#CCN/C=C\C(=N)[N+](=O)[O-]]

To a solution of 3-nitro-1H-pyrazole (2.0 g, 17.68 mmol) in anhydrous DMF (15 mL) was added sodium hydride (60% w/w, 840 mg, 20.8 mmol) at 0° C. Bromoacetonitrile (1.2 mL, 17.5 mmol) was added to the mixture and stirred for 1 h at 0° C. followed by 1 h at RT. The mixture was partitioned between ethyl acetate and water. The organic layer was separated and washed and dried over anhydrous sodium sulfate. The solution was filtered, concentrated and the residue thus obtained was purified by silica gel column chromatography to yield 1.5 g of the desired compound.

¹H NMR (400 MHz, DMSO-d₆) δ 5.69 (s, 2H), 7.14 (s, 1H), 8.15 (s, 1H).

### Step 2: 1-(3-Nitro-1H-pyrazol-1-yl)cyclopropanecarbonitrile

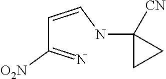 [SMILES: N#CC1(N/C=C\C(=N)[N+](=O)[O-])CC1]

To a solution of 2-(3-nitro-1H-pyrazol-1-yl)acetonitrile (step 1 intermediate) (1.5 g, 9.86 mmol) in DMSO (30 mL) was added sodium hydride (60% w/w, 1.76 g, 44.6 mmol) at 0° C. 1,2-Dibromoethane (2.5 mL, 29.5 mmol) was added to the mixture and stirred for 18 h at RT. Saturated ammonium chloride solution was added to the mixture and stirred at 0° C. for 15 min. The mixture was partitioned between ethyl acetate and water. The organic layer was separated and washed and dried over anhydrous sodium sulfate. The solution was filtered, concentrated and the residue thus obtained was purified by silica gel column chromatography to yield 700 mg of the desired compound. ¹H NMR (400 MHz, DMSO-d₆) δ 1.93-2.03 (m, 4H), 7.18 (s, 1H), 8.40 (s, 1H).

### Step 3: 1-(3-Amino-1H-pyrazol-1-yl)cyclopropanecarbonitrile

The titled compound was prepared by the reaction of 1-(3-nitro-1H-pyrazol-1-yl)cyclopropanecarbonitrile (step 2 intermediate) (700 mg, 3.93 mmol) with iron powder (870 mg, 15.7 mmol) and ammonium chloride (2.10 g, 39.3 mmol) in a mixture of ethyl acetate (30 mL) and water (30 mL) as per the procedure described in step 2 of Intermediate D7 to yield 350 mg of the compound. ¹H NMR (400 MHz, DMSO-d₆) δ 1.62-2.17 (m, 4H), 4.92 (s, 2H), 5.51 (s, 1H), 7.53 (s, 1H).

### Intermediate D18

1-(2-Morpholinoethyl)-1H-pyrazol-3-amine

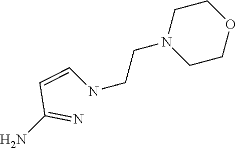 [SMILES: N=C(N)/C=C\NCCN1CCOCC1]

### Step 1: 4-(2-(3-Nitro-1H-pyrazol-1-yl)ethyl)morpholine

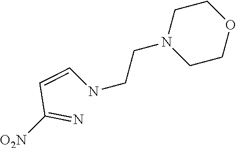 [SMILES: N=C(/C=C\NCCN1CCOCC1)[N+](=O)[O-]]

To a solution of 3-nitro-1H-pyrazole (1.0 g, 8.84 mmol) in DMF (10 mL) was added sodium hydride (60% w/w, 707 mg, 17.68 mmol) at 0° C. 4-(2-chloroethyl)morpholine (1.97 g, 10.6 mmol) was added to the mixture and stirred for 2 h at 80° C. Ice-cold water was added to the mixture and stirred at 0° C. for 15 min. The mixture was partitioned between ethyl acetate and water. The organic layer was separated and washed and dried over anhydrous sodium sulfate. The solution was filtered, concentrated and the residue thus obtained was purified by silica gel column chromatography to yield 700 mg of the desired compound. ¹H NMR (400 MHz, DMSO-d₆) δ 2.41 (t, J=4.4 Hz, 4H), 2.74 (t, J=6.0 Hz, 2H), 3.52 (t, J=4.4 Hz, 4H), 4.36 (t, J=6.0 Hz, 4H), 7.04 (d, J=2.8 Hz, 1H), 8.05 (d, J=2.8 Hz, 1H).

Step 2: 1-(2-Morpholinoethyl)-1H-pyrazol-3-amine

A solution of 4-(2-(3-nitro-1H-pyrazol-1-yl)ethyl)morpholine (step 1 intermediate) (700 mg, 3.09 mmol) in methanol (25 mL) was subjected to hydrogenation in the presence of palladium on carbon as catalyst under 35 psi of hydrogen pressure at RT for 3 h. The mixture was filtered and the filtrate was concentrated under reduced pressure to yield 650 mg of the desired compound. ¹H NMR (400 MHz, DMSO-d₆) δ 2.37 (t, J=4.4 Hz, 4H), 2.60 (t, J=6.8 Hz, 2H), 3.54 (t, J=4.8 Hz, 4H), 3.93 (t, J=6.8 Hz, 2H), 4.55 (br s, 2H), 5.34 (d, J=2.4 Hz, 1H), 7.30 (d, J=2.4 Hz, 1H).

The analytical data of the intermediates prepared by following the procedure described above are given in below Table 10.

TABLE 10
Analytical data of Intermediate D41 and D62

| Intermediate No. | Structure | Name and Analytical data |
|---|---|---|
| D41 | 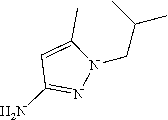 | 1-Isobutyl-5-methyl-1H-pyrazol-3- amine; ¹H NMR (400 MHz, DMSO- d₆) δ 0.81 (d, J = 6.8 Hz, 6H), 1.17- 2.06 (m, 1H), 2.09 (s, 3H), 3.52 (d, J = 7.2 Hz, 2H), 4.63 (br s, 2H), 5.20 (s, 1H). |
| D62 | 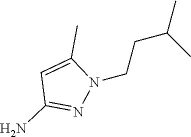 | 1-Isopentyl-5-methyl-1H-pyrazol-3- amine; ¹H NMR (400 MHz, DMSO- d₆) δ 0.89 (d, J = 6.8 Hz, 6H), 1.33- 1.53 (m, 2H), 2.09 (s, 3H), 3.35 (s, 1H), 3.69-3.75 (m, 2H), 4.36 (br s, 2H), 5.17 (s, 1H). |

### Intermediate D22

2-(3-Amino-1H-pyrazol-1-yl)-N,2-dimethylpropanamide

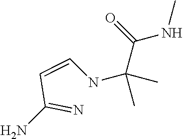

Step 1: N,2-Dimethyl-2-(3-nitro-1H-pyrazol-1-yl)propanamide

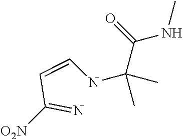

To a solution of 2-methyl-2-(3-nitro-1H-pyrazol-1-yl)propanoic acid (1.5 g, 7.5 mmol) in THF (10 mL) were added ethyl chloroformate (1.02 mL, 11.2 mmol), triethylamine (1.03 mL, 11.29 mmol) and methylamine (2M in THF, 10 mL) at 0° C. The resultant mixture was stirred at RT for 3 h. The mixture was diluted with ethyl acetate and washed with water followed by brine. The organic layer was dried over anhydrous sodium sulfate. The solution was filtered, concentrated under reduced pressure and the residue obtained was purified by silica gel column chromatography to yield 1.35 g of the desired product. ¹H NMR (400 MHz, DMSO-d₆) δ 1.76 (s, 6H), 2.59 (d, J=4.4 Hz, 3H), 4.04 (q, J=4.4 Hz, 1H), 7.10 (d, J=2.8 Hz, 1H), 8.16 (d, J=2.8 Hz, 1H).

Step 2: 2-(3-Amino-1H-pyrazol-1-yl)-N,2-dimethylpropanamide

A solution of N,2-dimethyl-2-(3-nitro-1H-pyrazol-1-yl)propanamide (step 1 intermediate) (1.35 g, 6.36 mmol) in methanol (50 mL) was subjected to hydrogenation in the presence of palladium on carbon as catalyst under 35 psi of hydrogen pressure at RT for 3 h. The mixture was filtered and the filtrate was concentrated under reduced pressure to yield 650 mg of the desired compound. ¹H NMR (400 MHz, DMSO-d₆) δ 1.58 (s, 6H), 2.55 (d, J=4.4 Hz, 3H), 3.37 (q, J=4.4 Hz, 1H), 5.54 (d, J=2.8 Hz, 1H), 7.10-7.11 (br s, 2H), 7.50 (d, J=2.8 Hz, 1H).

### Intermediate D23

### (R)-1-(3-Amino-1H-pyrazol-1-yl)propan-2-ol

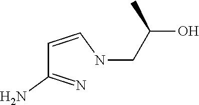

### Step 1: (R)-1-(3-Nitro-1H-pyrazol-1-yl)propan-2-ol

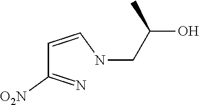

To a solution of 3-nitro-1H-pyrazole (1.0 g, 8.79 mmol) in DMF (10 mL) were added potassium carbonate (1.82 g, 13.1 mmol) followed by (R)-2-methyloxirane (1.02 g, 17.5 mmol) and the mixture was stirred at 80° C. for 5 h. The mixture was diluted with ethyl acetate and the organic mixture was washed with water followed by brine. The organic layer was dried over anhydrous sodium sulfate, filtered and concentrated to give 1.05 g of the desired compound. ¹H NMR (400 MHz, DMSO-d₆) δ 1.08 (d, J=6.4 Hz, 3H), 4.00-4.11 (m, 2H), 4.16-4.22 (m, 1H), 5.04 (d, J=5.2 Hz, 1H), 7.04 (d, J=2.4 Hz, 1H), 7.96 (d, J=2.4 Hz, 1H).

### Step 2: (R)-1-(3-Amino-1H-pyrazol-1-yl)propan-2-ol

A solution of (R)-1-(3-Nitro-1H-pyrazol-1-yl)propan-2-ol (step 1 intermediate) (1.35 g, 6.36 mmol) in methanol (50 mL) was subjected to hydrogenation in the presence of palladium on carbon as catalyst under 35 psi of hydrogen pressure at RT for 3 h. The mixture was filtered and the filtrate was concentrated under reduced pressure to yield 650 mg of the desired compound. ¹H NMR (400 MHz, DMSO-d₆) δ 0.99 (d, J=6.0 Hz, 3H), 3.72-3.82 (m, 2H), 3.86-3.93 (m, 1H), 4.87 (br s, 1H), 5.54 (d, J=5.2 Hz, 1H), 6.27 (d, J=2.4 Hz, 1H), 7.38 (d, J=2.4 Hz, 1H); ESI-MS (m/z) 168 (M+H)⁺.

The analytical data of the intermediates prepared by following the procedure described above are given in below Table 11.

TABLE 11
Analytical data of amine Intermediate D20 and D28

| Intermediate No. | Structure | Name and Analytical data |
|---|---|---|
| D20 | 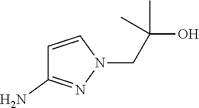 | 1-(3-Amino-1H-pyrazol-1-yl)-2- methylpropan-2-ol; ¹H NMR (400 MHz, DMSO-d₆) δ 1.02 (s, 6H), 3.73 (s, 2H), 4.62 (br s, 2H), 4.71 (s, 1H), 5.39 (d, J = 2.0 Hz, 1H), 7.28 (d, J = 2.0 Hz, 1H). |
| D28 | 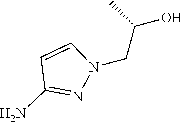 | (S)-1-(3-Amino-1H-pyrazol-1- yl)propan-2-ol; ¹H NMR (400 MHz, DMSO-d₆) δ 0.99 (d, J = 6.8 Hz, 3H), 3.17-3.35 (m, 2H), 3.65-3.89 (m, 1H), 4.60 (hump, 2H), 4.82 (s, 1H), 5.36 (s, 1H), 7.26 (s, 1H). |

### Intermediate D24

### (R)-2-(3-Amino-1H-pyrazol-1-yl)propan-1-ol

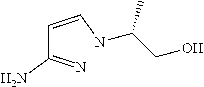

### Step 1: (R)-2-(3-Nitro-1H-pyrazol-1-yl)propan-1-ol

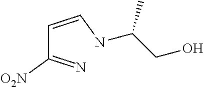

The titled compound was prepared by the reaction of 3-nitro-1H-pyrazole (1.0 g, 8.79 mmol) with methyl (S)-(+)-lactate (915 mg 8.79 mmol) in the presence of triphenylphosphine (2.76 g, 1.01 mmol) and DIAD (2.13 g, 10.5 mmol) in THF (10 mL) as per the procedure described in step 1 of Intermediate D10 to yield 1.25 g of the desired compound. ESI-MS (m/z) 172 (M+H)⁺.

### Step 2: (R)-2-(3-Amino-1H-pyrazol-1-yl)propan-1-ol

To a stirred solution of lithium aluminum hydride (990 mg, 26.1 mmol) in dry THF (10 mL) was dropwise added a solution of (R)-2-(3-nitro-1H-pyrazol-1-yl)propan-1-ol (step 1 intermediate) (2.0 g, 10.0 mmol) in THF (10 mL) at 0° C. and the mixture was stirred at 70° C. for 2 h. The mixture was cooled to 0° C. and quenched with ice-cooled water and 15% aq. sodium hydroxide solution. The mixture was diluted with ethyl acetate and filtered through celite. The filtrate was dried over anhydrous sodium sulfate, filtered and concentrated under reduced pressure to yield 500 mg of the desired compound. The crude compound as such used for the next step. ESI-MS (m/z) 142 (M+H)⁺.

The analytical data of the intermediates prepared by following the procedure described above are given in below Table 12.

TABLE 12
Analytical data of amine Intermediate D29

| Intermediate No. | Structure | Name and Analytical data |
|---|---|---|
| D29 | 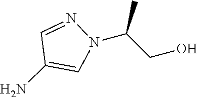 | (S)-2-(3-Amino-1H-pyrazol-1- yl)propan-1-ol; ESI-MS (m/z) 142 (M + H)⁺. |

### Intermediate D25

### 2-(3-Amino-1H-pyrazol-1-yl)-N-methylacetamide

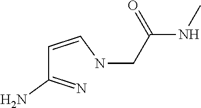

Step 1: Ethyl 2-(3-nitro-1H-pyrazol-1-yl)acetate

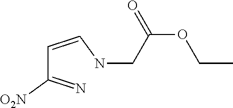

The titled compound was prepared by the reaction of 3-nitro-1H-pyrazole (2.0 g, 17.6 mmol) with ethyl bromoacetate (2.2 mL, 19.4 mmol) in the presence of sodium hydride (60% w/w, 849 mg, 21.2 mmol) in DMF (15 mL) as per the procedure described in step 1 of Intermediate D15 to yield 2.8 g of the desired compound. ¹H NMR (400 MHz, DMSO-d₆) δ 1.22 (d, J=7.2 Hz, 3H), 4.19 (q, J=7.2 Hz, 2H), 5.28 (s, 2H), 7.10 (d, J=2.4 Hz, 1H), 8.05 (d, J=2.4 Hz, 1H).

Step 2: N-Methyl-2-(3-nitro-1H-pyrazol-1-yl)acetamide

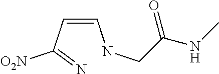

The titled compound was prepared by the reaction of ethyl 2-(3-nitro-1H-pyrazol-1-yl)acetate (step 1 intermediate) (300 mg, 1.50 mmol) with methylamine (33% in ethanol, 2.0 mL) as per the procedure described in step 2 of Intermediate D8 to yield 290 mg of the compound. ¹H NMR (400 MHz, DMSO-d₆) δ 2.64 (d, J=4.8 Hz, 3H), 4.95 (s, 2H), 7.06 (d, J=2.4 Hz, 1H), 8.01 (d, J=2.4 Hz, 1H), 8.20 (br s, 1H).

### Step 3: 2-(3-Amino-1H-pyrazol-1-yl)-N-methylacetamide

A solution of N-methyl-2-(3-nitro-1H-pyrazol-1-yl)acetamide (step 2 intermediate) (280 mg, 1.52 mmol) in methanol (25 mL) was subjected to hydrogenation in the presence of palladium on carbon as catalyst under 35 psi of hydrogen pressure at RT for 3 h. The mixture was filtered and the filtrate was concentrated under reduced pressure to yield 152 mg of the desired compound. ¹H NMR (400 MHz, DMSO-d₆) δ 2.60 (d, J=4.8 Hz, 3H), 4.43 (s, 2H), 4.60 (br s, 2H), 5.40 (d, J=2.0 Hz, 1H), 7.31 (d, J=2.0 Hz, 1H), 7.72 (br s, 1H).

### Intermediate D27

2-(3-Amino-1H-pyrazol-1-yl)-1-morpholinoethanone

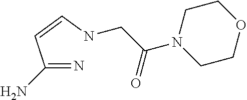 [SMILES: N=C(N)/C=C\NCC(=O)N1CCOCC1]

Step 1: 1-Morpholino-2-(3-nitro-1H-pyrazol-1-yl)ethanone

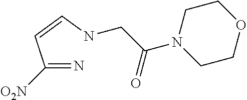 [SMILES: N=C(/C=C\NCC(=O)N1CCOCC1)[N+](=O)[O-]]

A mixture of ethyl 2-(3-nitro-1H-pyrazol-1-yl)acetate (500 mg, 2.51 mmol) and morpholine (0.5 mL) in 1,4-dioxane (3.0 mL) was heated in a sealed tube at 100° C. for 1 h. The mixture was cooled to RT and partitioned between ethyl acetate and water. The organic layer was dried over anhydrous sodium sulfate. The solution was filtered, concentrated and the residue obtained was purified by silica gel column chromatography to yield 300 mg of the desired compound. ¹H NMR (400 MHz, DMSO-d₆) δ 3.36-3.67 (m, 8H), 5.37 (s, 2H), 7.07 (d, J=2.4 Hz, 1H), 7.95 (d, J=2.4 Hz, 1H).

Step 2: 2-(3-Amino-1H-pyrazol-1-yl)-1-morpholinoethanone

A solution of 1-morpholino-2-(3-nitro-1H-pyrazol-1-yl)ethanone (step 1 intermediate) (300 mg, 1.25 mmol) in methanol (25 mL) was subjected to hydrogenation in the presence of palladium on carbon (10% w/w, wet) as catalyst under 35 psi of hydrogen pressure at RT for 4 h. The mixture was filtered and the filtrate was concentrated under reduced pressure to yield 215 mg of the desired compound. ¹H NMR (400 MHz, DMSO-d₆) δ 3.35-3.60 (m, 8H), 4.51-4.55 (m, 2H), 4.77-4.82 (m, 2H), 5.40-5.42 (m, 1H), 7.26 (d, J=2.4 Hz, 1H), 8.03 (s, 1H).

### Intermediate D30

2-(3-Amino-1H-pyrazol-1-yl)-2-methylpropan-1-ol

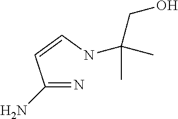

Step 1: Ethyl 2-methyl-2-(3-nitro-1H-pyrazol-1-yl)propanoate

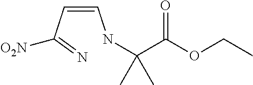 [SMILES: CCOC(=O)C(C)(C)N/C=C\C(=N)[N+](=O)[O-]]

To a solution of 3-nitro-1H-pyrazole (2.0 g, 17.6 mmol) in DMF (20 mL) were added potassium carbonate (4.86 g, 35.1 mmol) followed by ethyl 2-bromoisobutyrate (3.95 mL, 26.4 mmol) and the mixture was stirred at 80° C. for 5 h. The mixture was diluted with ethyl acetate and the organic mixture was washed with water followed by brine. The organic layer was dried over anhydrous sodium sulfate, filtered and concentrated to give 2.5 g of the desired compound. ESI-MS (m/z) 228 (M+H)⁺.

Step 2: 2-(3-Amino-1H-pyrazol-1-yl)-2-methylpropan-1-ol

The titled compound was prepared by the reaction of ethyl 2-methyl-2-(3-nitro-1H-pyrazol-1-yl)propanoate (2.5 g, 11.02 mmol) with lithium aluminum hydride (1.09 g, 28.6 mmol) in THF (30 mL) as per the procedure described in step 2 of Intermediate D24 to yield 1.2 g of the compound. The crude compound as such used for the next step.

### Intermediate D31

6,7-Dihydro-4H-pyrazolo[5,1-c][1,4]oxazin-2-amine

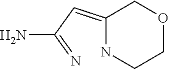

### Step 1: (3-Nitro-1H-pyrazol-5-yl)methanol

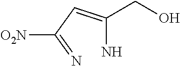

To a solution of 3-nitro-1H-pyrazole-5-carboxylic acid (3.0 g, 19.09 mmol) in THF (30 mL) was added borane-THF complex (1M, 57.2 mL, 57.2 mmol) at 0° C. and the mixture was stirred at 0° C-RT for 3 h. The mixture was quenched with saturated aqueous NaHCO3 and extracted twice with ethyl acetate. The combined organic layers were washed with brine, dried (Na₂SO₄), and concentrated. The resulting material was purified by silica gel column chromatography to yield 2.1 g of the desired compound. ESI-MS (m/z) 144 (M+H)⁺.

Step 2: (1-(2-Bromoethyl)-3-nitro-1H-pyrazol-5-yl)methanol

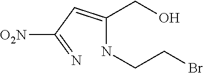 [SMILES: N=C(/C=C(/CO)NCCBr)[N+](=O)[O-]]

To a solution of (3-nitro-1H-pyrazol-5-yl)methanol (step 1 intermediate) (2.0 g, 13.9 mmol) in DMF (20 mL) were added cesium carbonate (5.5 g, 17.0 mmol) followed by dibromoethane (1.44 mL, 16.7 mmol) and the mixture was stirred at 80° C. for 5 h. The mixture was diluted with ethyl acetate and the organic mixture was washed with water followed by brine. The organic layer was dried over anhydrous sodium sulfate, filtered and concentrated to give 2.5 g of the desired compound. ¹H NMR (400 MHz, DMSO-d₆) δ 3.99-4.06 (m, 2H), 4.62-4.68 (m, 4H), 5.38-5.41 (m, 1H), 6.99 (s, 1H).

Step 3: 2-Nitro-6,7-dihydro-4H-pyrazolo[5,1-c][1,4]oxazine

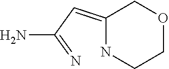

A mixture of (1-(2-bromoethyl)-3-nitro-1H-pyrazol-5-yl)methanol (step 2 intermediate) (500 mg, 2.00 mmol) and NMP (2.0 mL) was heated at 150° C. for 16 h. The mixture was cooled to RT and the mixture was partitioned between water and ethyl acetate. The organic layer was washed with water followed by brine. The organic layer was dried over anhydrous sodium sulfate, filtered and concentrated to give 160 mg of the desired compound. ¹H NMR (400 MHz, DMSO-d₆) δ 3.29-3.32 (m, 4H), 4.83 (s, 2H), 6.88 (s, 1H).

Step 4: 6,7-Dihydro-4H-pyrazolo[5,1-c][1,4]oxazin-2-amine

A solution of 2-nitro-6,7-dihydro-4H-pyrazolo[5,1-c][1,4]oxazine (step 3 intermediate) (150 mg, 0.88 mmol) in methanol (20 mL) was subjected to hydrogenation in the presence of palladium on carbon (10% w/w, wet) as catalyst under 35 psi of hydrogen pressure at RT for 4 h. The mixture was filtered and the filtrate was concentrated under reduced pressure to yield 100 mg of the desired compound. ESI-MS (m/z) 140 (M+H)⁺.

### Intermediate D32

### 2-Amino-5-methyl-4,5-dihydropyrazolo[1,5-a]pyrazin-6(7H)-one

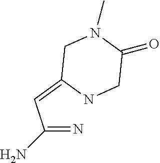

### Step 1: (5-Nitro-1H-pyrazol-3-yl)methanol

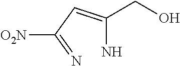

The titled compound was prepared by the reaction of 5-nitro-1H-pyrazole-3-carboxylic acid (5.0 g, 31.8 mmol) with borane-THF complex (1M, 95 mL, 95 mmol) in THF (75 mL) as per the procedure described in step 1 of Intermediate D31 to yield 4.5 g of the compound. ¹H NMR (400 MHz, DMSO-d₆) δ 4.53 (s, 2H), 5.63 (br s, 1H), 6.87 (s, 1H), 13.90 (s, 1H).

Step 2: Ethyl 2-(5-(hydroxymethyl)-3-nitro-1H-pyrazol-1-yl)acetate

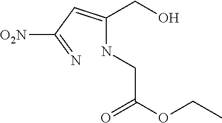 [SMILES: CCOC(=O)CN/C(=C\C(=N)[N+](=O)[O-])CO]

The titled compound was prepared by the reaction of (5-nitro-1H-pyrazol-3-yl)methanol (step 1 intermediate) (4.3 g, 30.04 mmol) with ethyl bromoacetate (4.15 mL, 35.9 mmol) in the presence of cesium carbonate (17.7 g, 36.0 mmol) in acetonitrile (100 mL) as per the procedure described in step 2 of D31 to give 3.2 g of the desired compound. ¹H NMR (400 MHz, DMSO-d₆) δ 1.21 (d, J=7.2 Hz, 3H), 3.64 (q, J=7.2 Hz, 2H), 4.14-4.21 (m, 2H), 5.25 (s, 2H), 5.63 (s, 1H), 6.99 (s, 1H).

Step 3: Ethyl 2-(5-(chloromethyl)-3-nitro-1H-pyrazol-1-yl)acetate

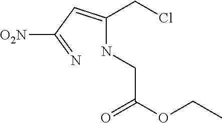 [SMILES: CCOC(=O)CN/C(=C\C(=N)[N+](=O)[O-])CCl]

To a cooled (0° C.) solution of ethyl 2-(5-(hydroxymethyl)-3-nitro-1H-pyrazol-1-yl)acetate (step 2 intermediate) (3.1 g, 13.5 mmol) in chloroform (30 mL) was added thionyl chloride (2.9 mL, 40.5 mmol) by maintaining the temperature 0-5° C. The mixture was warmed to 50° C. and stirred for 3 h. The mixture was cooled to 0° C. and quenched with water. The organic layer was separated and concentrated under reduced pressure. The residue thus obtained was purified by silica gel column chromatography to yield 400 mg of the desired compound. ¹H NMR (400 MHz, DMSO-d₆) δ 1.21 (t, J=7.2 Hz, 3H), 4.18 (q, J=7.2 Hz, 2H), 4.98 (s, 2H), 5.35 (s, 2H), 7.22 (s, 1H).

Step 4: 5-Methyl-2-nitro-4,5-dihydropyrazolo[1,5-a]pyrazin-6(7H)-one

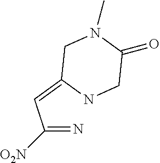

To a solution of ethyl 2-(5-(chloromethyl)-3-nitro-1H-pyrazol-1-yl)acetate (step 3 intermediate) (400 mg, 1.61 mmol) in a mixture of THF (5.0 mL) and dichloromethane (10 mL) was added methylamine (33% in ethanol, 450 g, 4.84 mmol) and the mixture was stirred for 48 h at RT. The mixture was partitioned between water and dichloromethane. The organic layer was separated. The solution was concentrated under vacuum and purified by silica gel column chromatography to yield 150 mg of the desired compound. ¹H NMR (400 MHz, DMSO-d₆) δ 3.01 (s, 3H), 4.66 (s, 2H), 4.90 (s, 2H), 7.03 (s, 1H).

Step 4: 2-Amino-5-methyl-4,5-dihydropyrazolo[1,5-a]pyrazin-6(7H)-one

A solution of 5-methyl-2-nitro-4,5-dihydropyrazolo[1,5-a]pyrazin-6(7H)-one (step 3 intermediate) (150 mg, 0.76 mmol) in methanol (10 mL) was subjected to hydrogenation in the presence of palladium on carbon (10% w/w, wet) as catalyst under 35 psi of hydrogen pressure at RT for 3 h. The mixture was filtered and the filtrate was concentrated under reduced pressure to yield 70 mg of the desired compound. ¹H NMR (400 MHz, DMSO-d₆) δ 2.96 (s, 3H), 4.46 (d, J=9.2 Hz, 4H), 4.68 (s, 2H), 5.33 (s, 1H); ESI-MS (m/z) 167 (M+H)⁺.

### Intermediate D34

2-Amino-5-isopropyl-4,5-dihydropyrazolo[1,5-a]pyrazin-6(7H)-one

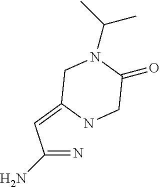

Step 1: Ethyl 2-(5-(bromomethyl)-3-nitro-1H-pyrazol-1-yl)acetate

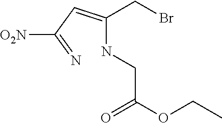 [SMILES: CCOC(=O)CN/C(=C\C(=N)[N+](=O)[O-])CBr]

To a solution of ethyl 2-(5-(hydroxymethyl)-3-nitro-1H-pyrazol-1-yl)acetate (step 2 of Intermediate D32) (2.0 g, 8.72 mmol) in chloroform (20 mL) was slowly added a solution of phosphorous tribromide (2.36 g, 8.72 mmol) in chloroform (10 mL) at 0° C. The mixture was stirred at 0-5° C. for 1 h. The mixture was diluted with dichloromethane and basified with sodium bicarbonate solution. The layers was separated and the aqueous layer was extracted twice with dichloromethane. The combined organic layers were dried (Na₂SO₄), filtered and concentrated. The residue was purified by silica gel column chromatography to yield 1.0 g of the desired compound. ESI-MS (m/z) 293 (M+H)⁺.

Step 2: Ethyl 2-(5-((isopropylamino)methyl)-3-nitro-1H-pyrazol-1-yl)acetate

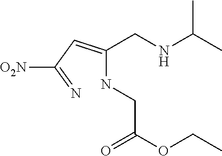 [SMILES: CCOC(=O)CN/C(=C\C(=N)[N+](=O)[O-])CNC(C)C]

A mixture of ethyl 2-(5-(bromomethyl)-3-nitro-1H-pyrazol-1-yl)acetate (step 1 intermediate) (1.0 g, 3.42 mmol) and isopropyl amine (0.44 mL, 5.07 mmol) in dichloromethane (10 mL) was stirred at RT for 3 h. The mixture was concentrated under vacuum and dried well to yield 600 mg of the desired compound. ESI-MS (m/z) 271 (M+H)⁺.

Step 3: 5-Isopropyl-2-nitro-4,5-dihydropyrazolo[1,5-a]pyrazin-6(7H)-one

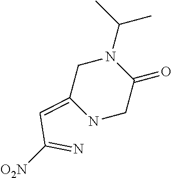 [SMILES: CC(C)N1C/C(=C/C(=N)[N+](=O)[O-])NCC1=O]

A mixture of ethyl 2-(5-((isopropylamino)methyl)-3-nitro-1H-pyrazol-1-yl)acetate (step 2 intermediate) (600 mg, 2.22 mmol) and methanol (10 mL) was stirred at 50° C. for 15 h. The mixture was concentrated under vacuum. The residue was diluted with water and extracted with ethyl acetate. The organic layer was dried (Na₂SO₄), filtered and concentrated. The residue was purified by silica gel column chromatography to yield 300 mg of the desired compound. ¹H NMR (400 MHz, DMSO-d₆) δ 1.17 (d, J=6.8 Hz, 6H), 4.57 (s, 2H), 4.74-4.82 (m, 1H), 5.22 (s, 2H), 6.99 (s, 1H).

Step 4: 2-Amino-5-isopropyl-4,5-dihydropyrazolo[1,5-a]pyrazin-6(7H)-one

A solution of 5-isopropyl-2-nitro-4,5-dihydropyrazolo[1,5-a]pyrazin-6(7H)-one (step 3 intermediate) (300 mg, 1.33 mmol) in methanol (5.0 mL) and THF (5.0 mL) was subjected to hydrogenation in the presence of palladium on carbon as catalyst under 35 psi of hydrogen pressure at RT for 3 h. The mixture was filtered and the filtrate was concentrated under reduced pressure to yield 200 mg of the desired compound. ¹H NMR (400 MHz, DMSO-d₆) δ 1.13 (d, J=6.8 Hz, 6H), 4.37 (s, 2H), 4.44 (s, 2H), 4.68 (s, 1H), 4.73-4.78 (m, 2H), 5.37 (s, 1H); ESI-MS (m/z) 195 (M+H)⁺.

### Intermediate D35

2-(3-Amino-1-methyl-1H-pyrazol-5-yl)propan-2-ol

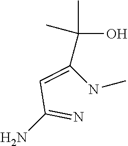

Step 1: 3-(2,5-Dimethyl-1H-pyrrol-1-yl)-1-methyl-1H-pyrazole

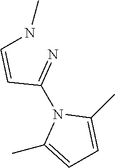

To a mixture of 1-methyl-1H-pyrazol-3-amine (5.0 g, 51.5 mmol) and 2,5-hexanedione (6.0 mL, 51.4 mmol) in toluene (100 mL) was added acetic acid (0.92 mL, 0.92 mmol) and the mixture was stirred at 130-140° C. for 16 h. The mixture was cooled to RT and concentrated under vacuum. The residue was purified by column chromatography to yield 6.5 g of the desired compound. ¹H NMR (400 MHz, CDCl₃) δ 2.13 (s, 6H), 3.94 (s, 3H), 5.87 (s, 2H), 6.18 (d, J=2.4 Hz, 1H), 7.41 (d, J=2.4 Hz, 1H); ESI-MS (m/z) 176 (M+H)⁺.

Step 2: 2-(3-(2,5-Dimethyl-1H-pyrrol-1-yl)-1-methyl-1H-pyrazol-5-yl)propan-2-ol

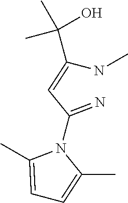

At −78° C., to a solution of 3-(2,5-dimethyl-1H-pyrrol-1-yl)-1-methyl-1H-pyrazole (step 1 intermediate) (2.0 g, 11.4 mmol) was added n-butyl lithium (1.6 M, 10.55 mL, 16.8 mmol) and the mixture was continued to stir at −78° C. for 30 min. The mixture was stirred for 2 h at 0° C. and added acetone (1.28 mL, 17.5 mmol) to the solution. The mixture was stirred at RT for 3 h. The mixture was quenched with water and extracted with ethyl acetate. The organic phase was washed with brine and dried over anhydrous sodium sulfate. The solution was filtered, concentrated and the residue obtained was purified by column chromatography to yield 1.0 g of the desired compound. ¹H NMR (400 MHz, DMSO-d₆) δ 1.54 (s, 6H), 2.04 (s, 6H), 3.98 (s, 3H), 5.42 (s, 1H), 5.73 (s, 2H), 6.10 (s, 1H).

Step 3: 2-(3-Amino-1-methyl-1H-pyrazol-5-yl)propan-2-ol

To a stirred solution of hydroxylamine HCl (1.8 g, 26.1 mmol) in ethanol (25 mL) were added a solution of potassium hydroxide (740 mg, 13.2 mmol) in water (25 mL) followed by a solution of 2-(3-(2,5-Dimethyl-1H-pyrrol-1-yl)-1-methyl-1H-pyrazol-5-yl)propan-2-ol (step 2 intermediate) (1.0 g, 4.28 mmol) in ethanol (25 mL). The mixture was stirred at RT for 24 h and then for 6 h at 80° C. The mixture was cooled to RT, quenched with water and extracted thrice with ethyl acetate. The combined organic extracts were washed with brine and dried over anhydrous sodium sulfate. The solution was filtered, concentrated and the residue obtained was purified by column chromatography to yield 300 mg of the desired compound. ¹H NMR (400 MHz, CDCl₃) δ 1.56 (s, 6H), 3.46 (br s, 2H), 3.82 (s, 3H), 5.38 (s, 1H); ESI-MS (m/z) 156 (M+H)⁺.

### Intermediate D47

1-(Difluoromethyl)-5-methyl-1H-pyrazol-3-amine

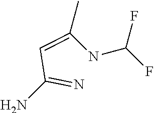

Step 1: 5-Methyl-3-nitro-1H-pyrazole

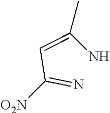

To a solution of 3-amino-5-methylpyrazole (5.0 g, 51.4 mmol) in water (250 mL) was portion wise added oxone (39.6 g, 128 mmol) at 0° C. and the mixture was stirred at 0° C. to RT for 18 h. The mixture was extracted twice with ethyl acetate. The combined organic extracts were washed with water and dried over anhydrous sodium sulfate. The solvent was removed under reduced pressure to yield 1.43 g of the desired compound. ¹H NMR (400 MHz, DMSO-d₆) δ 2.30 (s, 3H), 6.78 (s, 1H).

Step 2: 1-(Difluoromethyl)-5-methyl-3-nitro-1H-pyrazole

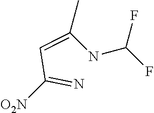

To a solution of 5-methyl-3-nitro-1H-pyrazole (step 1 intermediate) (300 mg, 2.36 mmol) in DMF (10 mL) was added sodium 2-chloro-2,2-difluoroacetate (1.4 g, 9.2 mmol) and potassium carbonate (358 mg, 2.59 mmol) followed by TBAB (87 mg, 0.23 mmol) and the mixture was stirred overnight at 110° C. The mixture was cooled to RT and extracted twice with ethyl acetate. The combined organic extracts were washed with water and dried over anhydrous sodium sulfate. The solvent was removed under reduced pressure to yield 109 mg of the desired compound. ¹H NMR (400 MHz, DMSO-d₆) δ 2.51 (s, 3H), 7.10 (s, 1H), 8.01 (s, 1H).

Sep 3: 1-(Difluoromethyl)-5-methyl-1H-pyrazol-3-amine

A solution of 1-(difluoromethyl)-5-methyl-3-nitro-1H-pyrazole (step 2 intermediate) (100 mg, 0.56 mmol) in ethyl acetate (10 mL) was subjected to hydrogenation in the presence of palladium on carbon as catalyst under 35 psi of hydrogen pressure at RT for 3 h. The mixture was filtered and the filtrate was concentrated under reduced pressure to yield 96 mg of the desired compound. ESI-MS (m/z) 148 (M+H)⁺.

The analytical data of the intermediates prepared by following the procedure described above are given in below Table 13.

TABLE 13
Analytical data of amine Intermediate D50

| Intermediate No. | Structure | Name and Analytical data |
|---|---|---|
| D50 | 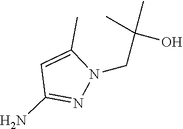 | 1-(3-Amino-5-methyl-1H-pyrazol-1- yl)-2-methylpropan-2-ol; ¹H NMR (400 MHz, DMSO-d₆) δ 1.03 (s, 6H), 2.10 (s, 3H), 3.51-3.56 (m, 2H), 4.47 (br s, 2H), 4.94 (s, 1H), 5.24 (s, 1H). |

### Intermediate D54

### 1-((3-Amino-1H-pyrazol-1-yl)methyl)cyclopropanol

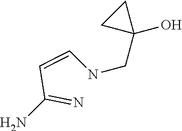 [SMILES: N=C(N)/C=C\NCC1(O)CC1]

Step 1: Methyl 1-((tetrahydro-2H-pyran-2-yl)oxy)cyclopropanecarboxylate

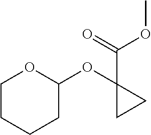

To a solution of methyl 1-hydroxycyclopropanecarboxylate (1.0 g, 8.62 mmol) and 3,4-dihydropyrane (0.86 mL, 9.42 mmol) in dichloromethane (20 mL) was added pyridinium-p-toluene sulfonic acid (216 mg, 0.86 mmol) at RT. The mixture was stirred overnight at RT. The solvent was removed under vacuum and the residue was purified by flash column chromatography to yield 990 mg of the desired product. ¹H NMR (400 MHz, CDCl₃) δ 1.30-1.32 (m, 2H), 1.54-1.58 (m, 6H), 1.82-1.85 (m, 2H), 3.47-3.53 (m, 1H), 3.74 (s, 3H), 3.83-3.89 (s, 1H), 4.89-4.91 (m, 1H).

### Step 2: (1-((Tetrahydro-2H-pyran-2-yl)oxy)cyclopropyl)methanol

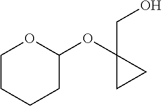 [SMILES: OCC1(OC2CCCCO2)CC1]

To a suspension of lithium aluminum hydride (360 mg, 9.47 mmol) in dry THF (20 mL) was added a solution of methyl 1-((tetrahydro-2H-pyran-2-yl)oxy)cyclopropanecarboxylate (step 1 intermediate) (950 mg, 4.70 mmol) in THF (20 mL) drop-wise at 0° C. and the stirred at RT for 18 h. The mixture was quenched with saturated sodium sulfate solution and stirred for 30 min. The suspension was filtered and the filtration bed was washed with ethyl acetate. The combined filtrates were concentrated under reduced pressure. The residue was purified by flash column chromatography to yield 816 mg of the desired product. ¹H NMR (400 MHz, DMSO-d₆) δ 0.49-0.60 (m, 2H), 0.63-0.68 (m, 1H), 0.81-0.86 (m, 1H), 1.36-1.49 (m, 4H), 1.54-1.60 (m, 1H), 1.66-1.70 (m, 1H), 3.40-3.48 (m, 2H), 3.55-3.59 (m, 1H), 3.78-3.83 (m, 1H), 4.55 (t, J=4.8 Hz, 1H), 4.78-4.80 (m, 1H).

### Step 3: 3-Nitro-1-((1-((tetrahydro-2H-pyran-2-yl)oxy)cyclopropyl)methyl)-1H-pyrazole

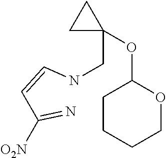 [SMILES: N=C(/C=C\NCC1(OC2CCCCO2)CC1)[N+](=O)[O-]]

The titled compound was prepared by the reaction of 3-nitro-1H-pyrazole (525 mg, 4.60 mmol) with (1-((tetrahydro-2H-pyran-2-yl)oxy)cyclopropyl)methanol (step 2 intermediate) (800 mg 4.60 mmol) in the presence of triphenylphosphine (1.3 g, 5.12 mmol) and DIAD (1.4 mL, 6.90 mmol) in THF (20 mL) as per the procedure described in step 1 of Intermediate D10 to yield 1.62 g of the desired compound. The crude compound was as such taken for the next step

### Step 4: 14(3-Nitro-1H-pyrazol-1-yl)methyl)cyclopropanol

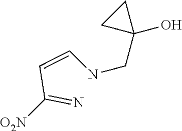 [SMILES: N=C(/C=C\NCC1(O)CC1)[N+](=O)[O-]]

To a solution of 3-nitro-1-((1-((tetrahydro-2H-pyran-2-yl)oxy)cyclopropyl)methyl)-1H-pyrazole (step 3 intermediate) (1.6 g, 5.66 mmol) in ethanol (50 mL) was added PTSA (284 mg, 1.49 mmol) and the mixture was stirred at RT for 4 h. The solvent was removed under reduced pressure and the residue was purified by silica gel column chromatography to yield 461 mg of the desired compound. ¹H NMR (400 MHz, DMSO-d₆) δ 0.67-0.71 (m, 2H), 0.74-0.77 (m, 2H), 4.28 (s, 2H), 5.65 (s, 1H), 7.07 (d, J=2.8 Hz, 1H), 8.01 (d, J=2.8 Hz, 1H).

### Step 5: 1-((3-Amino-1H-pyrazol-1-yl)methyl)cyclopropanol

A solution of 1-((3-nitro-1H-pyrazol-1-yl)methyl)cyclopropanol (step 4 intermediate) (150 mg, 0.82 mmol) in ethyl acetate (10 mL) was subjected to hydrogenation in the presence of palladium on carbon as catalyst under 35 psi of hydrogen pressure at RT for 3 h. The mixture was filtered and the filtrate was concentrated under reduced pressure to yield 96 mg of the desired compound. ¹H NMR (400 MHz, DMSO-d₆) δ 0.54-0.62 (m, 4H), 3.89 (s, 2H), 4.57 (br s, 2H), 5.38-5.41 (m, 2H), 7.31 (d, J=2.8 Hz, 1H); ESI-MS (m/z) 154 (M+H)⁺.

The analytical data of the intermediates prepared by following the procedure described above are given in below Table 14.

TABLE 14
Analytical data of amine Intermediate D55

| Intermediate No. | Structure | Name and Analytical data |
|---|---|---|
| D55 | 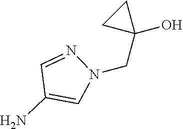 [SMILES: Nc1cnn(CC2(O)CC2)c1] | 1-((4-Amino-1H-pyrazol-1- yl)methyl)cyclopropanol; ¹H NMR (400 MHz, DMSO-d₆) δ 0.54-0.61 (m, 4H), 3.81-3.82 (m, 2H), 3.98 (s, 2H), 5.43 (s, 1H), 6.87 (s, 1H); 7.08 (s, 1H) ESI-MS (m/z) 154 (M + H)⁺. |

### Intermediate D56

1-(Ethylsulfonyl)-5-methyl-1H-pyrazol-3-amine

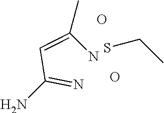

Step 1: 1-(Ethylsulfonyl)-5-methyl-3-nitro-1H-pyrazole

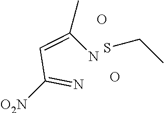

To a solution of 5-methyl-3-nitro-1H-pyrazole (250 mg, 1.96 mmol) in dichloromethane (10 mL) were added ethyl sulfonyl chloride (0.21 mL, 2.16 mmol), triethylamine (0.30 mL, 2.16 mmol) at 0° C. The resultant mixture was stirred at RT for 3 h. The mixture was diluted with ethyl acetate and washed with water followed by brine. The organic layer was dried over anhydrous sodium sulfate. The solution was filtered, concentrated under reduced pressure and the residue obtained was purified by silica gel column chromatography to yield 375 mg of the desired product. ¹H NMR (400 MHz, DMSO-d₆) δ 1.20 (t, J=7.6 Hz, 3H), 2.57 (s, 3H), 3.90 (q, J=7.6 Hz, 2H), 7.17 (s, 1H).

Step 2: 1-(Ethylsulfonyl)-5-methyl-1H-pyrazol-3-amine

A solution of 1-(ethylsulfonyl)-5-methyl-3-nitro-1H-pyrazole (step 1 intermediate) (360 mg, 1.64 mmol) in ethyl acetate (10 mL) was subjected to hydrogenation in the presence of palladium on carbon as catalyst under 35 psi of hydrogen pressure at RT for 3 h. The mixture was filtered and the filtrate was concentrated under reduced pressure to yield 292 mg of the desired compound. ¹H NMR (400 MHz, DMSO-d₆) δ 1.04 (d, J=7.6 Hz, 3H), 2.50 (s, 3H), 3.49 (q, J=4.4 Hz, 2H), 4.48 (s, 2H), 7.35 (s, 1H).

### EXAMPLES

### General Procedures:

### Method G

Synthesis of 5-(2-fluoro-6-methoxyphenyl)-3-((methylamino)(phenyl)methylene)indolin-2-one (Example 1)

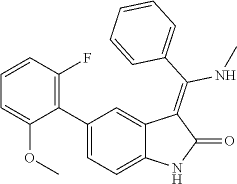 [SMILES: CN/C(=C1\C(=O)Nc2ccc(-c3c(F)cccc3OC)cc21)c1ccccc1]

Step 1: 1-(2-Chloroacetyl)-5-(2-fluoro-6-methoxyphenyl)indolin-2-one

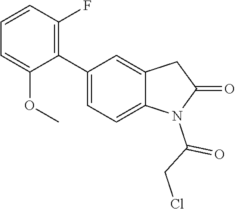 [SMILES: COc1cccc(F)c1-c1ccc2c(c1)CC(=O)N2C(=O)CCl]

A mixture of 5-(2-fluoro-6-methoxyphenyl)indolin-2-one (Intermediate B2) (900 mg, 3.58 mmol) and chloroacetyl chloride (10 mL) was refluxed at 110° C. for 1 h. The mixture was cooled to RT and diluted with hexane. The mixture was stirred for 1 h at RT. The solid was filtered, washed with hexane and dried to afford 915 mg of the desired compound. ¹H NMR (400 MHz, DMSO-d₆) δ 3.74 (s, 3H), 3.89 (s, 2H), 5.00 (s, 2H), 6.88-6.95 (m, 1H), 6.98 (d, J=8.8 Hz, 1H), 7.28-7.33 (m, 2H), 7.35-7.43 (m, 1H), 8.42 (dd, J=9.2, 1.2 Hz, 1H).

Step 2: (Z)-1-(2-chloroacetyl)-5-(2-fluoro-6-methoxyphenyl)-3-(methoxy(phenyl)methylene)indolin-2-one

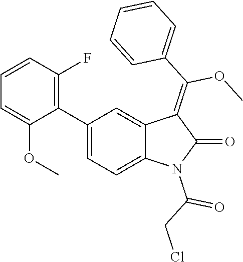 [SMILES: CO/C(=C1\C(=O)N(C(=O)CCl)c2ccc(-c3c(F)cccc3OC)cc21)c1ccccc1]

To a solution of 1-(2-chloroacetyl)-5-(2-fluoro-6-methoxyphenyl)indolin-2-one (step 1 intermediate) (900 mg, 2.69 mmol) in toluene (2.0 mL) was added acetic anhydride (884 μL, 9.43 mmol) at RT and the mixture was heated to 150° C. Trimethyl orthobenzoate (1.15 mL, 6.74 mmol) was added to the mixture drop wise over a period of 40 min, and then heated at 150° C. for 16 h. The reaction mixture was cooled to RT and diluted with hexane. The solid was filtered, washed with hexane and dried to afford 261 mg of the desired compound.

¹H NMR (400 MHz, DMSO-d₆) δ 3.68 (s, 3H), 3.77 (s, 3H), 4.87 (s, 2H), 6.90-6.99 (m, 1H), 7.00 (d, J=8.8 Hz, 1H), 7.26 (dd, J=8.4, 1.2 Hz, 1H), 7.37-7.45 (m, 2H), 7.46-7.51 (m, 2H), 7.54-7.59 (m, 2H), 7.90 (s, 1H), 8.20 (d, J=8.4 Hz, 1H).

Step 3: (Z)-5-(2-Fluoro-6-methoxyphenyl)-3-(methoxy(phenyl)methylene)indolin-2-one

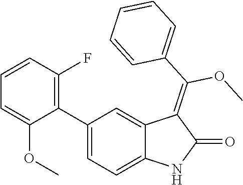 [SMILES: CO/C(=C1\C(=O)Nc2ccc(-c3c(F)cccc3OC)cc21)c1ccccc1]

To a stirred suspension of 1-(2-chloroacetyl)-5-(2-fluoro-6-methoxyphenyl)-3-(methoxy(phenyl)methylene)indolin-2-one (step 2 intermediate) (250 mg, 0.55 mmol) in methanol (2.5 mL) was added potassium hydroxide (9.0 mg, 0.17 mmol) and the mixture was heated at 63° C. for 1 h. The mixture was cooled to RT and then to 0° C., filtered the solid and washed with methanol to yield 140 mg of the desired compound.

¹H NMR (400 MHz, DMSO-d₆) δ 3.57 (s, 3H), 3.76 (s, 3H), 6.83 (d, J=8.0 Hz, 1H), 6.90 (t, J=8.4 Hz, 1H), 6.97 (d, J=8.4 Hz, 1H), 7.05 (d, J=7.6 Hz, 1H), 7.36 (q, J=7.2 Hz, 1H), 7.42-7.53 (m, 5H), 7.67 (s, 1H), 10.23 (s, 1H).

Step 4: 5-(2-Fluoro-6-methoxyphenyl)-3-((methylamino)(phenyl)methylene)indolin-2-one

To a solution of 5-(2-fluoro-6-methoxyphenyl)-3-(methoxy(phenyl)methylene)indolin-2-one (step 3 intermediate) (80 mg, 0.21 mmol) in methanol (1.0 mL) and DMF (0.2 mL) was added a 2M solution of methylamine (44 mg, 0.42 mmol) in THF. The reaction mixture was heated at 65° C. for 2 h. The mixture was concentrated under reduced pressure and the residue was purified by silica gel column chromatography to yield 40 mg of the desired product. ¹H NMR (400 MHz, DMSO-d₆) δ 2.76 (d, J=5.2 Hz, 3H), 3.61 (s, 3H), 5.58 (s, 1H), 6.70-6.80 (m, 4H), 7.16-7.24 (m, 1H), 7.39-7.43 (m, 2H), 7.49-7.61 (m, 3H), 10.08 (q, J=5.2 Hz, 1H), 10.44 (s, 1H).

### Method H

Synthesis of (Z)-5-(2-fluoro-6-methoxyphenyl)-3-(1-((4-(4-methylpiperazin-1-yl)phenyl)amino)ethylidene)-1H-pyrrolo[2,3-c]pyridin-2(3H)-one (Example 14)

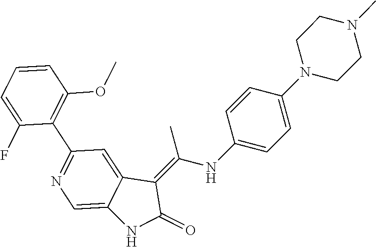 [SMILES: COc1cccc(F)c1-c1cc2c(cn1)NC(=O)/C2=C(/C)Nc1ccc(N2CCN(C)CC2)cc1]

Step 1: (Z)-3-(1-Ethoxyethylidene)-5-(2-fluoro-6-methoxyphenyl)-1H-pyrrolo[2,3-c]pyridin-2(3H)-one

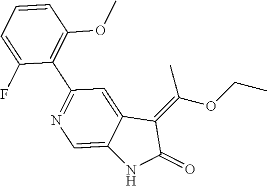 [SMILES: CCO/C(C)=C1\C(=O)Nc2cnc(-c3c(F)cccc3OC)cc21]

A mixture of 5-(2-fluoro-6-methoxyphenyl)-1H-pyrrolo[2,3-c]pyridin-2(3H)-one (Intermediate B7) (90 mg, 0.35 mmol) and triethyl orthoacetate (2.0 mL) was heated at 130° C. for 1 h. The reaction mixture was concentrated under reduced pressure to afford 52 mg of the desired compound. ¹H NMR (400 MHz, DMSO-d₆) δ 1.34 (t, J=6.8 Hz, 3H), 2.78 (s, 3H), 3.73 (s, 3H), 4.40 (q, J=6.8 Hz, 2H), 6.88 (t, J=8.8 Hz, 1H), 6.96 (d, J=8.4 Hz, 1H), 7.34-7.42 (m, 1H), 7.51 (s, 1H), 8.12 (s, 1H), 10.49 (s, 1H).

Step 2: (Z)-5-(2-fluoro-6-methoxyphenyl)-3-(1-((4-(4-methylpiperazin-1-yl)phenyl)amino)ethylidene)-1H-pyrrolo[2,3-c]pyridin-2(3H)-one

To a solution of (Z)-3-(1-ethoxyethylidene)-5-(2-fluoro-6-methoxyphenyl)-1H-pyrrolo[2,3-c]pyridin-2(3H)-one (step 1 intermediate) (50 mg, 0.15 mmol) in methanol (1.0 mL) was added a 4-(4-methylpiperazin-1-yl)aniline (29 mg, 0.15 mmol). The reaction mixture was heated at 70° C. for 1 h. The mixture was concentrated under reduced pressure and the residue was purified by silica gel column chromatography to yield 35 mg of the desired product. ¹H NMR (400 MHz, DMSO-d₆) δ 2.22 (s, 3H), 2.44-2.46 (m, 7H), 3.18 (t, J=3.6 Hz, 4H), 3.71 (s, 3H), 6.86 (t, J=8.8 Hz, 2H), 6.94 (d, J=8.8 Hz, 2H), 7.01 (d, J=9.2 Hz, 1H), 7. 18 (d, J=9.2 Hz, 1H), 7.26 (s, 1H), 7.37 (q, J=7.2 Hz, 1H), 8.16 (s, 1H), 10.78 (s, 1H), 12.31 (s, 1H); ESI-MS (m/z) 474 (M+H)⁺

### Method I

Synthesis of (Z)-5-(4-methoxypyridin-3-yl)-3-(1-((1-methyl-1H-pyrazol-4-yl)amino)ethylidene)-1H-pyrrolo[2,3-c]pyridin-2(3H)-one (Example 33)

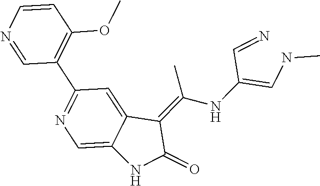 [SMILES: CN/C=C(\C=N)N/C(C)=C1\C(=O)Nc2cnc(-c3cnccc3OC)cc21]

Step 1: (Z)-5-Chloro-3-(1-ethoxyethylidene)-1H-pyrrolo[2,3-c]pyridin-2(3H)-one

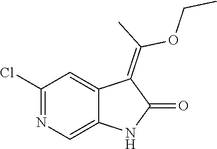 [SMILES: CCO/C(C)=C1\C(=O)Nc2cnc(Cl)cc21]

A mixture of 5-chloro-1H-pyrrolo[2,3-c]pyridin-2(3H)-one (Step 3 of Intermediate B7) (750 mg, 4.44 mmol) and triethyl orthoacetate (10 mL) was heated at 130° C. for 1 h. The reaction mixture was concentrated under reduced pressure and the solid was stirred with diethyl ether. The compound was filtered and dried to yield 800 mg of the desired compound. ¹H NMR (400 MHz, DMSO-d₆) δ 1.44 (t, J=7.2 Hz, 3H), 2.78 (s, 3H), 4.45 (q, J=7.2 Hz, 2H), 7.41 (s, 1H), 7.82 (s, 1H), 10.58 (s, 1H).

Step 2: (Z)-5-Chloro-3-(1-((1-methyl-1H-pyrazol-4-yl)amino)ethylidene)-1H-pyrrolo[2,3-c]pyridin-2(3H)-one

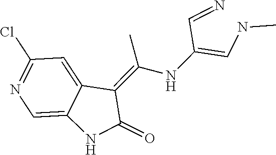 [SMILES: CN/C=C(\C=N)N/C(C)=C1\C(=O)Nc2cnc(Cl)cc21]

To a solution of (Z)-5-chloro-3-(1-ethoxyethylidene)-1H-pyrrolo[2,3-c]pyridin-2(3H)-one (step 1 intermediate) (800 mg, 3.35 mmol) in methanol (10 mL) was added a 1-methyl-1H-pyrazol-4-amine (488 mg, 5.02 mmol). The reaction mixture was stirred at RT for 3 h. The mixture was filtered; the solid was washed with methanol followed by diethyl ether and dried well to yield 740 mg of the desired product. ¹H NMR (400 MHz, DMSO-d₆) δ 2.51 (s, 3H), 3.86 (s, 1H), 7.33 (s, 1H), 7.58 (s, 1H), 7.85 (s, 1H), 7.97 (s, 1H), 10.88 (s, 1H), 12.16 (s, 1H).

Step 3: (Z)-5-(4-Methoxypyridin-3-yl)-3-(1-((1-methyl-1H-pyrazol-4-yl)amino)ethylidene)-1H-pyrrolo[2,3-c]pyridin-2(3H)-one

To a degassed mixture of 1,4-dioxane (20 mL) and water (3.0 mL) were added (Z)-5-Chloro-3-(1-((1-methyl-1H-pyrazol-4-yl)amino)ethylidene)-1H-pyrrolo[2,3-c]pyridin-2(3H)-one (step 2 intermediate) (100 mg, 0.34 mmol) and 3-methoxy-2-(4,4,5,5-tetramethyl-1,3,2-dioxaborolan-2-yl)pyridine (121 mg, 0.51 mmol) and the mixture was evacuated for 15 min. XPhos Pd G2 (27 mg, 0.03 mmol), XPhos (32 mg, 0.07 mmol) and potassium acetate (84 mg, 0.85 mmol) were added to the mixture. The resulting reaction mixture was heated on a pre-heated oil bath at 180° C. for 5 h. The mixture was cooled to RT and concentrated under reduced pressure. The crude material was purified by silica gel column chromatography to yield 25 mg of the desired compound. ¹H NMR (400 MHz, DMSO-d₆) δ 2.50 (s, 3H), 3.86 (s, 3H), 3.90 (s, 3H), 7.16 (d, J=5.6 Hz, 1H), 7.59 (s, 1H), 7.79 (s, 1H), 7.96 (s, 1H), 8.21 (s, 1H), 8.43 (d, J=5.2 Hz, 1H), 8.71 (s, 1H), 10.83 (s, 1H), 12.08 (s, 1H); ESI-MS (m/z) 363 (M+H)⁺. (*potassium phosphate can also be used in place of potassium acetate in same equivalent quantities)

### Method K

Synthesis of (Z)-7-(1-((1-methyl-1H-pyrazol-3-yl)amino)ethylidene)-2-(4-methylpyridin-3-yl)-5H-pyrrolo[3,2-d]pyrimidin-6(7H)-one (Example 217)

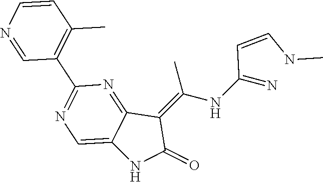 [SMILES: CN/C=C\C(=N)N/C(C)=C1\C(=O)Nc2cnc(-c3cnccc3C)nc21]

### Step 1: 2-Chloro-5H-pyrrolo[3,2-d]pyrimidine

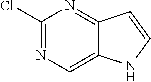

To a stirred solution of 2,4-dichloro-5H-pyrrolo[2,3-d]pyrimidine (1.0 g, 5.32 mmol) in methanol (25 mL) were added acetic acid (1.91 g, 31.8 mmol) followed by zinc powder (1.4 g, 21.3 mmol) and the mixture was heated to 90° C. for 3 h. The mixture was filtered and the filtrate was concentrated. The residue was diluted with water and the precipitated solid was collected through filtration. The solid was dried well to yield 640 mg of the desired compound. ¹H NMR (400 MHz, DMSO-d₆) δ 6.61 (d, J=3.2 Hz, 1H), 8.03 (d, J=3.2 Hz, 1H), 8.84 (s, 1H), 12.09 (s, 1H).

Step 2: 7,7-Dibromo-2-chloro-5H-pyrrolo[3,2-d]pyrimidin-6(7H)-one

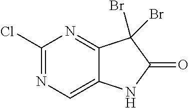 [SMILES: O=C1Nc2cnc(Cl)nc2C1(Br)Br]

To a stirred solution of 2-chloro-5H-pyrrolo[3,2-d]pyrimidine (step 1 intermediate) (1.0 g, 6.57 mmol) in tert-butanol (50 mL) was added pyridinium perbromide (6.29 g, 19.7 mmol) and the mixture was heated to 40° C. for 3 h. The mixture was diluted with water and extracted with ethyl acetate. The organic extract was washed with brine and dried over anhydrous sodium sulfate. The solution was filtered, concentrated and the residue thus obtained was purified by flash column chromatography to yield 227 mg of the desired compound. ¹H NMR (400 MHz, DMSO-d₆) δ 8.47 (s, 1H), 11.88 (s, 3H).

### Step 3: 2-Chloro-5H-pyrrolo[3,2-d]pyrimidin-6(7H)-one

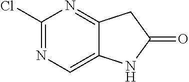 [SMILES: O=C1Cc2nc(Cl)ncc2N1]

To a stirred solution of 7,7-dibromo-2-chloro-5H-pyrrolo[3,2-d]pyrimidin-6(7H)-one (step 2 intermediate) (650 mg, 1.98 mmol) in THF (10 mL) was added zinc powder (1.29 g, 19.8 mmol) followed by an aqueous solution of ammonium chloride (1.0 mL) and the mixture was stirred at 100° C. for 48 h. The mixture was filtered and concentrated. The residue was diluted with ethyl acetate and water. The organic layer was separated, washed with water and brine. The solution was dried over anhydrous sodium sulfate, filtered and concentrated under reduced pressure. The residue was purified by column chromatography to yield 54 mg of the desired compound. ¹H NMR (400 MHz, DMSO-d₆) δ 3.74 (s, 2H), 8.11 (s, 1H), 3.90 (s, 1H), 12.71 (br s, 1H); ESI-MS (m/z) 170 (M+H)⁺.

Step 4: (Z)-2-Chloro-7-(1-ethoxyethylidene)-5H-pyrrolo[3,2-d]pyrimidin-6(7H)-one

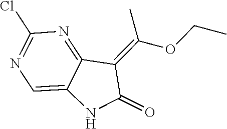 [SMILES: CCO/C(C)=C1\C(=O)Nc2cnc(Cl)nc21]

A mixture of 2-chloro-5H-pyrrolo[3,2-d]pyrimidin-6(7H)-one (Step 3 intermediate) (100 mg, 0.59 mmol) and triethyl orthoacetate (0.4 mL) was heated at 70° C. for 1 h. The reaction mixture was concentrated under reduced pressure and the solid was stirred with diethyl ether. The compound was filtered and dried to yield 7.0 mg of the desired compound. ¹H NMR (400 MHz, DMSO-d₆) δ 1.39 (t, J=6.8 Hz, 3H), 2.85 (s, 3H), 4.50 (q, J=6.8 Hz, 2H), 7.99 (s, 1H), 10.66 (s, 1H).

Step 5: (Z)-2-Chloro-7-(1-((1-methyl-1H-pyrazol-3-yl)amino)ethylidene)-5H-pyrrolo[3,2-d]pyrimidin-6(7H)-one

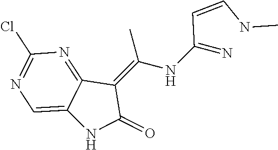 [SMILES: CN/C=C\C(=N)N/C(C)=C1\C(=O)Nc2cnc(Cl)nc21]

To a solution of (Z)-2-chloro-7-(1-ethoxyethylidene)-5H-pyrrolo[3,2-d]pyrimidin-6(7H)-one (step 4 intermediate) (50 mg, 0.21 mmol) in methanol (1.0 mL) was added 1-methyl-1H-pyrazol-4-amine (40 mg, 0.42 mmol). The reaction mixture was stirred at 90° C. for 5 h. The mixture was filtered; the solid was washed with methanol followed by diethyl ether and dried well to yield 39 mg of the desired product. ¹H NMR (400 MHz, DMSO-d₆) δ 3.17 (s, 3H), 3.84 (s, 3H), 6.40 (s, 1H), 7.81 (s, 1H), 8.00 (s, 1H), 11.05 (s, 1H), 12.74 (s, 1H); ESI-MS (m/z) 291 (M+H)⁺.

Step 6: (Z)-7-(1-((1-Methyl-1H-pyrazol-3-yl)amino)ethylidene)-2-(4-methylpyridin-3-yl)-5H-pyrrolo[3,2-d]pyrimidin-6(7H)-one

To a degassed mixture of 1,4-dioxane (10 mL) and water (2.0 mL) were added (Z)-2-chloro-7-(1-((1-methyl-1H-pyrazol-3-yl)amino)ethylidene)-5H-pyrrolo[3,2-d]pyrimidin-6(7H)-one (step 5 intermediate) (35 mg, 0.12 mmol) and (4-methylpyridin-3-yl)boronic acid pinacol ester (53 mg, 0.24 mmol) and the mixture was evacuated for 15 min. XPhos Pd G2 (8.0 mg, 0.01 mmol) and potassium phosphate (76 mg, 0.36 mmol) were added to the mixture. The resulting reaction mixture was heated on a pre-heated oil bath at 180° C. for 5 h. The mixture was cooled to RT and concentrated under reduced pressure. The crude material was purified by silica gel column chromatography to yield 20 mg of the desired compound. ¹H NMR (400 MHz, DMSO-d₆) δ 2.58 (s, 3H), 3.04 (s, 3H), 3.84 (s, 3H), 6.39 (d, J=2.0 Hz, 1H), 7.33 (d, J=4.8 Hz, 1H), 7.80 (d, J=2.0 Hz, 1H), 8.29 (s, 1H), 8.44 (d, J=4.8 Hz, 1H), 8.93 (s, 1H), 10.99 (s, 1H), 12.65 (s, 1H); ESI-MS (m/z) 348 (M+H)⁺.

The details of synthesis and analytical data of the examples prepared from the above-mentioned methods are given below in Table 15.

TABLE 15
Structure, chemical name, method, intermediate used and analytical data of
the Example 3, 5-6, 9-22, 24-31, 33-51, 53-74, 76-161 and 163-281.

| | Structure and Chemical | Method and | |
|---|---|---|---|
| No. | Name | Intermediate | Analytical Data |
| 2 | 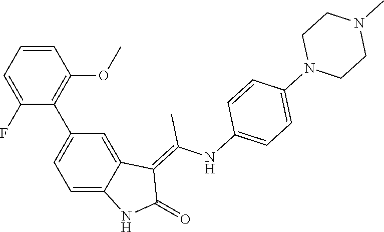 [SMILES: COc1cccc(F)c1-c1ccc2c(c1)/C(=C(\C)Nc1ccc(N3CCN(C)CC3)cc1)C(=O)N2] (Z)-5-(2-Fluoro-6- methoxyphenyl)-3-(1-((4- (4-methylpiperazin-1- yl)phenyl)amino)ethylidene) indolin-2-one | Intermediate B2 and triethylorthoacetate, 4-(4-methylpiperazin- 1-yl)aniline Method G | ¹H NMR (400 MHz, DMSO-d₆) δ 2.22 (s, 3H), 2.38 (s, 3H), 2.45 (d, J = 5.6 Hz, 4H), 3.16 (t, J = 5.6 Hz, 4H), 3.73 (s, 3H), 6.87-7.01 (m, 5H), 7.14 (d, J = 8.8 Hz, 2H), 7.25 (s, 2H), 7.31- 7.35 (m, 1H), 10.58 (s, 1H), 12.02 (s, 1H); ESI-MS (rn/z) 473 (M + H)⁺. |
| 3 | 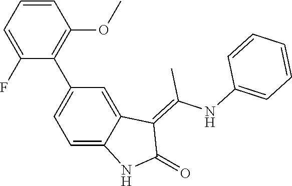 [SMILES: COc1cccc(F)c1-c1ccc2c(c1)/C(=C(\C)Nc1ccccc1)C(=O)N2] (Z)-5-(2-Fluoro-6- methoxyphenyl)-3-(1- (phenylamino)ethylidene) indolin-2-one | Intermediate B2 and triethylorthoacetate, aniline Method G | ¹H NMR (400 MHz, DMSO-d₆) δ 2.46 (s, 3H), 3.74 (s, 3H), 6.85-6.97 (m, 4H), 7.24-7.37 (m, 5H), 7.42-7.48 (m, 2H), 10.65 (s, 1H), 12.20 (s, 1H); ESI-MS (m/z) 375 (M + H)⁺. |
| 4 | 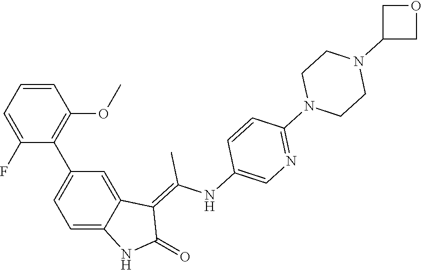 [SMILES: COc1cccc(F)c1-c1ccc2c(c1)/C(=C(\C)Nc1ccc(N3CCN(C4COC4)CC3)nc1)C(=O)N2] (Z)-5-(2-Fluoro-6- methoxyphenyl)-3-(1-((6- (4-(oxetan-3-yl)piperazin- 1-yl)pyridin-3- yl)amino)ethylidene)indolin- 2-one | Intermediate B2 and triethylorthoacetate, 6-(4-(oxetan-3- yl)piperazin-1- yl)pyridin-3-amine (D5) Method G | ¹H NMR (400 MHz, DMSO-d₆) δ 2.33-2.37 (m, 7H), 3.36-3.47 (m, 1H), 3.51-3.56 (m, 4H), 3.73 (s, 3H), 4.45-4.49 (m, 2H), 4.54-4.59 (m, 2H), 6.84- 6.96 (m, 4H), 7.25 (s, 1H), 7.29-7.37 (m, 2H), 7.51- 7.56 (m, 1H), 8.09 (d, J = 2.8 Hz, 1H), 10.62 (s, 1H), 11.87 (s, 1H); ESI-MS (m/z) 516 (M + H)⁺. |
| 5 | 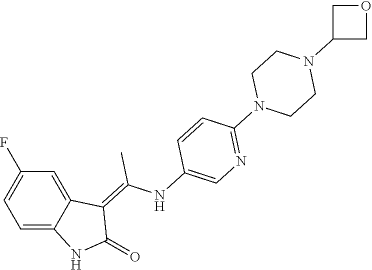 [SMILES: C/C(Nc1ccc(N2CCN(C3COC3)CC2)nc1)=C1/C(=O)Nc2ccc(F)cc21] (Z)-5-Fluoro-3-(1-((6-(4- (oxetan-3-yl)piperazin-1- yl)pyridin-3- yl)amino)ethylidene)indolin- 2-one | 5-fluoroindolin-2- one, triethylorthoacetate, 6-(4-(oxetan-3- yl)piperazin-1- yl)pyridin-3-amine (D5) Method G | ¹H NMR (400 MHz, DMSO-d₆) δ 2.35-2.38 (m, 7H), 3.45 (m, 1H), 3.54 (t, J = 4.8 Hz, 4H), 4.48 (t, J = 6.0 Hz, 2H), 4.57 (t, J = 6.4 Hz, 2H), 6.75-6.80 (m, 2H), 6.92 (d, J = 9.2 Hz, 1H), 7.15 (dd, J = 10.0 Hz, 2.4 Hz, 1H), 7.55 (dd, J = 8.8 Hz, 2.8 Hz, 1H), 8.09 (s, 1H), 10.55 (s, 1H), 11.96 (s, 1H); ESI-MS (m/z) 410 (M + H)⁺. |
| 6 | 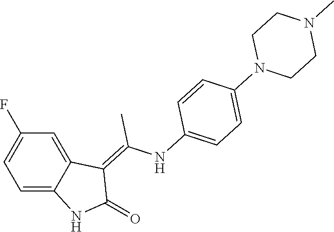 [SMILES: C/C(Nc1ccc(N2CCN(C)CC2)cc1)=C1/C(=O)Nc2ccc(F)cc21] (Z)-5-Fluoro-3-(1-((4-(4- methylpiperazin-1- yl)phenyl)amino)ethylidene) indolin-2-one | 5-fluoroindolin-2- one, triethylorthoacetate, 4-(4-methylpiperazin- 1-yl)aniline Method G | ¹H NMR (400 MHz, DMSO-d₆) δ 2.23 (s, 3H), 2.49-2.51 (m, 7H), 3.17 (t, J = 4.8 Hz, 4H), 6.77 (t, J = 7.2 Hz, 1H), 6.83 (dd, J = 8.8 Hz, 4.8 Hz, 1H), 7.00 (d, J = 8.8 Hz, 2H), 7.12-7.15 (m, 3H), 10.52 (s, 1H), 12.11 (s, 1H); ESI-MS (m/z) 367 (M + H)⁺. |
| 7 | 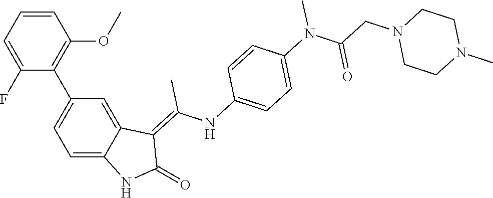 [SMILES: COc1cccc(F)c1-c1ccc2c(c1)/C(=C(\C)Nc1ccc(N(C)C(=O)CN3CCN(C)CC3)cc1)C(=O)N2] (Z)-N-(4-((1-(5-(2-Fluoro- 6-methoxyphenyl)-2- oxoindolin-3- ylidene)ethyl)amino)phenyl)- N-methyl-2-(4- methylpiperazin-1- yl)acetamide | Intermediate B2 and triethylorthoacetate, N-(4-aminophenyl)- N-methyl-2-(4- methylpiperazin-1- yl)acetamide Method G | ¹H NMR (400 MHz, DMSO-d₆) δ 2.28-2.44 (m, 14H), 3.33-3.39 (m, 5H), 3.74 (s, 3H), 6.89 (t, J = 9.2 Hz, 2H), 6.95 (d, J = 9.2 Hz, 2H), 7.29-7.41 (m, 6H), 10.68 (s, 1H), 12.21 (s, 1H); ESI-MS (m/z) 544 (M + H)⁺. |
| 8 | 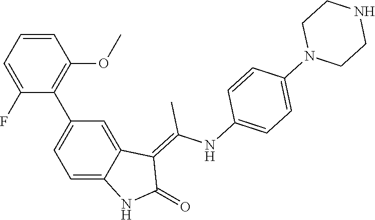 [SMILES: COc1cccc(F)c1-c1ccc2c(c1)/C(=C(\C)Nc1ccc(N3CCNCC3)cc1)C(=O)N2] (Z)-5-(2-Fluoro-6- methoxyphenyl)-3-(1-((4- (piperazin-1- yl)phenyl)amino)ethylidene) indolin-2-one | Intermediate B2 and triethylorthoacetate, N-(4-aminophenyl)- N-methyl-2-(4- methylpiperazin-1- yl)acetamide Method G followed by Boc deprotection using PTSA | ¹H NMR (400 MHz, DMSO-d₆) δ 2.49 (s, 3H), 3.22-3.39 (m, 8H), 3.73 (s, 3H), 6.88 (t, J = 8.8 Hz, 6H), 7.06 (d, J = 8.8 Hz, 1H), 7.21 (d, J = 8.2 Hz, 1H), 7.25 (s, 1H), 7.34 (q, J = 7.2 Hz, 1H), 8.88 (s, 1H), 10.61 (s, 1H), 12.05 (s, 1H); ESI-MS (m/z) 459 (M + H)⁺. |
| 9 | 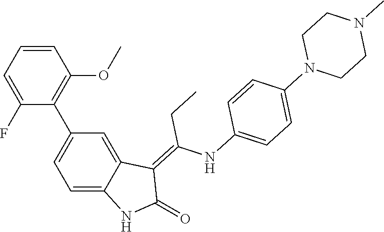 [SMILES: CC/C(Nc1ccc(N2CCN(C)CC2)cc1)=C1/C(=O)Nc2ccc(-c3c(F)cccc3OC)cc21] (Z)-5-(2-Fluoro-6- methoxyphenyl)-3-(1-((4- (4-methylpiperazin-1- yl)phenyl)amino)propylidene) indolin-2-one | Intermediate B2 and triethylorthopropionate, 4-(4- methylpiperazin-1- yl)aniline Method G | ¹H NMR (400 MHz, DMSO-d₆) δ 1.20 (t, J = 7.6 Hz, 3H), 2.46 (t, J = 4.8 Hz, 3H), 2.47-2.51 (m, 4H), 2.64-2.68 (m, 2H), 3.17 (t, J = 4.8 Hz, 4H), 3.73 (s, 1H), 6.86-6.90 (m, 4H), 6.94 (d, J = 5.2 Hz, 1H), 7.16 (d, J = 8.8 Hz, 1H), 7.20 (s, 1H), 7.29-7.35 (m, 2H), 7.96 (s, 1H), 10.60 (s, 1H), 11.94 (s, 1H); ESI-MS (m/z) 487 (M + H)⁺. |
| 10 | 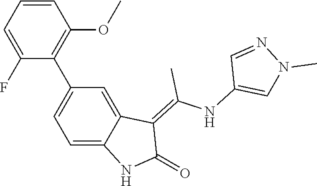 [SMILES: COc1cccc(F)c1-c1ccc2c(c1)/C(=C(\C)Nc1cnn(C)c1)C(=O)N2] (Z)-5-(2-Fluoro-6- methoxyphenyl)-3-(1-((1- methyl-1H-pyrazol-4- yl)amino)ethylidene)indolin- 2-one | Intermediate B2 and triethylorthoacetate, 1-methyl-1H-pyrazol- 4-amine Method G | ¹H NMR (400 MHz, DMSO-d₆) δ 2.39 (s, 3H), 3.73 (s, 3H), 3.84 (s, 3H), 6.88 (t, J = 9.2 Hz, 2H), 6.95 (s, 2H), 7.27 (s, 1H), 7.33 (q, J = 6.8 Hz, 1H), 7.52 (s, 1H), 7.88 (s, 1H), 10.59 (s, 1H), 11.75 (s, 1H); ESI-MS (m/z) 379 (M + H)⁺. |
| 11 | 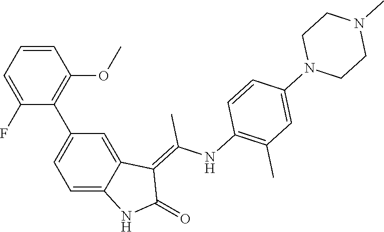 [SMILES: COc1cccc(F)c1-c1ccc2c(c1)/C(=C(\C)Nc1ccc(N3CCN(C)CC3)cc1C)C(=O)N2] (Z)-5-(2-Fluoro-6- methoxyphenyl)-3-(1-((2- methyl-4-(4- methylpiperazin-1- yl)phenyl)amino)ethylidene) indolin-2-one | Intermediate B2 and triethylorthoacetate, 2-methyl-4-(4- methylpiperazin-1- yl)aniline (D4) Method G | ¹H NMR (400 MHz, DMSO-d₆) δ 2.21 (d, J = 8.0 Hz, 9H), 2.44 (t, J = 4.4 Hz, 4H), 3.15 (t, J = 4.8 Hz, 4H), 3.73 (s, 3H), 6.81-6.95 (m, 6H), 7.08 (d, J = 8.8 Hz, 1H), 7.29 (s, 1H), 7.34 (q, J = 1.6 Hz, 1H), 10.55 (s, 1H), 11.86 (s, 1H); ESI-MS (m/z) 487 (M + H)⁺. |
| 12 | 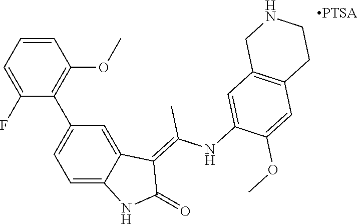 [SMILES: COc1cc2c(cc1N/C(C)=C1\C(=O)Nc3ccc(-c4c(F)cccc4OC)cc31)CNCC2] (Z)-5-(2-Fluoro-6- methoxyphenyl)-3-(1-((6- methoxy-1,2,3,4- tetrahydroisoquinolin-7- yl)amino)ethylidene)indolin- 2-one. PTSA salt | Intermediate B2, triethylorthoacetate, tert-butyl 7-amino-6- methoxy-3,4- dihydroisoquinoline- 2(1H)-carboxylate Method G followed by Step 3-Method D | ¹H NMR (400 MHz, DMSO-d₆) δ 2.29 (s, 3H), 2.37 (s, 3H), 3.01 (br t, 2H), 3.41-3.46 (m, 2H), 3.74 (s, 3H), 3.84 (s, 3H), 4.23 (br t, 2H), 6.88 (t, J = 9.2 Hz, 1H), 6.94-6.96 (m, 4H), 7.02 (s, 1H), 7.11 (d, J = 8.0 Hz, 1H), 7.20 (s, 1H), 7.27 (s, 1H), 7.34 (q, J = 7.2 Hz, 1H), 7.47 (d, J = 8.0 Hz, 2H), 8.95 (br s, 2H), 10.60 (s, 1H), 11.90 (s, 1H); ESI- MS (m/z) 460 (M + H)⁺. |
| 13 | 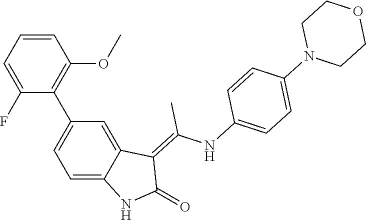 [SMILES: COc1cccc(F)c1-c1ccc2c(c1)/C(=C(\C)Nc1ccc(N3CCOCC3)cc1)C(=O)N2] (Z)-5-(2-Fluoro-6- methoxyphenyl)-3-(1-((4- morpholinophenyl)amino) ethylidene)indolin-2-one | Intermediate B2, triethylorthoacetate, 4-morpholinoaniline Method G | ¹H NMR (400 MHz, DMSO-d₆) δ 2.39 (s, 3H), 3.13 (t, J = 4.8 Hz, 4H), 3.74 (t, J = 4.4 Hz, 7H), 6.85 (t, J = 8.4 Hz, 1H), 6.90-6.95 (m, 4H), 6.99 (d, J = 8.8 Hz, 2H), 7.16 (d, J = 8.8 Hz, 1H), 7.25 (s, 1H), 7.34 (dt, J = 6.8 Hz, 1H), 10.59 (s, 1H), 12.03 (s, 1H); ESI-MS (m/z) 460 (M + H)⁺. |
| 15 | 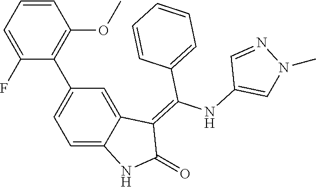 [SMILES: COc1cccc(F)c1-c1ccc2c(c1)/C(=C(/Nc1cnn(C)c1)c1ccccc1)C(=O)N2] (Z)-5-(2-Fluoro-6- methoxyphenyl)-3-(((1- methyl-1H-pyrazol-4- yl)amino)(phenyl)methylene) indolin-2-one | Intermediate B2, trimethyl orthobenzoate, 1- methyl-1H-pyrazol-4- amine Method G | ¹H NMR (400 MHz, DMSO-d₆) δ 3.62 (s, 3H), 3.63 (s, 3H), 5.73 (s, 1H), 6.75-6.88 (m, 5H), 7.21 (q, J = 6.8 Hz, 2H), 7.43 (dd, J = 7.6 Hz, 2.4 Hz, 2H), 7.51- 7.55 (m, 3H), 10.71 (s, 1H), 11.68 (s, 1H); ESI-MS (m/z) 441 (M + H)⁺. |
| 16 | 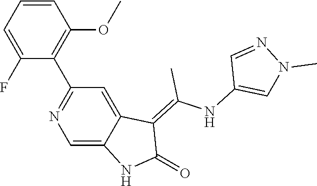 [SMILES: COc1cccc(F)c1-c1cc2c(cn1)NC(=O)/C2=C(/C)Nc1cnn(C)c1] (Z)-5-(2-Fluoro-6- methoxyphenyl)-3-(1-((1- methyl-1H-pyrazol-4- yl)amino)ethylidene)-1H- pyrrolo[2,3-c]pyridin- 2(3H)-one | Intermediate B7, 1- methyl-1H-pyrazol-4- amine Method H | ¹H NMR (400 MHz, DMSO-d₆) δ 2.45 (s, 3H), 3.71 (s, 3H), 3.85 (s, 3H), 6.86 (t, J = 8.8 Hz, 1H), 6.94 (d, J = 8.4 Hz, 1H), 7.29 (s, 1H), 7.38 (q, J = 6.8 Hz, 1H), 7.57 (s, 1H), 7.95 (s, 1H), 8.16 (s, 1H), 10.80 (s, 1H), 12.09 (s, 1H); ESI- MS (m/z) 380 (M + H)⁺. |
| 17 | 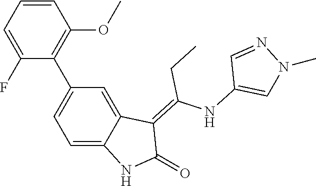 [SMILES: CC/C(Nc1cnn(C)c1)=C1/C(=O)Nc2ccc(-c3c(F)cccc3OC)cc21] (Z)-5-(2-Fluoro-6- methoxyphenyl)-3-(1-((1- methyl-1H-pyrazol-4- yl)amino)propylidene)indolin- 2-one | Intermediate B2 and triethylorthopropionate, 1-methyl-1H- pyrazol-4-amine Method G | ¹H NMR (400 MHz, DMSO-d₆) δ 1.21 (t, J = 7.6 Hz, 3H), 2.68 (q, J = 7.6 Hz, 2H), 3.73 (s, 3H), 3.85 (s, 3H), 6.86-6.97 (m, 4H), 7.22 (s, 1H), 7.32 (q, J = 7.2 Hz, 1H), 7.53 (s, 1H), 7.92 (s, 1H), 10.61 (s, 1H), 11.63 (s, 1H). |
| 18 | 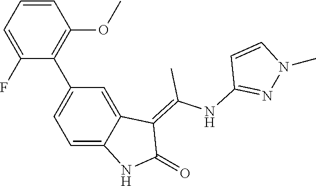 [SMILES: COc1cccc(F)c1-c1ccc2c(c1)/C(=C(\C)Nc1ccn(C)n1)C(=O)N2] (Z)-5-(2-Fluoro-6- methoxyphenyl)-3-(1-((1- methyl-1H-pyrazol-3- yl)amino)ethylidene)indolin- 2-one | Intermediate B2, triethyl orthoacetate, 1-methyl-1H-pyrazol- 3-amine Method G | ¹H NMR (400 MHz, DMSO-d₆) δ 2.58 (s, 3H), 3.74 (s, 3H), 3.80 (s, 3H), 6.20 (d, J = 2.4 Hz, 1H), 6.88 (t, J = 5.2 Hz, 2H), 6.95 (d, J = 4.8 Hz, 2H), 7.31-7.34 (m, 2H), 7.72 (s, 1H), 10.65 (s, 1H), 12.28 (s, 1H); ESI-MS (m/z) 379 (M+H)⁺. |
| 19 | 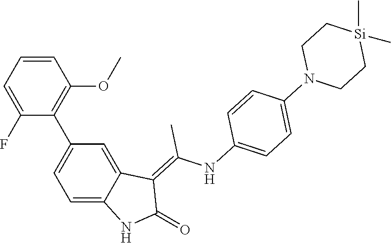 [SMILES: COc1cccc(F)c1-c1ccc2c(c1)/C(=C(\C)Nc1ccc(N3CC[Si](C)(C)CC3)cc1)C(=O)N2] (Z)-3-((3,5-Dimethyl-4-(1- methyl-1H-pyrazol-4-yl)- 1H-pyrrol-2-yl)methylene)- 5-(2-fluoro-6- methoxyphenyl)indolin-2- one | Intermediate B2, triethyl orthoacetate, 4-(4,4-dimethyl-1,4- azasilinan-1- yl)aniline (D3) Method G | ¹H NMR (400 MHz, DMSO-d₆) δ 0.08 (s, 6H), 0.73 (t, J = 6.0 Hz, 4H), 2.37 (s, 3H), 3.66 (t, J = 6.0 Hz, 4H), 3.73 (s, 3H), 6.85- 6.95 (m, 6H), 7.09 (d, J = 8.8 Hz, 2H), 7.30 (s, 1H), 7.32 (q, J = 6.8 Hz, 1H), 10.55 (s, 1H), 12.00 (s, 1H); ESI-MS (m/z) 501 (M + H)⁺. |
| 20 | 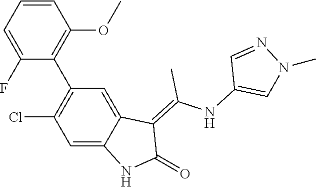 [SMILES: COc1cccc(F)c1-c1cc2c(cc1Cl)NC(=O)/C2=C(/C)Nc1cnn(C)c1] (Z)-3-(1-((4-(4,4-Dimethyl- 1,4-azasilinan-1- yl)phenyl)amino)ethylidene)- 5-(2-fluoro-6- methoxyphenyl)indolin-2- one | Intermediate B5 triethylorthoacetate, 1-methyl-1H-pyrazol- 4-amine Method G | ¹H NMR (400 MHz, DMSO-d₆) δ 2.51 (s, 3H), 3.72 (s, 3H), 3.64 (s, 3H), 6.88 (t, J = 8.8 Hz, 2H), 6.95 (d, J = 8.4 Hz, 1H), 7.19 (s, 1H), 7.40 (q, J = 1.2 Hz, 1H), 7.52 (s, 1H), 7.88 (s, 1H), 10.73 (s, 1H), 11.75 (s, 1H); ESI-MS (m/z) 413 (M + H)⁺. |
| 21 | 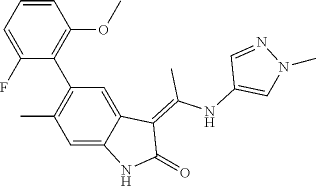 [SMILES: COc1cccc(F)c1-c1cc2c(cc1C)NC(=O)/C2=C(/C)Nc1cnn(C)c1] (Z)-5-(2-Fluoro-6- methoxyphenyl)-6-methyl- 3-(1-((1-methyl-1H- pyrazol-4- yl)amino)ethylidene)indolin- 2-one | Intermediate B6, triethylorthoacetate, 1-methyl-1H-pyrazol- 4-amine Method G | ¹H NMR (400 MHz, DMSO-d₆) δ 1.98 (s, 3H), 2.33 (s, 3H), 3.71 (s, 3H), 3.83 (s, 3H), 6.81 (s, 1H), 6.87 (t, J = 8.8 Hz, 1H), 6.94 (d, J = 8.4 Hz, 1H), 7.02 (s, 1H), 7.36 (q, J = 7.2 Hz, 1H), 7.49 (s, 1H), 7.85 (s, 1H), 10.51 (s, 1H), 11.63 (s, 1H); ESI-MS (m/z) 393 (M + H)⁺. |
| 22 | 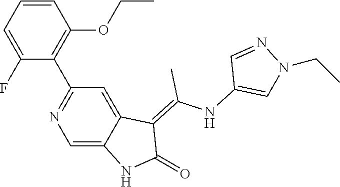 [SMILES: CCOc1cccc(F)c1-c1cc2c(cn1)NC(=O)/C2=C(/C)Nc1cnn(CC)c1] (Z)-5-(2-Ethoxy-6- fluorophenyl)-3-(1-((1- ethyl-1H-pyrazol-4- yl)amino)ethylidene)-1H- pyrrolo[2,3-c]pyridin- 2(3H)-one | Intermediate B8, triethylorthoacetate, 1-ethyl-1H-pyrazol- 4-amine Method G | ¹H NMR (400 MHz, DMSO-d₆) δ 1.17 (br s, 3H), 1.38 (br s, 3H), 2.48 (d, J = 12.8 Hz, 3H), 4.02 (br t, 2H), 4.13 (br t, 2H), 6.84-6.93 (m, 2H), 7.35 (s, 2H), 7.59 (s, 1H), 8.01 (s, 1H), 8.16 (s, 1H), 10.80 (s, 1H), 12.10 (s, 1H); ESI-MS (m/z) 408 (M + H)⁺. |
| 23 | 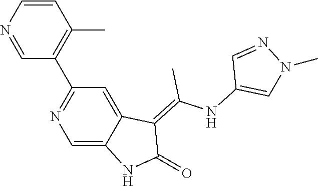 [SMILES: C/C(Nc1cnn(C)c1)=C1/C(=O)Nc2cnc(-c3cnccc3C)cc21] (Z)-3-(1-((1-methyl-1H- pyrazol-4- yl)amino)ethylidene)-5-(4- methylpyridin-3-yl)-1H- pyrrolo[2,3-c]pyridin- 2(3H)-one | Intermediate B9 and 1-methyl-1H-pyrazol- 4-amine Method H | ¹H NMR (400 MHz, DMSO-d₆) δ 2.32 (s, 3H), 2.51 (s, 3H), 3.86 (s, 3H), 7.30 (d, J = 5.2 Hz, 1H), 7.49 (s, 1H), 7.58 (s, 1H), 7.95 (s, 1H), 8.22 (s, 1H), 8.41 (d, J = 4.8 Hz, 1H), 8.56 (s, 1H), 10.85 (s, 1H), 12.13 (s, 1H); ESI-MS (m/z) 347 (M + H)⁺. |
| 24 | 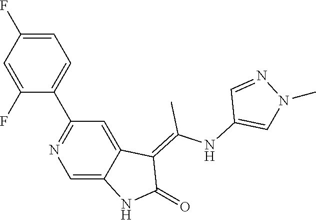 [SMILES: C/C(Nc1cnn(C)c1)=C1/C(=O)Nc2cnc(-c3ccc(F)cc3F)cc21] (Z)-5-(2,4-Difluorophenyl)- 3-(1-((1-methyl-1H- pyrazol-4- yl)amino)ethylidene)-1H- pyrrolo[2,3-c]pyridin- 2(3H)-one | Intermediate B10 and 1-methyl-1H-pyrazol- 4-amine Method H | ¹H NMR (400 MHz, DMSO-d₆) δ 2.57 (s, 3H), 3.86 (s, 3H), 7.14-7.21 (m, 1H), 7.28-7.36 (m, 1H), 7.59 (s, 1H), 7.68 (s, 1H), 7.89-7.97 (m, 2H), 8.23 (s, 1H), 10.87 (s, 1H), 12.12 (s, 1H); ESI-MS (m/z) 368 (M + H)⁺. |
| 25 | 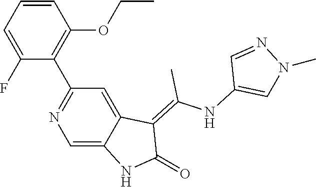 [SMILES: CCOc1cccc(F)c1-c1cc2c(cn1)NC(=O)/C2=C(/C)Nc1cnn(C)c1] (Z)-5-(2-Ethoxy-6- fluorophenyl)-3-(1-((1- methyl-1H-pyrazol-4- yl)amino)ethylidene)-1H- pyrrolo[2,3-c]pyridin- 2(3H)-one | Intermediate B8 and 1-methyl-1H-pyrazol- 4-amine Method H | ¹H NMR (400 MHz, DMSO-d₆) δ 1.18 (t, J = 6.8 Hz, 3H), 2.46 (s, 3H), 3.85 (s, 3H), 4.03 (q, J = 6.8 Hz, 2H), 6.84 (d, J = 8.8 Hz, 1H), 6.93 (d, J = 8.0 Hz, 1H), 7.30-7.38 (m, 2H), 7.58 (s, 1H), 7.95 (s, 1H), 8.16 (s, 1H), 10.79 (s, 1H), 12.07 (s, 1H); ESI-MS (m/z) 394 (M + H)⁺. |
| 26 | 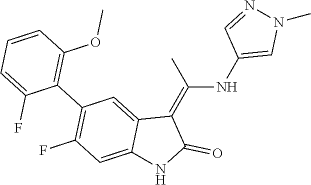 [SMILES: COc1cccc(F)c1-c1cc2c(cc1F)NC(=O)/C2=C(/C)Nc1cnn(C)c1] ((Z)-6-Fluoro-5-(2-fluoro- 6-methoxyphenyl)-3-(1- ((1-methyl-1H-pyrazol-4- yl)amino)ethylidene)indolin- 2-one | Intermediate B11, 1- (oxetan-3-yl)-1H- pyrazol-4-amine Method G | ¹H NMR (400 MHz, DMSO-d₆) δ 2.36 (s, 3H), 3.74 (s, 3H), 3.84 (s, 3H), 6.77 (d, J = 9.6 Hz, 1H), 6.90 (t, J = 8.4 Hz, 1H), 6.96 (d, J = 8.4 Hz, 1H), 7.19 (d, J = 6.8 Hz, 1H), 7.37-7.42 (m, 1H), 7.51 (s, 1H), 7.88 (s, 1H), 10.73 (s, 1H), 11.62 (s, 1H); ESI-MS (m/z) 397 (M + H)⁺. |
| 27 | 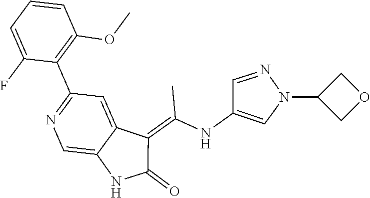 [SMILES: COc1cccc(F)c1-c1cc2c(cn1)NC(=O)/C2=C(/C)Nc1cnn(C2COC2)c1] (Z)-5-(2-Fluoro-6- methoxyphenyl)-3-(1-((1- (oxetan-3-yl)-1H-pyrazol- 4-yl)amino)ethylidene)-1H- pyrrolo[2,3-c]pyridin- 2(3H)-one | Intermediate B7, 1- (oxetan-3-yl)-1H- pyrazol-4-amine Method H | ¹H NMR (400 MHz, DMSO-d₆) δ 2.47 (s, 3H), 3.71 (s, 3H), 4.87-4.96 (m, 4H), 5.55-5.60 (m, 1H), 6.87 (t, J = 8.4 Hz, 1H), 6.94 (d, J = 8.8 Hz, 1H), 7.31 (s, 1H), 7.34-7.42 (m, 1H), 7.76 (s, 1H), 8.16 (s, 1H), 8.17 (s, 1H), 10.81 (s, 1H), 12.13 (s, 1H); ESI-MS (m/z) 421 (M + H)⁺. |
| 28 | 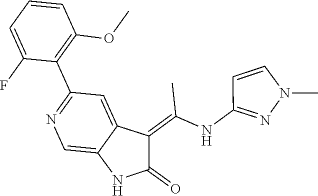 [SMILES: COc1cccc(F)c1-c1cc2c(cn1)NC(=O)/C2=C(/C)Nc1ccn(C)n1] (Z)-5-(2-Fluoro-6- methoxyphenyl)-3-(1-((1- methyl-1H-pyrazol-3- yl)amino)ethylidene)-1H- pyrrolo[2,3-c]pyridin- 2(3H)-one | Intermediate B7, 1-methyl-1H-pyrazol- 3-amine Method H | ¹H NMR (400 MHz, DMSO-d₆) δ 2.63 (s, 3H), 3.72 (s, 3H), 3.82 (s, 3H), 6.27 (d, J = 2.4 Hz, 1H), 6.87 (t, J = 8.4 Hz, 1H), 6.95 (d, J = 8.8 Hz, 1H), 7.33 (s, 1H), 7.32-7.42 (m, 1H), 7.76 (d, J = 2.0 Hz, 1H), 8.18 (s, 1H), 10.85 (s, 1H), 12.60 (s, 1H); ESI-MS (m/z) 380 (M + H)⁺. |
| 29 | 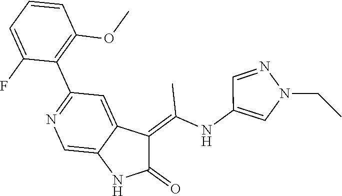 [SMILES: CCn1cc(N/C(C)=C2\C(=O)Nc3cnc(-c4c(F)cccc4OC)cc32)cn1] (Z)-3-(1-((1-Ethyl-1H- pyrazol-4- yl)amino)ethylidene)-5-(2- fluoro-6-methoxyphenyl)- 1H-pyrrolo[2,3-c]pyridin- 2(3H)-one | Intermediate B7, 1-ethyl-1H-pyrazol- 4-amine Method H | ¹H NMR (400 MHz, DMSO-d₆) δ 1.39 (t, J = 7.2 Hz, 3H), 2.46 (s, 3H), 3.72 (s, 3H), 4.14 (q, J = 7.2 Hz, 2H), 6.87 (d, J = 8.4 Hz, 1H), 6.94 (d, J = 8.4 Hz, 1H), 7.30 (s, 1H), 7.34-7.40 (m, 1H), 7.59 (s, 1H), 8.00 (s, 1H), 8.16 (s, 1H), 10.79 (s, 1H), 12.12 (s, 1H); ESI- MS (m/z) 394 (M + H)⁺. |
| 30 | 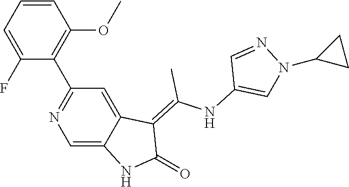 [SMILES: COc1cccc(F)c1-c1cc2c(cn1)NC(=O)/C2=C(/C)Nc1cnn(C2CC2)c1] (Z)-3-(1-((1-Cyclopropyl- 1H-pyrazol-4- yl)amino)ethylidene)-5-(2- fluoro-6-methoxyphenyl)- 1H-pyrrolo[2,3-c]pyridin- 2(3H)-one | Intermediate B7, 1-cyclopropyl-1H- pyrazol-4-amine (D6) Method H | ¹H NMR (400 MHz, DMSO-d₆) δ 0.98 (d, J = 5.6 Hz, 2H), 1.07 (d, J = 5.6 Hz, 2H), 2.45 (s, 3H), 3.72 (s, 3H), 3.73-3.76 (m, 1H), 6.86 (t, J = 8.8 Hz, 1H), 6.94 (d, J = 8.0 Hz, 1H), 7.30 (s, 1H), 7.36 (dd, J = 15.2, 7.2 Hz, 1H), 7.58 (s, 1H), 8.04 (s, 1H), 8.16 (s, 1H), 10.79 (s, 1H), 12.09 (s, 1H); ESI-MS (m/z) 406 (M + H)⁺. |
| 31 | 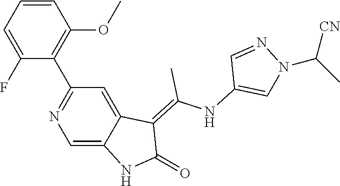 [SMILES: COc1cccc(F)c1-c1cc2c(cn1)NC(=O)/C2=C(/C)Nc1cnn(C(C)C#N)c1] (Z)-2-(4-((1-(5-(2-Fluoro- 6-methoxyphenyl)-2-oxo- 1H-pyrrolo[2,3-c]pyridin- 3(2H)- ylidene)ethyl)amino)-1H- pyrazol-1-yl)propanenitrile | Intermediate B7, 2-(4-amino-1H- pyrazol-1- yl)propanenitrile (D7) Method H | ¹H NMR (400 MHz, DMSO-d₆) δ 1.82 (d, J = 7.2 Hz, 3H), 2.48 (s, 3H), 2.72 (s, 3H), 5.83-5.90 (m, 1H), 6.87 (t, J = 8.8 Hz, 1H), 6.95 (d, J = 8.4 Hz, 1H), 7.33 (s, 1H), 7.35-7.43 (m, 1H), 7.81 (s, 1H), 8.18 (d, J = 7.2 Hz, 2H), 10.85 (s, 1H), 12.15 (s, 1H); ESI-MS (m/z) 419 (M + H)⁺. |
| 32 | 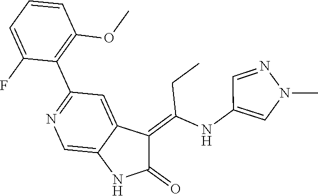 [SMILES: CC/C(Nc1cnn(C)c1)=C1/C(=O)Nc2cnc(-c3c(F)cccc3OC)cc21] (Z)-5-(2-Fluoro-6- methoxyphenyl)-3-(1-((1- methyl-1H-pyrazol-4- yl)amino)propylidene)-1H- pyrrolo[2,3-c]pyridin- 2(3H)-one | Triethylorthopropionate, 1-methyl-1H- pyrazol-4-amine Method I | ¹H NMR (400 MHz, DMSO-d₆) δ 1.20 (t, J = 7.2 Hz, 3H), 2.73 (q, J = 7.2 Hz, 2H), 3.72 (s, 3H), 3.86 (s, 3H), 6.87 (t, J = 8.4 Hz, 1H), 6.95 (d, J = 8.4 Hz, 1H), 7.23 (s, 1H), 7.34-7.42 (m, 1H), 7.58 (s, 1H), 7.98 (s, 1H), 8.17 (s, 1H), 10.83 (s, 1H), 11.96 (s, 1H); ESI- MS (m/z) 394 (M + H)⁺. |
| 34 | 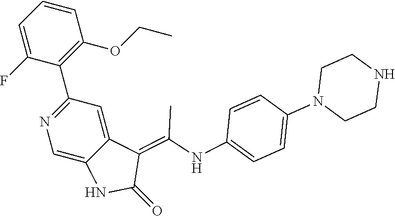 [SMILES: CCOc1cccc(F)c1-c1cc2c(cn1)NC(=O)/C2=C(/C)Nc1ccc(N2CCNCC2)cc1] (Z)-5-(2-Ethoxy-6- fluorophenyl)-3-(1-((4- (piperazin-1- yl)phenyl)amino)ethylidene)- 1H-pyrrolo[2,3-c]pyridin- 2(3H)-one | Step 1 of Method I, 4-(piperazin-1- yl)aniline and 2-(2-ethoxy-6- fluorophenyl)- 4,4,5,5-tetramethyl- 1,3,2-dioxaborolane Method I | ¹H NMR (400 MHz, DMSO-d₆) δ 1.18 (t, J = 6.8 Hz, 3H), 2.44 (s, 3H), 2.81- 2.85 (m, 4H), 3.06-3.10 (m, 4H), 4.04 (q, J = 6.8 Hz, 2H), 6.84 (t, J = 8.8 Hz, 1H), 6.93 (d, J = 8.4 Hz, 1H), 6.99 (d, J = 9.2 Hz, 2H), 7.31 (s, 2H), 7.32-7.38 (m, 3H), 8.16 (s, 1H), 10.78 (s, 1H), 12.30 (s, 1H); ESI- MS (m/z) 374 (M + H)⁺. |
| 35 | 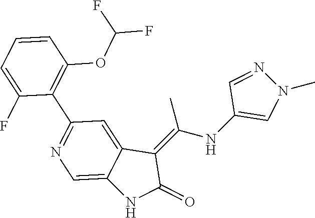 [SMILES: C/C(Nc1cnn(C)c1)=C1/C(=O)Nc2cnc(-c3c(F)cccc3OC(F)F)cc21] (Z)-5-(2- (Difluoromethoxy)-6- fluorophenyl)-3-(1-((1- methyl-1H-pyrazol-4- yl)amino)ethylidene)-1H- pyrrolo[2,3-c]pyridin- 2(3H)-one | Step 2 of Method I, and 2-(2- (difluoromethoxy)-6- fluorophenyl)- 4,4,5,5-tetramethyl- 1,3,2-dioxaborolane Method I | ¹H NMR (400 MHz, DMSO-d₆) δ 2.47 (s, 3H), 3.85 (s, 3H), 7.15 (d, J = 7.4 Hz, 1H), 7.18 (s, 1H), 7.21 (t, J = 8.4 Hz, 1H), 7.25 (s, 1H), 7.35-7.52 (m, 1H), 7.59 (s, 1H), 7.96 (s, 1H), 8.19 (s, 1H), 10.87 (s, 1H), 12.12 (s, 1H); ESI-MS (m/z) 416 (M + H)⁺. |
| 36 | 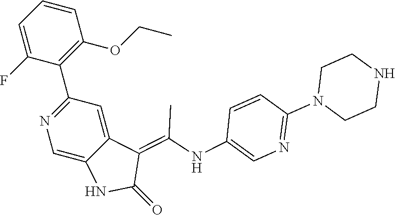 [SMILES: CCOc1cccc(F)c1-c1cc2c(cn1)NC(=O)/C2=C(/C)Nc1ccc(N2CCNCC2)nc1] (Z)-5-(2-Ethoxy-6- fluorophenyl)-3-(1-((6- (piperazin-1-yl)pyridin-3- yl)amino)ethylidene)-1H- pyrrolo[2,3-c]pyridin- 2(3H)-one | Step 1 of Method I, tert-butyl 4-(5- aminopyridin-2- yl)piperazine-1- carboxylate and (2- ethoxy-6- fluorophenyl)boronic acid Method I followed by Boc- deprotection using PTSA | ¹H NMR (400 MHz, DMSO-d₆) δ 1.17 (t, J = 6.8 Hz, 3H), 2.40 (s, 3H), 2.76- 2.78 (m, 4H), 3.41-3.46 (m, 5H), 4.02 (q, J = 6.8 Hz, 2H), 6.81-6.87 (m, 2H), 6.92 (d, J = 8.4 Hz, 1H), 7.30-7.38 (m, 2H), 7.56 (dd, J = 8.8, 2.4 Hz, 1H), 8.12 (d, J = 2.8 Hz, 1H), 8.17 (s, 1H), 10.81 (s, 1H), 12.13 (s, 1H); ESI-MS (m/z) 475 (M + H)⁺. |
| 37 | 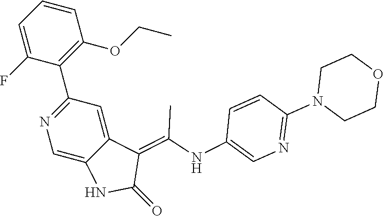 [SMILES: CCOc1cccc(F)c1-c1cc2c(cn1)NC(=O)/C2=C(/C)Nc1ccc(N2CCOCC2)nc1] (Z)-5-(2-Ethoxy-6- fluorophenyl)-3-(1-((6- morpholinopyridin-3- yl)amino)ethylidene)-1H- pyrrolo[2,3-c]pyridin- 2(3H)-one | Step 1 of Method I, 6-morpholinopyridin- 3-amine and (2- ethoxy-6- fluorophenyl)boronic acid Method I | ¹H NMR (400 MHz, DMSO-d₆) δ 1.18 (t, J = 6.8 Hz, 3H), 2.40 (s, 3H), 3.34- 3.40 (m, 4H), 3.68-3.73 (m, 4H), 4.02 (q, J = 6.8 Hz, 2H), 6.84 (t, J = 8.8 Hz, 1H), 6.92 (d, J = 8.4 Hz, 2H), 7.30-7.38 (m, 2H), 7.61 (dd, J = 9.2, 2.8 Hz, 1H), 8.14-8.18 (m, 2H), 10.82 (s, 1H), 12.15 (s, 1H); ESI-MS (m/z) 476 (M + H)⁺. |
| 38 | 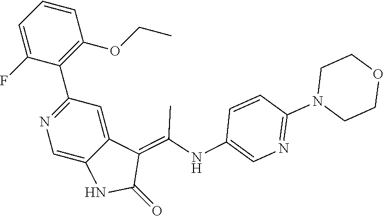 (Z)-5-(4-Methylpyridin-3- yl)-3-(1-((6- morpholinopyridin-3- yl)amino)ethylidene)-1H- pyrrolo[2,3-c]pyridin- 2(3H)-one | Step 1 of Method I, 6-morpholinopyridin- 3-amine and 4- methyl-3- pyridineboronic acid Method I | ¹H NMR (400 MHz, DMSO-d₆) δ 2.37 (s, 3H), 2.48 (s, 3H), 3.49 (t, J = 4.8 Hz, 4H), 3.71 (t, J = 4.8 Hz, 4H), 6.94 (d, J = 8.8 Hz, 1H), 7.31 (d, J = 5.2 Hz, 1H), 7.47 (s, 1H), 7.61 (dd, J = 8.8, 2.4 Hz, 1H), 8.15 (d, J = 2.4 Hz, 1H), 8.23 (s, 1H), 8.42 (d, J = 4.8 Hz, 1H), 8.56 (s, 1H), 10.87 (s, 1H), 12.20 (s, 1H); ESI-MS (m/z) 429 (M + H)⁺. |
| 39 | 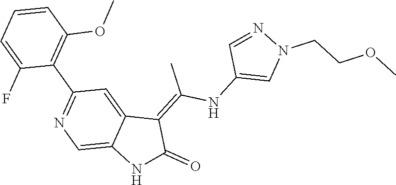 [SMILES: COCCn1cc(N/C(C)=C2\C(=O)Nc3cnc(-c4c(F)cccc4OC)cc32)cn1] (Z)-5-(2-Fluoro-6- methoxyphenyl)-3-(1-((1- (2-methoxyethyl)-1H- pyrazol-4- yl)amino)ethylidene)-1H- pyrrolo[2,3-c]pyridin- 2(3H)-one | Step 1 of Method I, 1-(2-methoxyethyl)- 1H-pyrazol-4-amine and (2-fluoro-6- methoxyphenyl) boronic acid Method I | ¹H NMR (400 MHz, DMSO-d₆) δ 2.46 (s, 3H), 3.24 (s, 3H), 3.70 (t, J = 5.6 Hz, 3H), 3.72 (s, 3H), 4.26 (t, J = 5.2 Hz, 3H), 6.87 (t, J = 8.4 Hz, 1H), 6.94 (d, J = 8.4 Hz, 1H), 7.30 (s, 1H), 7.35-7.40 (m, 1H), 7.61 (s, 1H), 7.97 (s, 1H), 8.16 (s, 1H), 10.81 (s, 1H), 12.11 (s, 1H); ESI-MS (m/z) 424 (M + H)⁺. |
| 40 | 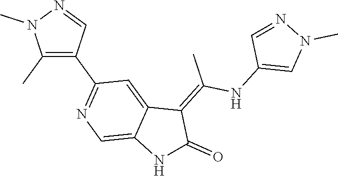 (Z)-5-(2-Fluoro-6- methoxyphenyl)-3-(2- methyl-1-((1-methyl-1H- pyrazol-4- yl)amino)propylidene)-1H- pyrrolo[2,3-c]pyridin- 2(3H)-one | 1,1,1-trimethoxy-2- methylpropane, 2- fluoro-6- methoxyphenylboronic acid Method I | ¹H NMR (400 MHz, DMSO-d₆) δ 1.23-1.28 (m, 6H), 2.48-2.52 (m, 1H), 3.72 (s, 3H), 3.86 (s, 3H), 6.88 (t, J = 8.4 Hz, 1H), 6.96 (d, J = 8.8 Hz, 1H), 7.33 (s, 1H), 7.34-7.39 (m, 1H), 7.41 (s, 1H), 7.55 (s, 1H), 8.19 (s, 1H), 10.95 (s, 1H), 12.61 (s, 1H); ESI-MS (m/z) 408 (M + H)⁺. |
| 41 | 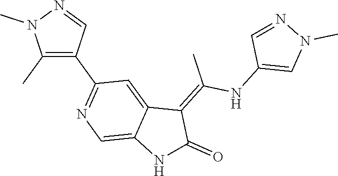 (Z)-5-(1,5-Dimethyl-1H- pyrazol-4-yl)-3-(1-((1- methyl-1H-pyrazol-4- yl)amino)ethylidene)-1H- pyrrolo[2,3-c]pyridin- 2(3H)-one | (1,5-dimethyl-1H- pyrazol-4-yl)boronic acid Method I | ¹H NMR (400 MHz, DMSO-d₆) δ 2.54 (s, 3H), 2.56 (s, 3H), 3.77 (s, 3H), 3.86 (s, 3H), 7.45 (s, 1H), 7.58 (s, 1H), 7.82 (s, 1H), 7.95 (s, 1H), 8.09 (s, 1H), 10.68 (s, 1H), 12.09 (s, 1H); ESI-MS (m/z) 350 (M + H)⁺. |
| 42 | 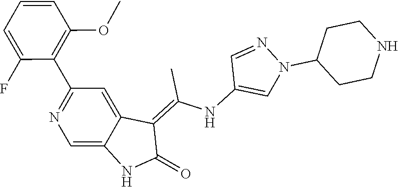 [SMILES: COc1cccc(F)c1-c1cc2c(cn1)NC(=O)/C2=C(/C)Nc1cnn(C2CCNCC2)c1] (Z)-5-(2-Fluoro-6- methoxyphenyl)-3-(1-((1- (piperidin-4-yl)-1H- pyrazol-4- yl)amino)ethylidene)-1H- pyrrolo[2,3-c]pyridin- 2(3H)-one | Step 1 of Method I, tert-butyl 4-(4-amino- 1H-pyrazol-1- yl)piperidine-1- carboxylate and (2- fluoro-6- methoxyphenyl)boronic acid Method I | ¹H NMR (400 MHz, DMSO-d₆) δ 1.93-2.05 (m, 4H), 2.46 (s, 3H), 2.53-2.61 (m, 1H), 3.35-3.51 (m, 4H), 3.72 (s, 3H), 4.16-4.20 (m, 1H), 6.87 (t, J = 8.8 Hz, 1H), 6.94 (d, J = 8.4 Hz, 1H), 7.29 (s, 1H), 7.38 (dd, J = 15.2, 8.4 Hz, 1H), 7.59- 7.63 (m, 1H), 8.03 (s, 1H), 8.16 (s, 1H), 10.80 (s, 1H), 12.12 (s, 1H); ESI-MS (m/z) 449 (M + H)⁺. |
| 43 | 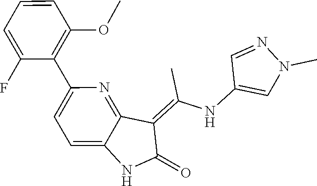 [SMILES: COc1cccc(F)c1-c1ccc2c(n1)/C(=C(\C)Nc1cnn(C)c1)C(=O)N2] (Z)-5-(2-Fluoro-6- methoxyphenyl)-3-(1-((1- methyl-1H-pyrazol-4- yl)amino)ethylidene)-1H- pyrrolo[3,2-b]pyridin- 2(3H)-one | Intermediate B12, 2- Fluoro-6- methoxyphenylboronic acid Method I | ¹H NMR (400 MHz, CDCl₃) δ 2.69 (s, 3H), 3.81 (s, 3H), 3.93 (s, 3H), 6.79- 6.85 (m, 2H), 7.12-7.19 (m, 2H), 7.29-7.34 (m, 2H), 7.36-7.48 (m, 1H), 7.95 (s, 1H), 11.92 (s, 1H); ESI-MS (m/z) 380 (M + H)⁺. |
| 44 | 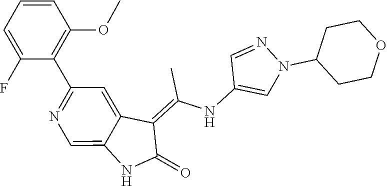 [SMILES: COc1cccc(F)c1-c1cc2c(cn1)NC(=O)/C2=C(/C)Nc1cnn(C2CCOCC2)c1] (Z)-5-(2-Fluoro-6- methoxyphenyl)-3-(1-((1- (tetrahydro-2H-pyran-4- yl)-1H-pyrazol-4- yl)amino)ethylidene)-1H- pyrrolo[2,3-c]pyridin- 2(3H)-one | Step 1 of Method I, 1-(tetrahydro-2H- pyran-4-yl)-1H- pyrazol-4-amine Method I | ¹H NMR (400 MHz, DMSO-d₆) δ 1.94-2.00 (m, 4H), 2.47 (s, 3H), 3.36-3.49 (m, 2H), 3.72 (s, 3H), 3.94- 3.99 (m, 2H), 4.35-4.44 (m, 1H), 6.87 (t, J = 8.4 Hz, 1H), 6.94 (d, J = 8.4 Hz, 1H), 7.30 (s, 1H), 7.34-7.42 (m, 1H), 7.62 (s, 1H), 8.08 (s, 1H), 8.16 (s, 1H), 10.80 (s, 1H), 12.13 (s, 1H); ESI- MS (m/z) 450 (M + H)⁺. |
| 45 | 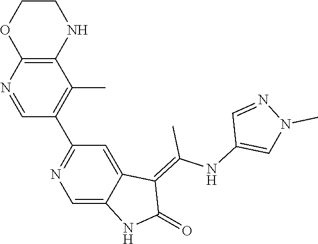 [SMILES: C/C(Nc1cnn(C)c1)=C1/C(=O)Nc2cnc(-c3cnc4c(c3C)NCCO4)cc21] (Z)-3-(1-((1-Methyl-1H- pyrazol-4- yl)amino)ethylidene)-5-(8- methyl-2,3-dihydro-1H- pyrido[2,3-b][1,4]oxazin-7- yl)-1H-pyrrolo[2,3- c]pyridin-2(3H)-one | Step 3 Intermediate of Method I, tert- butyl 7-(4,4,5,5- tetramethyl-1,3,2- dioxaborolan-2-yl)- 2,3-dihydro-1H- pyrido[2,3- b][1,4]oxazine-1- carboxylate Method 1 followed by Boc deprotection using PTSA | ¹H NMR (400 MHz, DMSO-d₆) δ 2.05 (s, 3H), 2.48-2.52 (m, 3H), 3.36- 3.40 (m, 2H), 3.86 (s, 3H), 4.25 (t, J = 3.6 Hz, 2H), 5.55 (s, 1H), 7.32 (s, 1H), 7.44 (s, 1H), 7.57 (s, 1H), 7.95 (s, 1H), 8.17 (s, 1H), 10.78 (s, 1H), 12.09 (s, 1H); ESI-MS (m/z) 404 (M + H)⁺. |
| 46 | 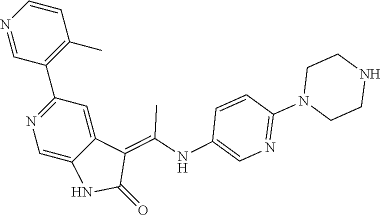 [SMILES: C/C(Nc1ccc(N2CCNCC2)nc1)=C1/C(=O)Nc2cnc(-c3cnccc3C)cc21] (Z)-5-(4-Methylpyridin-3- yl)-3-(1-((6-(piperazin-1- yl)pyridin-3- yl)amino)ethylidene)-1H- pyrrolo[2,3-c]pyridin- 2(3H)-one | Step 1 of Method I, tert-butyl 4-(5- aminopyridin-2- yl)piperazine-1- carboxylate and 4- Methylpyridine-3- boronic acid Method I | ¹H NMR (400 MHz, DMSO-d₆) δ 2.37 (s, 3H), 2.47 (s, 3H), 2.77-2.80 (m, 4H), 3.42-3.46 (m, 4H), 6.88 (d, J = 9.2 Hz, 1H), 7.31 (d, J = 5.2 Hz, 1H), 7.46 (s, 1H), 7.52-7.57 (m, 1H), 8.11 (d, J = 2.4 Hz, 1H), 8.23 (s, 1H), 8.42 (d, J = 4.8 Hz, 1H), 8.56 (s, 1H), 10.86 (s, 1H), 12.18 (s, 1H); ESI-MS (m/z) 428 (M + H)⁺. |
| 47 | 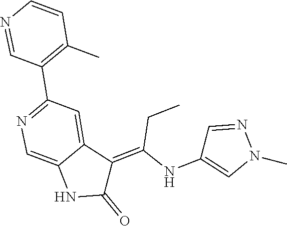 [SMILES: CC/C(Nc1cnn(C)c1)=C1/C(=O)Nc2cnc(-c3cnccc3C)cc21] (Z)-3-(1-((1-Methyl-1H- pyrazol-4- yl)amino)propylidene)-5- (4-methylpyridin-3-yl)-1H- pyrrolo[2,3-c]pyridin- 2(3H)-one | Step 3 of Intermediate B7, triethylorthopropionate, 4-Methylpyridine- 3-boronic acid Method I | ¹H NMR (400 MHz, DMSO-d₆) δ 1.25 (t, J = 7.6 Hz, 3H), 2.38 (s, 3H), 2.81 (q, J = 7.6 Hz, 2H), 3.87 (s, 3H), 7.32 (d, J = 5.2 Hz, 1H), 7.39 (s, 1H), 7.59 (s, 1H), 7.99 (s, 1H), 8.24 (s, 1H), 8.43 (d, J = 4.8 Hz, 1H), 8.56 (s, 1H), 10.90 (s, 1H), 12.03 (s, 1H); ESI-MS (m/z) 361 (M + H)⁺. |
| 48 | 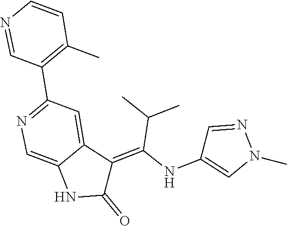 [SMILES: Cc1ccncc1-c1cc2c(cn1)NC(=O)/C2=C(\Nc1cnn(C)c1)C(C)C] (Z)-3-(2-Methyl-1-((1- methyl-1H-pyrazol-4- yl)amino)propylidene)-5- (4-methylpyridin-3-yl)-1H- pyrrolo[2,3-c]pyridin- 2(3H)-one | 1,1,1-trimethoxy-2- methylpropane, 4- Methylpyridine-3- boronic acid Method I | ¹H NMR (400 MHz, DMSO-d₆) δ 1.3-1.28 (m, 6H), 2.38 (s, 3H), 3.34 (br s, 1H), 3.87 (s, 3H), 7.32 (s, 1H), 7.43 (s, 1H), 7.56 (s, 1H), 7.97 (s, 1H), 8.26 (s, 1H), 8.43 (s, 1H), 8.55 (s, 1H), 11.0 (s, 1H), 12.68 (s, 1H); ESI-MS (m/z) 375 (M + H)⁺. |
| 49 | 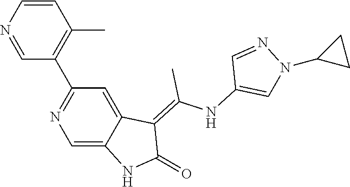 [SMILES: C/C(Nc1cnn(C2CC2)c1)=C1/C(=O)Nc2cnc(-c3cnccc3C)cc21] (Z)-3-(1-((1-Cyclopropyl- 1H-pyrazol-4- yl)amino)ethylidene)-5-(4- methylpyridin-3-yl)-1H- pyrrolo[2,3-c]pyridin- 2(3H)-one | Step 2 of Method I, 1-cyclopropyl-1H- pyrazol-4-amine, 4- Methylpyridine-3- boronic acid Method I | ¹H NMR (400 MHz, DMSO-d₆) δ 0.84-1.11 (m, 4H), 2.36 (s, 3H), 3.53 (s, 3H), 3.74-3.78 (m, 1H), 7.34 (d, J = 5.2 Hz, 1H), 7.54 (s, 1H), 7.60 (s, 1H), 8.08 (s, 1H), 8.23 (s, 1H), 8.45 (d, J = 4.8 Hz, 1H), 8.58 (s, 1H), 10.98 (s, 1H), 12.22 (s, 1H); ESI-MS (m/z) 373 (M + H)⁺. |
| 50 | 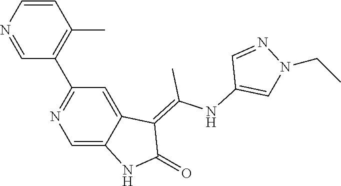 [SMILES: CCn1cc(N/C(C)=C2\C(=O)Nc3cnc(-c4cnccc4C)cc32)cn1] (Z)-3-(1-((1-Ethyl-1H- pyrazol-4- yl)amino)ethylidene)-5-(4- methylpyridin-3-yl)-1H- pyrrolo[2,3-c]pyridin- 2(3H)-one | Step 2 of Method I, 1-ethyl-1H-pyrazol- 4-amine, 4- Methylpyridine-3- boronic acid Method I | ¹H NMR (400 MHz, DMSO-d₆) δ 1.39 (t, J = 7.2 Hz, 3H), 2.37 (s, 3H), 2.55 (s, 3H), 4.14 (q, J = 7.2 Hz, 2H), 7.32 (d, J = 4.8 Hz, 1H), 7.51 (s, 1H), 7.60 (s, 1H), 8.02 (s, 1H), 8.22 (m, 1H), 8.43 (d, J = 4.8 Hz, 1H), 8.57 (s, 1H), 10.90 (s, 1H), 12.20 (s, 1H); ESI-MS (m/z) 361 (M + H)⁺. |
| 51 | 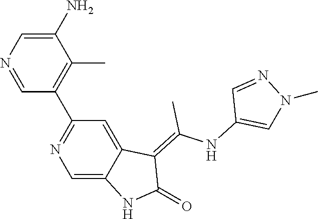 [SMILES: C/C(Nc1cnn(C)c1)=C1/C(=O)Nc2cnc(-c3cncc(N)c3C)cc21] (Z)-5-(5-Amino-4- methylpyridin-3-yl)-3-(1- ((1-methyl-1H-pyrazol-4- yl)amino)ethylidene)-1H- pyrrolo[2,3-c]pyridin- 2(3H)-one | Step-2 of Method I, (5-amino-4- methylpyridin-3- yl)boronic acid Method I | ¹H NMR (400 MHz, DMSO-d₆) δ 2.03 (s, 3H), 2.50 (s, 3H), 3.86 (s, 3H), 5.14 (s, 2H), 7.36 (s, 1H), 7.57 (s, 1H), 7.78 (s, 1H), 7.94 (d, J = 9.6 Hz, 2H), 8.19 (s, 1H), 10.82 (s, 1H), 12.11 (s, 1H). |
| 52 | 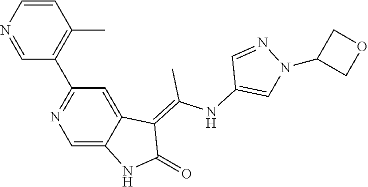 (Z)-5-(4-Methylpyridin-3- yl)-3-(1-((1-(oxetan-3-yl)- 1H-pyrazol-4- yl)amino)ethylidene)-1H- pyrrolo[2,3-c]pyridin- 2(3H)-one | Step-1 of Method I, 1-(oxetan-3-yl)-1H- pyrazol-4-amine, 4- methylpyridine-3- boronic acid Method I | ¹H NMR (400 MHz, DMSO-d₆) δ 2.37 (s, 3H), 2.57 (s, 3H), 4.89-4.95 (m, 4H), 5.57-5.60 (m, 1H), 7.34 (d, J = 4.8 Hz, 1H), 7.54 (s, 1H), 7.78 (s, 1H), 8.19 (s, 1H), 8.23 (s, 1H), 8.44 (d, J = 4.8 Hz, 1H), 8.57 (s, 1H), 10.95 (s, 1H), 12.24 (s, 1H); ESI-MS (m/z) 361 (M + H)⁺. |
| 53 | 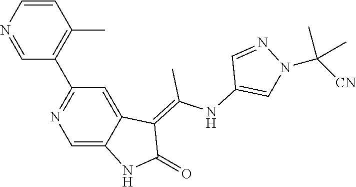 [SMILES: C/C(Nc1cnn(C(C)(C)C#N)c1)=C1/C(=O)Nc2cnc(-c3cnccc3C)cc21] (Z)-2-Methyl-2-(4-((1-(5- (4-methylpyridin-3-yl)-2- oxo-1H-pyrrolo[2,3- c]pyridin-3(2H)- ylidene)ethyl)amino)-1H- pyrazol-1-yl)propanenitrile | Step-1 of Method I, 2-(4-amino-1H- pyrazol-1-yl)-2- methylpropanenitrile, 4-methylpyridine-3- boronic acid Method I | ¹H NMR (400 MHz, DMSO-d₆) δ 2.00 (s, 6H), 2.37 (s, 3H), 2.57 (s, 3H), 7.32 (d, J = 4.8 Hz, 1H), 7.52 (s, 1H), 7.83 (s, 1H), 8.23 (s, 1H), 8.32 (s, 1H), 8.43 (d, J = 4.8 Hz, 1H), 8.57 (s, 1H), 10.90 (s, 1H), 12.19 (s, 1H); ESI-MS (m/z) 400 (M + H)⁺. |
| 54 | 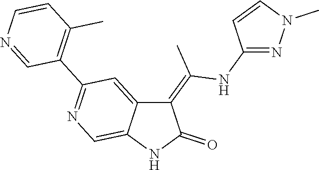 [SMILES: C/C(Nc1ccn(C)n1)=C1/C(=O)Nc2cnc(-c3cnccc3C)cc21] (Z)-3-(1-((1-methyl-1H- pyrazol-3- yl)amino)ethylidene)-5-(4- methylpyridin-3-yl)-1H- pyrrolo[2,3-c]pyridin- 2(3H)-one | Step-1 of Method I, 1-methyl-1H-pyrazol- 3-amine and 4- methylpyridine-3- boronic acid Method I | ¹H NMR (400 MHz, DMSO-d₆) δ 2.38 (s, 3H), 2.71 (s, 3H), 3.83 (s, 3H), 6.28 (d, J = 2.0 Hz, 1H), 7.32 (d, J = 5.2 Hz, 1H), 7.52 (s, 1H), 7.77 (d, J = 2.0 Hz, 1H), 8.24 (s, 1H), 8.42 (d, J = 4.8 Hz, 1H), 8.57 (s, 1H), 10.91 (s, 1H), 12.63 (s, 1H); ESI-MS (m/z) 347 (M + H)⁺. |
| 55 | 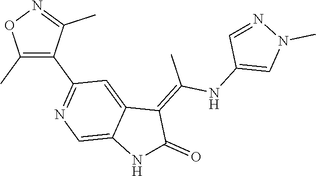 (Z)-5- (3,5dimethylisoxazol-4-yl)- 3-(1-((1-methyl-1H- pyrazol-4- yl)amino)ethylidene)-1H- pyrrolo[2,3-c]pyridin- 2(3H)-one | Step-2 of Method I and (3,5- dimethylisoxazol-4- yl)boronic acid Method I | ¹H NMR (400 MHz, DMSO-d₆) δ 2.34 (s, 3H), 2.54 (s, 6H), 3.84 (s, 3H), 7.42 (s, 1H), 7.59 (s, 1H), 7.97 (s, 1H), 8.19 (s, 1H), 10.85 (s, 1H), 12.14 (s, 1H); ESI-MS (m/z) 351 (M + H)⁺. |
| 56 | 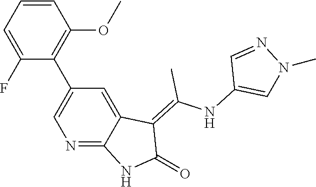 [SMILES: COc1cccc(F)c1-c1cnc2c(c1)/C(=C(\C)Nc1cnn(C)c1)C(=O)N2] (Z)-5-(2-Fluoro-6- methoxyphenyl)-3-(1-((1- methyl-1H-pyrazol-4- yl)amino)ethylidene)-1H- pyrrolo[2,3-b]pyridin- 2(3H)-one | Step 2 of Method I, 1-methyl-1H-pyrazol- 4-amine and 2-fluoro- 6- methoxyphenylboronic acid Method I | ¹H NMR (400 MHz, DMSO-d₆) δ 2.41 (s, 3H), 3.76 (s, 3H), 3.85 (s, 3H), 6.92 (t, J = 8.4 Hz, 1H), 6.98 (d, J = 8.4 Hz, 1H), 7.34-7.42 (m, 1H), 7.56 (d, J = 8.0 Hz, 2H), 7.83 (d, J = 1.6 Hz, 1H), 7.91 (s, 1H), 11.15 (s, 1H), 11.68 (s, 1H); ESI-MS (m/z) 380 (M + H)⁺. |
| 57 | 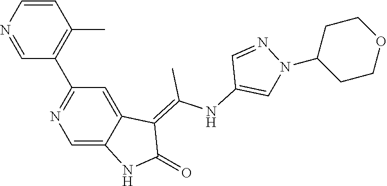 [SMILES: C/C(Nc1cnn(C2CCOCC2)c1)=C1/C(=O)Nc2cnc(-c3cnccc3C)cc21] (Z)-5-(4-Methylpyridin-3- yl)-3-(1-((1-(tetrahydro- 2H-pyran-4-yl)-1H- pyrazol-4- yl)amino)ethylidene)-1H- pyrrolo[2,3-c]pyridin- 2(3H)-one | Step 1 of Method 1, 1-(tetrahydro-2H- pyran-4-yl)-1H- pyrazol-4-amine and 4-methyl-3-(4,4,5,5- tetramethyl-1,3,2- dioxaborolan-2- yl)pyridine Method I | ¹H NMR (400 MHz, DMSO-d₆) δ 1.93-2.00 (m, 4H), 2.37 (s, 3H), 2.55 (s, 3H), 3.43-3.51 (m, 2H), 3.94-3.99 (m, 2H), 4.38- 4.43 (m, 1H), 7.32 (d, J = 5.2 Hz, 1H), 7.50 (s, 1H), 7.63 (s, 1H), 8.09 (s, 1H), 8.22 (s. 1H), 8.43 (d, J = 4.8 Hz, 1H), 8.57 (s, 1H), 10.87 (s, 1H), 12.18 (s, 1H); ESI- MS (m/z) 417 (M + H)⁺. |
| 58 | 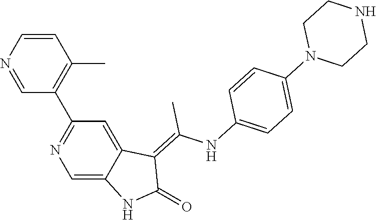 [SMILES: C/C(Nc1ccc(N2CCNCC2)cc1)=C1/C(=O)Nc2cnc(-c3cnccc3C)cc21] (Z)-5-(4-Methylpyridin-3- yl)-3-(1-((4-(piperazin-1- yl)phenyl)amino)ethylidene)- 1H-pyrrolo[2,3-c]pyridin- 2(3H)-one | Step 1 of Method I, tert-butyl 4-(4- aminophenyl)piperazine- 1-carboxylate and 4-methylpyridine-3- boronic acid Method I followed by Boc-deprotection using PTSA | ¹H NMR (400 MHz, DMSO-d₆) δ 2.37 (s, 3H), 2.51 (s, 3H), 2.96 (t, J = 4.8 Hz, 4H), 3.190, J = 4.4 Hz, 4H), 7.02 (d, J = 8.8 Hz, 2H), 7.20 (d, J = 8.8 Hz, 3H), 7.30 (d, J = 4.8 Hz, 1H), 7.45 (s,1H), 8.23 (s, 1H), 8.41 (d, J = 4.8 Hz, 1H), 8.56 (s, 1H), 10.85 (s, 1H), 12.35 (s, 1H); ESI-MS (m/z) 427 (M + H)⁺. |
| 59 | 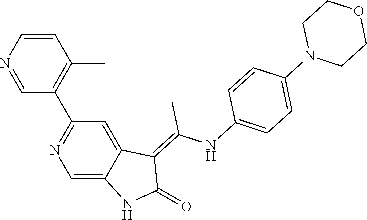 [SMILES: C/C(Nc1ccc(N2CCOCC2)cc1)=C1/C(=O)Nc2cnc(-c3cnccc3C)cc21] (Z)-5-(4-Methylpyridin-3- yl)-3-(1-((4- morpholinophenyl)amino) ethylidene)-1H-pyrrolo[2,3- c]pyridin-2(3H)-one | Step 1 of Method I, 4-morpholinoaniline and 4- methylpyridine-3- boronic acid Method I | ¹H NMR (400 MHz, DMSO-d₆) δ 2.37 (s, 3H), 2.51 (s, 3H), 3.15 (t, J = 4.8 Hz, 4H), 3.74 (t, J = 4.4 Hz, 4H), 7.03 (d, J = 8.8 Hz, 2H), 7.21 (d, J = 8.8 Hz, 2H), 7.30 (d, J = 5.2 Hz, 1H), 7.45 (s, 1H), 8.22 (s, 1H), 8.41 (d, J = 5.2 Hz, 1H), 8.56 (s, 1H), 10.85 (s, 1H), 12.36 (s, 1H); ESI-MS (m/z) 428 (M + H)⁺. |
| 60 | 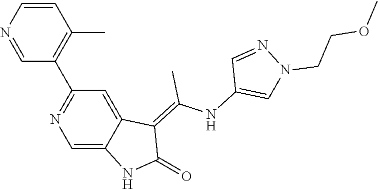 [SMILES: COCCn1cc(N/C(C)=C2\C(=O)Nc3cnc(-c4cnccc4C)cc32)cn1] (Z)-3-(1-((1-(2- Methoxyethyl)-1H-pyrazol- 4-yl)amino)ethylidene)-5- (4-methylpyridin-3-yl)-1H- pyrrolo[2,3-c]pyridin- 2(3H)-one | Step-1 of Method I, 1-(2-methoxyethyl)- 1H-pyrazol-4-amine, 4-methylpyridine-3- boronic acid Method I (Pd(dppf)₂Cl₂ was used as catalyst in step 3) | ¹H NMR (400 MHz, DMSO-d₆) δ 2.37 (s, 3H), 2.51 (s, 3H), 3.24 (s, 3H), 3.70 (d, J = 5.2 Hz, 2H), 4.27 (d, J = 5.2 Hz, 2H), 7.31 (d, J = 5.2 Hz, 1H), 7.49 (s, 1H), 7.61 (s, 1H), 7.98 (s, 1H), 8.22 (s, 1H), 8.41 (d, J = 5.2 Hz, 1H), 8.56 (s, 1H), 10.86 (s, 1H), 12.15 (s, 1H); ESI-MS (m/z) 391 (M + H)⁺. |
| 61 | 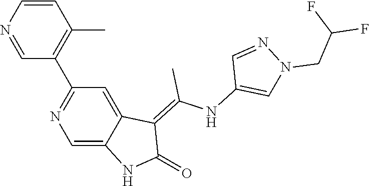 [SMILES: C/C(Nc1cnn(CC(F)F)c1)=C1/C(=O)Nc2cnc(-c3cnccc3C)cc21] (Z)-3-(1-((1-(2,2- Difluoroethyl)-1H-pyrazol 4-yl)amino)ethylidene)-5- (4-methylpyridin-3-yl)-1H pyrrolo[2,3-c]pyridin- 2(3H)-one | Step-1 of Method I, 1-(2,2-difluoroethyl)- 1H-pyrazol-4-amine and 4- methylpyridine-3- boronic acid Method I | ¹H NMR (400 MHz, DMSO-d₆) δ 2.37 (s, 3H), 2.54 (s, 3H), 4.60-4.70 (m, 2H), 6.26-6.53 (m, 1H), 7.32 (d, J = 4.8 Hz, 1H), 7.51 (s, 1H), 7.72 (s, 1H), 8.06 (s, 1H), 8.23 (s, 1H), 8.43 (d, J = 4.8 Hz, 1H), 8.57 (s, 1H), 10.88 (s, 1H), 12.16 (s, 1H); ESI-MS (m/z) 398 (M + H)⁺. |
| 62 | 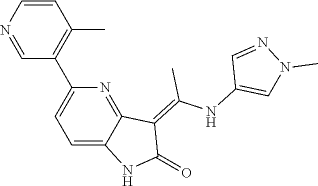 [SMILES: C/C(Nc1cnn(C)c1)=C1/C(=O)Nc2ccc(-c3cnccc3C)nc21] (Z)-3-(1-((1-Methyl-1H- pyrazol-4- yl)amino)ethylidene)-5-(4- methylpyridin-3-yl)-1H- pyrrolo[3,2-b]pyridin- 2(3H)-one | Intermediate B12, 1- methyl-1H-pyrazol-4- amine and 4- methylpyridine-3- boronic acid Method I (Pd(dppf)₂Cl₂ was used as catalyst in step 3) | ¹H NMR (400 MHz, DMSO-d₆) δ 2.46 (s, 3H), 2.63 (s, 3H), 3.86 (d, J = 7.6 Hz, 3H), 7.12-7.18 (m, 1H), 7.23-7.33 (m, 2H), 7.41- 7.58 (s, 1H), 7.58-7.62 (s, 1H), 8.42 (dd, J = 5.2, 1.2 Hz, 2H), 8.40-8.60 (m, 1H), 11.64-11.76 (s, 1H); ESI-MS (m/z) 347 (M + H)⁺. |
| 63 | 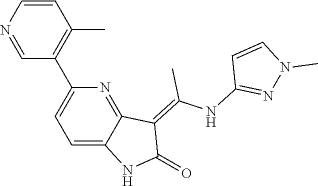 [SMILES: C/C(Nc1ccn(C)n1)=C1/C(=O)Nc2ccc(-c3cnccc3C)nc21] (Z)-3-(1-((1-Methyl-1H- pyrazol- 34yl)amino)ethylidene)-5- (4-methylpyridin-3-yl)-1H- pyrrolo[3,2-b]pyridin- 2(3H)-one | Intermediate B12, 1- methyl-1H-pyrazol-3- amine and 4- methylpyridine-3- boronic acid Method I (Pd(dppf)₂Cl₂ was used as catalyst in step 3) | ESI-MS (m/z) 347 (M + H)⁺. |
| 64 | 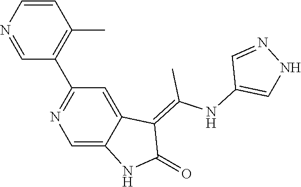 [SMILES: C/C(Nc1cn[nH]c1)=C1/C(=O)Nc2cnc(-c3cnccc3C)cc21] (Z)-3-(1-((1H-Pyrazol-4- yl)amino)ethylidene)-5-(4- methylpyridin-3-yl)-1H- pyrrolo[2,3-c]pyridin- 2(3H)-one | Step-1 of Method I, 1-(ethylsulfonyl)-1H- pyrazol-4-amine, 4- methylpyridine-3- boronic acid Method I | ¹H NMR (400 MHz, DMSO-d₆) δ 2.27 (s, 3H), 2.37 (s, 3H), 7.32 (d, J = 4.8 Hz, 1H), 7.50 (s, 1H), 7.65 (s, 1H), 8.01 (s, 1H), 8.22 (s, 1H), 8.43 (d, J = 4.8 Hz, 1H), 8.57 (s, 1H), 10.88 (s, 1H), 12.16 (s, 1H), 13.11 (s, 1H); ESI-MS (m/z) 332 (M)⁺. |
| 65 | 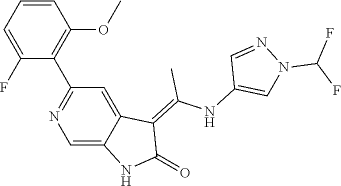 [SMILES: COc1cccc(F)c1-c1cc2c(cn1)NC(=O)/C2=C(/C)Nc1cnn(C(F)F)c1] (Z)-3-(1-((1- (Difluoromethyl)-1H- pyrazol-4- yl)amino)ethylidene)-5-(2- fluoro-6-methoxyphenyl)- 1H-pyrrolo[2,3-c]pyridin- 2(3H)-one | Step 1 of Method I, 1-(difluoromethyl)- 1H-pyrazol-4-amine and 2-fluoro-6- methoxyphenylboronic acid Method I | ¹H NMR (400 MHz, DMSO-d₆) δ 2.48 (s, 3H), 3.72 (s, 3H), 6.87 (t, J = 8.4 Hz, 1H), 6.95 (d, J = 8.4 Hz, 1H), 7.33 (s, 1H), 7.34-7.40 (m, 1H), 7.68-7.98 (m, 1H), 8.01 (s, 1H), 8.18 (s, 1H), 8.52 (s, 1H), 10.87 (m, 1H), 12.12 (m, 1H). |
| 66 | 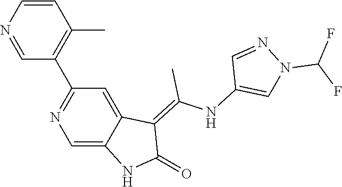 [SMILES: C/C(Nc1cnn(C(F)F)c1)=C1/C(=O)Nc2cnc(-c3cnccc3C)cc21] (Z)-3-(1-((1- (Difluoromethyl)-1H- pyrazol-4- yl)amino)ethylidene)-5-(4- methylpyridin-3-yl)-1H- pyrrolo[2,3-c]pyridin- 2(3H)-one | Step-1 of Method I, 1-(difluoromethyl)- 1H-pyrazol-4-amine, 4-methylpyridine-3- boronic acid Method I | ¹H NMR (400 MHz, DMSO-d₆) δ 2.37 (s, 3H), 2.56 (s, 3H), 7.32 (d, J = 5.2 Hz, 1H), 7.53 (s, 1H), 7.69- 7.98 (m, 1H), 8.02 (s, 1H), 8.24 (s, 1H), 8.43 (d, J = 4.8 Hz, 1H), 8.54 (s, 1H), 8.57 (s, 1H), 10.93 (s, 1H), 12.16 (s, 1H); ESI-MS (m/z) 383 (M + H)⁺. |
| 67 | 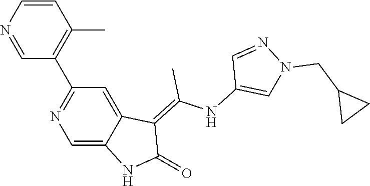 [SMILES: C/C(Nc1cnn(CC2CC2)c1)=C1/C(=O)Nc2cnc(-c3cnccc3C)cc21] (Z)-3-(1-((1- (Cyclopropylmethyl)-1H- pyrazol-4- yl)amino)ethylidene)-5-(4- methylpyridin-3-yl)-1H- pyrrolo[2,3-c]pyridin- 2(3H)-one | Step 1 of Method I, 1- (cyclopropylmethyl)- 1H-pyrazol-4-amine and 4- methylpyridine-3- boronic acid Method I | ¹H NMR (400 MHz, DMSO-d₆) δ 0.36-0.40 (m, 2H), 0.53-0.56 (m, 2H), 1.22-1.28 (m, 1H), 2.37 (s, 3H), 2.50-2.55 (m, 3H), 3.98 (d, J = 7.2 Hz, 2H), 7.31 (d, J = 4.8 Hz, 1H), 7.49 (s, 1H), 7.59 (s, 1H), 8.04 (s, 1H), 8.22 (s, 1H), 8.41 (d, J = 5.2 Hz, 1H), 8.57 (s, 1H), 10.85 (s, 1H), 12.17 (s, 1H); ESI-MS (m/z) 387 (M + H)⁺. |
| 68 | 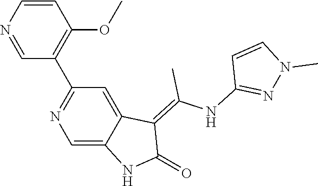 [SMILES: COc1ccncc1-c1cc2c(cn1)NC(=O)/C2=C(/C)Nc1ccn(C)n1] (Z)-5-(4-Methoxypyridin- 3-yl)-3-(1-((1-methyl-1H- pyrazol-3- yl)amino)ethylidene)-1H- pyrrolo[2,3-c]pyridin- 2(3H)-one | Step 1 of Method I, 1-methyl-1H-pyrazol- 3-amine and 4- methylpyridine-3- boronic acid Method I | ¹H NMR (400 MHz, DMSO-d₆) δ 2.70 (s, 3H), 3.83 (s, 3H), 3.91 (s, 3H), 6.29 (d, J = 2.4 Hz, 1H), 7.17 (d, J = 6.0 Hz, 1H), 7.77 (d, J = 2.0 Hz, 1H), 7.83 (s, 1H),8.23 (s, 1H), 8.43 (d, J = 4.8 Hz, 1H), 8.72 (s, 1H), 10.89 (s, 1H), 12.59 (s, 1H). |
| 69 | 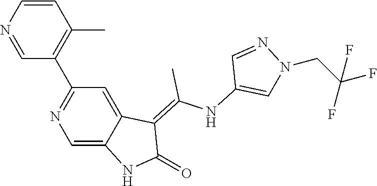 [SMILES: C/C(Nc1cnn(CC(F)(F)F)c1)=C1/C(=O)Nc2cnc(-c3cnccc3C)cc21] (Z)-5-(4-Methylpyridin-3- yl)-3-(1-((1-(2,2,2- trifluoroethyl)-1H-pyrazol- 4-yl)amino)ethylidene)-1H- pyrrolo[2,3-c]pyridin- 2(3H)-one | Step 1 of Method I, 1-(2,2,2- trifluoroethyl)-1H- pyrazol-4-amine and 4-methylpyridine-3- boronic acid Method I | ¹H NMR (400 MHz, DMSO-d₆) δ 2.37 (s, 3H), 2.54 (s, 3H), 5.13-5.22 (m, 2H), 7.51 (s, 1H), 7.78 (s, 1H), 8.13 (s, 1H), 8.23 (s, 1H), 8.32 (s, 1H), 8.50-8.61 (m, 2H), 10.89 (s, 1H), 12.14 (s, 1H); ESI-MS (m/z) 415 (M + H)⁺. |
| 70 | 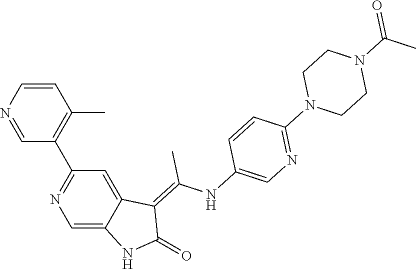 [SMILES: CC(=O)N1CCN(c2ccc(N/C(C)=C3\C(=O)Nc4cnc(-c5cnccc5C)cc43)cn2)CC1] (Z)-3-(1-((6-(4- Acetylpiperazin-1- yl)pyridin-3- yl)amino)ethylidene)-5-(4- methylpyridin-3-yl)-1H- pyrrolo[2,3-c]pyridin- 2(3H)-one | Step 1 of Method I, 1-(4-(5- aminopyridin-2- yl)piperazin-1- yl)ethanone and 4- methyl-3-(4,4,5,5- tetramethyl-1,3,2- dioxaborolan-2- yl)pyridine Method I | ¹H NMR (400 MHz, DMSO-d₆) δ 2.05 (s, 3H), 2.37 (s, 3H), 2.51 (s, 3H), 3.53-3.60 (m, 8H), 6.95 (d, J = 5.2 Hz, 1H), 7.30 (d, J = 5.2 Hz, 1H), 7.47 (s, 1H), 7.60 (dd, J₁ = 2.8 Hz, J₂ = 9.6 Hz, 1H), 8.14 (d, J = 2.4 Hz, 1H), 8.23 (s, 1H), 8.42 (d, J = 4.8 Hz, 1H), 8.56 (s, 1H), 10.86 (s, 1H), 12.20 (s, 1H); ESI-MS (m/z) 470 (M + H)⁺. |
| 71 | 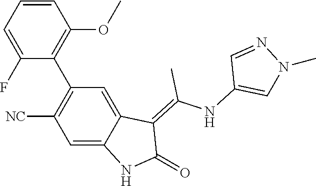 [SMILES: COc1cccc(F)c1-c1cc2c(cc1C#N)NC(=O)/C2=C(/C)Nc1cnn(C)c1] (Z)-5-(2-Fluoro-6- methoxyphenyl)-3-(1-((1- methyl-1H-pyrazol-4- yl)amino)ethylidene)-2- oxoindoline-6-carbonitrile | 5-Bromo-2- oxoindoline-6- carbonitrile, 1- methyl-1H-pyrazol-4- amine and (2-fluoro- 6- methoxyphenyl) boronic acid Method I | ¹H NMR (400 MHz, DMSO-d₆) δ 2.45 (s, 3H), 3.76 (s, 3H), 3.85 (s, 3H), 6.94 (t, J = 8.8 Hz, 1H), 7.01 (d, J = 8.4 Hz, 1H), 7.28 (s, 1H), 7.36 (s, 1H), 7.46 (q, J = 12 Hz, 1H), 7.57 (s, 1H), 7.94 (s, 1H), 10.97 (s, 1H), 12.11 (s, 1H); ESI-MS (m/z) 404 (M + H)⁺. |
| 72 | 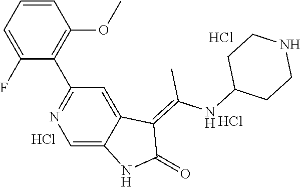 [SMILES: COc1cccc(F)c1-c1cc2c(cn1)NC(=O)/C2=C(/C)NC1CCNCC1 ; Cl ; Cl ; Cl] (Z)-5-(2-Fluoro-6- methoxyphenyl)-3-(1- (piperidin-4- ylamino)ethylidene)-1H- pyrrolo[2,3-c]pyridin- 2(3H)-one trihydrochloride | Step-1 of Method I, tert-butyl 4- aminopiperidine-1- carboxylate and 4- methylpyridine-3- boronic acid Method I Followed by Boc deprotection using HCl in EtOAc | ¹H NMR (400 MHz, DMSO-d₆) δ 1.83-2.00 (m, 2H), 2.12-2.17 (m, 2H), 2.69 (s, 3H), 3.01-3.06 (m, 2H), 3.71 (s, 3H), 3.36-3.41 (m, 2H), 7.02-7.14 (m, 2H), 7.58-7.83 (m, 1H), 8.24 (s, 1H), 8.34 (s, 1H), 9.26-9.34 (m, 2H), 11.60 (d, J = 8.4 Hz, 1H), 11.76 (s, 1H), 14.68 (s, 1H). |
| 73 | 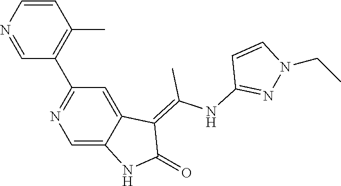 [SMILES: CCn1ccc(N/C(C)=C2\C(=O)Nc3cnc(-c4cnccc4C)cc32)n1] (Z)-3-(1-((1-Ethyl-1H- pyrazol-3- yl)amino)ethylidene)-5-(4- methylpyridin-3-yl)-1H- pyrrolo[2,3-c]pyridin- 2(3H)-one | Step 1 of Method I, 1-ethyl-1H-pyrazol- 3-amine and 4- methylpyridine-3- boronic acid Method I | ¹H NMR (400 MHz, DMSO-d₆) δ 1.38 (t, J = 7.2 Hz, 3H), 2.38 (s, 3H), 2.72 (s, 3H), 4.12 (q, J = 7.2 Hz, 2H), 6.29 (d, J = 2.0 Hz, 1H), 7.32 (d, J = 5.2 Hz, 1H), 7.52 (s, IH), 7.82 (d, J = 2.0 Hz, 1H), 8.24 (s, 1H), 8.43 (d, J = 4.8 Hz, 1H), 8.57 (s, 1H), 10.91 (s, 1H), 12.64 (s, 1H); ESI-MS (m/z) 361 (M + H)⁺. |
| 74 | 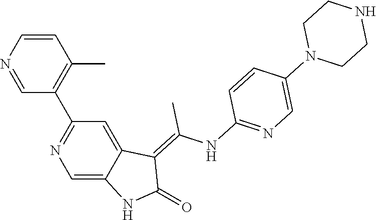 [SMILES: C/C(Nc1ccc(N2CCNCC2)cn1)=C1/C(=O)Nc2cnc(-c3cnccc3C)cc21] (Z)-5-(4-Methylpyridin-3- yl)-3-(1-((5-(piperazin-1- yl)pyridin-2- yl)amino)ethylidene)-1H- pyrrolo[2,3-c]pyridin- 2(3H)-one | Step 1 of Method I, tert-butyl 4-(6- aminopyridin-3- yl)piperazine-1- carboxylate and 4- methyl-3-(4,4,5,5- tetramethyl-1,3,2- dioxaborolan-2- yl)pyridine Method I followed by Boc- deprotection using PTSA | ¹H NMR (400 MHz, DMSO-d₆) δ 2.38 (s, 3H), 2.78 (s, 3H), 2.84 (t, J = 4.8 Hz, 4H), 3.11 (t, J = 4.4 Hz, 4H), 7.11 (d, J = 8.0 Hz, 1H), 7.20 (d, J = 9.6 Hz, 1H), 7.31 (d, J = 4.8 Hz, 1H), 7.44-7.48 (m, 1H), 7.52 (s, 1H), 8.14 (d, J = 2.4 Hz, 1H), 8.24 (s, 1H), 8.42 (d, J = 5.2 Hz, 1H), 8.58 (s, 1H), 10.93 (s, 1H), 12.74 (s, 1H); ESI-MS (m/z) 428 (M + H)⁺. |
| 75 | 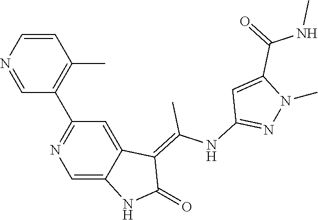 [SMILES: CNC(=O)c1cc(N/C(C)=C2\C(=O)Nc3cnc(-c4cnccc4C)cc32)nn1C] (Z)-N,1-Dimethyl-3-((1-(5- (4-methylpyridin-3-yl)-2- oxo-1H-pyrrolo[2,3- c]pyridin-3(2H)- ylidene)ethyl)amino)-1H- pyrazole-5-carboxamide | Step-1 of Method I, Intermediate D8, 4- methylpyridine-3- boronic acid Method I (Pd(dppf)₂Cl₂ was used as catalyst in step 3) | ¹H NMR (400 MHz, DMSO-d₆) δ 2.38 (s, 3H), 2.73 (s, 3H), 3.16 (s, 3H), 4.04 (s, 3H), 6.83 (s, 1H), 7.32 (d, J = 4.8 Hz, 1H), 7.57 (s, 1H), 8.25 (s, 1H), 8.43 (d, J = 4.8 Hz, 1H), 8.50 (s, 2H), 10.95 (s, 1H), 12.74 (s, 1H); ESI-MS (m/z) 404 (M + H)⁺. |
| 76 | 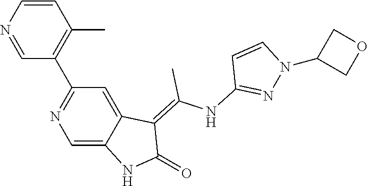 [SMILES: C/C(Nc1ccn(C2COC2)n1)=C1/C(=O)Nc2cnc(-c3cnccc3C)cc21] (Z)-5-(4-Methylpyridin-3- yl)-3-(1-((1-(oxctan-3-yl)- 1H-pyrazol-3- yl)amino)ethylidene)-1H- pyrrolo[2,3-c]pyridin- 2(3H)-one | Step-1 of Method I, Intermediate D9, 4- methylpyridine-3- boronic acid Method I | ¹H NMR (400 MHz, DMSO-d₆) δ 2.38 (s, 3H), 2.75 (s, 3H), 4.88-4.94 (m, 4H), 5.55-5.59 (m, 1H), 6.38 (s, 1H), 7.32 (d, J = 5.2 Hz, 1H), 7.55 (s, 1H), 7.95 (s, 1H), 8.25 (s, 1H), 8.42 (d, J = 5.2 Hz, 1H), 8.55 (s, 1H), 10.96 (s, 1H), 12.71 (s, 1H); ESI-MS (m/z) 389 (M + H)⁺. |
| 77 | 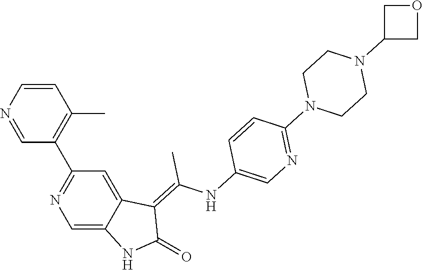 [SMILES: C/C(Nc1ccc(N2CCN(C3COC3)CC2)nc1)=C1/C(=O)Nc2cnc(-c3cnccc3C)cc21] (Z)-5-(4-Methylpyridin-3- yl)-3-(1-((6-(4-(oxetan-3- yl)piperazin-1-yl)pyridin-3- yl)amino)ethylidene)-1H- pyrrolo[2,3-c]pyridin- 2(3H)-one | Step-1 of Method I, Intermediate D5, 4- methylpyridine-3- boronic acid Method I | ¹H NMR (400 MHz, DMSO-d₆) δ 2.35-2.36 (m, 4H), 2.37 (s, 3H), 2.47 (s, 3H), 3.35-3.45 (m, 1H), 3.53-3.58 (m, 4H), 4.46- 4.51 (m, 2H), 4.54-4.59 (m, 2H), 6.94 (d, J = 8.8 Hz, 1H), 7.31 (d, J = 4.8 Hz, 1H), 7.47 (s, 1H), 7.58 (dd, J = 9.2, 2.8 Hz, 1H), 8.13 (d, J = 2.8 Hz, 1H), 8.23 (s, 1H), 8.42 (d, J = 4.8 Hz, 1H), 8.56 (s, 1H), 10.87 (s, 1H), 12.19 (s, 1H); ESI-MS (m/z) 485 (M + H)⁺. |
| 78 | 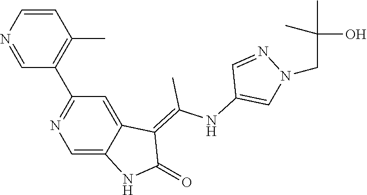 [SMILES: C/C(Nc1cnn(CC(C)(C)O)c1)=C1/C(=O)Nc2cnc(-c3cnccc3C)cc21] (Z)-3-(1-((1-(2-Hydroxy-2- methylpropyl)-1H-pyrazol- 4-yl)amino)ethylidene)-5- (4-methylpyridin-3-yl)-1H- pyrrolo[2,3-c]pyridin- 2(3H)-one | Step-1 of Method I, 1-(4-amino-1H- pyrazol-1-yl)-2- methylpropan-2-ol and 4- methylpyridine-3- boronic acid Method I | ¹H NMR (400 MHz, DMSO-d₆) δ 1.08 (s, 6H), 2.37 (s, 3H), 2.54 (s, 3H), 4.02 (s, 2H), 4.75 (s, 1H), 7.31 (d, J = 5.2 Hz, 1H), 7.49 (s, 1H), 7.59 (s, 1H), 7.89 (s, 1H), 8.22 (s, 1H), 8.42 (d, J = 4.8 Hz, 1H), 8.57 (s, 1H), 10.85 (s, 1H), 12.14 (s, 1H); ESI-MS (m/z) 405 (M + H)⁺. |
| 79 | 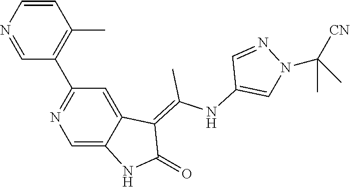 [SMILES: C/C(Nc1cnn(C(C)(C)C#N)c1)=C1/C(=O)Nc2cnc(-c3cnccc3C)cc21] (Z)-2-Methyl-2-(3-((1-(5- (4-methylpyridin-3-yl)-2- oxo-1H-pyrrolo[2,3- c]pyridin-3(2H)- ylidene)ethyl)amino)-1H- pyrazol-1-yl)propanenitrile | Step-1 of Method I, 2-(3-amino-1H- pyrazol-1-yl)-2- methylpropanenitrile (D11) and 4- methylpyridine-3- boronic acid Method I | ¹H NMR (400 MHz, DMSO-d₆) δ 1.99 (s, 6H), 2.38 (s, 3H), 2.75 (s, 3H), 6.50 (d, J = 2.4 Hz, 1H), 7.32 (d, J = 5.2 Hz, 1H), 7.56 (s, 1H), 8.11 (d, J = 2.4 Hz, 1H), 8.25 (s, 1H), 8.43 (d, J = 4.8 Hz, 1H), 8.58 (s, 1H), 10.98 (s, 1H), 12.70 (s, 1H). |
| 80 | 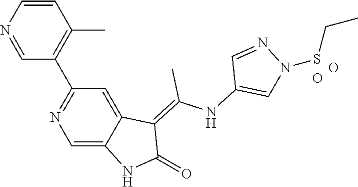 (Z)-3-(1-((1- (Ethylsulfonyl)-1H- pyrazol-4- yl)amino)ethylidene)-5-(4- methylpyridin-3-yl)-1H- pyrrolo[2,3-c]pyridin- 2(3H)-one | Intermediate B9 and 1-(ethylsulfonyl)-1H- pyrazol-4-amine, Method H | ¹H NMR (400 MHz, DMSO-d₆) δ 1.14 (t, J = 7.6 Hz, 3H), 2.37 (s, 3H), 2.58 (s, 3H), 3.76 (q, J = 7.6 Hz, 2H), 7.32 (d, J = 4.8 Hz, 1H), 7.54 (s, 1H), 8.20 (s, 1H), 8.24 (s, 1H), 8.43 (d, J = 4.8 Hz, 1H), 8.57 (s, 2H), 10.93 (s, 1H), 12.14 (s, 1H); ESI-MS (m/z) 425 (M + H)⁺. |
| 81 | 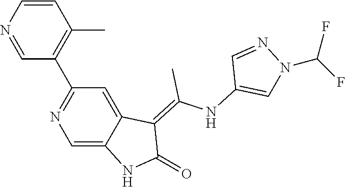 [SMILES: C/C(Nc1cnn(C(F)F)c1)=C1/C(=O)Nc2cnc(-c3cnccc3C)cc21] (Z)-3-(1-((1- (Difluoromethyl)-1H- pyrazol-3- yl)amino)ethylidene)-5-(4- methylpyridin-3-yl)-1H- pyrrolo[2,3-c]pyridin- 2(3H)-one | Step-1 of Method I, 1-(difluoromethyl)- 1H-pyrazol-3-amine and 4- methylpyridine-3- boronic acid Method I | ¹H NMR (400 MHz, DMSO-d₆) δ 2.38 (s, 3H), 2.79 (s, 3H), 6.64 (d, J = 2.4 Hz, 1H), 7.33 (d, J = 5.2 Hz, 1H), 7.58 (s, 1H), 7.64-7.93 (m, 1H), 8.27 (s, 1H), 8.32 (d, J = 2.8 Hz, 1H), 8.42 (d, J = 5.2 Hz, 1H), 8.58 (s, 1H), 11.02 (s, 1H), 12.77 (s, 1H); ESI-MS (m/z) 383 (M + H)⁺. |
| 82 | 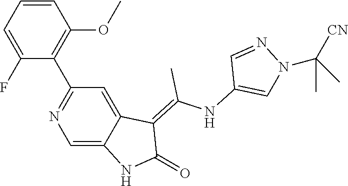 [SMILES: COc1cccc(F)c1-c1cc2c(cn1)NC(=O)/C2=C(/C)Nc1cnn(C(C)(C)C#N)c1] (Z)-2-(4-((1-(5-(2-Fluoro- 6-methoxyphenyl)-2-oxo- 1H-pyrrolo[2,3-c]pyridin- 3(2H)- ylidene)ethyl)amino)-1H- pyrazol-1-yl)-2- methylpropanenitrile | Step-1 of Method I, 2-(4-amino-1H- pyrazol-1-yl)-2- methylpropanenitrile (D16) and 4- methylpyridine-3- boronic acid Method I | ¹H NMR (400 MHz, DMSO-d₆) δ 1.99 (s, 6H), 2.49 (s, 3H), 3.72 (s, 3H), 6.87 (t, J = 8.4 Hz, 1H), 6.95 (d, J = 8.4 Hz, 1H), 7.32 (s, 1H), 7.34-7.42 (m, 1H), 7.83 (s, 1H), 8.17 (s, 1H), 8.32 (d, J = 5.6 Hz, 1H), 10.83 (s, 1H), 12.15 (s, 1H); ESI-MS (m/z) 434 (M + H)⁺. |
| 83 | 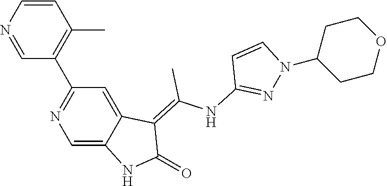 [SMILES: C/C(Nc1ccn(C2CCOCC2)n1)=C1/C(=O)Nc2cnc(-c3cnccc3C)cc21] (Z)-5-(4-Methylpyridin-3- yl)-3-(1-((1-(tetrahydro- 2H-pyran-4-yl)-1H- pyrazol-3- yl)amino)ethylidene)-1H- pyrrolo[2,3-c]pyridin- 2(3H)-one | Step-1 of Method I, Intermediate D10, 4- methylpyridine-3- boronic acid Method I | ¹H NMR (400 MHz, DMSO-d₆) δ 1.82-1.99 (m, 2H), 2.0-2.08 (m, 2H), 2.37 (s, 3H), 2.49 (s, 3H), 3.45 (t, J = 7.2 Hz, 2H), 3.94- 3.98 (m, 2H), 4.36-4.42 (m, 1H), 6.35 (s, 1H), 7.33 (s, 1H), 7.55 (d, J = 11.2 Hz, 2H), 8.28 (s, 1H), 8.48-8.59 (m, 2H), 11.01 (s, 1H), 12.32 (s, 1H); ESI-MS (m/z) 417 (M + H)⁺. |
| 84 | 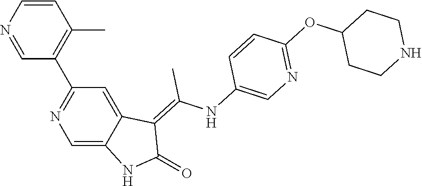 [SMILES: C/C(Nc1ccc(OC2CCNCC2)nc1)=C1/C(=O)Nc2cnc(-c3cnccc3C)cc21] (Z)-5-(4-Methylpyridin-3- yl)-3-(1-((6-(piperidin-4- yloxy)pyridin-3- yl)amino)ethylidene)-1H- pyrrolo[2,3-c]pyridin- 2(3H)-one | Step 1 of Method I, tert-butyl 4-((5- aminopyridin-2- yl)oxy)piperidine-1- carboxylate and 4- methyl-3-(4,4,5,5- tetramethyl-1,3,2- dioxaborolan-2- yl)pyridine Method I followed by Boc- deprotection using PTSA | ¹H NMR (400 MHz, DMSO-d₆) δ 1.50-1.55 (m, 2H), 1.96-1.99 (m, 2H), 2.48 (s, 3H), 2.63 (s, 3H), 2.96-2.99 (m, 2H), 3.26- 3.41 (m, 2H), 5.03-5.08 (m, 1H), 6.88 (d, J = 8.8 Hz, 1H), 7.31 (d, J = 4.8 Hz, 1H), 7.48 (s, 1H), 7.74 (dd, J₁ = 2.8 Hz, J₂ = 8.8 Hz, 1H), 8.18 (d, J = 2.4 Hz, 1H), 8.24 (s, 1H), 8.41 (d, J = 5.2 Hz, 1H), 8.56 (s, 1H), 10.90 (s, 1H), 12.24 (s, 1H); ESI-MS (m/z) 443 (M + H)⁺. |
| 85 | 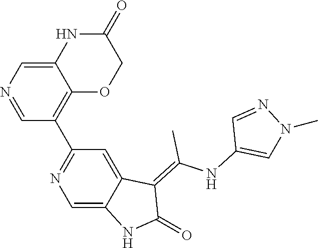 [SMILES: C/C(Nc1cnn(C)c1)=C1/C(=O)Nc2cnc(-c3cncc4c3OCC(=O)N4)cc21] (Z)-8-(3-(1-((1-Methyl-1H- pyrazol-4- yl)amino)ethylidene)-2- oxo-2,3-dihydro-1H- pyrrolo[2,3-c]pyridin-5-yl)- 2H-pyrido[4,3- b][1,4]oxazin-3(4H)-one | Step 2 of Method I and 8-(4,4,5,5- tetramethyl-1,3,2- dioxaborolan-2-yl)- 2H-pyrido[4,3- b][1,4]oxazin-3(4H)- one Method I | ¹H NMR (400 MHz, DMSO-d₆) δ 2.50 (s, 3H), 3.86 (s, 3H),4.79 (s, 2H), 7.59 (s, 1H),7.83 (s, 1H), 7.97 (s, 1H),8.06 (s, 1H), 8.22 (s, 1H),8.49 (s, 1H), 10.86 (s, 1H), 11.0 (s, 1H), 12.08 (s, 1H); ESI-MS (m/z) 404 (M + H)⁺. |
| 86 | 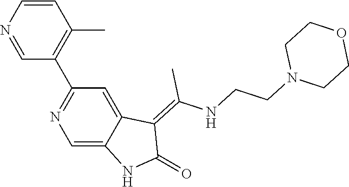 [SMILES: C/C(NCCN1CCOCC1)=C1/C(=O)Nc2cnc(-c3cnccc3C)cc21] (Z)-5-(4-Methylpyridin-3- yl)-3-(1-((2- morpholinoethyl)amino) ethylidene)-1H-pyrrolo[2,3- c]pyridin-2(3H)-one | Step 1 of Method I, 2- morpholinoethanamine and 4-methyl-3- (4,4,5,5-tetramethyl- 1,3,2-dioxaborolan-2- yl)pyridine Method I | ¹H NMR (400 MHz, DMSO-d₆) δ 2.37 (s, 3H), 2.45-2.58 (m, 9H), 3.58- 3.62 (m, 6H), 7.29 (d, J = 5.2 Hz, 1H), 7.42 (s, 1H), 8.15 (s, 1H), 8.40 (d, J = 4.8 Hz, 1H), 8.56 (s, 1H), 10.55 (s, 1H), 10.96 (t, J = 5.2 Hz, 1H); ESI-MS (m/z) 380 (M + H)⁺. |
| 87 | 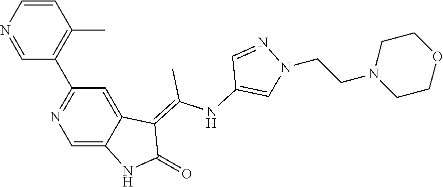 [SMILES: C/C(Nc1cnn(CCN2CCOCC2)c1)=C1/C(=O)Nc2cnc(-c3cnccc3C)cc21] (Z)-5-(4-Methylpyridin-3- yl)-3-(1-((1-(2- morpholinoethyl)-1H- pyrazol-4- yl)amino)ethylidene)-1H- pyrrolo[2,3-c]pyridin- 2(3H)-one | Step-1 of Method I, 1-(2- morpholinoethyl)- 1H-pyrazol-4-amine, 4-methylpyridine-3- boronic acid Method I | ¹H NMR (400 MHz, DMSO-d₆) δ 2.37 (s, 3H), 2.40 (s, 3H), 2.49-2.55 (m, 2H), 2.68-2.74 (m, 2H), 3.35-3.40 (m, 2H), 3.52- 3.56 (m, 4H), 4.21-4.26 (m, 2H), 7.31 (d, J = 2.0 Hz, 1H), 7.50 (s, 1H), 7.59 (s, 1H), 8.01 (s, 1H), 8.22 (s, 1H), 8.42 (d, J = 4.8 Hz, 1H), 8.56 (s, 1H), 10.86 (s, 1H), 12.12 (s, 1H); ESI-MS (m/z) 446 (M + H)⁺. |
| 88 | 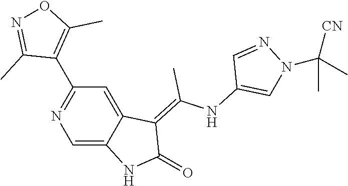 [SMILES: C/C(Nc1cnn(C(C)(C)C#N)c1)=C1/C(=O)Nc2cnc(-c3c(C)noc3C)cc21] (Z)-2-(4-((1-(5-(3,5- Dimethylisoxazol-4-yl)-2- oxo-1H-pyrrolo[2,3- c]pyridin-3(2H)- ylidene)ethyl)amino)-1H- pyrazol-1-yl)-2- methylpropanenitrile | Step-1 of Method I, 2-(4-amino-1H- pyrazol-1-yl)-2- methylpropanenitrile (D16) and (3,5- dimethylisoxazol-4- yl)boronic acid Method I | ¹H NMR (400 MHz, DMSO-d₆) δ 2.00 (s, 6H), 2.35 (s, 3H), 2.53 (s, 3H), 2.57 (s, 3H), 7.43 (s, 1H), 7.84 (s, 1H), 8.20 (s, 1H), 8.32 (s, 1H), 10.86 (s, 1H), 12.17 (s, 1H); ESI-MS (m/z) 404 (M + H)⁺. |
| 89 | 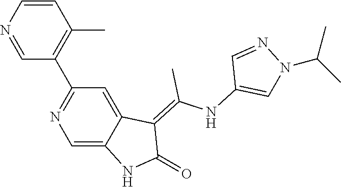 [SMILES: C/C(Nc1cnn(C(C)C)c1)=C1/C(=O)Nc2cnc(-c3cnccc3C)cc21] (Z)-3-(1-((1-Isopropyl-1H- pyrazol-4- yl)amino)ethylidene)-5-(4- methylpyridin-3-yl)-1H- pyrrolo[2,3-c]pyridin- 2(3H)-one | Step-1 of Method I, 1-isopropyl-1H- pyrazol-4-amine and 4-methylpyridine-3- boronic acid Method I | ¹H NMR (400 MHz, DMSO-d₆) δ 1.43 (s, 3H), 1.44 (s, 3H), 2.37 (s, 3H), 2.55 (s, 3H), 4.47-4.52 (m, 1H), 7.32 (d, J = 5.2 Hz, 1H), 7.49 (s, 1H), 7.59 (s, 1H), 8.05 (s, 1H), 8.22 (s, 1H), 8.43 (d, J = 5.2 Hz, 1H), 8.56 (s, 1H), 10.86 (s, 1H), 12.19 (s, 1H); ESI-MS (m/z) 375 (M + H)⁺. |
| 90 | 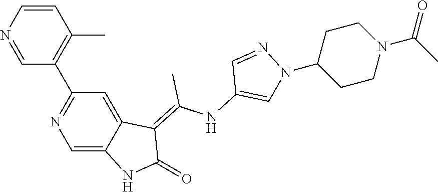 [SMILES: CC(=O)N1CCC(n2cc(N/C(C)=C3\C(=O)Nc4cnc(-c5cnccc5C)cc43)cn2)CC1] (Z)-3-(1-((1-(1- Acetylpiperidin-4-yl)-1H- pyrazol-4- yl)amino)ethylidene)-5-(4- methylpyridin-3-yl)-1H- pyrrolo[2,3-c]pyridin- 2(3H)-one | Step-1 of Method I, Intermediate D17 and 4-methylpyridine-3- boronic acid Method I | ¹H NMR (400 MHz, DMSO-d₆) δ 1.72-1.80 (m, 1H), 1.85-1.93 (m, 1H), 2.01-2.10 (m, 5H), 2.37 (s, 3H), 2.54 (s, 3H), 2.68-2.76 (m, 1H), 3.16-3.25 (m, 1H), 3.89-3.96 (m, 1H), 4.39- 4.48 (m, 2H), 7.32 (d, J = 4.8 Hz, 1H), 7.49 (s, 1H), 7.63 (s, 1H), 8.08 (s, s, 1H), 8.22 (s, 1H), 8.42 (d, J = 4.8 Hz, 1H), 8.56 (s, 1H), 10.86 (s, 1H), 12.12 (s, 1H); ESI- MS (m/z) 457 (M)⁺. |
| 91 | 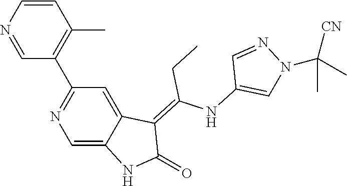 [SMILES: CC/C(Nc1cnn(C(C)(C)C#N)c1)=C1/C(=O)Nc2cnc(-c3cnccc3C)cc21] (Z)-2-Methyl-2-(4-((1-(5- (4-methylpyridin-3-yl)-2- oxo-1H-pyrrolo[2,3- c]pyridin-3(2H)- ylidene)propyl)amino)-1H- pyrazol-1-yl)propanenitrile | Step 3 of Intermediate B7, triethylorthopropionate, 2-(4-amino-1H- pyrazol-1-yl)-2- methylpropanenitrile (D16), 4- methylpyridine-3- boronic acid Method I | ¹H NMR (400 MHz, DMSO-d₆) δ 1.26 (t, J = 7.6 Hz, 3H), 2.00 (s, 6H), 2.38 (s, 3H), 2.82 (q, J = 7.6 Hz, 1H), 7.33 (d, J = 5.2 Hz, 1H), 7.42 (s, 1H), 7.84 (s, 1H), 8.25 (s, 1H), 8.33 (s, 1H), 8.43 (d, J = 5.2 Hz, 1H), 8.57 (s, 1H), 10.94 (s, 1H), 12.08 (s, 1H). |
| 92 | 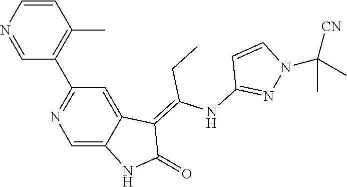 [SMILES: CC/C(Nc1ccn(C(C)(C)C#N)n1)=C1/C(=O)Nc2cnc(-c3cnccc3C)cc21] (Z)-2-Methyl-2-(3-((1-(5- (4-methylpyridin-3-yl)-2- oxo-1H-pyrrolo[2,3- c]pyridin-3(2H)- ylidene)propyl)amino)-1H- pyrazol-1-yl)propanenitrile | Step 3 of Intermediate B7, triethylorthopropionate, 2-(3-amino-1H- pyrazol-1-yl)-2- methylpropanenitrile (D11), 4- methylpyridine-3- boronic acid Method I | ¹H NMR (400 MHz, DMSO-d₆) δ 1.36 (t, J = 7.2 Hz, 3H), 2.00 (s, 6H), 2.39 (s, 3H), 3.11 (q, J = 7.2 Hz, 1H), 6.46 (d, J = 2.4 Hz, 1H), 7.34 (d, J = 4.8 Hz, 1H), 7.47 (s, 1H), 8.12 (s, 1H), 8.27 (s, 1H), 8.33 (s, 1H), 8.44 (d, J = 5.2 Hz, 1H), 8.58 (s, 1H), 11.04 (s, 1H), 12.69 (s, 1H); ESI-MS (m/z) 414 (M + H)⁺. |
| 93 | 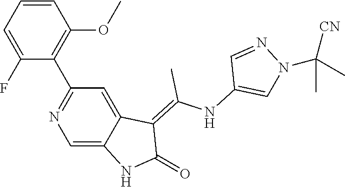 [SMILES: COc1cccc(F)c1-c1cc2c(cn1)NC(=O)/C2=C(/C)Nc1cnn(C(C)(C)C#N)c1] (Z)-2-(3-((1-(5-(2-Fluoro- 6-methoxyphenyl)-2-oxo- 1H-pyrrolo[2,3-c]pyridin- 3(2H)- ylidene)ethyl)amino)-1H- pyrazol-1-yl)-2- methylpropanenitrile | Step-1 of Method I, 2-(3-amino-1H- pyrazol-1-yl)-2- methylpropanenitrile (D11) and 2-fluoro-6- methoxyphenylboronic acid Method I | ¹H NMR (400 MHz, DMSO-d₆) δ 1.99 (s, 6H), 2.68 (s, 3H), 3.73 (s, 3H), 6.50 (d, J = 2.8 Hz, 1H), 6.88 (t, J = 8.8 Hz, 1H), 6.96 (d, J = 8.4 Hz, 1H), 7.36-7.44 (m, 2H), 8.11 (d, J = 2.4 Hz, 1H), 8.20 (s, 1H), 10.98 (s, 1H), 12.71 (s, 1H); ESI-MS (m/z) 433 (M + H)⁺. |
| 94 | 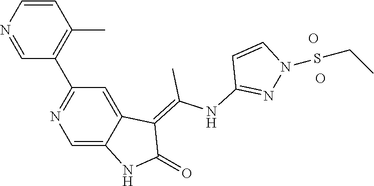 (Z)-3-(1-((1- (Ethylsulfonyl)-1H- pyrazol-3- yl)amino)ethylidene)-5-(4- methylpyridin-3-yl)-1H- pyrrolo[2,3-c]pyridin- 2(3H)-one | Intermediate B9 and 1-(ethylsulfonyl)-1H- pyrazol-3-amine, Method H | ¹H NMR (400 MHz, DMSO-d₆) δ 1.13 (t, J = 7.6 Hz, 3H), 2.38 (s, 3H), 2.81 (s, 3H), 3.74 (q, J = 7.6 Hz, 2H), 6.76 (d, J = 2.8 Hz, 1H), 7.33 (d, J = 5.2 Hz, 1H), 7.60 (s, 1H), 8.27 (s, 1H), 8.38 (s, 1H), 8.44 (d, J = 5.2 Hz, 1H), 8.58 (s, 2H), 11.07 (s, 1H), 12.79 (s, 1H); ESI-MS (m/z) 425 (M + H)⁺. |
| 95 | 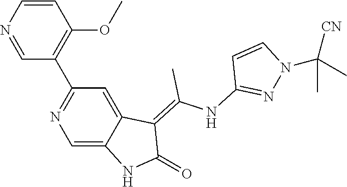 [SMILES: COc1ccncc1-c1cc2c(cn1)NC(=O)/C2=C(/C)Nc1ccn(C(C)(C)C#N)n1] (Z)-2-(3-((1-(5-(4- Methoxypyridin-3-yl)-2- oxo-1H-pyrrolo[2,3- c]pyridin-3(2H)- ylidene)ethyl)amino)-1H- pyrazol-1-yl)-2- methylpropanenitrile | Step 1 of Method I, 2-(3-amino-1H- pyrazol-1-yl)-2- methylpropanenitrile (D11) and 4-methyl- 3-(4,4,5,5- tetramethyl-1,3,2- dioxaborolan-2- yl)pyridine Method I | ¹H NMR (400 MHz, DMSO-d₆) δ 1.99 (s, 6H), 2.74 (s, 3H), 3.92 (s, 3H), 6.50 (s, 1H), 7.16 (d, J = 5.6 Hz, 1H), 7.86 (s, 1H), 8.11 (s, 1H), 8.24 (s, 1H), 8.43 (d, J = 5.6 Hz, 1H), 8.72 (s, 1H), 10.96 (s, 1H), 12.68 (s, 1H); ESI-MS (m/z) 416 (M + H)⁺. |
| 96 | 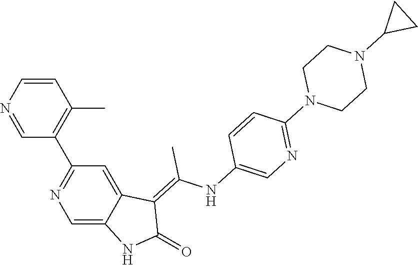 [SMILES: C/C(Nc1ccc(N2CCN(C3CC3)CC2)nc1)=C1/C(=O)Nc2cnc(-c3cnccc3C)cc21] (Z)-3-(1-((6-(4- Cyclopropylpiperazin-1- yl)pyridin-3- yl)amino)ethylidene)-5-(4- methylpyridin-3-yl)-1H- pyrrolo[2,3-c]pyridin- 2(3H)-one | Step 1 of Method I, 6-(4- cyclopropylpiperazin- 1-yl)pyridin-3-amine (D14) and 4-methyl- 3-(4,4,5,5- tetramethyl-1,3,2- dioxaborolan-2- yl)pyridine Method I | ¹H NMR (400 MHz, DMSO-d₆) δ 0.37-0.47 (m, 4H), 1.63-1.67 (m, 1H), 2.37 (s, 3H), 2.50 (s, 3H), 2.62 (t, J = 4.8 Hz, 4H), 3.49 (t, J = 4.8 Hz, 4H), 6.92 (d, J = 9.2 Hz, 1H), 7.30 (d, J = 4.8 Hz, 1H), 7.46 (s, 1H), 7.56 (dd, J₁ = 2.4 Hz, J₂ = 8.8 Hz, 1H), 8.11 (s, 1H), 8.23 (s, 1H), 8.41 (d, J = 5.2 Hz, 1H), 8.56 (s, 1H), 10.86 (s, 1H), 12.18 (s, 1H); ESI-MS (m/z) 468 (M + H)⁺. |
| 97 | 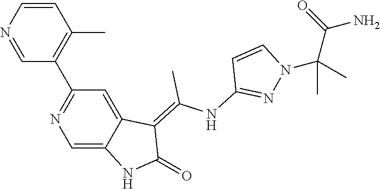 [SMILES: C/C(Nc1ccn(C(C)(C)C(N)=O)n1)=C1/C(=O)Nc2cnc(-c3cnccc3C)cc21] (Z)-2-Methyl-2-(3-((1-(5- (4-methylpyridin-3-yl)-2- oxo-1H-pyrrolo[2,3- c]pyridin-3(2H)- ylidene)ethyl)amino)-1H- pyrazol-1-yl)propanamide | Step 1 of Method I, 2-(3-amino-1H- pyrazol-1-yl)-2- methylpropanamide (D19) and 4-methyl- 3-(4,4,5,5- tetramethyl-1,3,2- dioxaborolan-2- yl)pyridine Method I | ¹H NMR (400 MHz, DMSO-d₆) δ 1.71 (s, 6H), 2.38 (s, 3H), 2.72 (s, 3H), 6.36 (d, J = 2.4 Hz, 1H), 6.94 (s, 1H), 7.23 (s, 1H), 7.32 (d, J = 5.2 Hz, 1H), 7.53 (s, 1H), 7.92 (d, J = 2.0 Hz, 1H), 8.24 (s, 1H), 8.43 (d, J = 4.8 Hz, 1H), 8.58 (s, 1H), 10.93 (s, 1H), 12.62 (s, 1H); ESI-MS (m/z) 419 (M + H)⁺. |
| 98 | 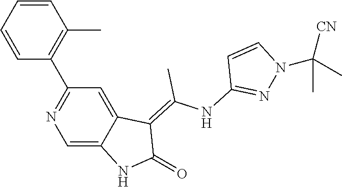 [SMILES: C/C(Nc1ccn(C(C)(C)C#N)n1)=C1/C(=O)Nc2cnc(-c3ccccc3C)cc21] (Z)-2-Methyl-2-(3-((1-(2- oxo-5-(o-tolyl)-1H- pyrrolo[2,3-c]pyridin- 3(2H)- ylidene)ethyl)amino)-1H- pyrazol-1-yl)propanenitrile | Step-1 of Method I, 2-(3-amino-1H- pyrazol-1-yl)-2- methylpropanenitrile (D11) and o- tolylboronic acid Method I | ¹H NMR (400 MHz, DMSO-d₆) δ 1.99 (s, 6H), 2.33 (s, 3H), 2.73 (s, 3H), 6.49 (s, 1H), 7.25-7.28 (m, 3H), 7.41 (d, J = 5.6 Hz, 1H), 7.45 (s, 1H), 8.11 (s, 1H), 8.21 (s, 1H), 10.93 (s, 1H), 12.68 (s, 1H); ESI-MS (m/z) 399 (M + H)⁺. |
| 99 | 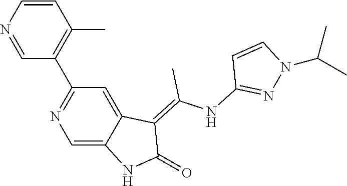 [SMILES: C/C(Nc1ccn(C(C)C)n1)=C1/C(=O)Nc2cnc(-c3cnccc3C)cc21] (Z)-3-(1-((1-Isopropyl-1H- pyrazol-3- yl)amino)ethylidene)-5-(4- methylpyridin-3-yl)-1H- pyrrolo[2,3-c]pyridin- 2(3H)-one | Step-1 of Method I, 1-isopropyl-1H- pyrazol-3-amine and 4-methylpyridine-3- boronic acid Method I | ¹H NMR (400 MHz, DMSO-d₆) δ 1.43 (d, J = 6.8 Hz, 6H), 2.38 (s, 3H), 2.72 (s, 3H), 4.44-4.52 (m, 1H), 6.29 (d, J = 2.4 Hz, 1H), 7.32 (d, J = 4.8 Hz, 1H), 7.53 (s, 1H), 7.85 (d, J = 2.4 Hz, 1H), 8.24 (s, 1H), 8.43 (d, J = 4.8 Hz, 1H), 8.58 (s, 1H), 10.92 (s, 1H), 12.65 (s, 1H). |
| 100 | 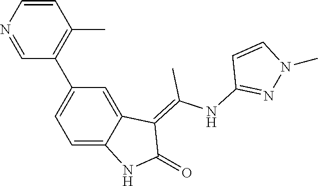 [SMILES: C/C(Nc1ccn(C)n1)=C1/C(=O)Nc2ccc(-c3cnccc3C)cc21] (Z)-3-(1-((1-Methyl-1H- pyrazol-3- yl)amino)ethylidene)-5-(4- methylpyridin-3-yl)indolin- 2-one | Step-1 of Method I, 1-methyl-1H-pyrazol- 3-amine and 4- methyl-3-(4,4,5,5- tetramethyl-1,3,2- dioxaborolan-2- yl)pyridine Method I | ¹H NMR (400 MHz, DMSO-d₆) δ 2.30 (s, 3H), 2.63 (s, 3H), 3.81 (s, 3H), 6.20 (d, J = 2.4 Hz, 1H), 6.97-7.04 (m, 2H), 7.32 (d, J = 4.8 Hz, 1H), 7.35 (s, 1H), 7.73 (d, J = 2.4 Hz, 1H), 8.39 (d, J = 4.8 Hz, 1H), 8.41 (s, 1H), 10.71 (s, 1H), 12.32 (s, 1H). |
| 101 | 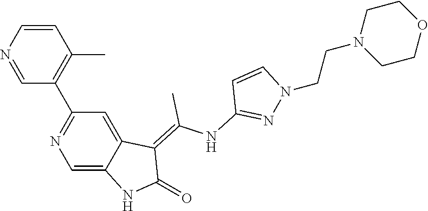 [SMILES: C/C(Nc1ccn(CCN2CCOCC2)n1)=C1/C(=O)Nc2cnc(-c3cnccc3C)cc21] (Z)-5-(4-Methylpyridin-3- yl)-3-(1-((1-(2- morpholinoethyl)-1H- pyrazol-3- yl)amino)ethylidene)-1H- pyrrolo[2,3-c]pyridin- 2(3H)-one | Step-1 of Method I, 1-(2- morpholinoethyl)- 1H-pyrazol-3-amine (D18) and 4-methyl- 3-(4,4,5,5- tetramethyl-1,3,2- dioxaborolan-2- yl)pyridine Method I | ¹H NMR (400 MHz, DMSO-d₆) δ 2.38 (s, 3H), 2.69-2.74 (m, 4H), 3.32 (s, 3H), 3.52-3.57 (m, 4H), 4.05 (s, 3H), 4.21 (t, J = 6.4 Hz, 1H), 6.29 (d, J = 2.4 Hz, 1H), 7.32 (d, J = 4.8 Hz, 1H), 7.52 (s, 1H), 7.83 (d, J = 2.4 Hz, 1H), 8.24 (s, 1H), 8.43 (d, J = 5.2 Hz, 1H), 8.57 (s, 1H), 10.92 (s, 1H), 12.62 (s, 1H). |
| 102 | 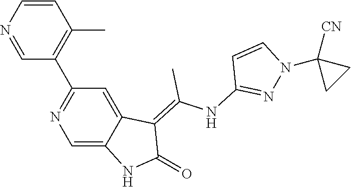 [SMILES: C/C(Nc1ccn(C2(C#N)CC2)n1)=C1/C(=O)Nc2cnc(-c3cnccc3C)cc21] (Z)-1-(3-((1-(5-(4- Methylpyridin-3-yl)-2-oxo- 1H-pyrrolo[2,3-c]pyridin- 3(2H)- ylidene)ethyl)amino)-1H- pyrazol-1- yl)cyclopropanecarbonitrile | Step 1 of Method I, D15 and 4-methyl-3- (4,4,5,5-tetramethyl- 1,3,2-dioxaborolan-2- yl)pyridine Method I | ¹H NMR (400 MHz, DMSO-d₆) δ 1.86-1.91 (m, 4H), 2.38 (s, 3H), 2.76 (s, 3H), 8.46 (s, 1H), 7.32 (d, J = 5.2 Hz, 1H), 7.56 (s, 1H), 8.12 (s, 1H), 8.26 (s, 1H), 8.43 (d, J = 4.8 Hz, 1H), 8.58 (s, 1H), 10.99 (s, 1H), 12.71 (s, 1H). |
| 103 | 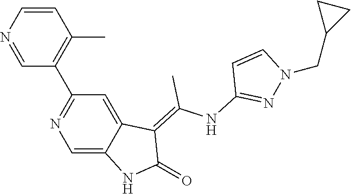 [SMILES: C/C(Nc1ccn(CC2CC2)n1)=C1/C(=O)Nc2cnc(-c3cnccc3C)cc21] (Z)-3-(1-((1- (Cyclopropylmethyl)-1H- pyrazol-3- yl)amino)ethylidene)-5-(4- methylpyridin-3-yl)-1H- pyrrolo[2,3-c]pyridin- 2(3H)-one | Step 1 of Method I, 1- (cyclopropylmethyl)- 1H-pyrazol-3-amine (CA S #899899-07- 1) and 4-methyl-3- (4,4,5,5-tetramethyl- 1,3,2-dioxaborolan-2- yl)pyridine Method I | ¹H NMR (400 MHz, DMSO-d₆) δ 0.36-0.39 (m, 2H), 0.52-0.56 (m 2H), 1.23-1.27 (m, 1H), 2.38 (s, 3H), 2.72 (s, 3H), 3.95 (d J = 7.2 Hz, 2H), 6.30 (s, 1H), 7.31 (d, J = 4.8 Hz, 1H), 7.52 (s, 1H), 7.84 (s, 1H), 8.24 (s, 1H), 8.42 (d, J = 4.8 Hz, 1H), 8.58 (s, 1H), 10.91 (s, 1H), 12.65 (s, 1H). |
| 104 | 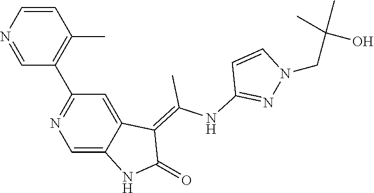 [SMILES: C/C(Nc1ccn(CC(C)(C)O)n1)=C1/C(=O)Nc2cnc(-c3cnccc3C)cc21] (Z)-3-(1-((1-(2-Hydroxy-2- methylpropyl)-1H-pyrazol- 3-yl)amino)ethylidene)-5- (4-methylpyridin-3-yl)-1H- pyrrolo[2,3-c]pyridin- 2(3H)-one | Step 1 of Method 1, 1-(3-amino-1H- pyrazol-1-yl)-2- methylpropan-2-ol (D20) and 4-methyl- 3-(4,4,5,5- tetramethyl-1,3,2- dioxaborolan-2- yl)pyridine Method I | ¹H NMR (400 MHz, DMSO-d₆) δ 1.09 (s, 6H), 2.38 (s, 3H), 2.71 (s, 3H), 3.99 (s, 2H), 4.73 (s, 1H), 6.31 (d, J = 4.8 Hz, 1H), 7.32 (d, J = 4.8 Hz, 1H), 7.53 (s, 1H), 7.73 (d, J = 2.4 Hz, 1H), 8.24 (s, 1H), 8.43 (d, J = 5.2 Hz, 1H), 8.58 (s, 1H), 10.93 (s, 1H), 12.60 (s, 1H); ESI-MS (m/z) (M + H)⁺. |
| 105 | 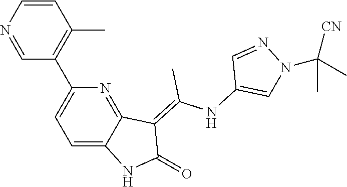 (Z)-2-Methyl-2-(4-((1-(5- (4-methylpyridin-3-yl)-2- oxo-1H-pyrrolo[3,2- b]pyridin-3(2H)- ylidene)ethyl)amino)-1H- pyrazol-1-yl)propanenitrile | Intermediate B12, 2- (4-amino-1H- pyrazol-1-yl)-2- methylpropanenitrile (D16) and 4-methyl- 3-(4,4,5,5- tetramethyl-1,3,2- dioxaborolan-2- yl)pyridine Method I | ESI-MS (m/z) 405 (M + H)⁺. |
| 106 | 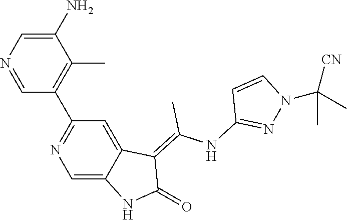 [SMILES: C/C(Nc1ccn(C(C)(C)C#N)n1)=C1/C(=O)Nc2cnc(-c3cncc(N)c3C)cc21] (Z)-2-(3-((1-(5-(5-Amino- 4-methylpyridin-3-yl)-2- oxo-1H-pyrrolo[2,3- c]pyridin-3(2H)- ylidene)ethyl)amino)-1H- pyrazol-1-yl)-2- methylpropanenitrile | Step-1 of Method I, Intermediate D11, 4- methylpyridine-3- boronic acid Method I | ¹H NMR (400 MHz, DMSO-d₆) δ 1.99 (s, 6H), 2.05 (s, 3H), 3.34 (s, 3H), 5.15 (br s, 2H), 6.48 (s 1H), 7.42 (s, 1H), 7.79 (s, 1H), 7.94 (s, 1H), 8.11 (s, 1H), 8.22 (s, 1H), 10.95 (s, 1H), 12.68 (s, 1H); ESI-MS (m/z) 415 (M + H)⁺. |
| 107 | 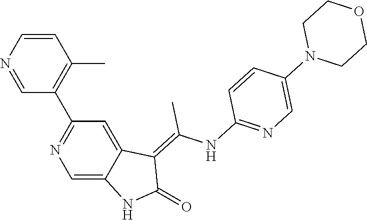 [SMILES: C/C(Nc1ccc(N2CCOCC2)cn1)=C1/C(=O)Nc2cnc(-c3cnccc3C)cc21] (Z)-5-(4-Methylpyridin-3- yl)-3-(1-((5- morpholinopyridin-2- yl)amino)ethylidene)-1H- pyrrolo[2,3-c]pyridin- 2(3H)-one | Step-1 of Method I, 5-morpholinopyridin- 2-amine, 4-methyl-3- (4,4,5,5-tetramethyl- 1,3,2-dioxaborolan-2- yl)pyridine Method I | ¹H NMR (400 MHz, DMSO-d₆) δ 2.38 (s, 3H), 2.79 (s, 3H), 3.19 (t, J = 4.4 Hz, 4H), 3.76 (t, J = 4.8 Hz, 4H), 7.23 (d, J = 9.2 Hz, 1H), 7.31 (d, J = 4.8 Hz, 1H), 7.48-7.52 (m, 2H), 8.17 (s, 1H), 8.24 (s, 1H), 8.43 (d, J = 4.8 Hz, 1H), 8.58 (s, 1H), 10.93 (s, 1H), 12.76 (s, 1H); ESI-MS (m/z) 430 (M + H)⁺. |
| 108 | 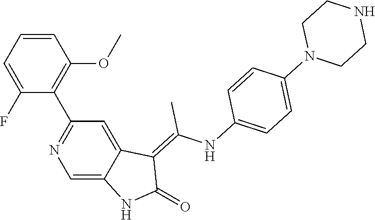 [SMILES: COc1cccc(F)c1-c1cc2c(cn1)NC(=O)/C2=C(/C)Nc1ccc(N2CCNCC2)cc1] (Z)-5-(2-Fluoro-6- methoxyphenyl)-3-(1-((4- (piperazin-1- yl)phenyl)amino)ethylidene)- 1H-pyrrolo[2,3-c]pyridin- 2(3H)-one | Step 1 of Method I, tert-butyl 4-(4- aminophenyl)piperazine- 1-carboxylate and 2-fluoro-6- methoxyphenylboronic acid Method I followed by Boc- deprotection using PTSA | ¹H NMR (400 MHz, DMSO-d₆) δ 2.43 (s, 3H), 2.48 (t, J = 5.2 Hz, 4H), 3.08 (t, J = 4.4 Hz, 4H), 3.71 (s, 3H), 6.86 (t, J = 8.8 Hz, 1H), 6.93-7.06 (m, 3H), 7.18 (d, J = 8.8 Hz, 3H), 7.35-7.40 (m, 1H), 8.16 (s, 1H), 10.79 (s, 1H), 12.31 (s, 1H); ESI-MS (m/z) 460 (M + H)⁺. |
| 109 | 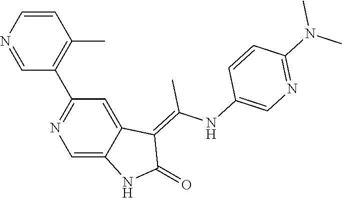 [SMILES: C/C(Nc1ccc(N(C)C)nc1)=C1/C(=O)Nc2cnc(-c3cnccc3C)cc21] (Z)-3-(1-((6- (Dimethylamino)pyridin-3- yl)amino)ethylidene)-5-(4- methylpyridin-3-yl)-1H- pyrrolo[2,3-c]pyridin- 2(3H)-one | Step 1 of Method I, N2,N2- dimethylpyridine-2,5- diamine and 4- methyl-3-(4,4,5,5- tetramethyl-1,3,2- dioxaborolan-2- yl)pyridine Method I | ¹H NMR (400 MHz, DMSO-d₆) δ 2.37 (s, 3H), 2.49 (s, 3H), 3.34 (s, 6H), 6.72 (d, J = 8.8 Hz, 1H), 7.30 (d, J = 5.2 Hz, 1H), 7.46 (s, 1H), 7.53 (dd, J₁ = 2.8 Hz, J₂ = 9.2 Hz, 1H), 8.09 (s, 1H), 8.22 (s, 1H), 8.41 (d, J = 5.2 Hz, 1H), 8.56 (s, 1H), 10.85 (s, 1H), 12.16 (s, 1H); ESI-MS (m/z) 387 (M + H)⁺. |
| 110 | 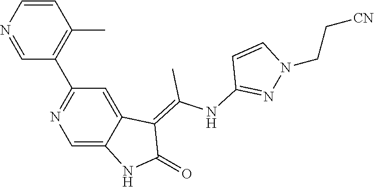 [SMILES: C/C(Nc1ccn(CCC#N)n1)=C1/C(=O)Nc2cnc(-c3cnccc3C)cc21] (Z)-N-(1-Hydroxy-2- methylpropan-2-yl)-2,4- dimethyl-5-((5-(4- methylpyridin-3-yl)-2-oxo- 1H-pyrrolo[2,3-c]pyridin- 3(2H)-ylidene)methyl)-1H- pyrrole-3-carboxamide | Step-1 of Method I, 3-(3-amino-1H- pyrazol-1- yl)propanenitrile and 4-methylpyridine-3- boronic acid Method I | ¹H NMR (400 MHz, DMSO-d₆) δ 2.38 (s, 3H), 2.73 (s, 3H), 3.09 (t, J = 6.4 Hz, 2H), 4.39 (t, J = 6.4 Hz, 2H), 6.36 (d, J = 2.4 Hz, 1H), 7.32 (d, J = 4.8 Hz, 1H), 7.54 (s, 1H), 7.91 (d, J = 2.4 Hz, 1H), 8.24 (s, 1H), 8.43 (d, J = 4.8 Hz, 1H), 8.58 (s, 1H), 10.94 (s, 1H), 12.66 (s, 1H); ESI-MS (m/z) 387 (M + H)⁺. |
| 111 | 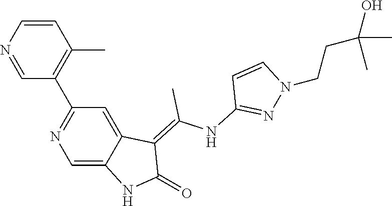 [SMILES: C/C(Nc1ccn(CCC(C)(C)O)n1)=C1/C(=O)Nc2cnc(-c3cnccc3C)cc21] (Z)-3-(3-((1-(5-(4- Methylpyridin-3-yl)-2-oxo- 1H-pyrrolo[2,3-c]pyridin- 3(2H)- ylidene)ethyl)amino)-1H- pyrazol-1-yl)propanenitrile | Step-1 of Method I, 4-(3-amino-1H- pyrazol-1-yl)-2- methylbutan-2-ol and 4-methyl-3-(4,4,5,5- tetramethyl-1,3,2- dioxaborolan-2- yl)pyridine Method I | ¹H NMR (400 MHz, DMSO-d₆) δ 1.13 (s, 6H), 1.88-1.92 (m, 2H), 2.38 (s, 3H), 2.71 (s, 3H), 4.13-4.19 (m, 2H), 4.47 (s, 1H), 6.28 (d, J = 2.4 Hz, 1H), 7.32 (d, J = 5.2 Hz, 1H), 7.52 (s, 1H), 7.83 (d, J = 2.4 Hz, 1H), 8.24 (s, 1H), 8.43 (d, J = 5.2 Hz, 1H), 8.57 (s, 1H), 10.91 (s, 1H), 12.63 (s, 1H); ESI-MS (m/z) 419 (M + H)⁺. |
| 112 | 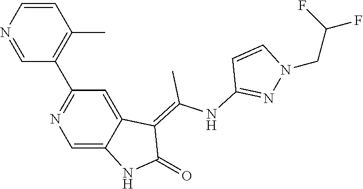 [SMILES: C/C(Nc1ccn(CC(F)F)n1)=C1/C(=O)Nc2cnc(-c3cnccc3C)cc21] (Z)-3-(1-((1-(3-Hydroxy-3- methylbutyl)-1H-pyrazol- 3-yl)amino)ethylidene)-5- (4-methylpyridin-3-yl)-1H- pyrrolo[2,3-c]pyridin- 2(3H)-one | Step-1 of Method I, 1-(2,2-difluoroethyl)- 1H-pyrazol-3-amine and 4-methyl-3- (4,4,5,5-tetramethyl- 1,3,2-dioxaborolan-2- yl)pyridine Method I | ¹H NMR (400 MHz, DMSO-d₆) δ 2.38 (s, 3H), 2.72 (s, 3H), 4.57-4.67 (m, 2H), 6.25-6.54 (m, 2H), 7.32 (d, J = 5.2 Hz, 1H), 7.54 (s, 1H), 7.88 (d, J = 2.4 Hz, 1H), 8.25 (s, 1H), 8.43 (d, J = 4.8 Hz, 1H), 8.58 (s, 1H), 10.94 (s, 1H), 12.67 (s, 1H). |
| 113 | 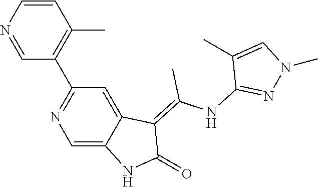 [SMILES: C/C(Nc1nn(C)cc1C)=C1/C(=O)Nc2cnc(-c3cnccc3C)cc21] (Z)-3-(1-((1-(2,2- Difluoroethyl)-1H-pyrazol- 3-yl)amino)ethylidene)-5- (4-methylpyridin-3-yl)-1H- pyrrolo[2,3-c]pyridin- 2(3H)-one | Step-1 of Method I, 1,4-dimethyl-1H- pyrazol-3-amine and 4-methyl-3-(4,4,5,5- tetramethyl-1,3,2- dioxaborolan-2- yl)pyridine Method I | ¹H NMR (400 MHz, DMSO-d₆) δ 1.99 (s, 3H), 2.38 (s, 3H), 2.70 (s, 3H), 3.79 (s, 3H), 7.32 (d, J = 4.8 Hz, 1H), 7.50 (s, 1H), 7.59 (s, 1H), 8.25 (s, 1H), 8.43 (d, J = 4.8 Hz, 1H), 8.58 (s, 1H), 10.90 (s, 1H), 12.45 (s, 1H); ESI-MS (m/z) 360 (M+H)⁺. |
| 114 | 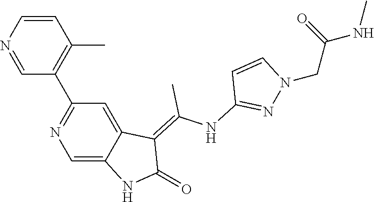 [SMILES: CNC(=O)Cn1ccc(N/C(C)=C2\C(=O)Nc3cnc(-c4cnccc4C)cc32)n1] (Z)-3-(1-((1,4-Dimethyl- 1H-pyrazol-3- yl)amino)ethylidene)-5-(4- methylpyridin-3-yl)-1H- pyrrolo[2,3-c]pyridin- 2(3H)-one | Step-1 of Method I, 2-(3-amino-1H- pyrazol-1-yl)-N- methylacetamide (D25) and 4-methyl- 3-(4,4,5,5- tetramethyl-1,3,2- dioxaborolan-2- yl)pyridine Method I | ¹H NMR (400 MHz, DMSO-d₆) δ 2.38 (s, 3H), 2.64 (s, 3H), 2.72 (s, 3H), 4.76 (s, 2H), 6.33 (d, J = 2.4 Hz, 1H), 7.32 (d, J = 4.8 Hz, 1H), 7.53 (s, 1H), 7.80 (d, J = 2.4 Hz, 1H), 8.00-8.04 (m, 1H), 8.24 (s, 1H), 8.43 (d,7 = 4.8 Hz, 1H), 8.58 (s, 1H), 10.92 (s, 1H), 12.64 (s, 1H); ESI-MS (m/z) 404 (M + H)⁺. |
| 115 | 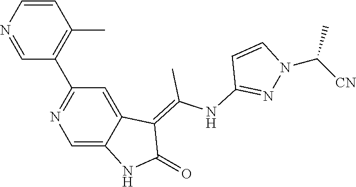 [SMILES: C/C(Nc1ccn([C@H](C)C#N)n1)=C1/C(=O)Nc2cnc(-c3cnccc3C)cc21] (Z)-N-Methyl-2-(3-((1-(5- (4-methylpyridin-3-yl)-2- oxo-1H-pyrrolo[2,3- c]pyridin-3(2H)- ylidene)ethyl)amino)-1H- pyrazol-1-yl)acetamide | Intermediate B9 and (R)-2-(3-amino-1H- pyrazol-1- yl)propanenitrile (D21) Method H | ¹H NMR (400 MHz, DMSO-d₆) δ 1.82 (d, J = 7.2 Hz, 3H), 2.38 (s, 3H), 2.75 (s, 3H), 5.85 (q, J = 7.2 Hz, 1H), 6.45 (d, J = 2.4 Hz, 1H), 7.32 (d, J = 5.2 Hz, 1H), 7.56 (s, 1H), 8.01 (d, J = 2.8 Hz, 1H), 8.25 (s, 1H), 8.43 (d, J = 4.8 Hz, 1H), 8.58 (s, 1H), 10.98 (s, 1H), 12.71 (s, 1H); ESI-MS (m/z) 386 (M + H)⁺. |
| 116 | 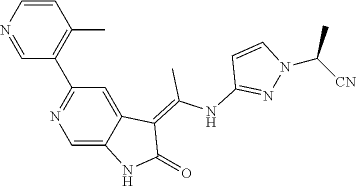 [SMILES: C/C(Nc1ccn([C@@H](C)C#N)n1)=C1/C(=O)Nc2cnc(-c3cnccc3C)cc21] (R,Z)-2-(3-((1-(5-(4- methylpyridin-3-yl)-2-oxo- 1H-pyrrolo[2,3-c]pyridin- 3(2H)- ylidene)ethyl)amino)-1H- pyrazol-1-yl)propanenitrile | 5-(4-methylpyridin-3- yl)-1H-pyrrolo[2,3- c]pyridin-2(3H)-one and Intermediate D12 Method H | ¹H NMR (400 MHz, DMSO-d₆) δ 1.81 (d, J = 2.0 Hz, 3H), 2.38 (s, 3H), 2.75 (s, 3H), 5.85 (q, J = 6.8 Hz, 1H), 6.45 (s, 1H), 7.31 (d, J = 4.8 Hz, 1H), 7.56 (s, 1H), 8.01 (s, 1H), 8.25 (s, 1H), 8.43 (s, 1H), 8.58 (s, 1H), 10.97 (s, 1H), 12.71 (s, 1H); ESI-MS (m/z) 386 (M + H)⁺. |
| 117 | 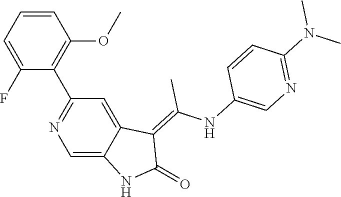 [SMILES: COc1cccc(F)c1-c1cc2c(cn1)NC(=O)/C2=C(/C)Nc1ccc(N(C)C)nc1] (Z)-3-(1-((6- (Dimethylamino)pyridin-3- yl)amino)ethylidene)-5-(2- fluoro-6-methoxyphenyl)- 1H-pyrrolo[2,3-c]pyridin- 2(3H)-one | Step 1 of Method I, N2,N2- dimethylpyridine-2,5- diamine and 2-fluoro- 6- methoxyphenylboronic acid Method I | ¹H NMR (400 MHz, DMSO-d₆) δ 2.38 (s, 3H), 3.34 (s, 6H), 3.71 (s, 3H), 7.10 (d, J = 9.2 Hz, 1H), 6.86 (d, J = 8.8 Hz, 1H), 6.94 (d, J = 8.4 Hz, 1H), 7.27 (s, 1H), 7.37 (q, J = 4.4 Hz, 1H), 7.53 (dd, J₁ = 2.8 Hz, J₂ = 9.2 Hz, 1H), 8.09 (s, 1H), 8.16 (s, 1H), 10.80 (s, 1H), 12.13 (s, 1H); ESI- MS (m/z) 420 (M + H)⁺. |
| 118 | 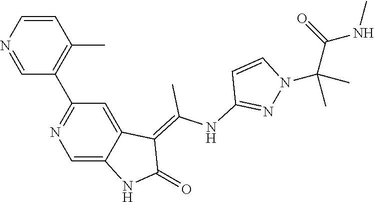 [SMILES: CNC(=O)C(C)(C)n1ccc(N/C(C)=C2\C(=O)Nc3cnc(-c4cnccc4C)cc32)n1] (Z)-N,2-dimethyl-2-(3-((1- (5-(4-methylpyridin-3-yl)- 2-oxo-1H-pyrrolo[2,3- c]pyridin-3(2H)- ylidene)ethyl)amino)-1H- pyrazol-1-yl)propanamide | Step-1 of Method I, 2-(3-amino-1H- pyrazol-1-yl)-N,2- dimethylpropanamide (D22) and 4-methyl- 3-(4,4,5,5- tetramethyl-1,3,2- dioxaborolan-2- yl)pyridine Method I | ¹H NMR (400 MHz, DMSO-d₆) δ 1.70 (s, 6H), 2.38 (s, 3H), 2.58 (d, J = 4.4 Hz, 3H), 2.72 (s, 3H), 6.37 (d, J = 2.4 Hz, 1H), 7.32 (d, J = 4.8 Hz, 2H), 7.53 (s, 1H), 7.93 (d, J = 2.4 Hz, 1H), 8.24 (s, 1H), 8.43 (d, J = 4.8 Hz, 1H), 8.58 (s, 1H), 10.94 (s, 1H), 12.61 (s, 1H); ESI-MS (m/z) 432 (M + H)⁺. |
| 119 | 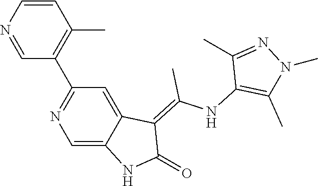 [SMILES: C/C(Nc1c(C)nn(C)c1C)=C1/C(=O)Nc2cnc(-c3cnccc3C)cc21] (Z)-5-(4-Methylpyridin-3- yl)-3-(1-((1,3,5-trimethyl- 1H-pyrazol-4- yl)amino)ethylidene)-1H- pyrrolo[2,3-c]pyridin- 2(3H)-one | Step-1 of Method I, 4-methyl-3-(4,4,5,5- tetramethyl-1,3,2- dioxaborolan-2- yl)pyridine and 1,3,5- trimethyl-1H- pyrazol-4-amine Method I | ¹H NMR (400 MHz, DMSO-d₆) δ 2.03 (s, 3H), 2.13 (s, 3H), 2.33 (s, 3H), 2.36 (s, 3H), 3.70 (s, 3H), 7.31 (d, J = 5.2 Hz, 1H), 7.44 (s, 1H), 8.23 (s, 1H), 8.42 (d, J = 5.2 Hz, 1H), 8.56 (s, 1H), 10.82 (s, 1H), 11.59 (s, 1H); ESI-MS (m/z) 376 (M + H)⁺. |
| 120 | 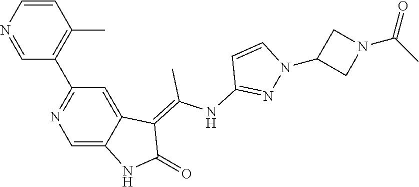 [SMILES: CC(=O)N1CC(n2ccc(N/C(C)=C3\C(=O)Nc4cnc(-c5cnccc5C)cc43)n2)C1] (Z)-3-(1-((1-(1- Acetylazetidin-3-yl)-1H- pyrazol-3- yl)amino)ethylidene)-5-(4- methylpyridin-3-yl)-1H- pyrrolo[2,3-c]pyridin- 2(3H)-one | Step-1 of Method I, 4-methyl-3-(4,4,5,5- tetramethyl-1,3,2- dioxaborolan-2- yl)pyridine and Intermediate D13 Method I | ¹H NMR (400 MHz, DMSO-d₆) δ 1.80 (s, 3H), 2.37 (s, 3H), 2.41 (s, 3H), 4.12-4.16 (m, 1H), 4.25- 4.30 (m, 1H), 4.40-4.44 (m, 1H), 4.53-4.58 (m, 1H), 5.13-5.17 (m, 1H), 6.40 (s, 1H), 7.32 (d, J = 5.2 Hz, 1H), 7.52 (s, 1H), 7.69 (d, J = 1.6 Hz, 1H), 8.27 (s, 1H), 8.43 (d, J = 5.2 Hz, 1H), 8.57 (s, 1H), 10.99 (s, 1H), 12.05 (s, 1H); ESI-MS (m/z) 430 (M + H)⁺. |
| 121 | 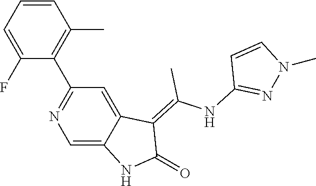 [SMILES: C/C(Nc1ccn(C)n1)=C1/C(=O)Nc2cnc(-c3c(C)cccc3F)cc21] (Z)-5-(2-Fluoro-6- methylphenyl)-3-(1-((1- methyl-1H-pyrazol-3- yl)amino)ethylidene)-1H- pyrrolo[2,3-c]pyridin- 2(3H)-one | Step-1 of Method I, 1-methyl-1H-pyrazol- 3-amine and 2-(2- fluoro-6- methylphenyl)- 4,4,5,5-tetramethyl- 1,3,2-dioxaborolane Method I | ¹H NMR (400 MHz, DMSO-d₆) δ 2.14 (s, 3H), 2.65 (s, 3H), 3.82 (s, 3H), 6.27 (s, 1H), 7.07-7.14 (m, 2H), 7.28-7.35 (m, 2H), 7.26 (s, 1H), 8.22 (s, 1H), 10.89 (s, 1H), 12.63 (s, 1H); ESI-MS (m/z) 365 (M)⁺². |
| 122 | 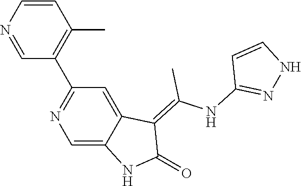 [SMILES: C/C(Nc1cc[nH]n1)=C1/C(=O)Nc2cnc(-c3cnccc3C)cc21] (Z)-3-(1-((1H-Pyrazol-3- yl)amino)ethylidene)-5-(4- methylpyridin-3-yl)-1H- pyrrolo[2,3-c]pyridin- 2(3H)-one | Step-1 of Method I, 1-cyclopropyl-1H- pyrazol-3-amine and 4-methyl-3-(4,4,5,5- tetramethyl-1,3,2- dioxaborolan-2- yl)pyridine Method I | ¹H NMR (400 MHz, DMSO-d₆) δ 2.38 (s, 3H), 3.34 (s, 3H), 6.61 (s, 1H), 7.31 (d, J = 5.2 Hz, 1H), 7.52 (s, 1H), 7.83 (s, 1H), 8.24 (s, 1H), 8.43 (s, 1H), 8.58 (s, 1H), 10.91 (s, 1H), 12.63 (s, 1H), 12.88 (s, 1H); ESI-MS (m/z) 333 (M + H)⁺. |
| 123 | 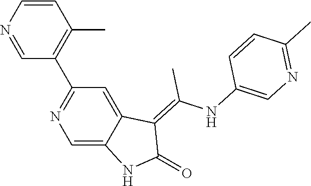 [SMILES: C/C(Nc1ccc(C)nc1)=C1/C(=O)Nc2cnc(-c3cnccc3C)cc21] (Z)-5-(4-Methylpyridin-3- yl)-3-(1-((6-methylpyridin- 3-yl)amino)ethylidene)-1H- pyrrolo[2,3-c]pyridin- 2(3H)-one | Intermediate B9, and 5-aminopicoline Method H | ¹H NMR (400 MHz, DMSO-d₆) δ 2.37 (s, 3H), 2.50 (s, 3H), 3.33 (s, 3H), 7.31 (s, 1H), 7.37 (d, J = 8.0 Hz, 1H), 7.50 (s, 1H), 7.72 (d, J = 7.6 Hz, 1H), 8.25 (s, 1H), 8.43-8.47 (m, 2H), 8.57 (s, 1H), 10.93 (s, 1H), 12.37 (s, 1H); ESI-MS (m/z) 358 (M + H)⁺. |
| 124 | 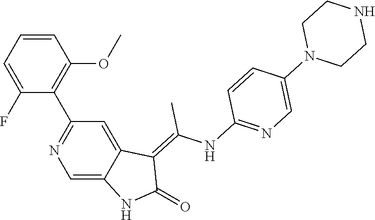 [SMILES: COc1cccc(F)c1-c1cc2c(cn1)NC(=O)/C2=C(/C)Nc1ccc(N2CCNCC2)cn1] (Z)-5-(2-Fluoro-6- methoxyphenyl)-3-(1-((5- (piperazin-1-yl)pyridin-2- yl)amino)ethylidene)-1H- pyrrolo[2,3-c]pyridin- 2(3H)-one | Step 1 of Method I, tert-butyl 4-(6- aminopyridin-3- yl)piperazine-1- carboxylate and 2- fluoro-6- methoxyphenylboronic acid Method I followed by Boc- deprotection using PTSA | ¹H NMR (400 MHz, DMSO-d₆) δ 2.87 (s, 3H), 3.12 (t, J = 4.4 Hz, 4H), 3.37 (t, J = 6.8 Hz, 4H), 3.72 (s, 3H), 6.87 (t, J = 8.8 Hz, 1H), 6.95 (d, J = 8.4 Hz, 1H), 7.20 (d, J = 9.2 Hz, 1H), 7.33-7.47 (m, 3H), 8.13 (s, 2H), 10.88 (s, 1H), 12.72 (s, 1H); ESI-MS (m/z) 461 (M + H)⁺. |
| 125 | 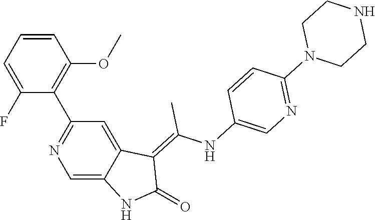 [SMILES: COc1cccc(F)c1-c1cc2c(cn1)NC(=O)/C2=C(/C)Nc1ccc(N2CCNCC2)nc1] (Z)-5-(2-Fluoro-6- methoxyphenyl)-3-(1-((6- (piperazin-1-yl)pyridin-3- yl)amino)ethylidene)-1H- pyrrolo[2,3-c]pyridin- 2(3H)-one | Step 1 of Method I, tert-butyl 4-(5- aminopyridin-2- yl)piperazine-1- carboxylate and 2- fluoro-6- methoxyphenylboronic acid Method I followed by Boc- deprotection using PTSA | ¹H NMR (400 MHz, DMSO-d₆) δ 2.39 (s, 3H), 2.86 (t, J = 4.8 Hz, 4H), 3.50 (t, J = 4.8 Hz, 4H), 3.71 (s, 3H), 6.84-6.95 (m, 4H), 7.35-7.41 (m, 1H), 7.57 (dd, J₁ = 2.8 Hz, J₂ = 9.2 Hz, 1H), 8.12 (s, 1H), 8.17 (s, 1H), 10.81 (s, 1H), 12.15 (s, 1H); ESI-MS (m/z) 461 (M + H)⁺. |
| 126 | 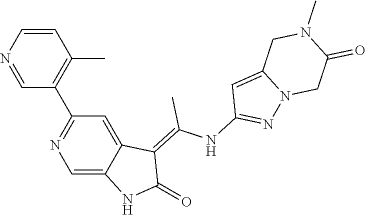 [SMILES: C/C(Nc1cc2n(n1)CC(=O)N(C)C2)=C1/C(=O)Nc2cnc(-c3cnccc3C)cc21] (Z)-5-Methyl-2-((1-(5-(4- methylpyridin-3-yl)-2-oxo- 1H-pyrrolo[2,3-c]pyridin- 3(2H)- ylidene)ethyl)amino)-4,5- dihydropyrazolo[1,5- a]pyrazin-6(7H)-one | Intermediate B9 and 2-amino-5-methyl- 4,5- dihydropyrazolo[1,5- a]pyrazin-6(7H)-one (D32) Method H | ¹H NMR (400 MHz, DMSO-d₆) δ 2.37 (s, 3H), 2.73 (s, 3H), 3.01 (s, 3H), 4.63 (s, 2H), 4.74 (s, 2H), 6.28 (s, 1H), 7.31 (d, J = 5.2 Hz, 1H), 7.53 (s, 1H), 8.24 (s, 1H), 8.42 (d, J = 4.8 Hz, 1H), 8.57 (s, 1H), 10.93 (s, 1H), 12.68 (s, 1H); ESI- MS (m/z) 416 (M + H)⁺. |
| 127 | 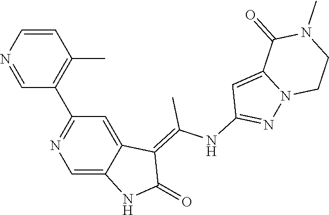 [SMILES: C/C(Nc1cc2n(n1)CCN(C)C2=O)=C1/C(=O)Nc2cnc(-c3cnccc3C)cc21] (Z)-5-Methyl-2-((1-(5-(4- methylpyridin-3-yl)-2-oxo- 1H-pyrrolo[2,3-c]pyridin- 3(2H)- ylidene)ethyl)amino)-6,7- dihydropyrazolo[1,5- a]pyrazin-4(5H)-onc | Step-1 of Method I, 2-amino-5-methyl- 6,7- dihydropyrazolo[1,5- a]pyrazin-4(5H)-one and 4-methyl-3- (4,4,5,5-tetramethyl- 1,3,2-dioxaborolan-2- yl)pyridine Method I | ¹H NMR (400 MHz, DMSO-d₆) δ 2.37 (s, 3H), 2.72 (s, 3H), 3.02 (s, 3H), 3.81 (t, J = 6.0 Hz, 2H), 4.37 (t, J = 6.0 Hz, 2H), 6.79 (s, 1H), 7.32 (d, J = 5.2 Hz, 1H), 7.54 (s, 1H), 8.25 (s, 1H), 8.43 (d, J = 5.2 Hz, 1H), 8.57 (s, 1H), 10.95 (s, 1H), 12.69 (s, 1H); ESI-MS (m/z) 417 (M + H)⁺. |
| 128 | 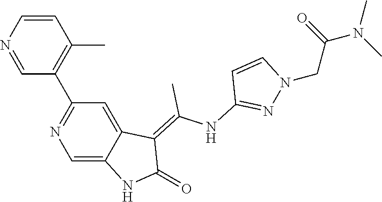 [SMILES: C/C(Nc1ccn(CC(=O)N(C)C)n1)=C1/C(=O)Nc2cnc(-c3cnccc3C)cc21] (Z)-N,N-Dimethyl-2-(3- ((1-(5-(4-methylpyridin-3- yl)-2-oxo-1H-pyrrolo[2,3- c]pyridin-3(2H)- ylidene)ethyl)amino)-1H- pyrazol-1-yl)acetamide | Step-1 of Method I, 2-(3-amino-1H- pyrazol-1-yl)-N,N- dimethylacetamide and 4-methyl-3- (4,4,5,5-tetramethyl- 1,3,2-dioxaborolan-2- yl)pyridine Method I | ¹H NMR (400 MHz, DMSO-d₆) δ 2.38 (s, 3H), 2.72 (s, 3H), 2.86 (s, 3H), 3.03 (s, 3H), 5.11 (s, 2H), 6.32 (d, J = 2.4 Hz, 1H), 7.32 (d, J = 4.8 Hz, 1H), 7.53 (s, 1H), 7.73 (d, J = 2.4 Hz, 1H), 8.24 (s, 1H), 8.43 (d, J = 4.8 Hz, 1H), 8.58 (s, 1H), 10.91 (s, 1H), 12.64 (s, 1H). |
| 129 | 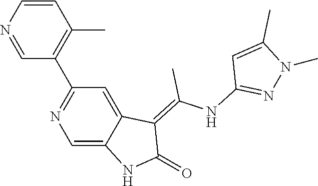 [SMILES: C/C(Nc1cc(C)n(C)n1)=C1/C(=O)Nc2cnc(-c3cnccc3C)cc21] (Z)-3-(1-((1,5-dimethyl- 1H-pyrazol-3- yl)amino)ethylidene)-5-(4- methylpyridin-3-yl)-1H- pyrrolo[2,3-c]pyridin- 2(3H)-one | Intermediate B9 and 1,5-dimethyl-1H- pyrazol-3-amine Method H | ¹H NMR (400 MHz, DMSO-d₆) δ 2.27 (s, 3H), 2.37 (s, 3H), 2.70 (s, 3H), 3.71 (s, 3H), 6.10 (s, 1H), 7.32 (d, J = 4.8 Hz, 1H), 7.51 (s, 1H), 8.23 (s, 1H), 8.43 (d, J = 5.2 Hz, 1H), 8.57 (s, 1H), 10.89 (s, 1H), 12.58 (s, 1H); ESI-MS (m/z) 362 (M + H)⁺. |
| 130 | 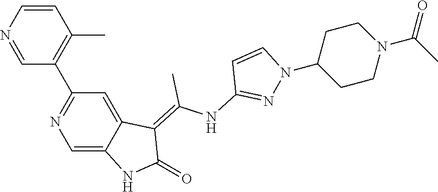 [SMILES: CC(=O)N1CCC(n2ccc(N/C(C)=C3\C(=O)Nc4cnc(-c5cnccc5C)cc43)n2)CC1] (Z)-3-(1-((1-(1- Acetylpiperidin-4-yl)-1H- pyrazol-3- yl)amino)ethylidene)-5-(4- methylpyridin-3-yl)-1H- pyrrolo[2,3-c]pyridin- 2(3H)-one | Step-1 of Method I, 1-(4-(3-amino-1H- pyrazol-1- yl)piperidin-1- yl)ethanone (D26) and 4-methyl-3- (4,4,5,5-tetramethyl- 1,3,2-dioxaborolan-2- yl)pyridine Method I | ¹H NMR (400 MHz, DMSO-d₆) δ 1.70-1.76 (m, 1H), 1.85-1.91 (m, 1H), 2.00-2.05 (m, 1H), 2.07 (s, 3H), 2.38 (s, 3H), 2.68 (s, 3H), 2.71-2.75 (m, 1H), 3.16-3.24 (m, 2H), 3.89- 3.95 (m, 1H), 4.37-4.49 (m, 2H), 6.32 (d, J = 2.4 Hz, 1H), 7.32 (d, J = 5.2 Hz, 1H), 7.52 (s, 1H), 7.89 (d, J = 2.0 Hz, 1H), 8.24 (s, 1H), 8.42 (d, J = 4.8 Hz, 1H), 8.57 (s, 1H), 10.92 (s, 1H), 12.65 (s, 1H); ESI-MS (m/z) 459 (M + H)⁺. |
| 131 | 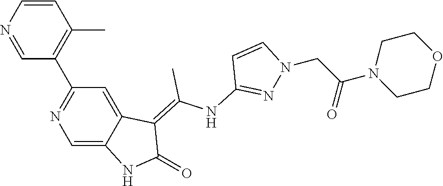 [SMILES: C/C(Nc1ccn(CC(=O)N2CCOCC2)n1)=C1/C(=O)Nc2cnc(-c3cnccc3C)cc21] (Z)-5-(4-Methylpyridin-3- yl)-3-(1-((1-(2-morpholino- 2-oxoethyl)-1H-pyrazol-3- yl)amino)ethylidene)-1H- pyrrolo[2,3-c]pyridin- 2(3H)-one | Step-1 of Method I, 2-(3-amino-1H- pyrazol-1-yl)-1- morpholinoethanone (D27) and 4-methyl- 3-(4,4,5,5- tetramethyl-1,3,2- dioxaborolan-2- yl)pyridine Method I | ¹H NMR (400 MHz, DMSO-d₆) δ 2.38 (s, 3H), 2.72 (s, 3H), 3.43-3.47 (m, 4H), 3.48-3.65 (m, 4H), 5.15 (s, 2H), 6.33 (d, J = 2.4 Hz, 1H), 7.32 (d, J = 4.8 Hz, 1H), 7.53 (s, 1H), 7.74 (d, J = 2.4 Hz, 1H), 8.24 (s, 1H), 8.43 (d, J = 4.8 Hz, 1H), 8.58 (s, 1H), 10.91 (s, 1H), 12.65 (s, 1H). |
| 132 | 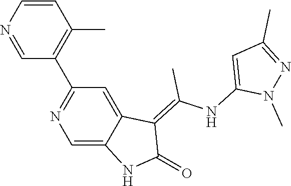 [SMILES: C/C(Nc1cc(C)nn1C)=C1/C(=O)Nc2cnc(-c3cnccc3C)cc21] (Z)-3-(1-((1,3-Dimethyl- 1H-pyrazol-5- yl)amino)ethylidene)-5-(4- methylpyridin-3-yl)-1H- pyrrolo[2,3-c]pyridin- 2(3H)-one | Intermediate B9 and 1,3-dimethyl-1H- pyrazol-5-amine Method H | ¹H NMR (400 MHz, DMSO-d₆) δ 2.16 (s, 3H), 2.37 (s, 3H), 3.35 (s, 3H), 3.66 (s, 3H), 6.12 (s, 1H), 7.32 (d, J = 4.8 Hz, 1H), 7.53 (s, 1H), 8.27 (s, 1H), 8.43 (d, J = 4.8 Hz, 1H), 8.57 (s, 1H), 11.00 (s, 1H), 11.29 (s, 1H); ESI-MS (m/z) 361 (M + H)⁺. |
| 133 | 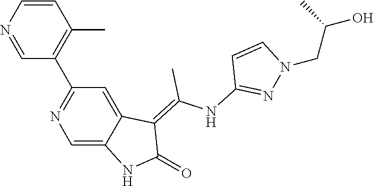 [SMILES: C/C(Nc1ccn(C[C@H](C)O)n1)=C1/C(=O)Nc2cnc(-c3cnccc3C)cc21] (S,Z)-3-(1-((1-(2- Hydroxypropyl)-1H- pyrazol-3- yl)amino)ethylidene)-5-(4- methylpyridin-3-yl)-1H- pyrrolo[2,3-c]pyridin- 2(3H)-one | Step-1 of Method I, (S)-1-(3-amino-1H- pyrazol-1-yl)propan- 2-ol (D28) and 4- methyl-3-(4,4,5,5- tetramethyl-1,3,2- dioxaborolan-2- yl)pyridine Method I | ¹H NMR (400 MHz, DMSO-d₆) δ 1.06 (d, J = 6.0 Hz, 3H), 2.38 (s, 3H), 2.71 (s, 3H), 3.98 (s, 3H), 4.94 (d, J = 5.2 Hz, 1H), 6.28 (s, 1H), 7.31 (d, J = 4.8 Hz, 1H), 7.52 (s, 1H), 7.75 (s, 1H), 8.24 (s, 1H), 8.43 (d, J = 4.8 Hz, 1H), 8.58 (s, 1H), 10.91 (s, 1H), 12.63 (s, 1H); ESI-MS (m/z) 391 (M + H)⁺. |
| 134 | 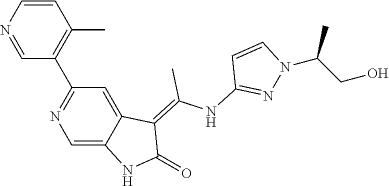 [SMILES: C/C(Nc1ccn([C@@H](C)CO)n1)=C1/C(=O)Nc2cnc(-c3cnccc3C)cc21] (S,Z)-3-(1-((1-(1- Hydroxypropan-2-yl)-1H- pyrazol-3- yl)amino)ethylidene)-5-(4- methylpyridin-3-yl)-1H- pyrrolo[2,3-c]pyridin- 2(3H)-one | Step-1 of Method I, (S)-2-(3-amino-1H- pyrazol-1-yl)propan- 1-ol (D29) and 4- methyl-3-(4,4,5,5- tetramethyl-1,3,2- dioxaborolan-2- yl)pyridine Method I | ¹H NMR (400 MHz, DMSO-d₆) δ 1.38 (d, J = 7.2 Hz, 3H), 2.38 (s, 3H), 2.72 (s, 3H), 3.59-3.69 (m, 2H), 4.28-4.33 (m, 1H), 4.95 (t, J = 5.6 Hz, 1H), 6.28 (d, J = 2.4 Hz, 1H), 7.32 (d, J = 5.2 Hz, 1H), 7.53 (s, 1H), 7.81 (d, J = 2.0 Hz, 1H), 8.24 (s, 1H), 8.43 (d, J = 5.2 Hz, 1H), 8.58 (s, 1H), 10.92 (s, 1H), 12.64 (s, 1H); ESI-MS (m/z) 391 (M + H)⁺. |
| 135 | 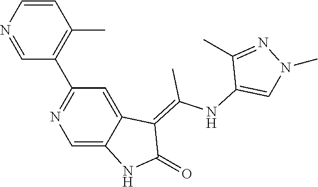 [SMILES: C/C(Nc1cn(C)nc1C)=C1/C(=O)Nc2cnc(-c3cnccc3C)cc21] (Z)-3-(1-((1,3-Dimethyl- 1H-pyrazol-4- yl)amino)ethylidene)-5-(4- methylpyridin-3-yl)-1H- pyrrolo[2,3-c]pyridin- 2(3H)-one | Step-1 of Method I, 1,3-dimethyl-1H- pyrazol-4-amine and 4-methyl-3-(4,4,5,5- tetramethyl-1,3,2- dioxaborolan-2- yl)pyridine Method I | ¹H NMR (400 MHz, DMSO-d₆) δ 2.11 (s, 3H), 2.37 (s, 3H), 2.48 (d, J = 7.6 Hz, 3H), 3.79 (s, 3H), 7.31 (d, J = 5.2 Hz, 1H), 7.48 (s, 1H), 7.87 (s, 1H), 8.23 (s, 1H), 8.41 (s, 1H), 8.57 (s, 1H), 10.85 (s, 1H), 12.04 (s, 1H); ESI-MS (m/z) 361 (M + H)⁺. |
| 136 | 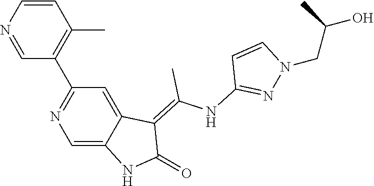 [SMILES: C/C(Nc1ccn(C[C@@H](C)O)n1)=C1/C(=O)Nc2cnc(-c3cnccc3C)cc21] (R,Z)-3-(1-((1-(2- Hydroxypropyl)-1H- pyrazol-3- yl)amino)ethylidene)-5-(4- methylpyridin-3-yl)-1H- pyrrolo[2,3-c]pyridin- 2(3H)-one | Step-1 of Method I, (R)-1-(3-amino-1H- pyrazol-1-yl)propan- 2-ol (D23) and 4- methyl-3-(4,4,5,5- tetramethyl-1,3,2- dioxaborolan-2- yl)pyridine Method I | ¹H NMR (400 MHz, DMSO-d₆) δ 1.06 (d, J = 5.6 Hz, 3H), 2.38 (s, 3H), 2.72 (s, 3H), 3.95-4.01 (m, 3H), 4.95 (d, J = 4.0 Hz, 1H), 6.29 (d, J = 2.0 Hz, 1H), 7.32 (d, J = 4.8 Hz, 1H), 7.52 (s, 1H), 7.76 (d, J = 2.4 Hz, 1H), 8.24 (s, 1H), 8.43 (d, J = 4.8 Hz, 1H), 8.57 (s, 1H), 10.91 (s, 1H), 12.63 (s, 1H); ESI-MS (m/z) 391 (M + H)⁺. |
| 137 | 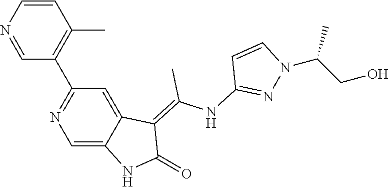 [SMILES: C/C(Nc1ccn([C@H](C)CO)n1)=C1/C(=O)Nc2cnc(-c3cnccc3C)cc21] (R,Z)-3-(1-((1-(1- Hydroxypropan-2-yl)-1H- pyrazol-3- yl)amino)ethylidene)-5-(4- methylpyridin-3-yl)-1H- pyrrolo[2,3-c]pyridin- 2(3H)-one | Step-1 of Method I, (R)-2-(3-amino-1H- pyrazol-1-yl)propan- 1-ol (D24) and 4- methyl-3-(4,4,5,5- tetramethyl-1,3,2- dioxaborolan-2- yl)pyridine Method I | ¹H NMR (400 MHz, DMSO-d₆) δ 1.38 (d, J = 6.8 Hz, 3H), 2.38 (s, 3H), 2.72 (s, 3H), 3.59-3.69 (m, 2H), 4.29-4.33 (m, 1H), 4.93- 4.96 (m, 1H), 6.28 (d, J = 2.0 Hz, 1H), 7.32 (d, J = 5.2 Hz, 1H), 7.53 (s, 1H), 7.81 (d, J = 2.4 Hz, 1H), 8.24 (s, 1H), 8.43 (d, J = 4.8 Hz, 1H), 8.58 (s, 1H), 10.91 (s, 1H), 12.64 (s, 1H); ESI-MS (m/z) 392 (M + H)⁺. |
| 138 | 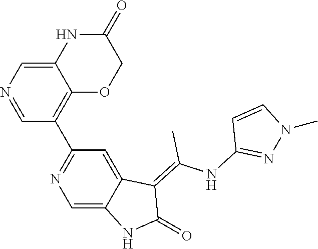 [SMILES: C/C(Nc1ccn(C)n1)=C1/C(=O)Nc2cnc(-c3cncc4c3OCC(=O)N4)cc21] (Z)-8-(3-(1-((1-Methyl-1H- pyrazol-3- yl)amino)ethylidene)-2- oxo-2,3-dihydro-1H- pyrrolo[2,3-c]pyridin-5-yl)- 2H-pyrido[4,3- b][1,4]oxazin-3(4H)-one | Step-1 of Method I, (R)-1-methyl-1H- pyrazol-3-amine and 8-(4,4,5,5- tetramethyl-1,3,2- dioxaborolan-2-yl)- 2H-pyrido[4,3- b][1,4]oxazin-3(4H)- one (A5) Method I | ¹H NMR (400 MHz, DMSO-d₆) δ 2.71 (s, 3H), 3.83 (s, 3H), 4.80 (s, 2H), 6.30 (s, 1H),7.77 (s, 1H), 7.87 (s, 1H), 8.07 (s, 1H), 8.24 (s, 1H), 8.50 (s, 1H), 10.92 (s, 1H), 11.01 (s, 1H), 12.59 (s, 1H); ESI-MS (m/z) 404 (M + H)⁺. |
| 139 | 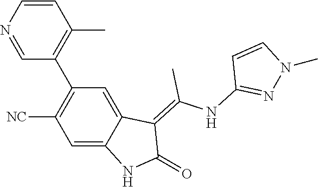 [SMILES: C/C(Nc1ccn(C)n1)=C1/C(=O)Nc2cc(C#N)c(-c3cnccc3C)cc21] (Z)-3-(1-((1-Methyl-1H- pyrazol-3- yl)amino)ethylidene)-5-(4- methylpyridin-3-yl)-2- oxoindoline-6-carbonitrile | 5-bromo-2- oxoindoline-6- carbonitrile, 1- methyl-1H-pyrazol-3- amine and 4-methyl- 3-(4,4,5,5- tetramethyl-1,3,2- dioxaborolan-2- yl)pyridine Method I | ¹H NMR (400 MHz, DMSO-d₆) δ 2.20 (s, 3H), 2.66 (s, 3H), 3.82 (s, 3H), 6.26 (s, 1H), 7.39 (d, J = 5.2 Hz, 2H), 7.43 (s, 1H), 7.76 (s, 1H), 8.51 (d, J = 5.2 Hz, 2H), 10.08 (s, 1H), 12.64 (s, 1H); ESI-MS (m/z) 371 (M + H)⁺. |
| 140 | 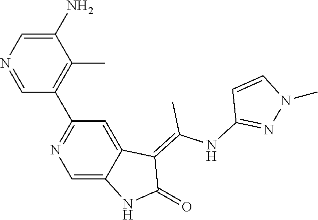 [SMILES: C/C(Nc1ccn(C)n1)=C1/C(=O)Nc2cnc(-c3cncc(N)c3C)cc21] (Z)-5-(5-Amino-4- methylpyridin-3-yl)-3-(1- ((1-methyl-1H-pyrazol-3- yl)amino)ethylidene)-1H- pyrrolo[2,3-c]pyridin- 2(3H)-one | Step-1 of Method I, 1-methyl-1H-pyrazol- 3-amine and (5- amino-4- methylpyridin-3- yl)boronic acid Method I | ¹H NMR (400 MHz, DMSO-d₆) δ 2.04 (s, 3H), 2.69 (s, 3H), 3.83 (s, 3H), 5.14 (s, 2H), 6.27 (s, 1H), 7.39 (s, 1H), 7.77 (s, 2H), 7.93 (s, 1H), 8.21 (s, 1H), 10.87 (s, 1H), 12.61 (s, 1H); ESI-MS (m/z) 362 (M + H)⁺. |
| 141 | 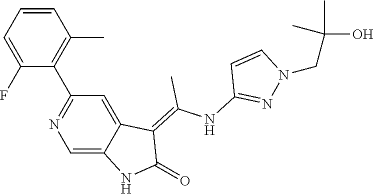 [SMILES: C/C(Nc1ccn(CC(C)(C)O)n1)=C1/C(=O)Nc2cnc(-c3c(C)cccc3F)cc21] (Z)-5-(2-Fluoro-6- methylphenyl)-3-(1-((1-(2- hydroxy-2-methylpropyl)- 1H-pyrazol-3- yl)amino)ethylidene)-1H- pyrrolo[2,3-c]pyridin- 2(3H)-one | Step-1 of Method I, 1-(3-amino-1H- pyrazol-1-yl)-2- methylpropan-2-ol (D20) and 2-(2- fluoro-6- methylphenyl)- 4,4,5,5-tetramethyl- 1,3,2-dioxaborolane Method I | ¹H NMR (400 MHz, DMSO-d₆) δ 1.23 (s, 6H), 2.14 (s, 3H), 2.64 (s, 3H), 3.99 (s, 2H), 4.71 (s, 1H), 6.30 (s, 1H), 7.07-7.14 (m, 2H), 7.28-7.36 (m, 2H), 7.72 (s, 1H), 8.22 (s, 1H), 10.90 (s, 1H), 12.59 (s, 1H); ESI-MS (m/z) 422 (M + H)⁺. |
| 142 | 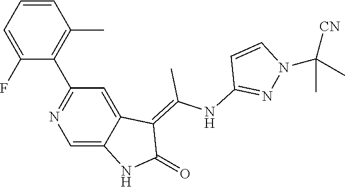 [SMILES: C/C(Nc1ccn(C(C)(C)C#N)n1)=C1/C(=O)Nc2cnc(-c3c(C)cccc3F)cc21] (Z)-2-(3-((1-(5-(2-Fluoro- 6-methylphenyl)-2-oxo- 1H-pyrrolo[2,3-c]pyridin- 3(2H)- ylidene)ethyl)amino)-1H- pyrazol-1-yl)-2- methylpropanenitrile | Step-1 of Method I, 2-(3-amino-1H- pyrazol-1-yl)-2- methylpropanenitrile (D11) and 2-(2- fluoro-6- methylphenyl)- 4,4,5,5-tetramethyl- 1,3,2-dioxaborolane Method I | ¹H NMR (400 MHz, DMSO-d₆) δ 1.99 (s, 6H), 2.15 (s, 3H), 2.60 (s, 3H), 6.49 (s, 1H), 7.10-7.15 (m, 2H), 7.31-7.40 (m, 2H), 8.11 (s, 1H), 8.23 (s, 1H), 10.98 (s, 1H), 12.70 (s, 1H); ESI-MS (m/z) 417 (M + H)⁺. |
| 143 | 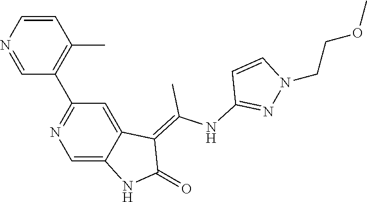 [SMILES: COCCn1ccc(N/C(C)=C2\C(=O)Nc3cnc(-c4cnccc4C)cc32)n1] (Z)-3-(1-((1-(2- Methoxyethyl)-1H-pyrazol- 3-yl)amino)ethylidene)-5- (4-methylpyridin-3-yl)-1H- pyrrolo[2,3-c]pyridin- 2(3H)-one | Step-1 of Method I, 1-(2-methoxyethyl)- 1H-pyrazol-3-amine and 4-methyl-3- (4,4,5,5-tetramethyl- 1,3,2-dioxaborolan-2- yl)pyridine Method I | ¹H NMR (400 MHz, DMSO-d₆) δ 2.38 (s, 3H), 2.71 (s, 3H), 3.24 (s, 3H), 3.69 (d, J = 5.2 Hz, 2H), 4.24 (d, J = 4.8 Hz, 2H), 6.29 (s, 1H), 7.31 (d, J = 4.8 Hz, 1H), 7.53 (s, 1H), 7.79 (s, 1H), 8.24 (s, 1H), 8.42 (d, J = 4.8 Hz, 1H), 8.58 (s, 1H), 10.92 (s, 1H), 12.65 (s, 1H); ESI-MS (m/z) 392 (M + H)⁺. |
| 144 | 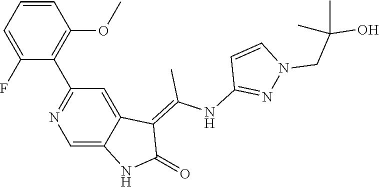 [SMILES: COc1cccc(F)c1-c1cc2c(cn1)NC(=O)/C2=C(/C)Nc1ccn(CC(C)(C)O)n1] (Z)-5-(2-Fluoro-6- methoxyphenyl)-3-(1-((1- (2-hydroxy-2- methylpropyl)-1H-pyrazol- 3-yl)amino)ethylidene)-1H- pyrrolo[2,3-c]pyridin- 2(3H)-one | Step-1 of Method I, 1-(3-amino-1H- pyrazol-1-yl)-2- methylpropan-2-ol (D20) and 2-fluoro-6- methoxyphenylboronic acid Method I | ¹H NMR (400 MHz, DMSO-d₆) δ 1.07 (s, 6H), 2.63 (s, 3H), 3.72 (s, 3H), 3.99 (s, 2H), 4.72 (s, 1H), 6.30 (d, J = 2.4 Hz, 1H), 6.86 (t, J = 8.4 Hz, 1H), 6.95 (d, J = 8.4 Hz, 1H), 7.34-7.41 (m, 2H), 7.72 (d, J = 2.4 Hz, 1H), 8.18 (s, 1H), 10.87 (s, 1H), 12.58 (s, 1H); ESI-MS (m/z) 438 (M + H)⁺. |
| 145 | 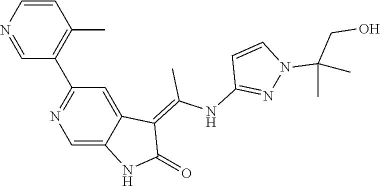 [SMILES: C/C(Nc1ccn(C(C)(C)CO)n1)=C1/C(=O)Nc2cnc(-c3cnccc3C)cc21] (Z)-3-(1-((1-(1-Hydroxy-2- methylpropan-2-yl)-1H- pyrazol-3- yl)amino)ethylidene)-5-(4- methylpyridin-3-yl)-1H- pyrrolo[2,3-c]pyridin- 2(3H)-one | Step-1 of Method I, 2-(3-amino-1H- pyrazol-1-yl)-2- methylpropan-1-ol (D30) and 4-methyl- 3-(4,4,5,5- tetramethyl-1,3,2- dioxaborolan-2- yl)pyridine Method I | ¹H NMR (400 MHz, DMSO-d₆) δ 1.47 (s, 6H), 2.38 (s, 3H), 2.71 (s, 3H), 3.58 (d, J = 6.0 Hz, 2H), 5.00 (d, J = 6.0 Hz, 1H), 5.76 (s, 1H), 6.28 (d, J = 2.4 Hz, 1H), 7.32 (d, J = 5.2 Hz, 1H), 7.52 (s, 1H), 7.84 (d, J = 2.4 Hz, 1H), 8.24 (s, 1H), 8.58 (s, 1H), 10.92 (s, 1H), 12.62 (s, 1H); ESI-MS (m/z) 405 (M + H)⁺. |
| 146 | 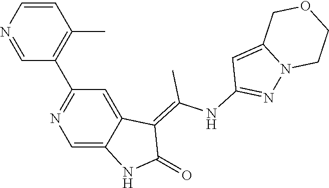 [SMILES: C/C(Nc1cc2n(n1)CCOC2)=C1/C(=O)Nc2cnc(-c3cnccc3C)cc21] (Z)-3-(1-((6,7-Dihydro-4H- pyrazolo[5,1-c][1,4]oxazin- 2-yl)amino)ethylidene)-5- (4-methylpyridin-3-yl)-1H- pyrrolo[2,3-c]pyridin- 2(3H)-one | Intermediate B9 and 6,7-dihydro-4H- pyrazolo[5,1- c][1,4]oxazin-2- amine (D31) Method H | ¹H NMR (400 MHz, DMSO-d₆) δ 2.36 (s, 3H), 2.73 (s, 3H), 4.09 (s, 3H), 4.10-4.12 (m, 1H), 4.80 (s, 2H), 6.14 (s, 1H), 7.33-7.35 (m, 1H), 7.53 (s, 1H), 8.25 (s, 1H), 8.30 (s, 2H), 10.92 (s, 1H), 12.66 (s, 1H); ESI- MS (m/z) 390 (M + H)⁺. |
| 147 | 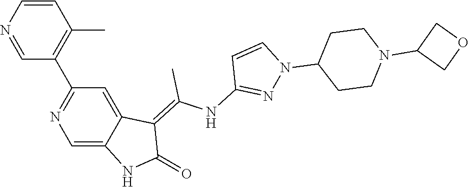 [SMILES: C/C(Nc1ccn(C2CCN(C3COC3)CC2)n1)=C1/C(=O)Nc2cnc(-c3cnccc3C)cc21] (Z)-5-(4-Methylpyridin-3- yl)-3-(1-((1-(1-(oxetan-3- yl)piperidin-4-yl)-1H- pyrazol-3- yl)amino)ethylidene)-1H- pyrrolo[2,3-c]pyridin- 2(3H)-one | Step-1 of Method I, 1-(1-(oxetan-3- yl)piperidin-4-yl)- 1H-pyrazol-3-amine (D33) and 4-methyl- 3-(4,4,5,5- tetramethyl-1,3,2- dioxaborolan-2- yl)pyridine Method I | ¹H NMR (400 MHz, DMSO-d₆) δ 1.89-2.02 (m, 6H), 2.38 (s, 3H), 2.73 (s, 3H), 2.77-2.81 (m, 2H), 3.35-3.46 (m, 1H), 4.14 (br s, 1H), 4.41-4.46 (m, 2H), 4.52-4.57 (m, 2H), 6.31 (d, J = 2.4 Hz, 1H), 7.32 (d, J = 4.8 Hz, 1H), 7.52 (s, 1H), 7.89 (d, J = 2.0 Hz, 1H), 8.24 (s, 1H), 8.43 (d, J = 4.8 Hz, 1H), 8.58 (s, 1H), 10.92 (s, 1H), 12.66 (s, 1H); ESI- MS (m/z) 472 (M + H)⁺. |
| 148 | 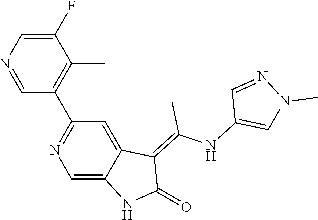 [SMILES: C/C(Nc1cnn(C)c1)=C1/C(=O)Nc2cnc(-c3cncc(F)c3C)cc21] (Z)-5-(5-Fluoro-4- methylpyridin-3-yl)-3-(1- ((1-methyl-1H-pyrazol-4- yl)amino)ethylidene)-1H- pyrrolo[2,3-c]pyridin- 2(3H)-one | Step 1 of Method I, 1-methyl-1H-pyrazol- 4-amine and 3-fluoro- 4-methyl-5-(4,4,5,5- tetramethyl-1,3,2- dioxaborolan-2- yl)pyridine Method I | ¹H NMR (400 MHz, DMSO-d₆) δ 2.31 (s, 3H), 2.54 (s, 3H), 3.86 (s, 3H), 7.54 (s, 1H), 7.59 (s, 1H), 7.97 (s, 1H), 8.24 (s, 1H), 8.49 (s, 2H), 10.90 (s, 1H), 12.15 (s, 1H); ESI-MS (m/z) 365 (M + H)⁺. |
| 149 | 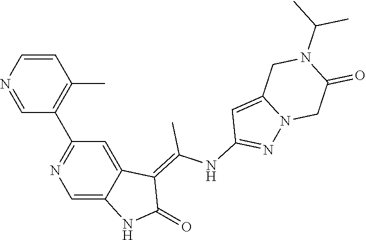 [SMILES: C/C(Nc1cc2n(n1)CC(=O)N(C(C)C)C2)=C1/C(=O)Nc2cnc(-c3cnccc3C)cc21] (Z)-5-Isopropyl-2-((1-(5- (4-methylpyridin-3-yl)-2- oxo-1H-pyrrolo[2,3- c]pyridin-3(2H)- ylidene)ethyl)amino)-4,5- dihydropyrazolo[1,5- a]pyrazin-6(7H)-one | Intermediate B9 and 2-amino-5-isopropyl- 4,5- dihydropyrazolo[1,5- a]pyrazin-6(7H)-one (D34) Method H | ¹H NMR (400 MHz, DMSO-d₆) δ 1.17 (d, J = 6.8 Hz, 6H), 3.38 (s, 3H), 2.74 (s, 3H), 4.55 (s, 2H), 4.74 (s, 2H), 4.76-4.82 (m, 1H), 6.28 (s, 1H), 7.32 (d, J = 4.8 Hz, 1H), 7.54 (s, 1H), 8.25 (s, 1H), 8.43 (d, J = 4.8 Hz, 1H), 8.58 (s, 1H), 10.94 (s, 1H), 12.68 (s, 1H); ESI-MS (m/z) 444 (M + H)⁺. |
| 150 | 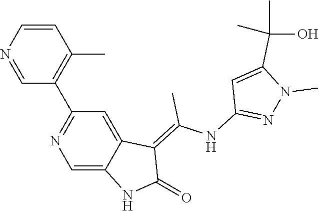 [SMILES: C/C(Nc1cc(C(C)(C)O)n(C)n1)=C1/C(=O)Nc2cnc(-c3cnccc3C)cc21] (Z)-3-(1-((5-(2- Hydroxypropan-2-yl)-1- methyl-1H-pyrazol-3- yl)amino)ethylidene)-5-(4- methylpyridin-3-yl)-1H- pyrrolo[2,3-c]pyridin- 2(3H)-one | Intermediate B9 and 2-(3-amino-1-methyl- 1H-pyrazol-5- yl)propan-2-ol (D35) Method H | ¹H NMR (400 MHz, DMSO-d₆) δ 1.58 (s, 6H), 2.38 (s, 3H), 2.72 (s, 3H), 3.95 (s, 3H), 5.43 (s, 1H), 6.14 (s, 1H), 7.32 (d, J = 4.8 Hz, 1H), 7.51 (s, 1H), 8.24 (s, 1H), 8.43 (d, J = 4.8 Hz, 1H), 8.57 (s, 1H), 10.90 (s, 1H), 12.59 (s, 1H); ESI-MS (m/z) 405 (M + H)⁺. |
| 151 | 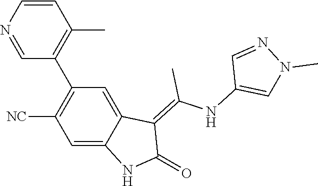 [SMILES: C/C(Nc1cnn(C)c1)=C1/C(=O)Nc2cc(C#N)c(-c3cnccc3C)cc21] (Z)-3-(1-((1-Methyl-1H- pyrazol-4- yl)amino)ethylidene)-5-(4- methylpyridin-3-yl)-2- oxoindoline-6-carbonitrile | 5-Bromo-2- oxoindoline-6- carbonitrile, 1- methyl-1H-pyrazol-4- amine and 4-methyl- 3-(4,4,5,5- tetramethyl-1,3,2- dioxaborolan-2- yl)pyridine Method I | ¹H NMR (400 MHz, DMSO-d₆) δ 2.20 (s, 3H), 2.48 (s, 3H), 3.85 (s, 3H), 7.31 (s, 1H), 7.36-7.38 (m, 1H), 7.40 (s, 1H), 7.56 (s, 1H), 7.94 (s, 1H), 8.41 (s, 1H), 8.50 (d, J = 4.8 Hz, 1H), 11.02 (s, 1H), 12.15 (s, 1H); ESI-MS (m/z) 371 (M + H)⁺. |
| 152 | 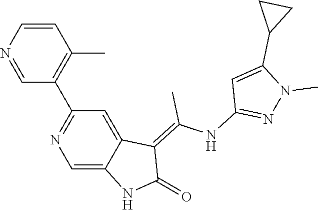 [SMILES: C/C(Nc1cc(C2CC2)n(C)n1)=C1/C(=O)Nc2cnc(-c3cnccc3C)cc21] (Z)-3-(1-((5-Cyclopropyl- 1-methyl-1H-pyrazol-3- yl)amino)ethylidene)-5-(4- methylpyridin-3-yl)-1H- pyrrolo[2,3-c]pyridin- 2(3H)-one | Step 1 of Method I, 5-cyclopropyl-1- methyl-1H-pyrazol-3- amine and (4- methylpyridin-3- yl)boronic acid Method I | ¹H NMR (400 MHz, DMSO-d₆) δ 0.67-0.72 (m, 2H), 0.94-1.01 (m, 2H), 1.89-1.95 (m, 1H), 2.37 (s, 3H), 2.69 (s, 3H), 3.81 (s, 3H), 5.6 (s, 1H), 7.32 (d, J = 4.8 Hz, 1H), 7.50 (s, 1H), 8.23 (s, 1H), 8.43 (d, J = 4.8 Hz, 1H), 8.57 (s, 1H), 10.89 (s, 1H), 12.58 (s, 1H); ESI- MS (m/z) 386 (M + H)⁺. |
| 153 | 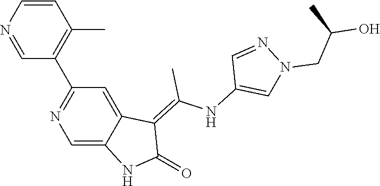 [SMILES: C/C(Nc1cnn(C[C@@H](C)O)c1)=C1/C(=O)Nc2cnc(-c3cnccc3C)cc21] (R,Z)-3-(1-((1-(2- Hydroxypropyl)-1H- pyrazol-4- yl)amino)ethylidene)-5-(4- methylpyridin-3-yl)-1H- pyrrolo[2,3-c]pyridin- 2(3H)-one | Step 1 of Method I, (R)-1-(4-amino-1H- pyrazol-1-yl)propan- 2-ol (D48) and (4- methylpyridin-3- yl)boronic acid pinacol ester Method I | ¹H NMR (400 MHz, DMSO-d6) δ 1.07 (d, J = 4.4 Hz, 3H), 2.37 (s, 3H), 2.54 (s, 3H), 3.35-3.42 (m, 2H), 3.95-4.05 (m, 1H), 4.97 (d, J = 4.4 Hz, 1H), 7.32 (d, J = 5.2 Hz, 1H), 7.49 (s, 1H), 7.60 (s, 1H), 7.94 (s, 1H), 8.22 (s, 1H), 8.43 (d, J = 4.8 Hz, 1H), 8.57 (s, 1H), 10.86 (s, 1H), 12.15 (s, 1H); ESI-MS (m/z) 391 (M + H)+. |
| 154 | 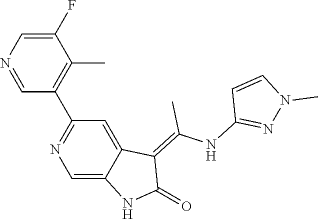 [SMILES: C/C(Nc1ccn(C)n1)=C1/C(=O)Nc2cnc(-c3cncc(F)c3C)cc21] (Z)-5-(5-Fluoro-4- methylpyridin-3-yl)-3-(1- ((1-methyl-1H-pyrazol-3- yl)amino)ethylidene)-1H- pyrrolo[2,3-c]pyridin- 2(3H)-one | Step 1 of Method I, 1-methyl-1H-pyrazol- 3-amine and (5- fluoro-4- methylpyridin-3- yl)boronic acid Method I | 1H NMR (400 MHz, DMSO-d6) δ 2.32 (s, 3H), 2.72 (s, 3H), 3.83 (s, 3H), 6.30 (s, 1H), 7.58 (s, 1H), 7.78 (s, 1H), 8.26 (s, 1H), 8.50 (s, 2H), 10.96 (s, 1H), 12.65 (s, 1H); ESI-MS (m/z) 365 (M + H)+. |
| 155 | 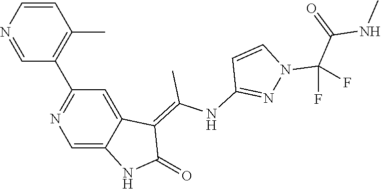 [SMILES: CNC(=O)C(F)(F)n1ccc(N/C(C)=C2\C(=O)Nc3cnc(-c4cnccc4C)cc32)n1] (Z)-2,2-Difluoro-N-methyl- 2-(3-((1-(5-(4- methylpyridin-3-yl)-2-oxo- 1H-pyrrolo[2,3-c]pyridin- 3(2H)- ylidene)ethyl)amino)-1H- pyrazol-1-yl)acetamide | Step 1 of method I, 2-(3-amino-1H- pyrazol-1-yl)-2,2- difluoro-N- methylacetamide (D43) and 4-methyl- 3-(4,4,5,5- tetramethyl-1,3,2- dioxaborolan-2- yl)pyridine Method I | ¹H NMR (400 MHz, DMSO-d₆) δ 2.38 (s, 3H), 2.75 (s, 3H), 2.79 (d, J = 3.6 Hz, 3H), 6.69 (s, 1H), 7.32 (d, J = 4.8 Hz, 1H), 7.58 (s, 1H), 8.27 (s, 1H), 8.37 (s, 1H), 8.43 (d, J = 4.8 Hz, 1H), 8.58 (s, 1H), 9.30 (s, 1H), 11.04 (s, 1H), 12.73 (s, 1H); ESI-MS (m/z) 440 (M + H)⁺. |
| 156 | 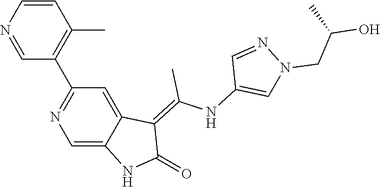 [SMILES: C/C(Nc1cnn(C[C@H](C)O)c1)=C1/C(=O)Nc2cnc(-c3cnccc3C)cc21] (S,Z)-3-(1-((1-(2- Hydroxypropyl)-1H- pyrazol-4- yl)amino)ethylidene)-5-(4- methylpyridin-3-yl)-1H- pyrrolo[2,3-c]pyridin- 2(3H)-one | Step 1 of method I, (S)-1-(4-amino-1H- pyrazol-1-yl)propan- 2-ol (D44) and 4- methyl-3-(4,4,5,5- tetramethyl-1,3,2- dioxaborolan-2- yl)pyridine Method I | ¹H NMR (400 MHz, DMSO-d₆) δ 0.97 (d, J = 6.0 Hz, 3H), 2.37 (s, 3H), 2.54 (s, 3H), 3.98-4.03 (m, 3H), 4.97 (d, J = 4.4 Hz, 1H), 7.32 (d, J = 4.8 Hz, 1H), 7.49 (s, 1H), 7.60 (s, 1H), 7.94 (s, 1H), 8.22 (s, 1H), 8.43 (d, J = 4.8 Hz, 1H), 8.57 (s, 1H), 10.86 (s, 1H), 12.15 (s, 1H); ESI-MS (m/z) 391 (M + H)⁺. |
| 157 | 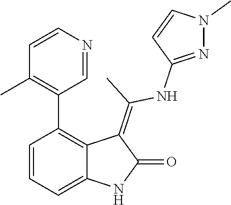 (Z)-3-(1-((1-Methyl-1H- pyrazol-3- yl)amino)ethylidene)-4-(4- methylpyridin-3-yl)indolin- 2-one | 4-Bromoindolin-2- one, 1-methyl-1H- pyrazol-3-amine and (4-methylpyridin-3- yl)boronic acid pinacol ester Method I | ¹H NMR (400 MHz, DMSO-d₆) δ 1.43 (s, 3H), 2.14 (s, 3H), 3.73 (s, 3H), 5.98 (s, 1H), 6.75-6.77 (m, 1H), 6.95 (dd, J = 7.6, 0.8 Hz, 1H), 7.12 (t, J = 8.0 Hz, 1H), 7.36 (d, J = 5.2 Hz, 1H), 7.63 (d, J = 2.4 Hz, 1H), 8.39 (s, 1H), 8.44 (d, J = 4.8 Hz, 1H), 10.82 (s, 1H), 12.77 (s, 1H); ESI-MS (m/z) 346 (M + H)⁺. |
| 158 | 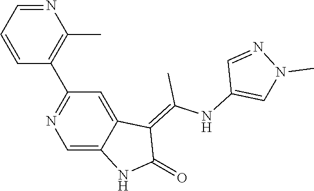 [SMILES: C/C(Nc1cnn(C)c1)=C1/C(=O)Nc2cnc(-c3cccnc3C)cc21] (Z)-3-(1-((1-Methyl-1H- pyrazol-4- yl)amino)ethylidene)-5-(2- methylpyridin-3-yl)-1H- pyrrolo[2,3-c]pyridin- 2(3H)-one | Step 1 of method I, 1-methyl-1H-pyrazol- 4-amine and 2- methyl-3-(4,4,5,5- tetramethyl-1,3,2- dioxaborolan-2- yl)pyridine | ¹H NMR (400 MHz, DMSO-d₆) δ 2.52 (s, 6H), 3.86 (s, 3H), 7.30 (dd, J = 7.2, 5.2 Hz, 1H), 7.47 (s, 1H), 7.58 (s, 1H), 7.79 (dd, J = 8.0, 2.0 Hz, 1H), 7.96 (s, 1H), 8.20 (s, 1H), 8.45 (dd, J = 4.8, 1.6 Hz, 1H), 10.85 (s, 1H), 12.13 (s, 1H); ESI- MS (m/z) 347 (M + H)⁺. |
| 159 | 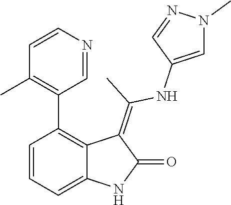 (Z)-3-(1-((1-Methyl-1H- pyrazol-4- yl)amino)ethylidene)-4-(4- methylpyridin-3-yl)indolin- 2-one | 4-Bromoindolin-2- one, 1-methyl-1H- pyrazol-4-amine and (4-methylpyridin-3- yl)boronic acid pinacol ester Method I | ¹H NMR (400 MHz, DMSO-d₆) δ 1.23 (s, 3H), 2.15 (s, 3H), 3.76 (s, 3H), 6.73 (dd, J = 7.6, 0.8 Hz, 1H), 6.96 (dd, J = 7.6, 0.8 Hz, 1H), 7.09 (t, J = 7.6 Hz, 1H), 7.32-7.34 (m, 2H), 7.73 (s, 1H), 8.39 (s, 1H), 8.43 (d, J = 4.8 Hz, 1H), 10.77 (s, 1H), 12.35 (s, 1H); ESI-MS (m/z) 346 (M + H)⁺. |
| 160 | 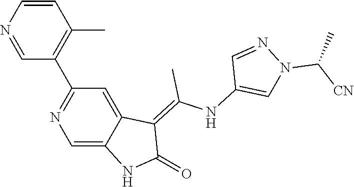 [SMILES: C/C(Nc1cnn([C@H](C)C#N)c1)=C1/C(=O)Nc2cnc(-c3cnccc3C)cc21] (R,Z)-2-(4-((1-(5-(4- Methylpyridin-3-yl)-2-oxo- 1H-pyrrolo[2,3-c]pyridin- 3(2H)- ylidene)ethyl)amino)-1H- pyrazol-1-yl)propanenitrile | Intermediate B9 and (R)-2-(4-amino-1H- pyrazol-1- yl)propanenitrile (D49) Method H | ¹H NMR (400 MHz, DMSO-d₆) δ 1.24 (d, J = 4.0 Hz, 3H), 2.37 (s, 3H), 3.56 (s, 3H), 5.84-5.90 (m, 1H), 7.32 (d, J = 4.8 Hz, 1H), 7.52 (s, 1H), 7.81 (s, 1H), 8.20 (s, 1H), 8.23 (s, 1H), 8.43 (d, J = 4.8 Hz, 1H), 8.57 (s, 1H), 10.90 (s, 1H), 12.18 (s, 1H); ESI-MS (m/z) 386 (M + H)⁺. |
| 161 | 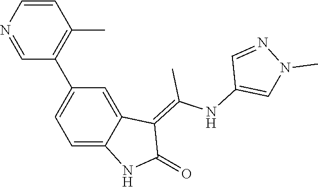 (Z)-3-(1-((1-Methyl-1H- pyrazol-4- yl)amino)ethylidene)-5-(4- methylpyridin-3-yl)indolin- 2-one | 5-Bromoindolin-2- one, 1-methyl-1H- pyrazol-4-amine and 4-methyl-3-(4,4,5,5- tetramethyl-1,3,2- dioxaborolan-2- yl)pyridine Method 1 | ¹H NMR (400 MHz, DMSO-d₆) δ 2.29 (s, 3H), 2.44 (s, 3H), 3.85 (s, 3H), 6.99 (s, 2H), 7.32 (d, J = 6.8 Hz, 2H), 7.52 (s, 1H), 7.89 (s, 1H), 8.38-8.40 (m, 2H), 10.65 (s, 1H), 11.80 (s, 1H); ESI-MS (m/z) 386 (M + H)⁺. |
| 162 | 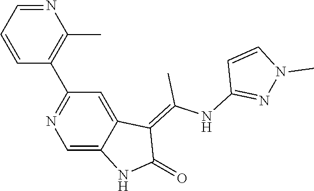 [SMILES: C/C(Nc1ccn(C)n1)=C1/C(=O)Nc2cnc(-c3cccnc3C)cc21] (Z)-3-(1-((1-Methyl-1H- pyrazol-3- yl)amino)ethylidene)-5-(2- methylpyridin-3-yl)-1H- pyrrolo[2,3-c]pyridin- 2(3H)-one | Step 1 of method I, 1-methyl-1H-pyrazol- 3-amine and 2- methyl-3-(4,4,5,5- tetramethyl-1,3,2- dioxaborolan-2- yl)pyridine | ¹H NMR (400 MHz, DMSO-d₆) δ 2.53 (s, 3H), 2.70 (s, 3H), 3.83 (s, 3H), 6.28 (s, 1H), 7.31 (dd, J = 7.6, 4.8 Hz, 1H), 7.50 (s, 1H), 7.72-7.81 (m, 2H), 8.22 (s, 1H), 8.46 (dd, J = 4.8, 1.6 Hz, 1H), 10.90 (s, 1H), 12.63 (s, 1H); ESI-MS (m/z) 347 (M + H)⁺. |
| 163 | 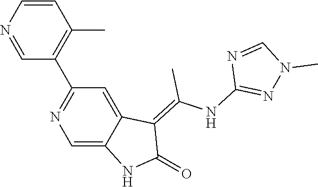 [SMILES: C/C(Nc1ncn(C)n1)=C1/C(=O)Nc2cnc(-c3cnccc3C)cc21] (Z)-3-(1-((1-Methyl-1H- 1,2,4-triazol-3- yl)amino)ethylidene)-5-(4- methylpyridin-3-yl)-1H- pyrrolo[2,3-c]pyridin- 2(3H)-one | Step 1 of Method I, 1-methyl-1H-1,2,4- triazol-3-amine and (4-methylpyridin-3- yl)boronic acid, pinacol ester Method I | ¹H NMR (400 MHz, DMSO-d₆) δ 2.38 (s, 3H), 2.95 (s, 3H), 3.86 (s, 3H), 7.33 (d, J = 4.8 Hz, 1H), 7.58 (s, 1H), 8.27 (s, 1H), 8.44 (d, J = 4.8 Hz, 1H), 8.47 (s, 1H), 8.58 (s, 1H), 11.03 (s, 1H), 12.95 (s, 1H); ESI-MS (m/z) 348 (M + H)⁺. |
| 164 | 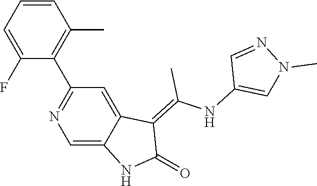 [SMILES: C/C(Nc1cnn(C)c1)=C1/C(=O)Nc2cnc(-c3c(C)cccc3F)cc21] (Z)-5-(2-Fluoro-6- methylphenyl)-3-(1-((1- methyl-1H-pyrazol-4- yl)amino)ethylidene)-1H- pyrrolo[2,3-c]pyridin- 2(3H)-one | Step 1 of Method I, 1-methyl-1H-pyrazol- 4-amine and (2- fluoro-6- methylphenyl)boronic acid, pinacol ester Method I | ¹H NMR (400 MHz, DMSO-d₆) δ 2.13 (s, 3H), 2.48 (s, 3H), 3.86 (s, 3H), 7.08-7.15 (m, 2H), 7.29- 7.35 (m, 2H), 7.57-7.59 (m, 1H), 7.95 (s, 1H), 8.20 (s, 1H), 10.84 (s, 1H), 12.12 (s, 1H); ESI-MS (m/z) 364 (M + H)⁺. |
| 165 | 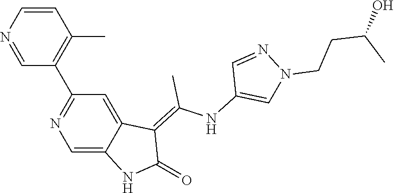 [SMILES: C/C(Nc1cnn(CC[C@@H](C)O)c1)=C1/C(=O)Nc2cnc(-c3cnccc3C)cc21] (R,Z)-3-(1-((1-(3- Hydroxybutyl)-1H-pyrazol- 4-yl)amino)ethylidene)-5- (4-methylpyridin-3-yl)-1H- pyrrolo[2,3-c]pyridin- 2(3H)-one | Step 1 of Method I, (R)-4-(4-amino-1H- pyrazol-1-yl)butan-2- ol (D36) and (4- methylpyridin-3- yl)boronic acid, pinacol ester Method I | ¹H NMR (400 MHz, DMSO-d₆) δ 1.08 (d, J = 6.4 Hz, 3H), 1.77-1.82 (m, 2H), 2.37 (s, 3H), 2.54 (s, 3H), 3.54-3.60 (m, 1H), 4.15- 4.20 (m, 2H), 4.65 (d, J = 4.8 Hz, 1H), 7.32 (d, J = 4.8 Hz, 1H), 7.49 (s, 1H), 7.59 (s, 1H), 8.00 (s, 1H), 8.22 (s, 1H), 8.42 (d, J = 4.8 Hz, 1H), 8.56 (s, 1H), 10.86 (s, 1H), 12.15 (s, 1H); ESI-MS (m/z) 405 (M + H)⁺. |
| 166 | 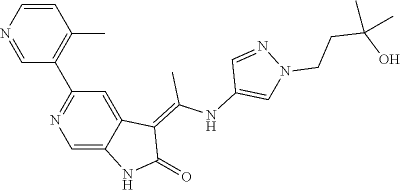 [SMILES: C/C(Nc1cnn(CCC(C)(C)O)c1)=C1/C(=O)Nc2cnc(-c3cnccc3C)cc21] (Z)-3-(1-((1-(3-Hydroxy-3- methylbutyl)-1H-pyrazol- 4-yl)amino)ethylidene)-5- (4-methylpyridin-3-yl)-1H- pyrrolo[2,3-c]pyridin- 2(3H)-one | Step 1 of Method I, 4-(4-amino-1H- pyrazol-1-yl)-2- methylbutan-2-ol (D37) and (4- methylpyridin-3- yl)boronic acid, pinacol ester Method I | ¹H NMR (400 MHz, DMSO-d₆) δ 1.13 (s, 6H), 1.89-1.95 (m, 2H), 2.37 (s, 3H), 2.53 (s, 3H), 4.16-4.22 (m, 2H), 4.47 (s, 1H), 7.31 (d, J = 4.8 Hz, 1H), 7.49 (s, 1H), 7.58 (s, 1H), 8.02 (s, 1H), 8.22 (s, 1H), 8.42 (d, J = 4.8 Hz, 1H), 8.56 (s, 1H), 10.85 (s, 1H), 12.14 (s, 1H); ESI-MS (m/z) 419 (M + H)⁺. |
| 167 | 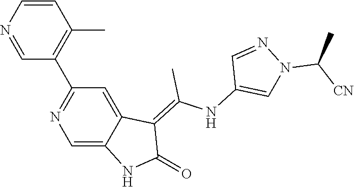 [SMILES: C/C(Nc1cnn([C@@H](C)C#N)c1)=C1/C(=O)Nc2cnc(-c3cnccc3C)cc21] (S,Z)-2-(4-((1-(5-(4- Methylpyridin-3-yl)-2-oxo- 1H-pyrrolo[2,3-c]pyridin- 3(2H)- ylidene)ethyl)amino)-1H- pyrazol-1-yl)propanenitrile | Intermediate B9 and (S)-2-(4-amino-1H- pyrazol-1- yl)propanenitrile (D45) Method H | ¹H NMR (400 MHz, DMSO-d₆) δ 1.82 (d, J = 7.2 Hz, 3H), 2.37 (s, 3H), 2.56 (s, 3H), 5.84-5.90 (m, 1H), 7.32 (d, J = 5.2 Hz, 1H), 7.52 (s, 1H), 7.81 (s, 1H), 8.20 (s, 1H), 8.23 (s, 1H), 8.43 (d, J = 4.8 Hz, 1H), 8.57 (s, 1H), 10.91 (s, 1H), 12.19 (s, 1H); ESI-MS (m/z) 386 (M + H)⁺. |
| 168 | 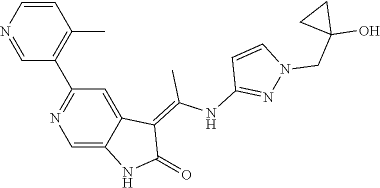 [SMILES: C/C(Nc1ccn(CC2(O)CC2)n1)=C1/C(=O)Nc2cnc(-c3cnccc3C)cc21] (Z)-3-(1-((1-((1- Hydroxycyclopropyl)methyl)- 1H-pyrazol-3- yl)amino)ethylidene)-5-(4- methylpyridin-3-yl)-1H- pyrrolo[2,3-c]pyridin- 2(3H)-one | Intermediate B9 and 1-((3-amino-1H- pyrazol-1- yl)methyl)cyclopropanol (D54) Method H | ¹H NMR (400 MHz, DMSO-d₆) δ 0.67-0.69 (m, 4H), 2.38 (s, 3H), 2.73 (s, 3H), 4.14 (s, 2H), 5.56 (s, 1H), 6.32 (d, J = 2.4 Hz, 1H), 7.32 (d, J = 4.8 Hz, 1H), 7.53 (s, 1H), 7.80 (s, 1H), 8.24 (s, 1H), 8.43 (d, J = 4.8 Hz, 1H), 8.58 (s, 1H), 10.91 (s, 1H), 12.64 (s, 1H); ESI-MS (m/z) 403 (M + H)⁺. |
| 169 | 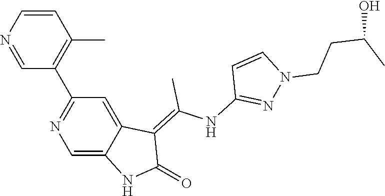 [SMILES: C/C(Nc1ccn(CC[C@@H](C)O)n1)=C1/C(=O)Nc2cnc(-c3cnccc3C)cc21] (R,Z)-3-(1-((1-(3- Hydroxybutyl)-1H-pyrazol- 3-yl)amino)ethylidene)-5- (4-methylpyridin-3-yl)-1H- pyrrolo[2,3-c]pyridin- 2(3H)-one | Step 1 of Method I, (R)-4-(3-amino-1H- pyrazol-1-yl)butan-2- ol (D38) and (4- methylpyridin-3- yl)boronic acid, pinacol ester Method I | ¹H NMR (400 MHz, DMSO-d₆) δ 1.07-1.12 (m, 3H), 1.74-1.95 (m, 2H), 2.38 (s, 3H), 2.71 (s, 3H), 3.38-3.42 (m, 2H), 4.15 (t, J = 6.8 Hz, 1H), 4.65 (d, J = 4.8 Hz, 1H), 6.28 (d, J = 2.0 Hz, 1H), 7.32 (d, J = 4.8 Hz, 1H), 7.52 (s, 1H), 7.81 (s, 1H), 8.24 (s, 1H), 8.32 (s, 1H), 8.43 (d, J = 4.8 Hz, 1H), 8.58 (s, 1H), 10.91 (s, 1H), 12.63 (s, 1H); ESI-MS (m/z) 405 (M + H)⁺. |
| 170 | 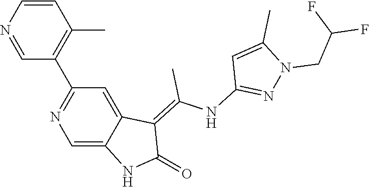 [SMILES: C/C(Nc1cc(C)n(CC(F)F)n1)=C1/C(=O)Nc2cnc(-c3cnccc3C)cc21] (Z)-3-(1-((1-(2,2- Difluoroethyl)-5-methyl- 1H-pyrazol-3- yl)amino)ethylidene)-5-(4- methylpyridin-3-yl)-1H- pyrrolo[2,3-c]pyridin- 2(3H)-one | Step 1 of Method I, 1-(2,2-difluoroethyl)- 5-methyl-1H-pyrazol- 3-amine (D39) and (4-methylpyridin-3- yl)boronic acid, pinacol ester Method I | ¹H NMR (400 MHz, DMSO-d₆) δ 2.31 (s, 3H), 2.37 (s, 3H), 2.72 (s, 3H), 4.50-4.60 (m, 2H), 6.21 (s, 1H), 6.38 (t, J = 3.6 Hz, 1H), 7.32 (d, J = 4.8 Hz, 1H), 7.53 (s, 1H), 8.24 (s, 1H), 8.43 (d, J = 4.8 Hz, 1H), 8.57 (s, 1H), 10.92 (s, 1H), 12.62 (s, 1H); ESI-MS (m/z) 411 (M + H)⁺. |
| 171 | 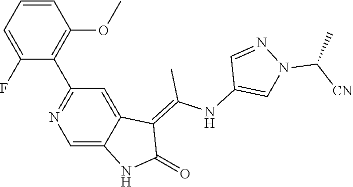 [SMILES: COc1cccc(F)c1-c1cc2c(cn1)NC(=O)/C2=C(/C)Nc1cnn([C@H](C)C#N)c1] (R,Z)-2-(4-((1-(5-(2- Fluoro-6-methoxyphenyl)- 2-oxo-1H-pyrrolo[2,3- c]pyridin-3(2H)- ylidene)ethyl)amino)-1H- pyrazol-1-yl)propanenitrile | Intermediate B7 and (R)-2-(4-amino-1H- pyrazol-1- yl)propanenitrile (D49) Method H | ¹H NMR (400 MHz, DMSO-d₆) δ 1.82 (d, J = 7.2 Hz, 3H), 2.48 (s, 3H), 3.72 (s, 3H), 5.83-5.89 (m, 1H), 6.87 (t, J = 8.4 Hz, 1H), 6.95 (d, J = 8.4 Hz, 1H), 7.33-7.42 (m, 2H), 7.80 (s, 1H), 8.18 (d, J = 6.0 Hz, 2H), 10.86 (s, 1H), 12.15 (s, 1H); ESI-MS (m/z) 419 (M + H)⁺. |
| 172 | 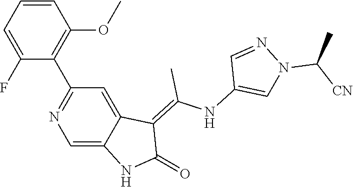 [SMILES: COc1cccc(F)c1-c1cc2c(cn1)NC(=O)/C2=C(/C)Nc1cnn([C@@H](C)C#N)c1] (S,Z)-2-(4-((1-(5-(2- Fluoro-6-methoxyphenyl)- 2-oxo-1H-pyrrolo[2,3- c]pyridin-3(2H)- ylidene)ethyl)amino)-1H- pyrazol-1-yl)propanenitrile | Intermediate B7 and (S)-2-(4-amino-1H- pyrazol-1- yl)propanenitrile (D45) Method H | ¹H NMR (400 MHz, DMSO-d₆) δ 1.82 (d, J = 7.2 Hz, 3H), 2.47 (s, 3H), 3.72 (s, 3H), 5.86 (d, J = 7.2 Hz, 1H), 6.87 (t, J = 8.4 Hz, 1H), 6.94 (d, J = 8.4 Hz, 1H), 7.32 (s, 1H), 7.37-7.39 (m, 1H), 7.80 (s, 1H), 8.18 (d, J = 6.4 Hz, 2H), 10.83 (s, 1H), 12.13 (s, 1H); ESI-MS (m/z) 419 (M + H)⁺. |
| 173 | 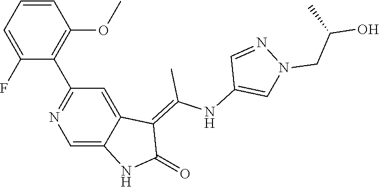 [SMILES: COc1cccc(F)c1-c1cc2c(cn1)NC(=O)/C2=C(/C)Nc1cnn(C[C@H](C)O)c1] (S,Z)-5-(2-Fluoro-6- methoxyphenyl)-3-(1-((1- (2-hydroxypropyl)-1H- pyrazol-4- yl)amino)ethylidene)-1H- pyrrolo[2,3-c]pyridin- 2(3H)-one | Step 1 of Method I, (S)-1-(4-amino-1H- pyrazol-1-yl)propan- 2-ol (D44) and 2- fluoro-6- methoxyphenylboronic acid Method I | ¹H NMR (400 MHz, DMSO-d₆) δ 1.04 (d, J = 7.2 Hz, 3H), 2.45 (s, 3H), 3.99- 4.00 (m, 3H), 5.03 (d, J = 4.4 Hz, 1H), 6.86 (t, J = 8.4 Hz, 1H), 6.94 (d, J = 8.4 Hz, 1H), 7.29 (s, 1H), 7.34-7.40 (m, 1H), 7.58 (s, 1H), 7.91 (s, 1H), 8.16 (s, 1H), 10.80 (s, 1H), 12.08 (s, 1H); ESI- MS (m/z) 424 (M + H)⁺. |
| 174 | 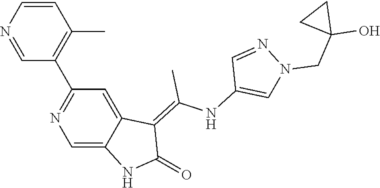 [SMILES: C/C(Nc1cnn(CC2(O)CC2)c1)=C1/C(=O)Nc2cnc(-c3cnccc3C)cc21] (Z)-3-(1-((1-((1- Hydroxycyclopropyl)methyl)- 1H-pyrazol-4- yl)amino)ethylidene)-5-(4- methylpyridin-3-yl)-1H- pyrrolo[2,3-c]pyridin- 2(3H)-one | Intermediate B9 and 1-((4-amino-1H- pyrazol-1- yl)methyl)cyclopropanol (D55) Method H | ¹H NMR (400 MHz, DMSO-d₆) δ 0.67-0.69 (m, 4H), 2.37 (s, 3H), 2.56 (s, 3H), 4.17 (s, 2H), 5.58 (s, 1H), 7.32 (d, J = 4.8 Hz, 1H), 7.49 (s, 1H), 7.60 (s, 1H), 7.98 (s, 1H), 8.22 (s, 1H), 8.42 (d, J = 4.8 Hz, 1H), 8.57 (s, 1H), 10.85 (s, 1H), 12.17 (s, 1H); ESI-MS (m/z) 403 (M + H)⁺. |
| 175 | 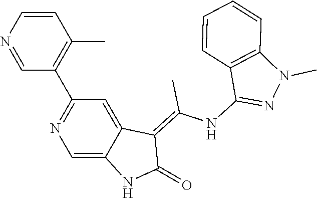 [SMILES: C/C(Nc1nn(C)c2ccccc12)=C1/C(=O)Nc2cnc(-c3cnccc3C)cc21] (Z)-3-(1-((1-Methyl-1H- indazol-3- yl)amino)ethylidene)-5-(4- methylpyridin-3-yl)-1H- pyrrolo[2,3-c]pyridin- 2(3H)-one | Step 1 of Method I, 1-methylindazol-3- amine and (4- methylpyridin-3- yl)boronic acid, pinacol ester Method I | ¹H NMR (400 MHz, DMSO-d₆) δ 2.39 (s, 3H), 2.85 (s, 3H),4.06 (s, 3H), 7.25 (t, J = 7.6 Hz, 1H), 7.33 (d, J = 4.8 Hz, 1H), 7.51 (t, J = 8.4 Hz, 1H), 7.57 (s, 1H), 7.71 (t, J = 6.4 Hz, 2H), 8.29 (s, 1H), 8.44 (d, J = 4.8 Hz, 1H), 8.60 (s, 1H), 11.04 (s, 1H), 13.09 (s, 1H); ESI-MS (m/z) 397 (M + H)⁺. |
| 176 | 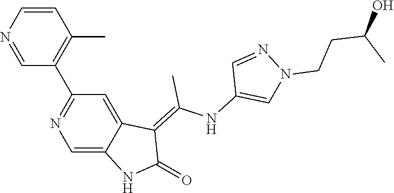 [SMILES: C/C(Nc1cnn(CC[C@H](C)O)c1)=C1/C(=O)Nc2cnc(-c3cnccc3C)cc21] (S,Z)-3-(1-((1-(3- Hydroxybutyl)-1H-pyrazol- 4-yl)amino)ethylidene)-5- (4-methylpyridin-3-yl)-1H- pyrrolo[2,3-c]pyridin- 2(3H)-one | Intermediate B9 and (S)-4-(4-amino-1H- pyrazol-1-yl)butan-2-ol (D57) Method H | ¹H NMR (400 MHz, DMSO-d₆) δ 1.08 (d, J = 6.4 Hz, 3H), 7.76-1.91 (m, 2H), 2.37 (s, 3H), 2.54 (s, 3H), 3.57 (br s, 1H), 4.17 (t, J = 6.8 Hz, 2H), 4.65 (d, J = 4.8 Hz, 1H), 7.32 (d, J = 4.8 Hz, 1H), 7.51 (s, 1H), 7.59 (s, 1H), 8.00 (s, 1H), 8.22 (s, 1H), 8.43 (d, J = 4.8 Hz, 1H), 8.57 (s, 1H), 10.88 (s, 1H), 12.18 (s, 1H); ESI-MS (m/z) 405 (M + H)⁺. |
| 177 | 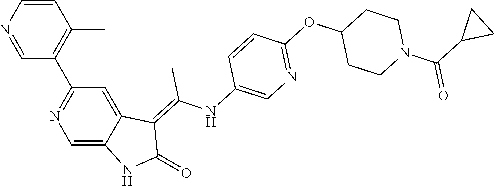 [SMILES: C/C(Nc1ccc(OC2CCN(C(=O)C3CC3)CC2)nc1)=C1/C(=O)Nc2cnc(-c3cnccc3C)cc21] (Z)-3-(1-((6-((1- (Cyclopropanecarbonyl) piperidin-4-yl)oxy)pyridin-3- yl)amino)ethylidene)-5-(4- methylpyridin-3-yl)-1H- pyrrolo[2,3-c]pyridin- 2(3H)-one | Intermediate B9 and (4-((5-aminopyridin- 2-yl)oxy)piperidin-1- yl)(cyclopropyl) methanone (D58) Method H | ¹H NMR (400 MHz, DMSO-d₆) δ 0.70-0.74 (m, 4H), 1.56-1.67 (m, 2H), 1.98-2.06 (m, 3H), 2.37 (s, 3H), 2.51 (s, 3H), 3.21-3.23 (m, 1H), 3.37-3.39 (m, 1H), 3.98-4.02 (m, 2H), 5.24- 5.28 (m, 1H), 6.92 (d, J = 8.4 Hz, 1H), 7.32 (d, J = 4.8 Hz, 1H), 7.49 (s, 1H), 7.75- 7.78 (m, 1H), 8.21 (s, 1H), 8.24 (s, 1H), 8.43 (d, J = 4.8 Hz, 1H), 8.57 (s, 1H), 10.89 (s, 1H), 12.58 (s, 1H); ESI- MS (m/z) 511 (M + H)⁺. |
| 178 | 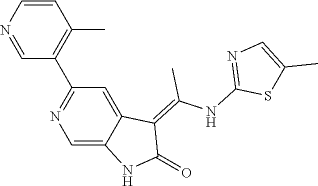 [SMILES: C/C(Nc1ncc(C)s1)=C1/C(=O)Nc2cnc(-c3cnccc3C)cc21] (Z)-5-(4-Methylpyridin-3- yl)-3-(1-((5-methylthiazol- 2-yl)amino)ethylidene)-1H- pyrrolo[2,3-c]pyridin- 2(3H)-one | Intermediate B9 and 5-methylthiazol-2- amine Method H | ¹H NMR (400 MHz, DMSO-d₆) δ 2.37 (s, 3H), 2.42 (s, 3H), 2.89 (s, 3H), 7.30-7.33 (m, 2H), 7.61 (s, 1H), 8.28 (s, 1H), 8.43 (d, J = 4.8 Hz, 1H), 8.58 (s, 1H), 11.12 (s, 1H), 13.34 (s, 1H); ESI-MS (m/z) 364 (M + H)⁺. |
| 179 | 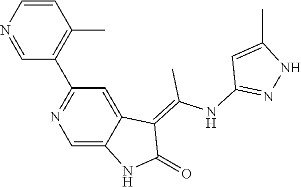 [SMILES: C/C(Nc1cc(C)[nH]n1)=C1/C(=O)Nc2cnc(-c3cnccc3C)cc21] (Z)-3-(1-((5-Methyl-1H- pyrazol-3- yl)amino)ethylidene)-5-(4- methylpyridin-3-yl)-1H- pyrrolo[2,3-c]pyridin- 2(3H)-one | Step 1 of Method I, 5-methyl-1H-pyrazol- 3-amine (CAS # 31230-17-8) and (4- methylpyridin-3- yl)boronic acid, pinacol ester Method I | ¹H NMR (400 MHz, DMSO-d₆) δ 2.26 (s, 3H), 2.69 (s, 3H), 2.37 (s, 1H), 2.70 (s, 3H), 6.07 (s, 1H), 7.32 (d, J = 4.8 Hz, 1H), 7.51 (s, 1H), 8.23 (s, 1H), 8.43 (d, J = 4.8 Hz, 1H), 8.57 (s, 1H), 10.87 (s, 1H), 12.55-12.57 (m, 2H); ESI- MS (m/z) 347 (M + H)⁺. |
| 180 | 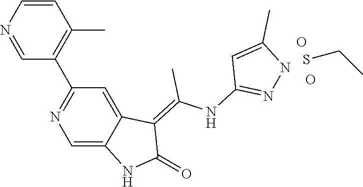 (Z)-3-(1-((1- (Ethylsulfonyl)-5-methyl- 1H-pyrazol-3- yl)amino)ethylidene)-5-(4- methylpyridin-3-yl)-1H- pyrrolo[2,3-c]pyridin- 2(3H)-one | Intermediate B9 and 1-(ethylsulfonyl)-5- methyl-1H-pyrazol-3- amine (D56) Method H | ¹H NMR (400 MHz, DMSO-d₆) δ 1.16 (t, J = 7.2 Hz, 3H), 2.38 (s, 3H), 2.50 (s, 3H), 2.80 (s, 3H), 3.70 (q, J = 7.2 Hz, 2H), 6.54 (s, 1H), 7.33 (d, J = 4.8 Hz, 1H), 7.59 (s, 1H), 8.27 (s, 1H), 8.44 (d, J = 4.8 Hz, 1H), 8.58 (s, 1H), 11.06 (s, 1H), 12.74 (s, 1H); ESI-MS (m/z) 439 (M + H)⁺. |
| 181 | 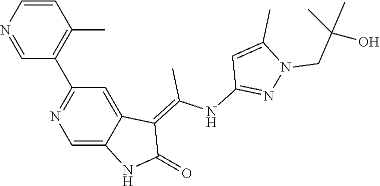 [SMILES: C/C(Nc1cc(C)n(CC(C)(C)O)n1)=C1/C(=O)Nc2cnc(-c3cnccc3C)cc21] (Z)-3-(1-((1-(2-Hydroxy-2- methylpropyl)-5-methyl- 1H-pyrazol-3- yl)amino)ethylidene)-5-(4- methylpyridin-3-yl)-1H- pyrrolo[2,3-c]pyridin- 2(3H)-one | Step 1 of Method I, 1-(3-amino-5-methyl- 1H-pyrazol-1-yl)-2- methylpropan-2-ol (D50) and (4- methylpyridin-3- yl)boronic acid, pinacol ester Method I | ¹H NMR (400 MHz, DMSO-d₆) δ 1.14 (s, 6H), 2.31 (s, 3H), 2.37 (s, 3H), 2.71 (s, 3H), 3.91 (s, 2H), 4.68 (s, 1H), 6.10 (s, 1H), 7.32 (d, J = 4.8 Hz, 1H), 7.52 (s, 1H), 8.23 (s, 1H), 8.43 (d, J = 4.8 Hz, 1H), 8.57 (s, 1H), 10.91 (s, 1H), 12.56 (s, 1H); ESI-MS (m/z) 419 (M + H)⁺. |
| 182 | 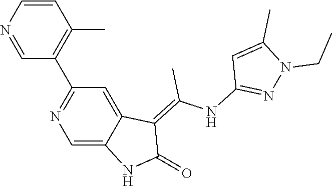 [SMILES: CCn1nc(N/C(C)=C2\C(=O)Nc3cnc(-c4cnccc4C)cc32)cc1C] (Z)-3-(1-((1-Ethyl-5- methyl-1H-pyrazol-3- yl)amino)ethylidene)-5-(4- methylpyridin-3-yl)-1H- pyrrolo[2,3-c]pyridin- 2(3H)-one | Step 1 of Method I, 1-ethyl-5-methyl-1H- pyrazol-3-amine (CAS # 956364-46-8) and (4- methylpyridin-3- yl)boronic acid, pinacol ester Method I | ¹H NMR (400 MHz, DMSO-d₆) δ 1.30 (t, J = 7.2 Hz, 3H), 2.28 (s, 3H), 2.36 (s, 3H), 2.69 (s, 3H), 3.98- 4.05 (m, 2H), 6.09 (s, 1H), 7.32 (d, J = 4.8 Hz, 1H), 7.50 (s, 1H), 8.23 (s, 1H), 8.42 (d, J = 4.8 Hz, 1H), 8.55 (s, 1H), 10.90 (s, 1H), 12.57 (s, 1H); ESI-MS (m/z) 375 (M + H)⁺. |
| 183 | 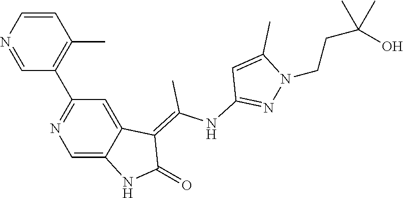 [SMILES: C/C(Nc1cc(C)n(CCC(C)(C)O)n1)=C1/C(=O)Nc2cnc(-c3cnccc3C)cc21] (Z)-3-(1-((1-(3-Hydroxy-3- methylbutyl)-5-methyl-1H- pyrazol-3- yl)amino)ethylidene)-5-(4- methylpyridin-3-yl)-1H- pyrrolo[2,3-c]pyridin- 2(3H)-one | Step 1 of Method I, 4-(3-amino-5-methyl- 1H-pyrazol-1-yl)-2- methylbutan-2-ol (D40) and (4- methylpyridin-3- yl)boronic acid, pinacol ester Method I | ¹H NMR (400 MHz, DMSO-d₆) δ 1.14 (s, 6H), 1.79-1.85 (m, 2H), 2.31 (s, 3H), 2.37 (s, 3H), 2.78 (s, 3H), 4.05-4.11 (m, 2H), 6.18 (s, 1H), 7.45 (d, J = 5.2 Hz, 1H), 7.53 (s, 1H), 7.73 (s, 1H), 8.27 (s, 1H), 8.55 (d, J = 5.2 Hz, 1H), 8.63 (s, 1H), 11.38 (s, 1H), 11.92 (s, 1H); ESI-MS (m/z) 433 (M + H)⁺. |
| 184 | 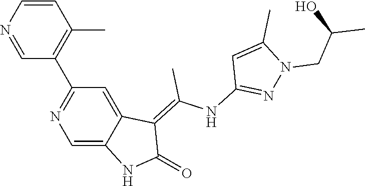 [SMILES: C/C(Nc1cc(C)n(C[C@H](C)O)n1)=C1/C(=O)Nc2cnc(-c3cnccc3C)cc21] (S,Z)-3-(1-((1-(2- Hydroxypropyl)-5-methyl- 1H-pyrazol-3- yl)amino)ethylidene)-5-(4- methylpyridin-3-yl)-1H- pyrrolo[2,3-c]pyridin- 2(3H)-one | Step 1 of Method I, (S)-1-(3-amino-5- methyl-1H-pyrazol-1- yl)propan-2-ol (D46) and (4- methylpyridin-3- yl)boronic acid, pinacol ester Method I | ¹H NMR (400 MHz, DMSO-d₆) δ 1.09 (d, J = 6.4 Hz, 3H), 2.30 (s, 3H), 2.38 (s, 3H), 2.71 (s, 3H), 3.88- 3.91 (m, 2H), 3.97-4.00 (m, 1H), 4.91 (d, J = 5.2 Hz, 1H), 6.09 (s, 1H), 7.32 (d, J = 4.8 Hz, 1H), 7.52 (s, 1H), 8.23 (s, 1H), 8.43 (d, J = 4.8 Hz, 1H), 8.57 (s, 1H), 10.90 (s, 1H), 12.60 (s, 1H); ESI- MS (m/z) 405 (M + H)⁺. |
| 185 | 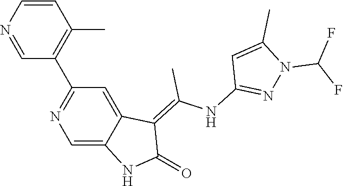 [SMILES: C/C(Nc1cc(C)n(C(F)F)n1)=C1/C(=O)Nc2cnc(-c3cnccc3C)cc21] (Z)-3-(1-((1- (Difluoromethyl)-5-methyl- 1H-pyrazol-3- yl)amino)ethylidene)-5-(4- methylpyridin-3-yl)-1H- pyrrolo[2,3-c]pyridin- 2(3H)-one | Step 1 of Method I, 1-(difluoromethyl)-5- methyl-1H-pyrazol-3- amine (D47) and (4- methylpyridin-3- yl)boronic acid, pinacol ester Method I | ¹H NMR (400 MHz, DMSO-d₆) δ 2.13 (s, 3H), 2.37 (s, 3H), 2.77 (s, 3H), 6.43 (s, 1H), 7.33 (d, J = 5.2 Hz, 1H), 7.57 (s, 1H), 7.64- 7.93 (m, 1H), 8.26 (s, 1H), 8.43 (d, J = 5.2 Hz, 1H), 8.58 (s, 1H), 11.01 (s, 1H), 12.72 (s, 1H); ESI-MS (m/z) 397 (M + H)⁺. |
| 186 | 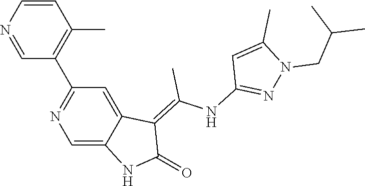 [SMILES: C/C(Nc1cc(C)n(CC(C)C)n1)=C1/C(=O)Nc2cnc(-c3cnccc3C)cc21] (Z)-3-(1-((1-Isobutyl-5- methyl-1H-pyrazol-3- yl)amino)ethylidene)-5-(4- methylpyridin-3-yl)-1H- pyrrolo[2,3-c]pyridin- 2(3H)-one | Step 1 of Method I, 1-isobutyl-5-methyl- 1H-pyrazol-3-amine (D41) and (4- methylpyridin-3- yl)boronic acid, pinacol ester Method I | ¹H NMR (400 MHz, DMSO-d₆) δ 0.87 (d, J = 6.8 Hz, 6H), 2.08-2.16 (m, 1H), 2.28 (s, 3H), 2.38 (s, 3H), 2.70 (s, 3H), 3.81 (d, J = 7.2 Hz, 2H), 6.10 (s, 1H), 7.32 (d, J = 4.8 Hz, 1H), 7.52 (s, 1H), 8.23 (s, 1H), 8.43 (d, J = 4.8 Hz, 1H), 8.58 (s, 1H), 10.91 (s, 1H), 12.58 (s, 1H); ESI-MS (m/z) 404 (M + H)⁺. |
| 187 | 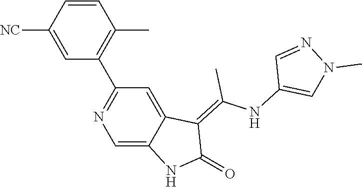 [SMILES: C/C(Nc1cnn(C)c1)=C1/C(=O)Nc2cnc(-c3cc(C#N)ccc3C)cc21] (Z)-4-Methyl-3-(3-(1-((1- methyl-1H-pyrazol-4- yl)amino)ethylidene)-2- oxo-2,3-dihydro-1H- pyrrolo[2,3-c]pyridin-5- yl)benzonitrile | Step 1 of Method I, 1-methyl-1H-pyrazol- 4-amine and 4- methyl-3-(4,4,5,5- tetramethyl-1,3,2- dioxaborolan-2- yl)benzonitrile Method I | ¹H NMR (400 MHz, DMSO-d₆) δ 1.07 (s, 3H), 2.41 (s, 3H), 3.86 (s, 3H), 7.49 (t, J = 2.4 Hz, 2H), 7.58 (s, 1H), 7.72 (d, J = 2.0 Hz, 1H), 7.85 (d, J = 2.0 Hz, 1H), 7.96 (s, 1H), 8.20 (s, 1H), 10.86 (s, 1H), 12.14 (s, 1H); ESI-MS (m/z) 370 (M + H)⁺. |
| 188 | 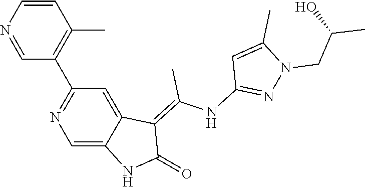 [SMILES: C/C(Nc1cc(C)n(C[C@@H](C)O)n1)=C1/C(=O)Nc2cnc(-c3cnccc3C)cc21] (R,Z)-3-(1-((1-(2- Hydroxypropyl)-5-methyl 1H-pyrazol-3- yl)amino)ethylidene)-5-(4- methylpyridin-3-yl)-1H- pyrrolo[2,3-c]pyridin- 2(3H)-one | Step 1 of Method I, (R)-1-(3-amino-5- methyl-1H-pyrazol-1- yl)propan-2-ol (D51) and (4- methylpyridin-3- yl)boronic acid, pinacol ester Method I | ¹H NMR (400 MHz, DMSO-d₆) δ 1.09 (d, J = 6.0 Hz, 3H), 2.30 (s, 3H), 2.38 (s, 3H), 2.71 (s, 3H), 3.88- 3.91 (m, 2H), 3.94-4.00 (m, 1H), 4.92 (d, J = 5.2 Hz, 1H), 6.09 (s, 1H), 7.32 (d, J = 4.8 Hz, 1H), 7.52 (s, 1H), 8.23 (s, 1H), 8.43 (d, J = 4.8 Hz, 1H), 8.57 (s, 1H), 10.91 (s, 1H), 12.60 (s, 1H); ESI- MS (m/z) 405 (M + H)⁺. |
| 189 | 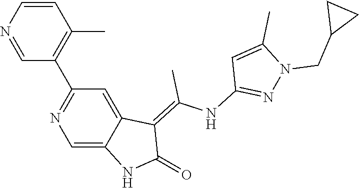 [SMILES: C/C(Nc1cc(C)n(CC2CC2)n1)=C1/C(=O)Nc2cnc(-c3cnccc3C)cc21] (Z)-3-(1-((1- (cyclopropylmethyl)-5- methyl-1H-pyrazol-3- yl)amino)ethylidene)-5-(4- methylpyridin-3-yl)-1H- pyrrolo[2,3-c]pyridin- 2(3H)-one | Step 1 of Method I, 1- (cyclopropylmethyl)- 5-methyl-1H-pyrazol- 3-amine (D59) and (4-methylpyridin-3- yl)boronic acid, pinacol ester Method I | ¹H NMR (400 MHz, DMSO-d₆) δ 0.32-0.35 (m, 2H), 0.52-0.55 (m, 2H), 1.21-1.25 (m, 1H), 2.30 (s, 3H), 2.38 (s, 3H), 2.71 (s, 3H), 3.89 (d, J = 6.8 Hz, 2H), 6.11 (s, 1H), 7.32 (d, J = 4.8 Hz, 1H), 7.51 (s, 1H), 8.23 (s, 1H), 8.43 (d, J = 4.8 Hz, 1H), 8.57 (s, 1H), 10.89 (s, 1H), 12.59 (s, 1H); ESI- MS (m/z) 401 (M + H)⁺. |
| 190 | 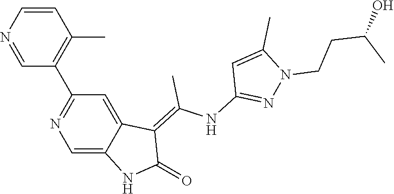 [SMILES: C/C(Nc1cc(C)n(CC[C@@H](C)O)n1)=C1/C(=O)Nc2cnc(-c3cnccc3C)cc21] (R,Z)-3-(1-((1-(3- Hydroxybutyl)-5-methyl- 1H-pyrazol-3- yl)amino)ethylidene)-5-(4- methylpyridin-3-yl)-1H- pyrrolo[2,3-c]pyridin- 2(3H)-one | Step 1 of Method I, (R)-4-(3-amino-5- methyl-1H-pyrazol-1- yl)butan-2-ol (D42) and (4- methylpyridin-3- yl)boronic acid, pinacol ester Method I | ¹H NMR (400 MHz, DMSO-d₆) δ 1.06 (d, J = 6.0 Hz, 3H), 1.69-1.76 (m, 2H), 2.17 (s, 3H), 2.37 (s, 3H), 2.49 (s, 3H), 3.34-3.40 (m, 1H), 4.00 (q, J = 7.6 Hz, 2H), 4.55 (d, J = 4.4 Hz, 1H), 6.12 (s, 1H), 7.32 (d, J = 4.8 Hz, 1H), 7.54 (s, 1H), 8.27 (s, 1H), 8.44 (d, J = 4.8 Hz, 1H), 8.57 (s, 1H), 11.00 (s, 1H), 12.32 (s, 1H); ESI- MS (m/z) 419 (M + H)⁺. |
| 191 | 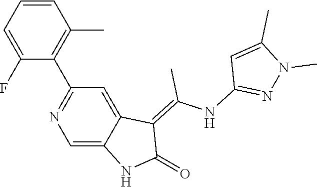 [SMILES: C/C(Nc1cc(C)n(C)n1)=C1/C(=O)Nc2cnc(-c3c(C)cccc3F)cc21] (Z)-3-(1-((1,5-dimethyl- 1H-pyrazol-3- yl)amino)ethylidene)-5-(2- fluoro-6-methylphenyl)- 1H-pyrrolo[2,3-c]pyridin- 2(3H)-one | Step 1 of Method I, 1,5-dimethyl-1H- pyrazol-3-amine and 2-(2-fluoro-6- methylphenyl)- 4,4,5,5-tetramethyl- 1,3,2-dioxaborolane Method 1 | ¹H NMR (400 MHz, DMSO-d₆) δ 2.14 (s, 3H), 2.27 (s, 3H), 2.64 (s, 3H), 3.70 (s, 3H), 6.10 (s, 1H), 7.09-7.14 (m, 2H), 7.31- 7.33 (m, 1H), 7.35 (s, 1H), 8.22 (s, 1H), 10.89 (s, 1H), 12.60 (s, 1H); ESI-MS (m/z) 378 (M + H)⁺. |
| 192 | 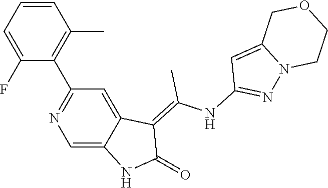 [SMILES: C/C(Nc1cc2n(n1)CCOC2)=C1/C(=O)Nc2cnc(-c3c(C)cccc3F)cc21] (Z)-3-(1-((6,7-Dihydro-4H- pyrazolo[5,1-c][1,4]oxazin- 2-yl)amino)ethylidene)-5- (2-fluoro-6-methylphenyl)- 1H-pyrrolo[2,3-c]pyridin- 2(3H)-one | Step 1 of Method I, 6,7-dihydro-4H- pyrazolo[5,1- c][1,4]oxazin-2- amine (D31) and 2- fluoro-6- methoxyphenylboronic acid Method I | ¹H NMR (400 MHz, DMSO-d₆) δ 2.14 (s, 3H), 2.72 (s, 3H), 4.08 (s, 4H), 4.79 (s, 2H), 6.13 (s, 1H), 7.07-7.14 (m, 2H), 7.29- 7.36 (m, 2H), 8.22 (s, 1H), 10.91 (s, 1H), 12.66 (s, 1H); ESI-MS (m/z) 406 (M + H)⁺. |
| 193 | 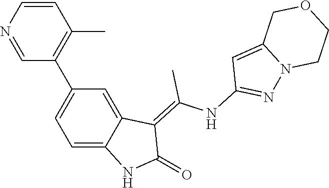 [SMILES: C/C(Nc1cc2n(n1)CCOC2)=C1/C(=O)Nc2ccc(-c3cnccc3C)cc21] (Z)-3-(1-((6,7-Dihydro-4H- pyrazolo[5,1-c][1,4]oxazin- 2-yl)amino)ethylidene)-5- (4-methylpyridin-3- yl)indolin-2-one | 5-bromoindolin-2- one, 6,7-dihydro-4H- pyrazolo[5,1- c][1,4]oxazin-2- amine (D31) and (4- methylpyridin-3- yl)boronic acid, pinacol ester Method I | ¹H NMR (400 MHz, DMSO-d₆) δ 2.30 (s, 3H), 2.64 (s, 3H), 4.06-4.10 (m, 4H), 4.78 (s, 2H), 6.05 (s, 1H), 6.98-7.04 (m, 2H), 7.31-7.35 (m, 2H), 8.39- 8.41 (m, 2H), 10.71 (s, 1H), 12.35 (s, 1H); ESI-MS (m/z) 388 (M + H)⁺. |
| 194 | 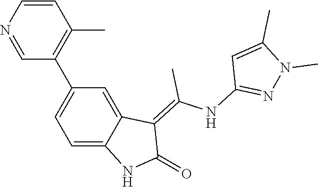 [SMILES: C/C(Nc1cc(C)n(C)n1)=C1/C(=O)Nc2ccc(-c3cnccc3C)cc21] (Z)-3-(1-((1,5-Dimethyl- 1H-pyrazol-3- yl)amino)ethylidene)-5-(4- methylpyridin-3-yl)indolin- 2-one | 5-bromoindolin-2- one, 1,5-dimethyl- 1H-pyrazol-3-amine and (4- methylpyridin-3- yl)boronic acid, pinacol ester Method I | ¹H NMR (400 MHz, DMSO-d₆) δ 2.26 (s, 3H), 2.30 (s, 3H), 2.62 (s, 3H), 3.68 (s, 3H), 6.02 (s, 1H), 6.99-7.01 (m, 2H), 7.32 (d, J = 4.8 Hz, 1H), 7.34 (s, 1H), 8.38 (s, 1H), 8.43 (d, J = 4.8 Hz, 1H), 10.68 (s, 1H), 12.27 (s, 1H); ESI-MS (m/z) 360 (M + H)⁺. |
| 195 | 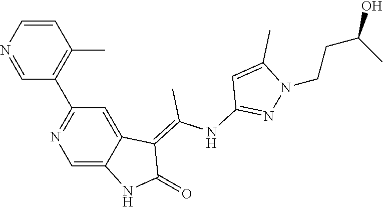 [SMILES: C/C(Nc1cc(C)n(CC[C@H](C)O)n1)=C1/C(=O)Nc2cnc(-c3cnccc3C)cc21] (S,Z)-3-(1-((1-(3- Hydroxybutyl)-5-methyl- 1H-pyrazol-3- yl)amino)ethylidene)-5-(4- methylpyridin-3-yl)-1H- pyrrolo[2,3-c]pyridin- 2(3H)-one | Step 1 of Method I, (S)-4-(3-amino-5- methyl-1H-pyrazol-1- yl)butan-2-ol (D60) and (4- methylpyridin-3- yl)boronic acid, pinacol ester Method I | ¹H NMR (400 MHz, DMSO-d₆) δ 1.06 (d, J = 6.4 Hz, 3H), 1.70-1.75 (m, 1H), 1.81-1.86 (m, 1H), 2.17 (s, 3H), 2.37 (s, 3H), 2.50 (s, 3H), 3.54-3.58 (m, 1H), 4.01 (t, J = 6.4 Hz, 2H), 4.57 (d, J = 4.8 Hz, 1H), 6.11 (s, 1H), 7.32 (d, J = 4.8 Hz, 1H), 7.53 (s, 1H), 8.27 (s, 1H), 8.43 (d, J = 4.8 Hz, 1H), 8.57 (s, 1H), 10.98 (s, 1H), 12.30 (s, 1H); ESI-MS (m/z) 419 (M + H)⁺. |
| 196 | 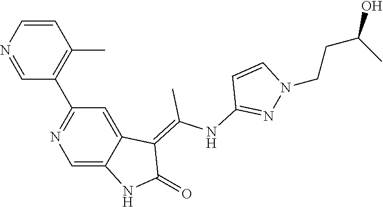 [SMILES: C/C(Nc1ccn(CC[C@H](C)O)n1)=C1/C(=O)Nc2cnc(-c3cnccc3C)cc21] (S,Z)-3-(1-((1-(3- hydroxybutyl)-1H-pyrazol- 3-yl)amino)ethylidene)-5- (4-methylpyridin-3-yl)-1H- pyrrolo[2,3-c]pyridin- 2(3H)-one | Step 1 of Method I, (S)-4-(3-amino-1H- pyrazol-1-yl)butan-2- ol (D61) and (4- methylpyridin-3- yl)boronic acid, pinacol ester Method I | ¹H NMR (400 MHz, DMSO-d₆) δ 1.08 (d, J = 6.4 Hz, 3H), 1.75-1.91 (m, 2H), 2.38 (s, 3H), 2.72 (s, 3H), 3.55-3.58 (m, 1H), 4.15 (t, J = 6.4 Hz, 2H), 4.66 (d, J = 4.8 Hz, 1H), 6.29 (s, 1H), 7.32 (d, J = 4.8 Hz, 1H), 7.55 (s, 1H), 7.81 (s, 1H), 8.24 (s, 1H), 8.44 (d, J = 4.8 Hz, 1H), 8.58 (s, 1H), 10.98 (s, 1H), 12.67 (s, 1H); ESI- MS (m/z) 405 (M + H)⁺. |
| 197 | 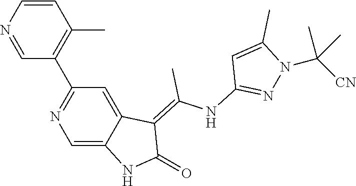 [SMILES: C/C(Nc1cc(C)n(C(C)(C)C#N)n1)=C1/C(=O)Nc2cnc(-c3cnccc3C)cc21] (Z)-2-Methyl-2-(5-methyl- 3-((1-(5-(4-Methylpyridin- 3-yl)-2-oxo-1H- pyrrolo[2,3-c]pyridin- 3(2H)- ylidene)ethyl)amino)-1H- pyrazol-1-yl)propanenitrile | Step 1 of Method I, 2-(3-amino-5-methyl- 1H-pyrazol-1-yl)-2- methylpropanenitrile (D53) and (4- methylpyridin-3- yl)boronic acid, pinacol ester Method I | ¹H NMR (400 MHz, DMSO-d₆) δ 1.98 (s, 6H), 2.38 (s, 3H), 2.53 (s, 3H), 2.73 (s, 3H), 6.35 (s, 1H), 7.32 (d, J = 4.8 Hz, 1H), 7.54 (s, 1H), 8.24 (s, 1H), 8.43 (d, J = 4.8 Hz, 1H), 8.58 (s, 1H), 10.96 (s, 1H), 12.61 (s, 1H); ESI-MS (m/z) 414 (M + H)⁺. |
| 198 | 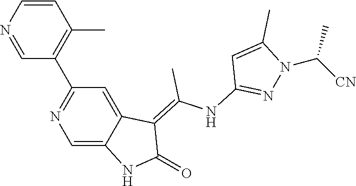 [SMILES: C/C(Nc1cc(C)n([C@H](C)C#N)n1)=C1/C(=O)Nc2cnc(-c3cnccc3C)cc21] (R,Z)-2-(5-Methyl-3-((1- (5-(4-methylpyridin-3-yl)- 2-oxo-1H-pyrrolo[2,3- c]pyridin-3(2H)- ylidene)ethyl)amino)-1H- pyrazol-1-yl)propanenitrile | Intermediate B9 and (R)-2-(3-amino-5- methyl-1H-pyrazol-1- yl)propanenitrile (D52) Method H | ¹H NMR (400 MHz, DMSO-d₆) δ 1.76 (d, J = 7.2 Hz, 3H), 2.35 (s, 3H), 2.38 (s, 3H), 2.74 (s, 3H), 5.84 (q, J = 7.2 Hz, 1H), 6.25 (s, 1H), 7.33 (d, J = 4.8 Hz, 1H), 7.56 (s, 1H), 8.25 (s, 1H), 8.44 (d, J = 4.8 Hz, 1H), 8.58 (s, 1H), 10.99 (s, 1H), 12.67 (s, 1H); ESI-MS (m/z) 400 (M + H)⁺. |
| 199 | 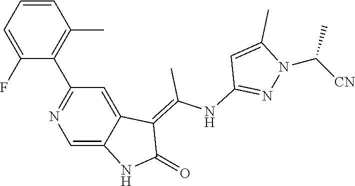 [SMILES: C/C(Nc1cc(C)n([C@H](C)C#N)n1)=C1/C(=O)Nc2cnc(-c3c(C)cccc3F)cc21] (R,Z)-2-(3-((1-(5-(2- Fluoro-6-methylphenyl)-2- oxo-1H-pyrrolo[2,3- c]pyridin-3(2H)- ylidene)ethyl)amino)-5- methyl-1H-pyrazol-1- yl)propanenitrile | Intermediate B13 and (R)-2-(3-amino-5- methyl-1H-pyrazol-1- yl)propanenitrile (D52) Method H | ¹H NMR (400 MHz, DMSO-d₆) δ 1.76 (d, J = 7.2 Hz, 3H), 2.14 (s, 3H), 2.34 (s, 3H), 2.67 (s, 3H), 5.84 (q, J = 7.2 Hz, 1H), 6.24 (s, 1H), 7.07-7.15 (m, 2H), 7.29-7.38 (m, 2H), 8.23 (s, 1H), 10.96 (s, 1H), 12.67 (s, 1H); ESI-MS (m/z) 417 (M + H)⁺. |
| 200 | 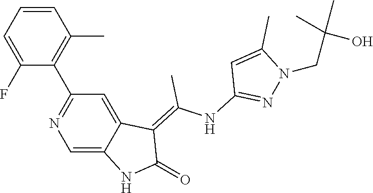 [SMILES: C/C(Nc1cc(C)n(CC(C)(C)O)n1)=C1/C(=O)Nc2cnc(-c3c(C)cccc3F)cc21] (Z)-5-(2-Fluoro-6- methylphenyl)-3-(1-((1-(2- hydroxy-2-methylpropyl)- 5-methyl-1H-pyrazol-3- yl)amino)ethylidene)-1H- pyrrolo[2,3-c]pyridin- 2(3H)-one | Intermediate B13 and 1-(3-amino-5-methyl- 1H-pyrazol-1-yl)-2- methylpropan-2-ol (D50) Method H | ¹H NMR (400 MHz, DMSO-d₆) δ 1.13 (s, 6H), 2.14 (s, 3H), 2.31 (s, 3H), 2.64 (s, 3H), 3.91 (s, 2H), 4.67 (s, 1H), 6.10 (s, 1H), 7.07-7.14 (m, 2H), 7.28- 7.35 (m, 2H), 8.21 (s, 1H), 10.89 (s, 1H), 12.56 (s, 1H); ESI-MS (m/z) 436 (M + H)⁺. |
| 201 | 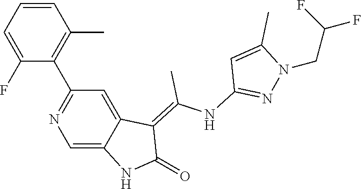 [SMILES: C/C(Nc1cc(C)n(CC(F)F)n1)=C1/C(=O)Nc2cnc(-c3c(C)cccc3F)cc21] (Z)-3-(1-((1-(2,2- Difluoroethyl)-5-methyl- 1H-pyrazol-3- yl)amino)ethylidene)-5-(2- fluoro-6-methylphenyl)- 1H-pyrrolo[2,3-c]pyridin- 2(3H)-one | Intermediate B13 and 1-(2,2-difluoroethyl)- 5-methyl-1H-pyrazol- 3-amine (D39) Method H | ¹H NMR (400 MHz, DMSO-d₆) δ 2.14 (s, 3H), 2.31 (s, 3H), 2.65 (s, 3H), 4.49-4.60 (m, 2H), 6.20 (s, 1H), 6.38 (t, J = 3.6 Hz, 1H), 7.06-7.15 (m, 2H), 7.27-7.37 (m, 2H), 8.22 (s, 1H), 10.92 (s, 1H), 12.64 (s, 1H); ESI-MS (m/z) 428 (M + H)⁺. |
| 202 | 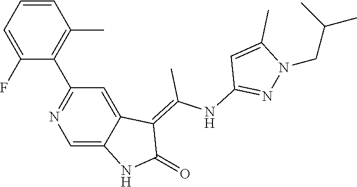 [SMILES: C/C(Nc1cc(C)n(CC(C)C)n1)=C1/C(=O)Nc2cnc(-c3c(C)cccc3F)cc21] (Z)-5-(2-Fluoro-6- methylphenyl)-3-(1-((1- isobutyl-5-methyl-1H- pyrazol-3- yl)amino)ethylidene)-1H- pyrrolo[2,3-c]pyridin- 2(3H)-one | Intermediate B13 and 1-isobutyl-5-methyl- 1H-pyrazol-3-amine (D41) Method H | ¹H NMR (400 MHz, DMSO-d₆) δ 0.87 (d, J = 6.8 Hz, 6H), 2.09-2.13 (m, 1H), 2.14 (s, 3H), 2.27 (s, 3H), 2.64 (s, 3H), 3.80 (d, J = 7.2 Hz, 2H), 6.09 (s, 1H), 7.08- 7.15 (m, 2H), 7.29-7.36 (m, 2H), 8.21 (s, 1H), 10.89 (s, 1H), 12.58 (s, 1H); ESI-MS (m/z) 420 (M + H)⁺. |
| 203 | 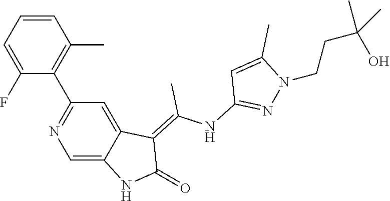 [SMILES: C/C(Nc1cc(C)n(CCC(C)(C)O)n1)=C1/C(=O)Nc2cnc(-c3c(C)cccc3F)cc21] (Z)-5-(2-Fluoro-6- methylphenyl)-3-(1-((1-(3- hydroxy-3-methylbutyl)-5- methyl-1H-pyrazol-3- yl)amino)ethylidene)-1H- pyrrolo[2,3-c]pyridin- 2(3H)-one | Intermediate B13 and 4-(3-amino-5-methyl- 1H-pyrazol-1-yl)-2- methylbutan-2-ol (D40) Method H | ¹H NMR (400 MHz, DMSO-d₆) δ 1.38 (s, 6H), 1.81 (t, J = 8.0 Hz, 2H), 2.14 (s, 3H), 2.28 (s, 3H), 2.63 (s, 3H), 4.05 (t, J = 8.0 Hz, 2H), 4.47 (s, 1H), 6.09 (s, 1H), 7.06-7.15 (m, 2H), 7.27-7.33 (m, 1H), 7.34 (s, 1H), 8.21 (s, 1H), 10.89 (s, 1H), 12.60 (s, 1H); ESI-MS (m/z) 450 (M + H)⁺. |
| 204 | 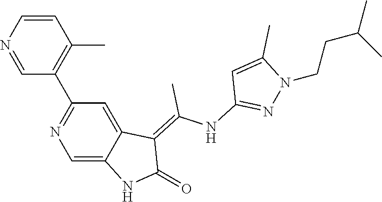 [SMILES: C/C(Nc1cc(C)n(CCC(C)C)n1)=C1/C(=O)Nc2cnc(-c3cnccc3C)cc21] (Z)-3-(1-((1-Isopentyl-5- methyl-1H-pyrazol-3- yl)amino)ethylidene)-5-(4- methylpyridin-3-yl)-1H- pyrrolo[2,3-c]pyridin- 2(3H)-one | Step 1 of Method I, 1-isopentyl-5-methyl- 1H-pyrazol-3-amine (D62) and (4- methylpyridin-3- yl)boronic acid, pinacol ester Method I | ¹H NMR (400 MHz, DMSO-d₆) δ 0.93 (t, J = 6.4 Hz, 6H), 1.53-1.65 (m, 3H), 2.29 (s, 3H), 2.37 (s, 3H), 2.70 (s, 3H), 4.00 (t, J = 6.4 Hz, 2H), 6.10 (s, 1H), 7.32 (d, J = 4.8 Hz, 1H), 7.52 (s, 1H), 8.23 (s, 1H), 8.43 (d, J = 4.8 Hz, 1H), 8.57 (s, 1H), 10.91 (s, 1H), 12.58 (s, 1H); ESI-MS (m/z) 417 (M + H)⁺. |
| 205 | 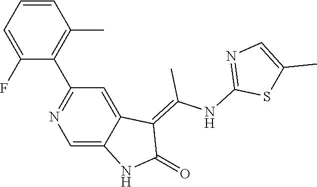 [SMILES: C/C(Nc1ncc(C)s1)=C1/C(=O)Nc2cnc(-c3c(C)cccc3F)cc21] (Z)-5-(2-Fluoro-6- methylphenyl)-3-(1-((5- methylthiazol-2- yl)amino)ethylidene)-1H- pyrrolo[2,3-c]pyridin- 2(3H)-one | Intermediate B13 and 5-methylthiazol-2- amine Method H | ¹H NMR (400 MHz, DMSO-d₆) δ 2.14 (s, 3H), 2.32 (s, 3H), 2.82 (s, 3H), 7.08-7.15 (m, 2H), 7.29- 7.33 (m, 2H), 7.46 (s, 1H), 8.27 (s, 1H), 11.13 (s, 1H), 13.36 (s, 1H); ESI-MS (m/z) 380 (M + H)⁺. |
| 206 | 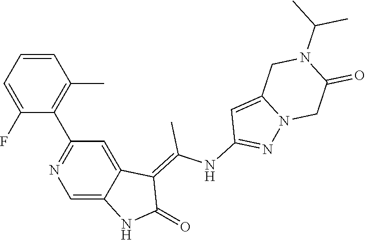 [SMILES: C/C(Nc1cc2n(n1)CC(=O)N(C(C)C)C2)=C1/C(=O)Nc2cnc(-c3c(C)cccc3F)cc21] (Z)-2-((1-(5-(2-Fluoro-6- methylphenyl)-2-oxo-1H- pyrrolo[2,3-c]pyridin- 3(2H)- ylidene)ethyl)amino)-5- isopropyl-4,5- dihydropyrazolo[1,5- a]pyrazin-6(7H)-one | Intermediate B13 and 2-amino-5-isopropyl- 4,5- dihydropyrazolo[1,5- a]pyrazin-6(7H)-one (D34) Method H | ¹H NMR (400 MHz, DMSO-d₆) δ 1.17 (d, J = 6.8 Hz, 6H), 2.14 (s, 3H), 2.68 (s, 3H), 4.54 (s, 2H), 4.74 (s, 2H), 4.75-4.81 (m, 1H), 6.27 (s, 1H), 7.07-7.14 (m, 2H), 7.30-7.32 (m, 1H), 7.38 (s, 1H), 8.23 (s, 1H), 10.93 (s, 1H), 12.69 (s, 1H); ESI-MS (m/z) 461 (M + H)⁺. |
| 207 | 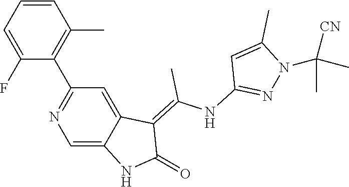 [SMILES: C/C(Nc1cc(C)n(C(C)(C)C#N)n1)=C1/C(=O)Nc2cnc(-c3c(C)cccc3F)cc21] (Z)-2-(3-((1-(5-(2-Fluoro- 6-methylphenyl)-2-oxo- 1H-pyrrolo[2,3-c]pyridin- 3(2H)- ylidene)ethyl)amino)-5- methyl-1H-pyrazol-1-yl)-2- methylpropanenitrile | Step 1 of Method I, 2-(3-amino-5-methyl- 1H-pyrazol-1-yl)-2- methylpropanenitrile (D53) and 2-(2- fluoro-6- methylphenyl)- 4,4.5,5-tetramethyl- 1,3,2-dioxaborolane Method I | ¹H NMR (400 MHz, DMSO-d₆) δ 1.97 (s, 6H), 2.14 (s, 3H), 2.53 (s, 3H), 2.66 (s, 3H), 6.34 (s, 1H), 7.07-7.15 (m, 2H), 7.29- 7.34 (s, 1H), 7.38 (s, 1H), 8.22 (s, 1H), 10.94 (s, 1H), 12.61 (s, 1H); ESI-MS (m/z) 431 (M + H)⁺. |
| 208 | 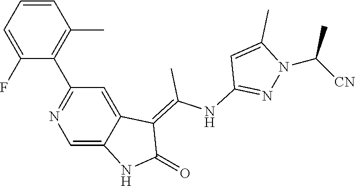 [SMILES: C/C(Nc1cc(C)n([C@@H](C)C#N)n1)=C1/C(=O)Nc2cnc(-c3c(C)cccc3F)cc21] (S,Z)-2-(3-((1-(5-(2- Fluoro-6-methylphenyl)-2- oxo-1H-pyrrolo[2,3- c]pyridin-3(2H)- ylidene)ethyl)amino)-5- methyl-1H-pyrazol-1- yl)propanenitrile | Intermediate B13 and (S)-2-(3-amino-5- methyl-1H-pyrazol-1- yl)propanenitrile (D63) Method H | ¹H NMR (400 MHz, DMSO-d₆) δ 1.75 (d, J = 6.8 Hz, 3H), 2.14 (s, 3H), 2.34 (s, 3H), 2.67 (s, 3H), 5.84 (q, J = 7.2 Hz, 1H), 6.24 (s, 1H), 7.10-7.15 (m, 2H), 7.30-7.32 (m, 1H), 7.38 (s, 1H), 8.23 (s, 1H), 10.96 (s, 1H), 12.67 (s, 1H); ESI-MS (m/z) 417 (M + H)⁺. |
| 209 | 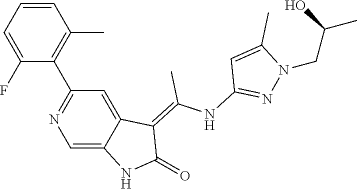 [SMILES: C/C(Nc1cc(C)n(C[C@H](C)O)n1)=C1/C(=O)Nc2cnc(-c3c(C)cccc3F)cc21] (S,Z)-5-(2-Fluoro-6- methylphenyl)-3-(1-((1-(2- hydroxypropyl)-5-methyl- 1H-pyrazol-3- yl)amino)ethylidene)-1H- pyrrolo[2,3-c]pyridin- 2(3H)-one | Intermediate B13 and (S)-1-(3-amino-5- methyl-1H-pyrazol-1- yl)propan-2-ol (D46) Method H | ¹H NMR (400 MHz, DMSO-d₆) δ 1.09 (d, J = 6.0 Hz, 3H), 2.14 (s, 3H), 2.29 (s, 3H), 2.65 (s, 3H), 3.87- 3.90 (m, 2H), 3.97-3.99 (m, 1H), 4.92 (d, J = 4.8 Hz, 1H), 6.08 (s, 1H), 7.07-7.14 (m, 2H), 7.28-7.35 (m, 2H), 8.21 (s, 1H), 10.89 (s, 1H), 12.61 (s, 1H); ESI-MS (m/z) 422 (M + H)⁺. |
| 210 | 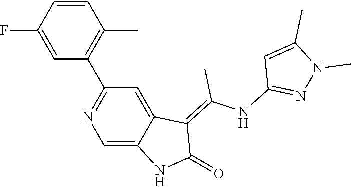 [SMILES: C/C(Nc1cc(C)n(C)n1)=C1/C(=O)Nc2cnc(-c3cc(F)ccc3C)cc21] (Z)-3-(1-((1,5-Dimethyl- 1H-pyrazol-3- yl)amino)ethylidene)-5-(5- fluoro-2-methylphenyl)- 1H-pyrrolo[2,3-c]pyridin- 2(3H)-one | Step 1 of Method I, 1,5-dimethyl-1H- pyrazol-3-amine and 2-(2-fluoro-6- methylphenyl)- 4,4,5,5-tetramethyl- 1,3,2-dioxaborolane Method I | ¹H NMR (400 MHz, DMSO-d₆) δ 2.27 (s, 3H), 2.30 (s, 3H), 2.69 (s, 3H), 3.70 (s, 3H), 6.10 (s, 1H), 7.08-7.13 (m, 1H), 7.22- 7.25 (m, 1H), 7.30-7.32 (m, 1H), 7.45 (s, 1H), 8.19 (s, 1H), 10.88 (s, 1H), 12.57 (s, 1H); ESI-MS (m/z) 378 (M + H)⁺. |
| 211 | 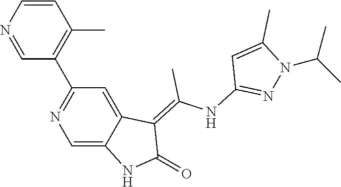 [SMILES: C/C(Nc1cc(C)n(C(C)C)n1)=C1/C(=O)Nc2cnc(-c3cnccc3C)cc21] (Z)-3-(1-((1-Isopropyl-5- methyl-1H-pyrazol-3- yl)amino)ethylidene)-5-(4- methylpyridin-3-yl)-1H- pyrrolo[2,3-c]pyridin- 2(3H)-one | Step 1 of Method I, 1-isopropyl-5- methyl-1H-pyrazol-3- amine and (4- methylpyridin-3- yl)boronic acid, pinacol ester Method I | ¹H NMR (400 MHz, DMSO-d₆) δ 1.37 (t, J = 6.4 Hz, 6H), 2.13 (s, 3H), 2.29 (s, 3H), 2.70 (s, 3H), 4.47- 4.53 (m, 1H), 6.08 (s, 1H), 7.32 (d, J = 4.8 Hz, 1H), 7.51 (s, 1H), 8.23 (s, 1H), 8.43 (d, J = 4.8 Hz, 1H), 8.57 (s, 1H), 10.91 (s, 1H), 12.59 (s, 1H); ESI-MS (m/z) 389 (M + H)⁺. |
| 212 | 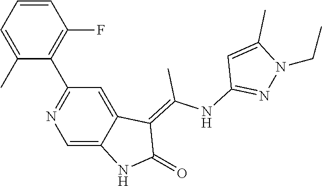 [SMILES: CCn1nc(N/C(C)=C2\C(=O)Nc3cnc(-c4c(C)cccc4F)cc32)cc1C] (Z)-3-(1-((1-Ethyl-5- methyl-1H-pyrazol-3- yl)amino)ethylidene)-5-(2- fluoro-6-methylphenyl)- 1H-pyrrolo[2,3-c]pyridin- 2(3H)-one | Step 1 of Method I, 1-ethyl-5-methyl-1H- pyrazol-3-amine and 2-(2-fluoro-6- methylphenyl)- 4,4,5,5-tetramethyl- 1,3,2-dioxaborolane Method I | ¹H NMR (400 MHz, DMSO-d₆) δ 1.31 (t, J = 7.2 Hz, 3H), 2.14 (s, 3H), 2.28 (s, 3H), 2.64 (s, 3H), 4.02 (q, J = 7.2 Hz, 2H), 6.09 (s, 1H), 7.07-7.14 (m, 2H), 7.28-7.35 (m, 2H), 8.21 (s, 1H), 10.89 (s, 1H), 12.61 (s, 1H); ESI-MS (m/z) 392 (M + H)⁺. |
| 213 | 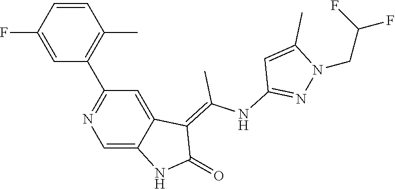 [SMILES: C/C(Nc1cc(C)n(CC(F)F)n1)=C1/C(=O)Nc2cnc(-c3cc(F)ccc3C)cc21] (Z)-3-(1-((1-(2,2- Difluoroethyl)-5-methyl- 1H-pyrazol-3- yl)amino)ethylidene)-5-(5- fluoro-2-methylphenyl)- 1H-pyrrolo[2,3-c]pyridin- 2(3H)-one | Step 1 of Method I, 1-(2,2-difluoroethyl)- 5-methyl-1H-pyrazol- 3-amine (D39) and 5- fluoro-2- methylboronic acid Method I | ¹H NMR (400 MHz, DMSO-d₆) δ 2.31 (s, 6H), 2.71 (s, 3H), 4.51-4.59 (s, 2H), 6.20 (s, 1H), 6.39 (t, J = 4.8 Hz, 1H), 7.08-7.13 (m, 1H), 7.22-7.25 (m, 1H), 7.29-7.32 (m, 1H), 7.47 (s, 1H), 8.20 (s, 1H), 10.91 (s, 1H), 12.61 (s, 1H); ESI-MS (m/z) 428 (M + H)⁺. |
| 214 | 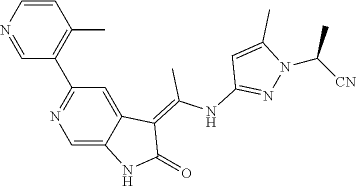 [SMILES: C/C(Nc1cc(C)n([C@@H](C)C#N)n1)=C1/C(=O)Nc2cnc(-c3cnccc3C)cc21] (S,Z)-2-(5-Methyl-3-((1-(5- (4-methylpyridin-3-yl)-2- oxo-1H-pyrrolo[2,3- c]pyridin-3(2H)- ylidene)ethyl)amino)-1H- pyrazol-1-yl)propanenitrile | Intermediate B9 and (S)-2-(3-amino-5- methyl-1H-pyrazol-1- yl)propanenitrile (D63) Method H | ¹H NMR (400 MHz, DMSO-d₆) δ 1.22 (s, 3H), 1.76 (d, J = 6.0 Hz, 3H), 2.38 (s, 3H), 2.74 (s, 3H), 5.83-5.85 (m, 1H), 6.24 (s, 1H), 7.32 (d, J = 4.8 Hz, 1H), 7.55 (s, 1H), 8.25 (s, 1H), 8.43 (d, J = 4.8 Hz, 1H), 8.58 (s, 1H), 10.98 (s, 1H), 12.66 (s, 1H); ESI-MS (m/z) 400 (M + H)⁺. |
| 215 | 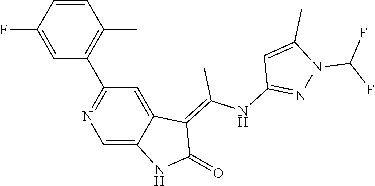 [SMILES: C/C(Nc1cc(C)n(C(F)F)n1)=C1/C(=O)Nc2cnc(-c3cc(F)ccc3C)cc21] (Z)-3-(1-((1- (Difluoromethyl)-5-methyl- 1H-pyrazol-3- yl)amino)ethylidene)-5-(5- fluoro-2-methylphenyl)- 1H-pyrrolo[2,3-c]pyridin- 2(3H)-one | Step 1 of Method I, 1-(difluoromethyl)-5- methyl-1H-pyrazol-3- amine (D47) and 2- (2-fluoro-6- methylphenyl)- 4,4,5,5-tetramethyl- 1,3,2-dioxaborolane Method I | ¹H NMR (400 MHz, DMSO-d₆) δ 2.30 (s, 3H), 2.44 (s, 3H), 2.76 (s, 3H), 6.43 (s, 1H), 7.10-7.14 (m, 1H), 7.24 (dd, J = 10.0, 2.8 Hz, 1H), 7.29-7.33 (m, 1H), 7.51 (s, 1H), 7.64-7.93 (m, 1H), 8.22 (s, 1H), 10.98 (s, 1H), 12.70 (s, 1H); ESI-MS (m/z) 414 (M + H)⁺. |
| 216 | 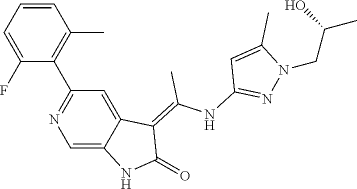 [SMILES: C/C(Nc1cc(C)n(C[C@@H](C)O)n1)=C1/C(=O)Nc2cnc(-c3c(C)cccc3F)cc21] (R,Z)-5-(2-Fluoro-6- methylphenyl)-3-(1-((1-(2- hydroxypropyl)-5-methyl- 1H-pyrazol-3- yl)amino)ethylidene)-1H- pyrrolo[2,3-c]pyridin- 2(3H)-one | Intermediate B13 and (R)-1-(3-amino-5- methyl-1H-pyrazol-1- yl)propan-2-ol (D51) Method H | ¹H NMR (400 MHz, DMSO-d₆) δ 1.09 (d, J = 6.0 Hz, 3H), 2.14 (s, 3H), 2.29 (s, 3H), 2.65 (s, 3H), 3.89 (q, J = 4.8 Hz, 2H), 3.98 (t, J = 6.0 Hz, 1H), 4.92 (d, J = 5.2 Hz, 1H), 6.08 (s, 1H), 7.07-7.14 (m, 2H), 7.28- 7.35 (m, 2H), 8.21 (s, 1H), 10.89 (s, 1H), 12.60 (s, 1H), ESI-MS (m/z) 422 (M + H)⁺. |
| 217 | 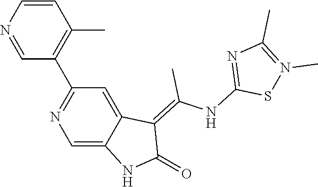 (Z)-3-(1-((4,5- Dimethylthiazol-2- yl)amino)ethylidene)-5-(4- methylpyridin-3-yl)-1H- pyrrolo[2,3-c]pyridin- 2(3H)-one | Step 1 of Method I, 4,5-dimethylthiazol- 2-amine and (4- methylpyridin-3- yl)boronic acid, pinacol ester Method I | ¹H NMR (400 MHz, DMSO-d₆) δ 2.22 (s, 3H), 2.32 (s, 3H), 2.37 (s, 3H), 2.87 (s, 3H), 7.33 (d, J = 4.8 Hz, 1H), 7.61 (s, IH), 8.28 (s, 1H), 8.44 (d, J = 5.2 Hz, 1H), 8.58 (s, 1H), 11.13 (s, 1H), 13.34 (s, 1H); ESI-MS (m/z) 378 (M + H)⁺. |
| 218 | 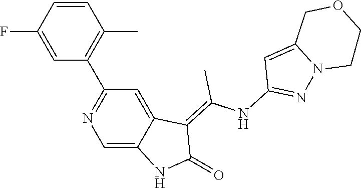 [SMILES: C/C(Nc1cc2n(n1)CCOC2)=C1/C(=O)Nc2cnc(-c3cc(F)ccc3C)cc21] (Z)-3-(1-((6,7-Dihydro-4H- pyrazolo[5,1-c][1,4]oxazin- 2-yl)amino)ethylidene)-5- (5-fluoro-2-methylphenyl)- 1H-pyrrolo[2,3-c]pyridin- 2(3H)-one | Step 1 of Method I, 6,7-dihydro-4H- pyrazolo[5,1- c][1,4]oxazin-2- amine (D31) and 2- (2-fluoro-6- methylphenyl)- 4,4,5,5-tetramethyl- 1,3,2-dioxaborolane Method I | ¹H NMR (400 MHz, DMSO-d₆) δ 2.30 (s, 3H), 2.72 (s, 3H), 4.09 (s, 4H), 4.80 (s, 2H), 6.14 (s, 1H), 7.09-7.14 (m, 1H), 7.23- 7.26 (m, 1H), 7.29-7.33 (m, 1H), 7.48 (s, 1H), 8.21 (s, 1H), 10.93 (s, 1H), 12.67 (s, 1H); ESI-MS (m/z) 406 (M+H)⁺. |

### Pharmacological Activity

### FRET Assay for HPK1 (MAP4K1):

This is a one step binding assay based on the binding and displacement of the labeled tracer, where compound addition is followed by addition of the anti-GST tagged europium (Eu) as the donor and Alexa Fluor-labeled tracer as the acceptor. Simultaneous binding of both the tracer and GST-antibody to the kinase domain of HPK1 results in a high degree of FRET (fluorescence resonance energy transfer) from the anti-GST tagged europium (Eu) fluorophore to the Alexa Fluor® 647 fluorophore on the kinase tracer and this signal is reduced in presence of the inhibitor that can be measured.

Test compounds or reference compounds such as Sunitinib (Sigma) were dissolved in dimethylsulfoxide (DMSO) to prepare 10.0 mM stock solutions and diluted to the desired concentration. The final concentration of DMSO in the reaction was 3% (v/v). The assay mixture was prepared by mixing 4 nM of the Eu-Anti-GST Antibody and 10 nM MAP4K-1 enzyme in the Kinase buffer containing 50mM HEPES (pH 7.5), 10 mM MgCl₂, 1 mM EGTA, 0.01% Brij-35 with or without the desired concentration of the compound. The reaction was incubated on ice for 15 mins. The pre-incubation step was followed by addition of the 20 nM Kinase Tracer 222 into the reaction mixture. After shaking for 5 min the reaction was further incubated for 1 hour at room temperature and this was kept at 4° C. and read on ARTEMIS reader as per the kit instructions (Thermo). The inhibition of test compound was calculated based on the FRET ratio of 665/620. The activity was calculated as percent of control reaction. IC50 values were calculated from dose response curve by nonlinear regression analysis using GraphPad Prism software.

The compounds prepared were tested using the above assay procedure and the results obtained are given in Table 16. Percentage inhibition at concentrations of 1.0 μM and 10.0 μM are given in the table along with IC₅₀ (nM) details for selected examples.

The IC₅₀ (nM) values are set forth in Table 16 wherein “A” refers to an IC₅₀ value of less than 50 nM, “B” refers to IC₅₀ value in range of 50.01 to 100.0 nM, “C” refers to IC₅₀ values more than 100.01 to 500 nM and “D” refers to IC₅₀ values more than 500 nM.

TABLE 16

| | |
|---|---|
| % Inhibition at | IC₅₀ value |

| Sr. no. | Compound No. | 1 μM | 10 μM | (nM) |
|---|---|---|---|---|
| 1. | Example 1 | 69.8 | 84.05 | C |
| 2. | Example 2 | 88.64 | 82.45 | A |
| 3. | Example 3 | 62.52 | — | C |
| 4. | Example 4 | 90.09 | 90.95 | A |
| 5. | Example 5 | 76.07 | 85.51 | C |
| 6. | Example 6 | 85.47 | 87.31 | A |
| 7. | Example 7 | 82.39 | 87.14 | A |
| 8. | Example 8 | 84.8 | 83.8 | A |
| 9. | Example 9 | 84.86 | 84.38 | A |
| 10. | Example 10 | 82.64 | 88.25 | A |
| 11. | Example 11 | 73.18 | 84.44 | C |
| 12. | Example 12 | 48.01 | 55.76 | D |
| 13. | Example 13 | 73.35 | ppt | B |
| 14. | Example 14 | 86.32 | 88.33 | A |
| 15. | Example 15 | 85.2 | 87.02 | A |
| 16. | Example 16 | 85.31 | 88.92 | A |
| 17. | Example 17 | 85.72 | 83.29 | A |
| 18. | Example 18 | 89.81 | 85.39 | A |
| 19. | Example 19 | 37.49 | — | — |
| 20. | Example 20 | 85.2 | 80.1 | A |
| 21. | Example 21 | 83.1 | 80 | A |
| 22. | Example 22 | 86.6 | 90.5 | A |
| 23. | Example 23 | 84.62 | 86.95 | A |
| 24. | Example 24 | 70.9 | 79.7 | C |
| 25. | Example 25 | 87.1 | 85.3 | A |
| 26. | Example 26 | 88.77 | 93.55 | A |
| 27. | Example 27 | 88.37 | 96.28 | A |
| 28. | Example 28 | 84.82 | 89.43 | A |
| 29. | Example 29 | 87.42 | 85.1 | A |
| 30. | Example 30 | 89.93 | 92.82 | A |
| 31. | Example 31 | 86.92 | 92.31 | A |
| 32. | Example 32 | 90.33 | 100 | A |
| 33. | Example 33 | 91.58 | 87.79 | A |
| 34. | Example 34 | 81.72 | 80.05 | A |
| 35. | Example 35 | 87.95 | 100 | A |
| 36. | Example 36 | 87.79 | 85.06 | A |
| 37. | Example 37 | 84.38 | 91.17 | B |
| 38. | Example 38 | 93.48 | ppt | A |
| 39. | Example 39 | 72.25 | 86.63 | A |
| 40. | Example 40 | 81.31 | 92.05 | C |
| 41. | Example 41 | 69.94 | 77.01 | B |
| 42. | Example 43 | 86.25 | 89.16 | A |
| 43. | Example 44 | 91.43 | 93.14 | A |
| 44. | Example 45 | 72.42 | 85.95 | C |
| 45. | Example 46 | 85.26 | 87.32 | A |
| 46. | Example 47 | 83.45 | 82.5 | A |
| 47. | Example 48 | 77.24 | 84.78 | B |
| 48. | Example 49 | 64.27 | 74.22 | A |
| 49. | Example 50 | 82.83 | 87.87 | A |
| 50. | Example 51 | 81.17 | 84.23 | A |
| 51. | Example 52 | 86.11 | 84.92 | A |
| 52. | Example 53 | 87.08 | 85.36 | A |
| 53. | Example 54 | 88.47 | 86.11 | A |
| 54. | Example 55 | 77.96 | 90.05 | B |
| 55. | Example 56 | 66.78 | 75.15 | C |
| 56. | Example 57 | 80.94 | 94.14 | A |
| 57. | Example 58 | 70.99 | 72.66 | A |
| 58. | Example 59 | 78.05 | 78.18 | A |
| 59. | Example 60 | 73.99 | 72.68 | A |
| 60. | Example 61 | 71.64 | 81.27 | A |
| 61. | Example 62 | 80.31 | 94.7 | A |
| 62. | Example 63 | 89.84 | 97.08 | A |
| 63. | Example 64 | 83.31 | 91.19 | A |
| 64. | Example 65 | 79.67 | 75.45 | B |
| 65. | Example 66 | 74.07 | 76.19 | A |
| 66. | Example 67 | 73.99 | 71.43 | A |
| 67. | Example 68 | 77.53 | 75.29 | A |
| 68. | Example 69 | 67.02 | 65.49 | — |
| 69. | Example 70 | 69.55 | 69.94 | — |
| 70. | Example 71 | 71.68 | 72.71 | A |
| 71. | Example 72 | 39.76 | 68.63 | — |
| 72. | Example 73 | 69.64 | 63.88 | A |
| 73. | Example 74 | 76.33 | ppt | A |
| 74. | Example 75 | 71.03 | 73.99 | A |
| 75. | Example 76 | 70.29 | 69.01 | A |
| 76. | Example 77 | 73.01 | 80.35 | A |
| 77. | Example 78 | 84.24 | 83.11 | A |
| 78. | Example 79 | 78.55 | 77.37 | A |
| 79. | Example 80 | 75.09 | 78.05 | A |
| 80. | Example 81 | 73.77 | 82.24 | A |
| 81. | Example 82 | 70.59 | 79.88 | A |
| 82. | Example 83 | 27.73 | 43.51 | — |
| 83. | Example 84 | 75.16 | 70.36 | A |
| 84. | Example 85 | 78.21 | ppt | A |
| 85. | Example 86 | 51.45 | 74.04 | D |
| 86. | Example 87 | 83.56 | 84.85 | A |
| 87. | Example 88 | 74.28 | 87.64 | C |
| 88. | Example 89 | 77.26 | 76 | A |
| 89. | Example 90 | 72.67 | 75.57 | A |
| 90. | Example 91 | 79.68 | ppt | A |
| 91. | Example 92 | 77.04 | 80.31 | A |
| 92. | Example 93 | 73.75 | 76.43 | A |
| 93. | Example 94 | 66.82 | 75.39 | A |
| 94. | Example 95 | 75.46 | 75.66 | A |
| 95. | Example 96 | 68.56 | 73.46 | A |
| 96. | Example 97 | 58.99 | 59.86 | C |
| 97. | Example 98 | 60.97 | 69.04 | B |
| 98. | Example 99 | 70.11 | 78.63 | B |
| 99. | Example 100 | 72.85 | 76.95 | A |
| 100. | Example 101 | 68.01 | 81.8 | A |
| 101. | Example 102 | 81.58 | 89.66 | A |
| 102. | Example 103 | 74.52 | 83.39 | A |
| 103. | Example 104 | 74.64 | 79.79 | A |
| 104. | Example 105 | 79.82 | 84.36 | A |
| 105. | Example 106 | 79.97 | 92.4 | A |
| 106. | Example 107 | 72.33 | 80.05 | A |
| 107. | Example 108 | 74.25 | 77.05 | A |
| 108. | Example 109 | 72.38 | 83.39 | A |
| 109. | Example 110 | 72.66 | 79.37 | A |
| 110. | Example 111 | 74.44 | 74.27 | A |
| 111. | Example 112 | 71.28 | 89.5 | A |
| 112. | Example 113 | 64.76 | 70.3 | C |
| 113. | Example 114 | 73.31 | 75.91 | A |
| 114. | Example 115 | 69.19 | 73.98 | A |
| 115. | Example 116 | 75.93 | — | A |
| 116. | Example 117 | 54.75 | 56.44 | — |
| 117. | Example 118 | 78.37 | 83.34 | A |
| 118. | Example 119 | 52.61 | 66.98 | D |
| 119. | Example 120 | 2.17 | 49 | — |
| 120. | Example 121 | 78.89 | 85.65 | A |
| 121. | Example 122 | 87.32 | 92.62 | A |
| 122. | Example 123 | 81.85 | 88.25 | A |
| 123. | Example 124 | 79.63 | 89.68 | A |
| 124. | Example 125 | 77.69 | 84.18 | A |
| 125. | Example 126 | 82.41 | — | A |
| 126. | Example 127 | 75.13 | 81.5 | A |
| 127. | Example 128 | 79.25 | 89.78 | A |
| 128. | Example 129 | 76.44 | 82.23 | A |
| 129. | Example 130 | 79.38 | 84.82 | A |
| 130. | Example 131 | 82.2 | 83.74 | A |
| 131. | Example 132 | 81.04 | 80.88 | C |
| 132. | Example 133 | 79.92 | 87.98 | A |
| 133. | Example 134 | 83.63 | 85.24 | A |
| 134. | Example 135 | 78.55 | 83.92 | B |
| 135. | Example 136 | 82.34 | 78.92 | A |
| 136. | Example 137 | 80.71 | 80.15 | A |
| 137. | Example 138 | 90.65 | — | A |
| 138. | Example 139 | 77 | 84.47 | A |
| 139. | Example 140 | 79.85 | 91.31 | A |
| 140. | Example 141 | 80.04 | 85.88 | B |
| 141. | Example 142 | 77.93 | 80.83 | A |
| 142. | Example 143 | 80.36 | 83.77 | A |
| 143. | Example 144 | 80.46 | 84.58 | A |
| 144. | Example 145 | 79.4 | 83.87 | A |
| 145. | Example 146 | 80.23 | 88.69 | A |
| 146. | Example 147 | 74.87 | 75.92 | A |
| 147. | Example 148 | 82.43 | 93.97 | A |
| 148. | Example 149 | 78.69 | 81.7 | A |
| 149. | Example 150 | 86.68 | 86.08 | A |
| 150. | Example 151 | 85.24 | 65.22 | A |
| 151. | Example 152 | 74.35 | 70.37 | A |
| 152. | Example 153 | 72.55 | 80.88 | A |
| 153. | Example 154 | 68.97 | 82.5 | C |
| 154. | Example 155 | 65.06 | 68.8 | A |
| 155. | Example 156 | 60.6 | 80.02 | B |
| 156. | Example 157 | 54.54 | 66.5 | — |
| 157. | Example 158 | 74.68 | 91.13 | C |
| 158. | Example 159 | 62.77 | 87.4 | D |
| 159. | Example 160 | 85.05 | 78.44 | A |
| 160. | Example 161 | 81.23 | 81.04 | A |
| 161. | Example 162 | 76.21 | 92.91 | B |
| 162. | Example 163 | 77.32 | 81.45 | A |
| 163. | Example 164 | 60.89 | 75.19 | B |
| 164. | Example 165 | 79.34 | 71.85 | A |
| 165. | Example 166 | 62.86 | 75.72 | A |
| 166. | Example 167 | 69.24 | 67.46 | A |
| 167. | Example 168 | 67.63 | 76.68 | A |
| 168. | Example 169 | 71.43 | 84.26 | A |
| 169. | Example 170 | 70.08 | 70.3 | A |
| 170. | Example 171 | 63.44 | 77.05 | B |
| 171. | Example 172 | 79.23 | 80.53 | A |
| 172. | Example 173 | 66.77 | 77.37 | A |
| 173. | Example 174 | 55.08 | 64.81 | A |
| 174. | Example 175 | 72.56 | — | A |
| 175. | Example 176 | 75.4 | 62.33 | A |
| 176. | Example 177 | 69.4 | 66.62 | A |
| 177. | Example 178 | 68 | 77.77 | A |
| 178. | Example 179 | 66.93 | 77.61 | A |
| 179. | Example 180 | 73.01 | 76.23 | A |
| 180. | Example 181 | 76.82 | 80.75 | A |
| 181. | Example 182 | 78.81 | 72.97 | A |
| 182. | Example 183 | 72.52 | 79.53 | A |
| 183. | Example 184 | 70.27 | 75.17 | A |
| 184. | Example 185 | 73.23 | 73.87 | A |
| 185. | Example 186 | 70.92 | 72.18 | A |
| 186. | Example 187 | 66.38 | 19.62 | — |
| 187. | Example 188 | 70.98 | 75.97 | A |
| 188. | Example 189 | 73.48 | 76.89 | A |
| 189. | Example 190 | 52.41 | 68.63 | D |
| 190. | Example 191 | 69.63 | 82.15 | A |
| 191. | Example 192 | 62.12 | 68.32 | A |
| 192. | Example 193 | 78.25 | 74.47 | A |
| 193. | Example 194 | 68.56 | 66.64 | A |
| 194. | Example 195 | 25.05 | 61.09 | |
| 195. | Example 196 | 64.95 | 66.27 | A |
| 196. | Example 197 | 69.77 | 70.06 | A |
| 197. | Example 198 | 73.84 | 71.07 | A |
| 198. | Example 199 | 71.73 | — | A |
| 199. | Example 200 | 70.71 | 71.88 | A |
| 200. | Example 201 | 65.66 | 73.63 | A |
| 201. | Example 202 | 75.67 | 60.98 | A |
| 202. | Example 203 | 67.63 | 83.04 | A |
| 203. | Example 204 | 72.68 | 86.14 | A |
| 204. | Example 205 | 73.27 | — | A |
| 205. | Example 206 | 72.4 | 84.92 | A |
| 206. | Example 207 | 73.8 | — | A |
| 207. | Example 208 | 79.67 | — | A |
| 208. | Example 209 | 75.69 | 78.89 | A |
| 209. | Example 210 | 70.8 | — | A |
| 210. | Example 211 | 76.87 | 79.43 | A |
| 211. | Example 212 | 63.14 | 74.37 | A |
| 212. | Example 213 | 60.17 | 69.46 | A |
| 213. | Example 214 | 69.7 | 67.73 | A |
| 214. | Example 215 | 67.26 | — | A |
| 215. | Example 216 | 72.14 | 68.62 | A |
| 216. | Example 217 | 91 | — | A |
| 217. | Example 218 | 77.7 | — | A |

—: Not determined.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above.

All publications and patent applications cited in this application are herein incorporated by reference to the same extent as if each individual publication or patent application was specifically and individually indicated to be incorporated herein by reference.

1. A compound of formula (I) 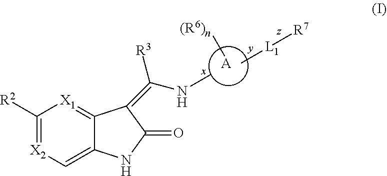
stereoisomer, diastereoisomer, enantiomer or a pharmaceutically acceptable salt thereof, wherein,
X¹ is selected from CH and N;
X² is selected from CH, CR¹ and N;
R¹ is selected from halogen, cyano and C₁₋₈alkyl;
R² is 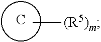
Ring C is selected from 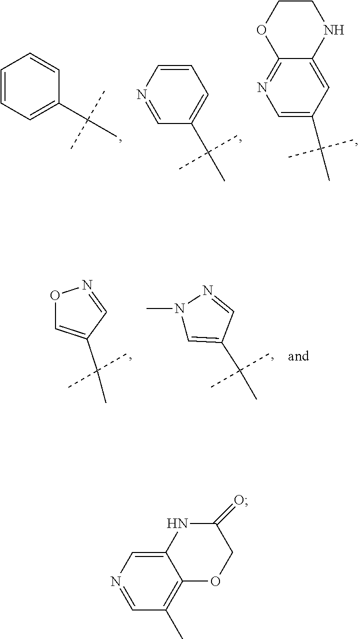
each occurrence of R⁵ is selected from cyano, halogen, C₁₋₈alkyl, C₁₋₈alkoxy, haloC₁₋₈alkoxy, C₃₋₁₂cycloalkyl, C₁₋₈alkoxy C₃₋₁₂cycloalkyl, hydroxyC₁₋₈alkyl and amino;
R³ is C₁₋₈alkyl; 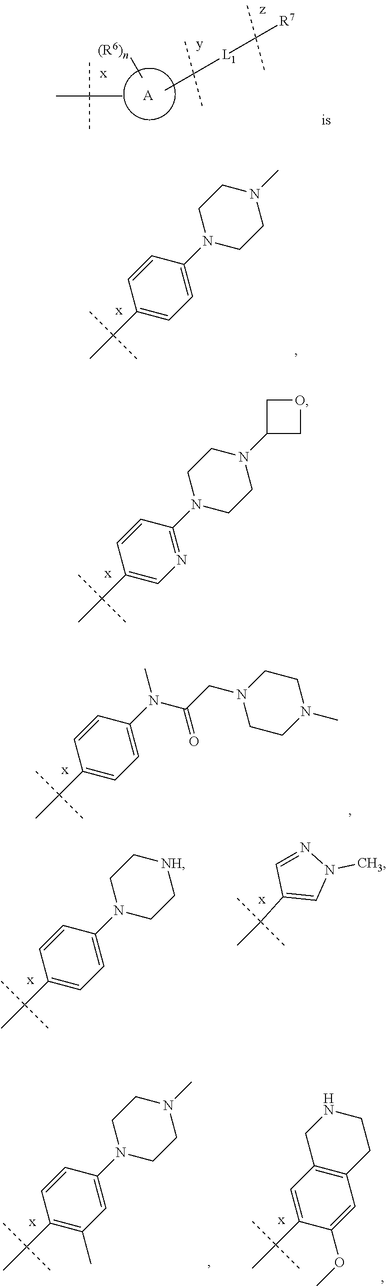 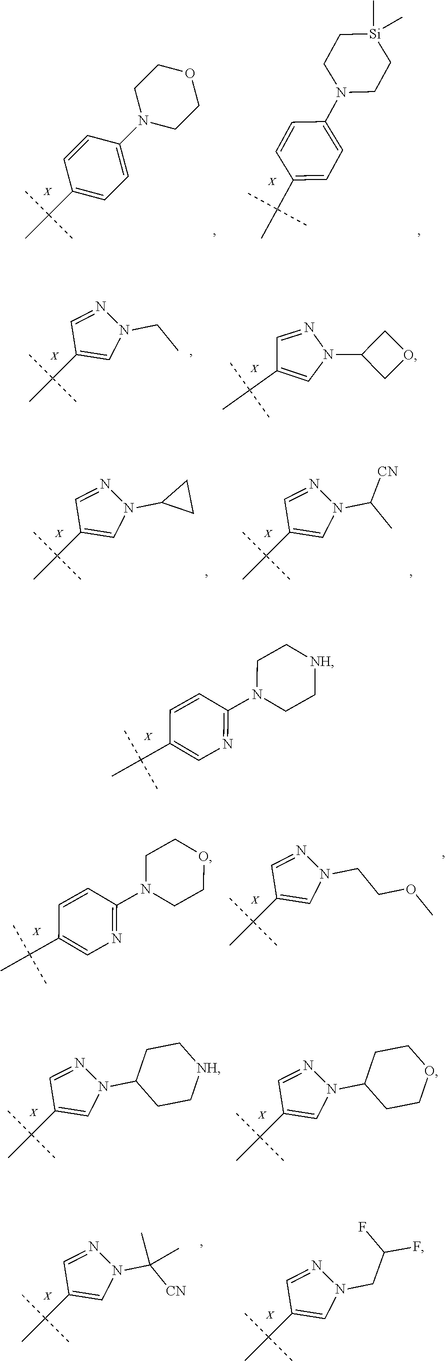 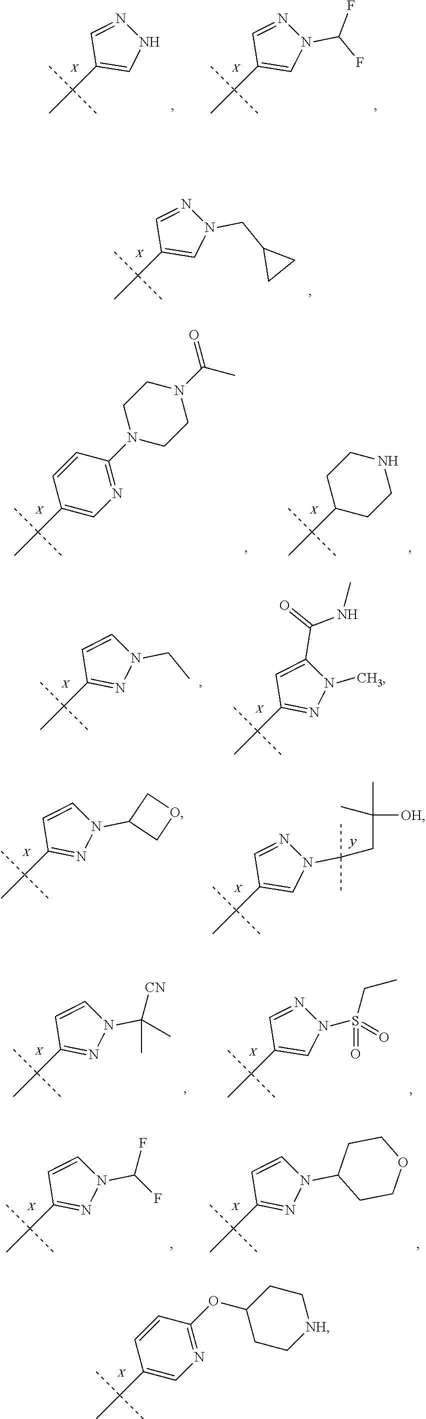 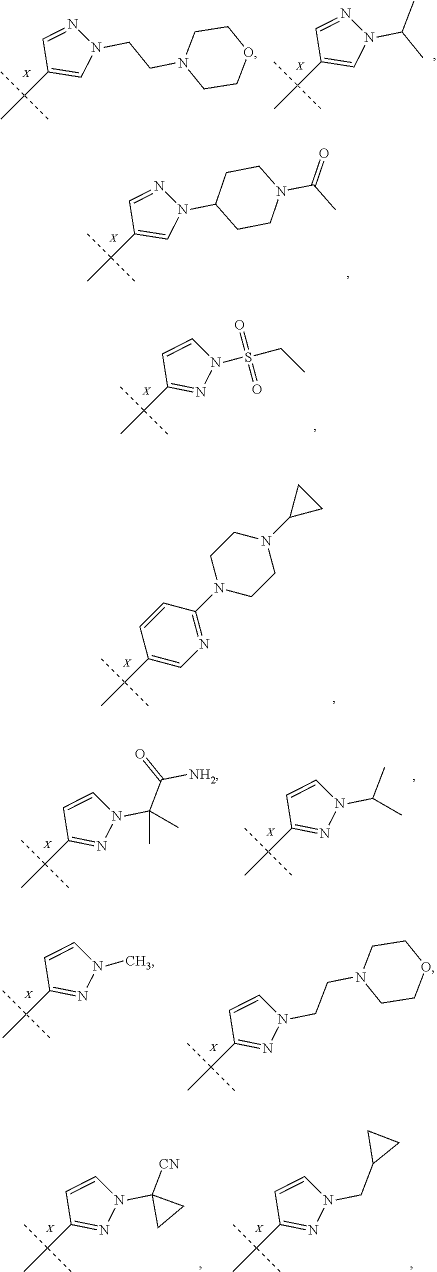 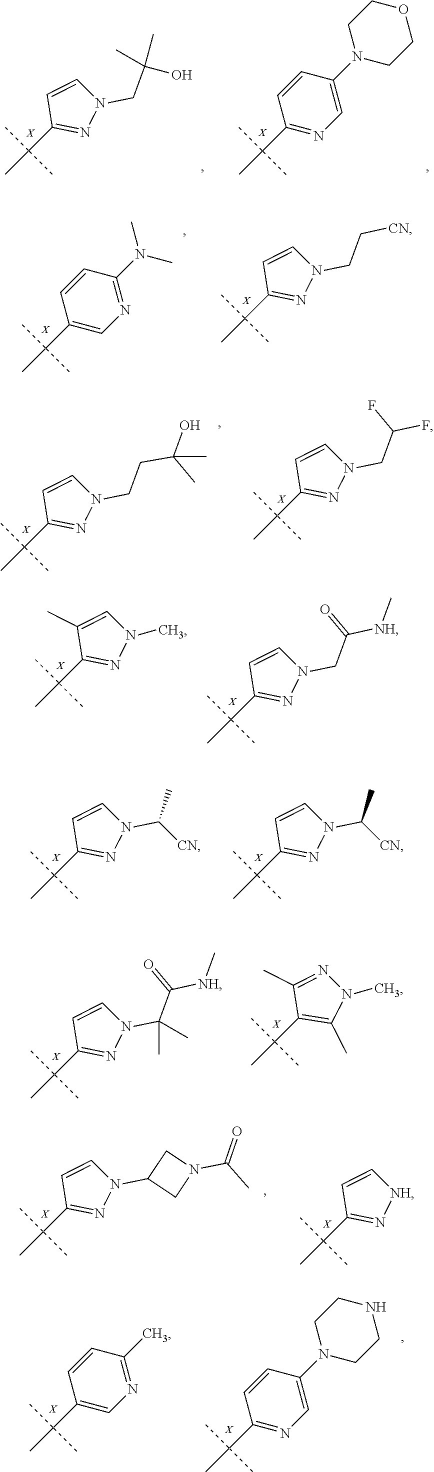 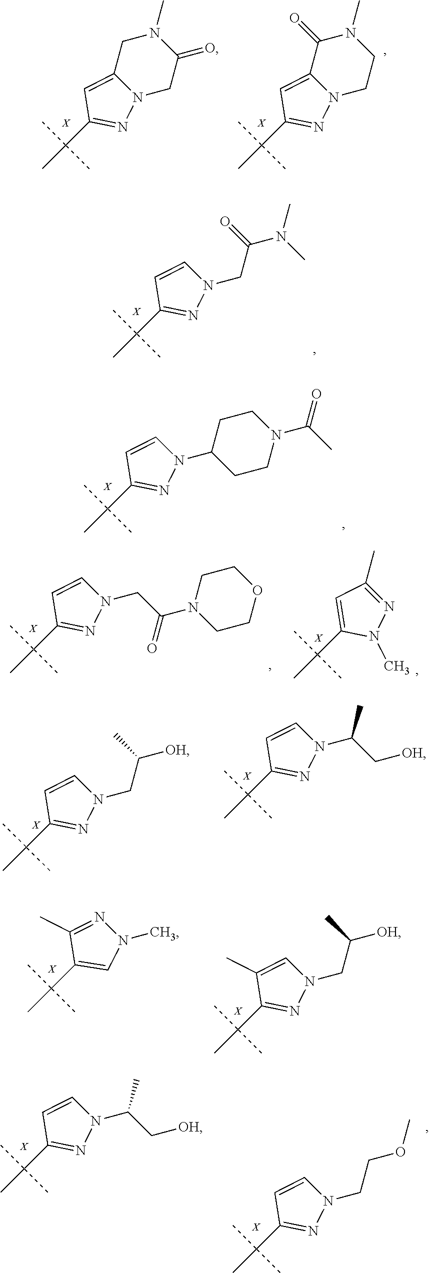 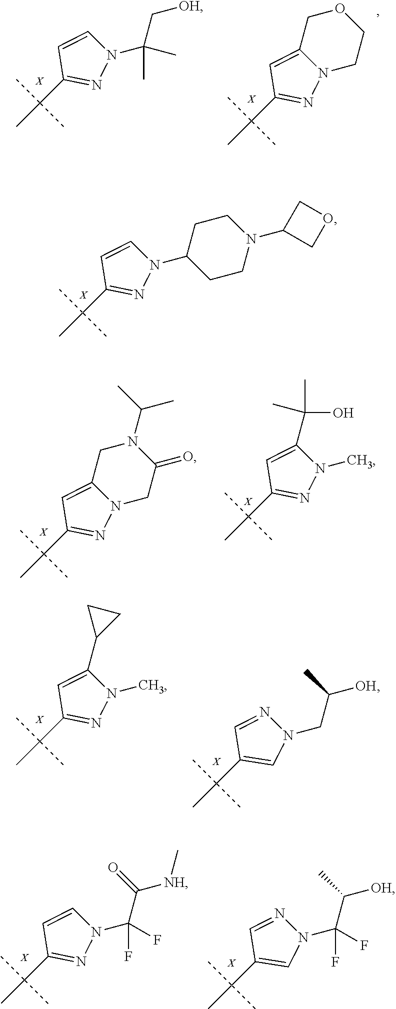 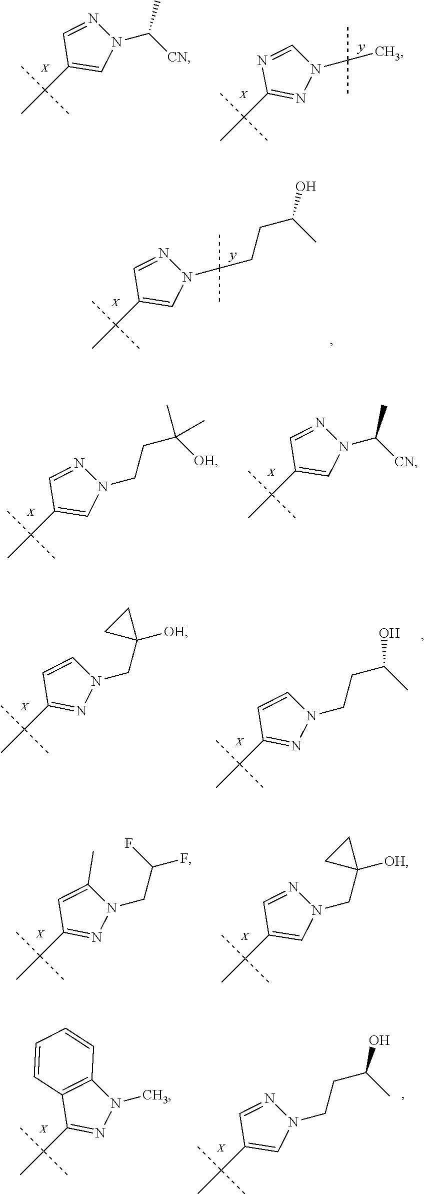 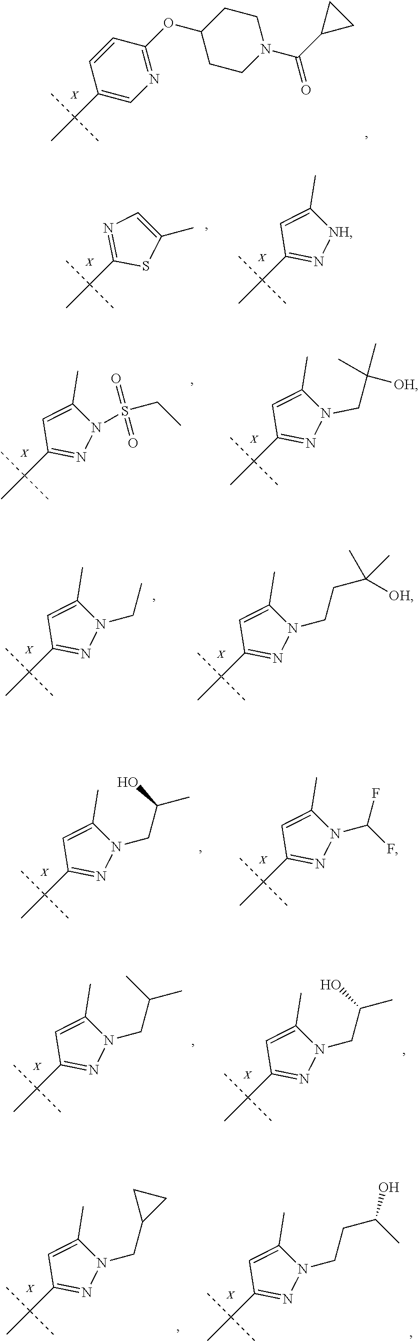 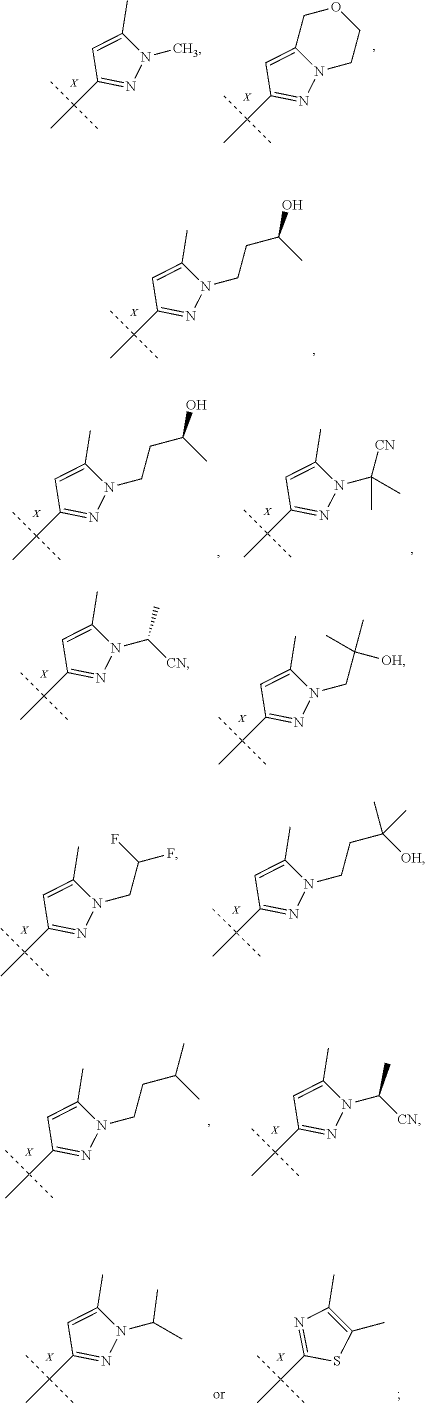
‘m’ is 0, 1 or 2; and
‘n’ is 0, 1 or 2,

2-11. (canceled)

12. The compound according to claim 1, wherein
X¹ is CH or N;
X² is CH, CR¹ or N;
R¹ is fluoro, chloro, methyl or cyano; 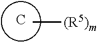
is 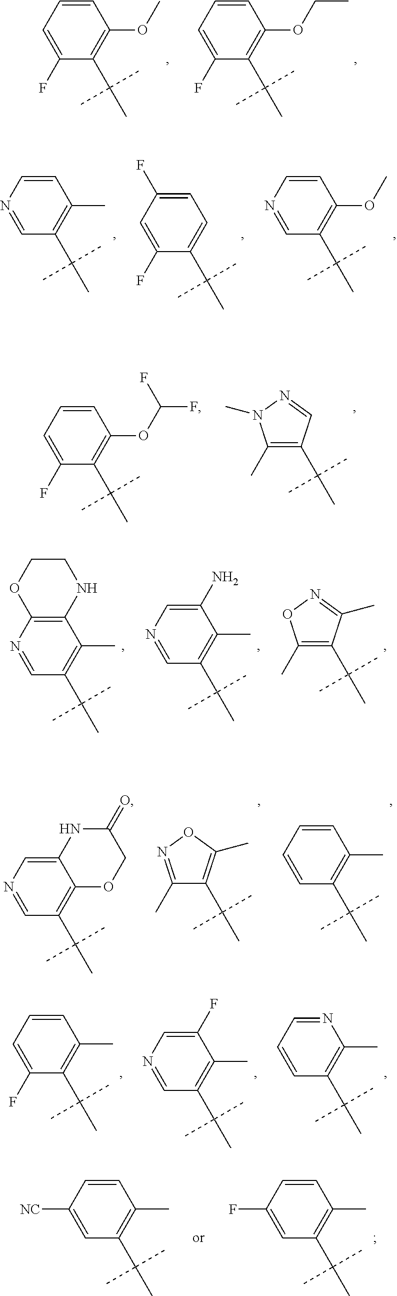
R³ is methyl, ethyl or isopropyl;

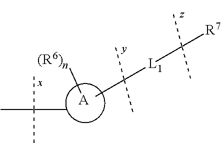

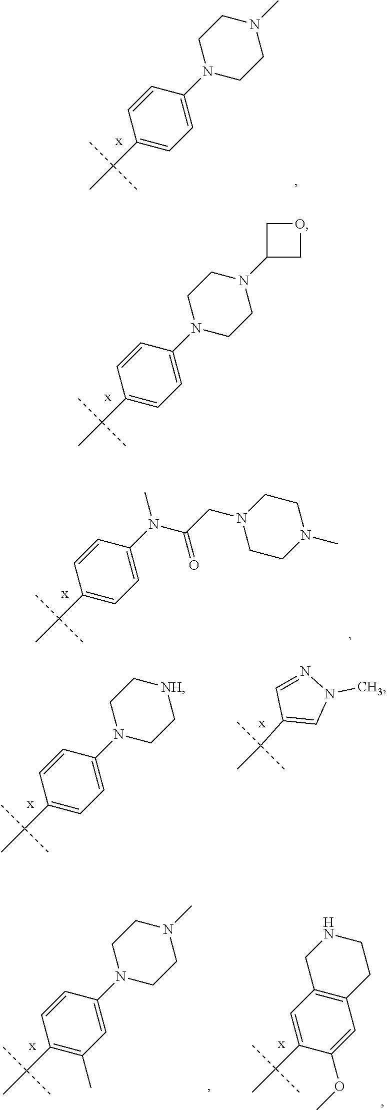

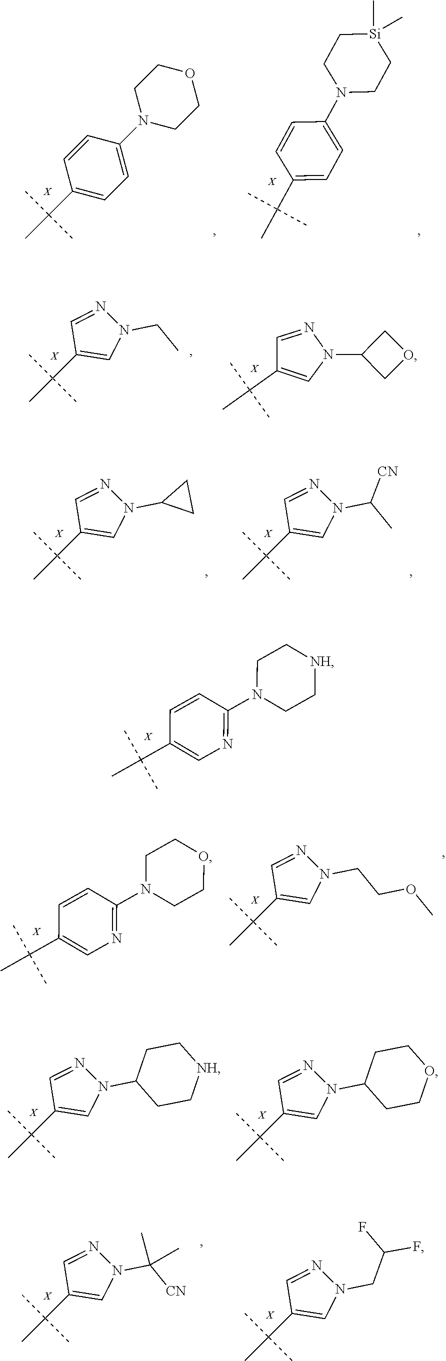

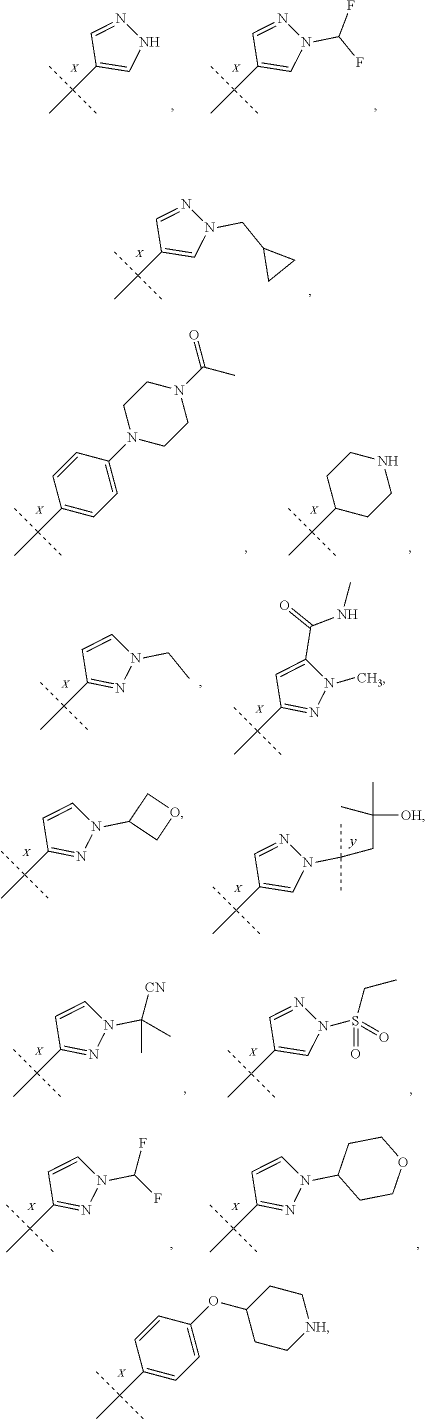

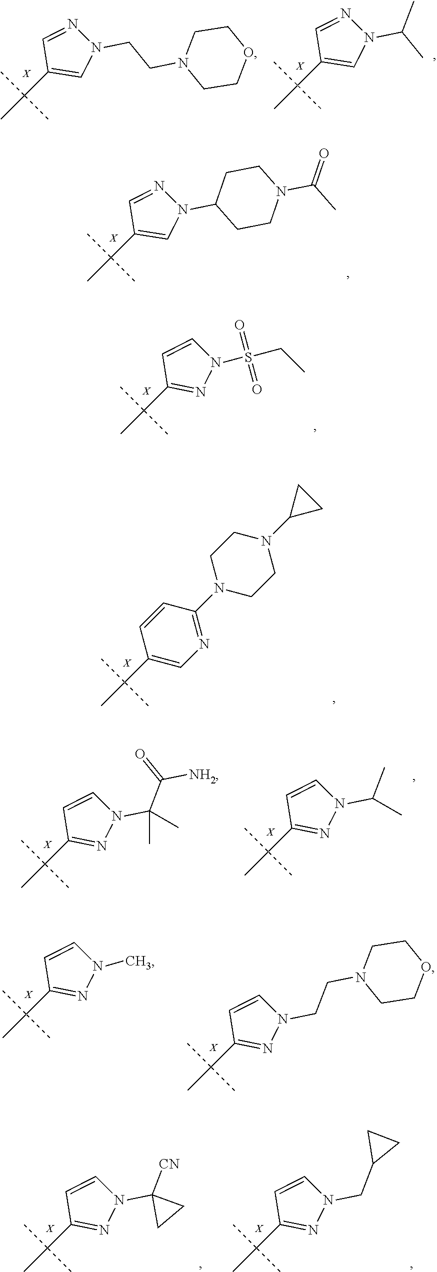

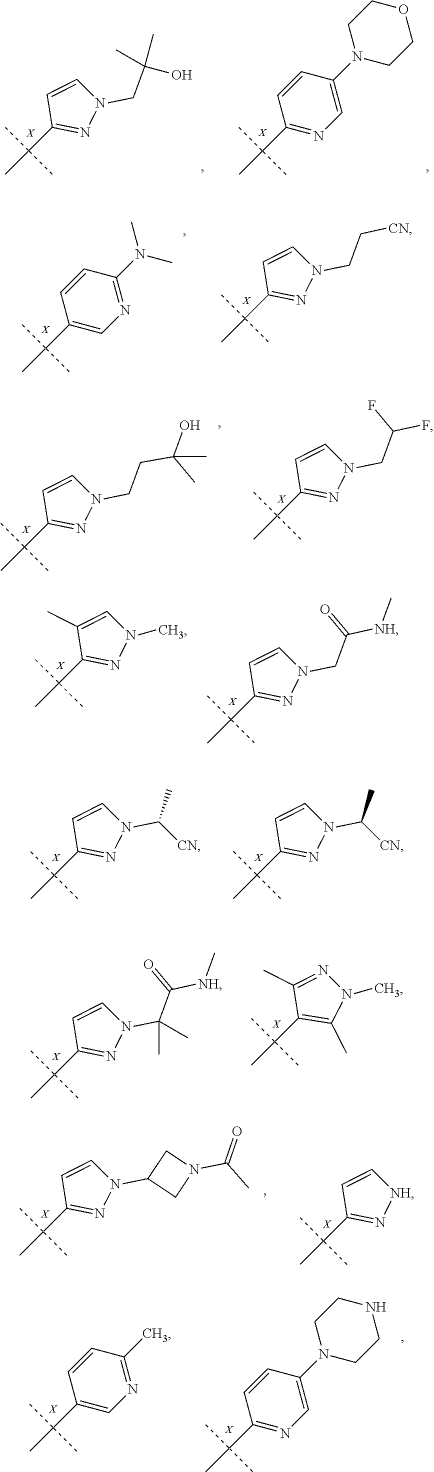

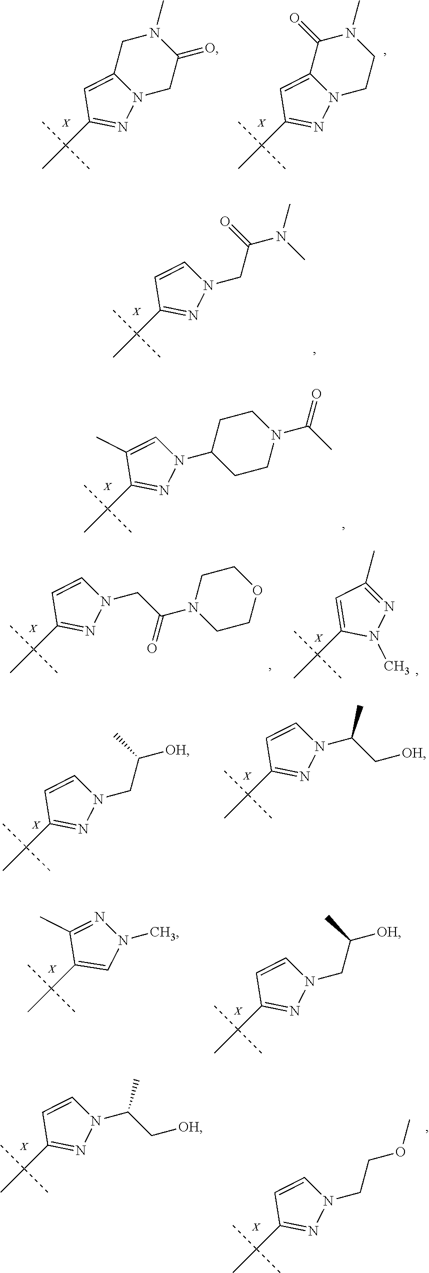

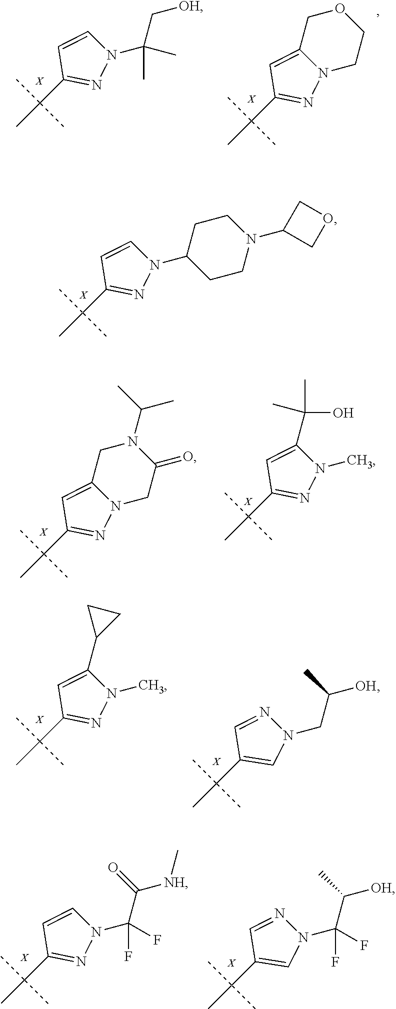

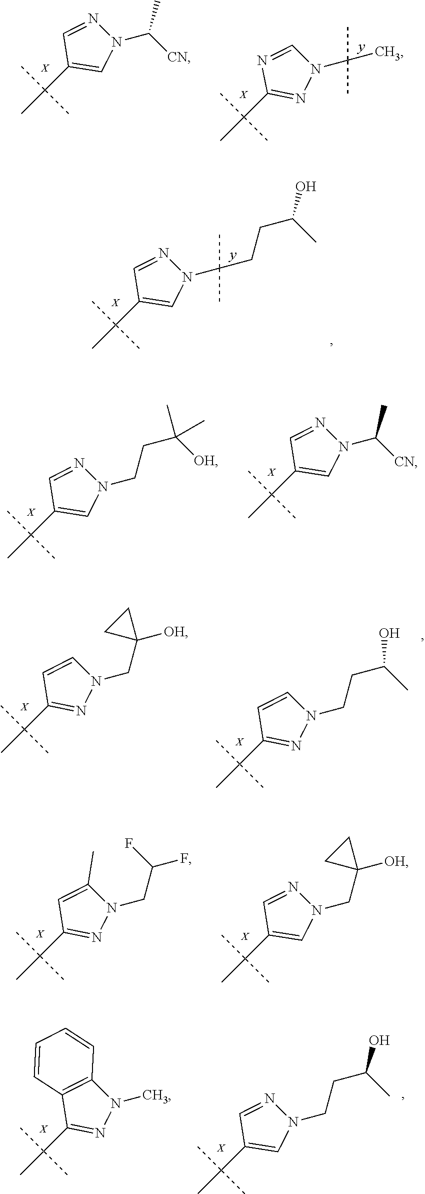

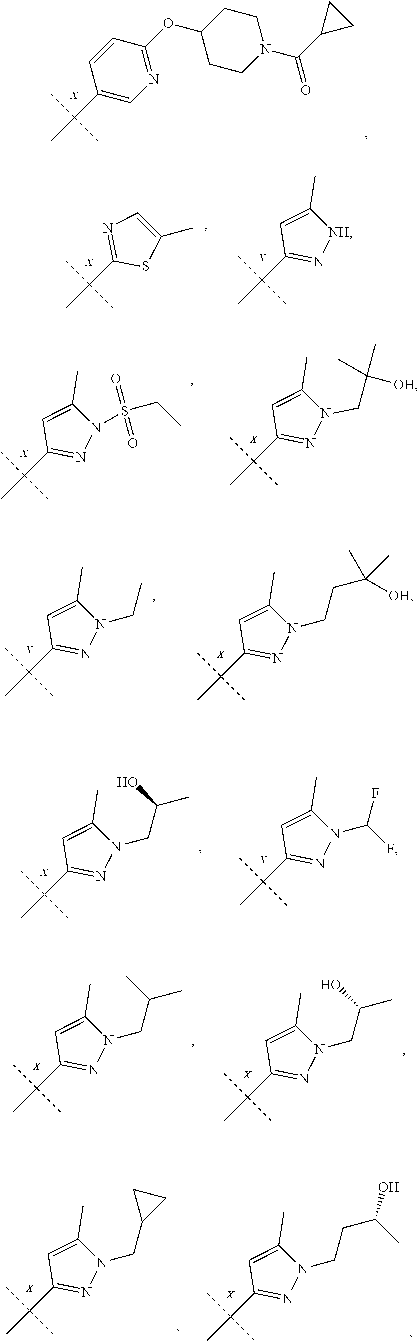

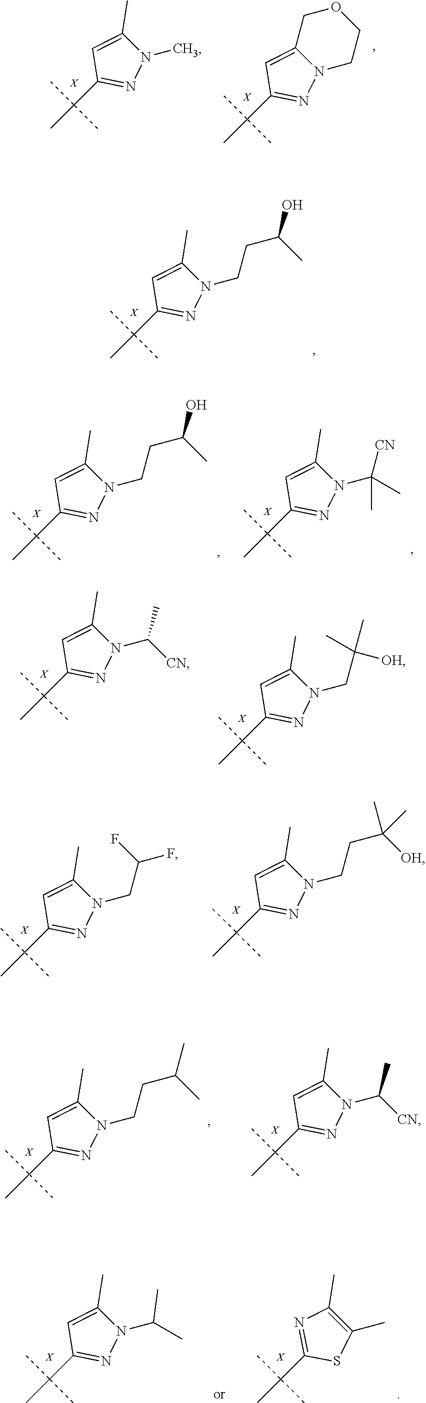

13. The compound according to claim 1 selected from
(Z)-5-(2-Fluoro-6-methoxyphenyl)-3-(1-((4-(4-methylpiperazin-1-yl)phenyl)amino)ethylidene)indolin-2-one;
(Z)-5-(2-Fluoro-6-methoxyphenyl)-3-(1-(phenylamino)ethylidene)indolin-2-one;
(Z)-5-(2-Fluoro-6-methoxyphenyl)-3-(1-((6-(4-(oxetan-3-yl)piperazin-1-yl)pyridin-3-yl)amino)ethylidene)indolin-2-one;
(Z)-5-Fluoro-3-(1-((6-(4-(oxetan-3-yl)piperazin-1-yl)pyridin-3-yl)amino)ethylidene)indolin-2-one;
(Z)-5-Fluoro-3-(1-((4-(4-methylpiperazin-1-yl)phenyl)amino)ethylidene) indolin-2-one;
(Z)-N-(4-((1-(5-(2-Fluoro-6-methoxyphenyl)-2-oxoindolin-3-ylidene)ethyl)amino)phenyl)-N-methyl-2-(4-methylpiperazin-1-yl)acetamide;
(Z)-5-(2-Fluoro-6-methoxyphenyl)-3-(1-((4-(piperazin-1-yl)phenyl)amino)ethylidene)indolin-2-one;
(Z)-5-(2-Fluoro-6-methoxyphenyl)-3-(1-((4-(4-methylpiperazin-1-yl)phenyl)amino)propylidene)indolin-2-one;
(Z)-5-(2-Fluoro-6-methoxyphenyl)-3-(1-((1-methyl-1H-pyrazol-4-yl)amino)ethylidene)indolin-2-one;
(Z)-5-(2-Fluoro-6-methoxyphenyl)-3-(1-((2-methyl-4-(4-methylpiperazin-1-yl)phenyl)amino)ethylidene)indolin-2-one;
(Z)-5-(2-Fluoro-6-methoxyphenyl)-3-(1-((6-methoxy-1,2,3,4-tetrahydroisoquinolin-7-yl)amino)ethylidene)indolin-2-one para toluenesulfonate;
(Z)-5-(2-Fluoro-6-methoxyphenyl)-3-(1-((4-morpholinophenyl)amino) ethylidene)indolin-2-one;
(Z)-5-(2-fluoro-6-methoxyphenyl)-3-(1-((4-(4-methylpiperazin-1-yl)phenyl)amino)ethylidene)-1H-pyrrolo[2,3-c]pyridin-2(3H)-one;
(Z)-5-(2-Fluoro-6-methoxyphenyl)-3-(1-((1-methyl-1H-pyrazol-4-yl)amino)ethylidene)-1H-pyrrolo[2,3-c]pyridin-2(3H)-one;
(Z)-5-(2-Fluoro-6-methoxyphenyl)-3-(1-((1-methyl-1H-pyrazol-4-yl)amino)propylidene)indolin-2-one;
(Z)-5-(2-Fluoro-6-methoxyphenyl)-3-(1-((1-methyl-1H-pyrazol-3-yl)amino)ethylidene)indolin-2-one;
(Z)-3-((3,5-Dimethyl-4-(1-methyl-1H-pyrazol-4-yl)-1H-pyrrol-2-yl)methylene)-5-(2-fluoro-6-methoxyphenyl)indolin-2-one;
(Z)-3-(1-((4-(4,4-Dimethyl-1,4-azasilinan-1-yl)phenyl)amino)ethylidene)-5-(2-fluoro-6-methoxyphenyl)indolin-2-one;
(Z)-5-(2-Fluoro-6-methoxyphenyl)-6-methyl-3-(1-((1-methyl-1H-pyrazol-4-yl)amino)ethylidene)indolin-2-one;
(Z)-5-(2-Ethoxy-6-fluorophenyl)-3-(1-((1-ethyl-1H-pyrazol-4-yl)amino)ethylidene)-1H-pyrrolo[2,3-c]pyridin-2(3H)-one;
(Z)-3-(1-((1-methyl-1H-pyrazol-4-yl)amino)ethylidene)-5-(4-methylpyridin-3-yl)-1H-pyrrolo[2,3-c]pyridin-2(3H)-one;
(Z)-5-(2,4-Difluorophenyl)-3-(1-((1-methyl-1H-pyrazol-4-yl)amino)ethylidene)-1H-pyrrolo[2,3-c]pyridin-2(3H)-one;
(Z)-5-(2-Ethoxy-6-fluorophenyl)-3-(1-((1-methyl-1H-pyrazol-4-yl)amino)ethylidene)-1H-pyrrolo[2,3-c]pyridin-2(3H)-one;
((Z)-6-Fluoro-5-(2-fluoro-6-methoxyphenyl)-3-(1-((1-methyl-1H-pyrazol-4-yl)amino)ethylidene)indolin-2-one;
(Z)-5-(2-Fluoro-6-methoxyphenyl)-3-(1-((1-(oxetan-3-yl)-1H-pyrazol-4-yl)amino)ethylidene)-1H-pyrrolo[2,3-c]pyridin-2(3H)-one;
(Z)-5-(2-Fluoro-6-methoxyphenyl)-3-(1-((1-methyl-1H-pyrazol-3-yl)amino)ethylidene)-1H-pyrrolo[2,3-c]pyridin-2(3H)-one;
(Z)-3-(1-((1-Ethyl-1H-pyrazol-4-yl)amino)ethylidene)-5-(2-fluoro-6-methoxyphenyl)-1H-pyrrolo[2,3-c]pyridin-2(3H)-one;
(Z)-3-(1-((1-Cyclopropyl-1H-pyrazol-4-yl)amino)ethylidene)-5-(2-fluoro-6-methoxyphenyl)-1H-pyrrolo[2,3-c]pyridin-2(3H)-one;
(Z)-2-(4-((1-(5-(2-Fluoro-6-methoxyphenyl)-2-oxo-1H-pyrrolo[2,3-c]pyridin-3(2H)-ylidene)ethyl)amino)-1H-pyrazol-1-yl)propanenitrile;
(Z)-5-(2-Fluoro-6-methoxyphenyl)-3-(1-((1-methyl-1H-pyrazol-4-yl)amino)propylidene)-1H-pyrrolo[2,3-c]pyridin-2(3H)-one;
(Z)-5-(4-methoxypyridin-3-yl)-3-(1-((1-methyl-1H-pyrazol-4-yl)amino)ethylidene)-1H-pyrrolo[2,3-c]pyridin-2(3H)-one;
(Z)-5-(2-Ethoxy-6-fluorophenyl)-3-(1-((4-(piperazin-1-yl)phenyl)amino)ethylidene)-1H-pyrrolo[2,3-c]pyridin-2(3H)-one;
(Z)-5-(2-(Difluoromethoxy)-6-fluorophenyl)-3-(1-((1-methyl-1H-pyrazol-4-yl)amino)ethylidene)-1H-pyrrolo[2,3-c]pyridin-2(3H)-one;
(Z)-5-(2-Ethoxy-6-fluorophenyl)-3-(1-((6-(piperazin-1-yl)pyridin-3-yl)amino)ethylidene)-1H-pyrrolo[2,3-c]pyridin-2(3H)-one;
(Z)-5-(2-Ethoxy-6-fluorophenyl)-3-(1-((6-morpholinopyridin-3-yl)amino)ethylidene)-1H-pyrrolo[2,3-c]pyridin-2(3H)-one;
(Z)-5-(4-Methylpyridin-3-yl)-3-(1-((6-morpholinopyridin-3-yl)amino)ethylidene)-1H-pyrrolo[2,3-c]pyridin-2(3H)-one;
(Z)-5-(2-Fluoro-6-methoxyphenyl)-3-(1-((1-(2-methoxyethyl)-1H-pyrazol-4-yl)amino)ethylidene)-1H-pyrrolo[2,3-c]pyridin-2(3H)-one;
(Z)-5-(2-Fluoro-6-methoxyphenyl)-3-(2-methyl-1-((1-methyl-1H-pyrazol-4-yl)amino)propylidene)-1H-pyrrolo[2,3-c]pyridin-2(3H)-one;
(Z)-5-(1,5-Dimethyl-1H-pyrazol-4-yl)-3-(1-((1-methyl-1H-pyrazol-4-yl)amino)ethylidene)-1H-pyrrolo[2,3-c]pyridin-2(3H)-one;
(Z)-5-(2-Fluoro-6-methoxyphenyl)-3-(1-((1-(piperidin-4-yl)-1H-pyrazol-4-yl)amino)ethylidene)-1H-pyrrolo[2,3-c]pyridin-2(3H)-one;
(Z)-5-(2-Fluoro-6-methoxyphenyl)-3-(1-((1-methyl-1H-pyrazol-4-yl)amino)ethylidene)-1H-pyrrolo[3,2-b]pyridin-2(3H)-one;
(Z)-5-(2-Fluoro-6-methoxyphenyl)-3-(1-((1-(tetrahydro-2H-pyran-4-yl)-1H-pyrazol-4-yl)amino)ethylidene)-1H-pyrrolo[2,3-c]pyridin-2(3H)-one;
(Z)-3-(1-((1-Methyl-1H-pyrazol-4-yl)amino)ethylidene)-5-(8-methyl-2,3-dihydro-1H-pyrido[2,3-b][1,4]oxazin-7-yl)-1H-pyrrolo[2,3-c]pyridin-2(3H)-one;
(Z)-5-(4-Methylpyridin-3-yl)-3-(1-((6-(piperazin-1-yl)pyridin-3-yl)amino)ethylidene)-1H-pyrrolo[2,3-c]pyridin-2(3H)-one;
(Z)-3-(1-((1-Methyl-1H-pyrazol-4-yl)amino)propylidene)-5-(4-methylpyridin-3-yl)-1H-pyrrolo[2,3-c]pyridin-2(3H)-one;
(Z)-3-(2-Methyl-1-((1-methyl-1H-pyrazol-4-yl)amino)propylidene)-5-(4-methylpyridin-3-yl)-1H-pyrrolo[2,3-c]pyridin-2(3H)-one;
(Z)-3-(1-((1-Cyclopropyl-1H-pyrazol-4-yl)amino)ethylidene)-5-(4-methylpyridin-3-yl)-1H-pyrrolo[2,3-c]pyridin-2(3H)-one;
(Z)-3-(1-((1-Ethyl-1H-pyrazol-4-yl)amino)ethylidene)-5-(4-methylpyridin-3-yl)-1H-pyrrolo[2,3-c]pyridin-2(3H)-one;
(Z)-5-(5-Amino-4-methylpyridin-3-yl)-3-(1-((1-methyl-1H-pyrazol-4-yl)amino)ethylidene)-1H-pyrrolo[2,3-c]pyridin-2(3H)-one;
(Z)-5-(4-Methylpyridin-3-yl)-3-(1-((1-(oxetan-3-yl)-1H-pyrazol-4-yl)amino)ethylidene)-1H-pyrrolo[2,3-c]pyridin-2(3H)-one;
(Z)-2-Methyl-2-(4-((1-(5-(4-methylpyridin-3-yl)-2-oxo-1H-pyrrolo[2,3-c]pyridin-3(2H)-ylidene)ethyl)amino)-1H-pyrazol-1-yl)propanenitrile;
(Z)-3-(1-((1-methyl-1H-pyrazol-3-yl)amino)ethylidene)-5-(4-methylpyridin-3-yl)-1H-pyrrolo[2,3-c]pyridin-2(3H)-one;
(Z)-5-(3,5-dimethylisoxazol-4-yl)-3-(1-((1-methyl-1H-pyrazol-4-yl)amino)ethylidene)-1H-pyrrolo[2,3-c]pyridin-2(3H)-one;
(Z)-5-(4-Methylpyridin-3-yl)-3-(1-((1-(tetrahydro-2H-pyran-4-yl)-1H-pyrazol-4-yl)amino)ethylidene)-1H-pyrrolo[2,3-c]pyridin-2(3H)-one;
(Z)-5-(4-Methylpyridin-3-yl)-3-(1-((4-(piperazin-1-yl)phenyl)amino) ethylidene)-1H-pyrrolo[2,3-c]pyridin-2(3H)-one;
(Z)-5-(4-Methylpyridin-3-yl)-3-(1-((4-morpholinophenyl)amino)ethylidene)-1H-pyrrolo[2,3-c]pyridin-2(3H)-one;
(Z)-3-(1-((1-(2-Methoxyethyl)-1H-pyrazol-4-yl)amino)ethylidene)-5-(4-methylpyridin-3-yl)-1H-pyrrolo[2,3-c]pyridin-2(3H)-one;
(Z)-3-(1-((1-(2,2-Difluoroethyl)-1H-pyrazol-4-yl)amino)ethylidene)-5-(4-methylpyridin-3-yl)-1H-pyrrolo[2,3-c]pyridin-2(3H)-one;
(Z)-3-(1-((1-Methyl-1H-pyrazol-4-yl)amino)ethylidene)-5-(4-methylpyridin-3-yl)-1H-pyrrolo[3,2-b]pyridin-2(3H)-one;
(Z)-3-(1-((1-Methyl-1H-pyrazol-3-yl)amino)ethylidene)-5-(4-methylpyridin-3-yl)-1H-pyrrolo[3,2-b]pyridin-2(3H)-one;
(Z)-3-(1-((1H-Pyrazol-4-yl)amino)ethylidene)-5-(4-methylpyridin-3-yl)-1H-pyrrolo[2,3-c]pyridin-2(3H)-one;
(Z)-3-(1-((1-(Difluoromethyl)-1H-pyrazol-4-yl)amino)ethylidene)-5-(2-fluoro-6-methoxyphenyl)-1H-pyrrolo[2,3-c]pyridin-2(3H)-one;
(Z)-3-(1-((1-(Difluoromethyl)-1H-pyrazol-4-yl)amino)ethylidene)-5-(4-methylpyridin-3-yl)-1H-pyrrolo[2,3-c]pyridin-2(3H)-one;
(Z)-3-(1-((1-(Cyclopropylmethyl)-1H-pyrazol-4-yl)amino)ethylidene)-5-(4-methylpyridin-3-yl)-1H-pyrrolo[2,3-c]pyridin-2(3H)-one;
(Z)-5-(4-Methoxypyridin-3-yl)-3-(1-((1-methyl-1H-pyrazol-3-yl)amino)ethylidene)-1H-pyrrolo[2,3-c]pyridin-2(3H)-one (Z)-5-(4-Methylpyridin-3-yl)-3-(1-((1-(2,2,2-trifluoroethyl)-1H-pyrazol-4-yl)amino)ethylidene)-1H-pyrrolo[2,3-c]pyridin-2(3H)-one;
(Z)-3-(1-((6-(4-Acetylpiperazin-1-yl)pyridin-3-yl)amino)ethylidene)-5-(4-methylpyridin-3-yl)-1H-pyrrolo[2,3-c]pyridin-2(3H)-one;
(Z)-5-(2-Fluoro-6-methoxyphenyl)-3-(1-((1-methyl-1H-pyrazol-4-yl)amino)ethylidene)-2-oxoindoline-6-carbonitrile;
(Z)-5-(2-Fluoro-6-methoxyphenyl)-3-(1-(piperidin-4-ylamino)ethylidene)-1H-pyrrolo[2,3-c]pyridin-2(3H)-one trihydrochloride;
(Z)-3-(1-((1-Ethyl-1H-pyrazol-3-yl)amino)ethylidene)-5-(4-methylpyridin-3-yl)-1H-pyrrolo[2,3-c]pyridin-2(3H)-one;
(Z)-5-(4-Methylpyridin-3-yl)-3-(1-((5-(piperazin-1-yl)pyridin-2-yl)amino)ethylidene)-1H-pyrrolo[2,3-c]pyridin-2(3H)-one;
(Z)-N,1-Dimethyl-3-((1-(5-(4-methylpyridin-3-yl)-2-oxo-1H-pyrrolo[2,3-c]pyridin-3(2H)-ylidene)ethyl)amino)-1H-pyrazole-5-carboxamide;
(Z)-5-(4-Methylpyridin-3-yl)-3-(1-((1-(oxetan-3-yl)-1H-pyrazol-3-yl)amino)ethylidene)-1H-pyrrolo[2,3-c]pyridin-2(3H)-one;
(Z)-5-(4-Methylpyridin-3-yl)-3-(1-((6-(4-(oxetan-3-yl)piperazin-1-yl)pyridin-3-yl)amino)ethylidene)-1H-pyrrolo[2,3-c]pyridin-2(3H)-one;
(Z)-3-(1-((1-(2-Hydroxy-2-methylpropyl)-1H-pyrazol-4-yl)amino)ethylidene)-5-(4-methylpyridin-3-yl)-1H-pyrrolo[2,3-c]pyridin-2(3H)-one;
(Z)-2-Methyl-2-(3-((1-(5-(4-methylpyridin-3-yl)-2-oxo-1H-pyrrolo[2,3-c]pyridin-3(2H)-ylidene)ethyl)amino)-1H-pyrazol-1-yl)propanenitrile;
(Z)-3-(1-((1-(Ethylsulfonyl)-1H-pyrazol-4-yl)amino)ethylidene)-5-(4-methylpyridin-3-yl)-1H-pyrrolo[2,3-c]pyridin-2(3H)-one;
(Z)-3-(1-((1-(Difluoromethyl)-1H-pyrazol-3-yl)amino)ethylidene)-5-(4-methylpyridin-3-yl)-1H-pyrrolo[2,3-c]pyridin-2(3H)-one;
(Z)-2-(4-((1-(5-(2-Fluoro-6-methoxyphenyl)-2-oxo-1H-pyrrolo[2,3-c]pyridin-3(2H)-ylidene)ethyl)amino)-1H-pyrazol-1-yl)-2-methylpropanenitrile;
(Z)-5-(4-Methylpyridin-3-yl)-3-(1-((1-(tetrahydro-2H-pyran-4-yl)-1H-pyrazol-3-yl)amino)ethylidene)-1H-pyrrolo[2,3-c]pyridin-2(3H)-one;
(Z)-5-(4-Methylpyridin-3-yl)-3-(1-((6-(piperidin-4-yloxy)pyridin-3-yl)amino)ethylidene)-1H-pyrrolo[2,3-c]pyridin-2(3H)-one;
(Z)-8-(3-(1-((1-Methyl-1H-pyrazol-4-yl)amino)ethylidene)-2-oxo-2,3-dihydro-1H-pyrrolo[2,3-c]pyridin-5-yl)-2H-pyrido[4,3-b][1,4]oxazin-3(4H)-one;
(Z)-5-(4-Methylpyridin-3-yl)-3-(1-((2-morpholinoethyl)amino)ethylidene)-1H-pyrrolo[2,3-c]pyridin-2(3H)-one;
(Z)-5-(4-Methylpyridin-3-yl)-3-(1-((1-(2-morpholinoethyl)-1H-pyrazol-4-yl)amino)ethylidene)-1H-pyrrolo[2,3-c]pyridin-2(3H)-one;
(Z)-2-(4-((1-(5-(3,5-Dimethylisoxazol-4-yl)-2-oxo-1H-pyrrolo[2,3-c]pyridin-3(2H)-ylidene)ethyl)amino)-1H-pyrazol-1-yl)-2-methylpropanenitrile;
(Z)-3-(1-((1-Isopropyl-1H-pyrazol-4-yl)amino)ethylidene)-5-(4-methylpyridin-3-yl)-1H-pyrrolo[2,3-c]pyridin-2(3H)-one;
(Z)-3-(1-((1-(1-Acetylpiperidin-4-yl)-1H-pyrazol-4-yl)amino)ethylidene)-5-(4-methylpyridin-3-yl)-1H-pyrrolo[2,3-c]pyridin-2(3H)-one;
(Z)-2-Methyl-2-(4-((1-(5-(4-methylpyridin-3-yl)-2-oxo-1H-pyrrolo[2,3-c]pyridin-3(2H)-ylidene)propyl)amino)-1H-pyrazol-1-yl)propanenitrile;
(Z)-2-Methyl-2-(3-((1-(5-(4-methylpyridin-3-yl)-2-oxo-1H-pyrrolo[2,3-c]pyridin-3(2H)-ylidene)propyl)amino)-1H-pyrazol-1-yl)propanenitrile;
(Z)-2-(3-((1-(5-(2-Fluoro-6-methoxyphenyl)-2-oxo-1H-pyrrolo[2,3-c]pyridin-3(2H)-ylidene)ethyl)amino)-1H-pyrazol-1-yl)-2-methylpropanenitrile;
(Z)-3-(1-((1-(Ethylsulfonyl)-1H-pyrazol-3-yl)amino)ethylidene)-5-(4-methylpyridin-3-yl)-1H-pyrrolo[2,3-c]pyridin-2(3H)-one;
(Z)-2-(3-((1-(5-(4-Methoxypyridin-3-yl)-2-oxo-1H-pyrrolo[2,3-c]pyridin-3(2H)-ylidene)ethyl)amino)-1H-pyrazol-1-yl)-2-methylpropanenitrile;
(Z)-3-(1-((6-(4-Cyclopropylpiperazin-1-yl)pyridin-3-yl)amino)ethylidene)-5-(4-methylpyridin-3-yl)-1H-pyrrolo[2,3-c]pyridin-2(3H)-one;
(Z)-2-Methyl-2-(3-((1-(5-(4-methylpyridin-3-yl)-2-oxo-1H-pyrrolo[2,3-c]pyridin-3(2H)-ylidene)ethyl)amino)-1H-pyrazol-1-yl)propanamide;
(Z)-2-Methyl-2-(3-((1-(2-oxo-5-(o-tolyl)-1H-pyrrolo[2,3-c]pyridin-3(2H)-ylidene)ethyl)amino)-1H-pyrazol-1-yl)propanenitrile;
(Z)-3-(1-((1-Isopropyl-1H-pyrazol-3-yl)amino)ethylidene)-5-(4-methylpyridin-3-yl)-1H-pyrrolo[2,3-c]pyridin-2(3H)-one;
(Z)-3-(1-((1-Methyl-1H-pyrazol-3-yl)amino)ethylidene)-5-(4-methylpyridin-3-yl)indolin-2-one;
(Z)-5-(4-Methylpyridin-3-yl)-3-(1-((1-(2-morpholinoethyl)-1H-pyrazol-3-yl)amino)ethylidene)-1H-pyrrolo[2,3-c]pyridin-2(3H)-one;
(Z)-1-(3-((1-(5-(4-Methylpyridin-3-yl)-2-oxo-1H-pyrrolo[2,3-c]pyridin-3(2H)-ylidene)ethyl)amino)-1H-pyrazol-1-yl)cyclopropanecarbonitrile;
(Z)-3-(1-((1-(Cyclopropylmethyl)-1H-pyrazol-3-yl)amino)ethylidene)-5-(4-methylpyridin-3-yl)-1H-pyrrolo[2,3-c]pyridin-2(3H)-one;
(Z)-3-(1-((1-(2-Hydroxy-2-methylpropyl)-1H-pyrazol-3-yl)amino)ethylidene)-5-(4-methylpyridin-3-yl)-1H-pyrrolo[2,3-c]pyridin-2(3H)-one;
(Z)-2-Methyl-2-(4-((1-(5-(4-methylpyridin-3-yl)-2-oxo-1H-pyrrolo[3,2-b]pyridin-3(2H)-ylidene)ethyl)amino)-1H-pyrazol-1-yl)propanenitrile;
(Z)-2-(3-((1-(5-(5-Amino-4-methylpyridin-3-yl)-2-oxo-1H-pyrrolo[2,3-c]pyridin-3(2H)-ylidene)ethyl)amino)-1H-pyrazol-1-yl)-2-methylpropanenitrile;
(Z)-5-(4-Methylpyridin-3-yl)-3-(1-((5-morpholinopyridin-2-yl)amino)ethylidene)-1H-pyrrolo[2,3-c]pyridin-2(3H)-one;
(Z)-5-(2-Fluoro-6-methoxyphenyl)-3-(1-((4-(piperazin-1-yl)phenyl)amino)ethylidene)-1H-pyrrolo[2,3-c]pyridin-2(3H)-one;
(Z)-3-(1-((6-(Dimethylamino)pyridin-3-yl)amino)ethylidene)-5-(4-methylpyridin-3-yl)-1H-pyrrolo[2,3-c]pyridin-2(3H)-one;
(Z)-N-(1-Hydroxy-2-methylpropan-2-yl)-2,4-dimethyl-5-((5-(4-methylpyridin-3-yl)-2-oxo-1H-pyrrolo[2,3-c]pyridin-3(2H)-ylidene)methyl)-1H-pyrrole-3-carboxamide;
(Z)-3-(3-((1-(5-(4-Methylpyridin-3-yl)-2-oxo-1H-pyrrolo[2,3-c]pyridin-3(2H)-ylidene)ethyl)amino)-1H-pyrazol-1-yl)propanenitrile;
(Z)-3-(1-((1-(3-Hydroxy-3-methylbutyl)-1H-pyrazol-3-yl)amino)ethylidene)-5-(4-methylpyridin-3-yl)-1H-pyrrolo[2,3-c]pyridin-2(3H)-one;
(Z)-3-(1-((1-(2,2-Difluoroethyl)-1H-pyrazol-3-yl)amino)ethylidene)-5-(4-methylpyridin-3-yl)-1H-pyrrolo[2,3-c]pyridin-2(3H)-one;
(Z)-3-(1-((1,4-Dimethyl-1H-pyrazol-3-yl)amino)ethylidene)-5-(4-methylpyridin-3-yl)-1H-pyrrolo[2,3-c]pyridin-2(3H)-one;
(Z)-N-Methyl-2-(3-((1-(5-(4-methylpyridin-3-yl)-2-oxo-1H-pyrrolo[2,3-c]pyridin-3(2H)-ylidene)ethyl)amino)-1H-pyrazol-1-yl)acetamide;
(R,Z)-2-(3-((1-(5-(4-methylpyridin-3-yl)-2-oxo-1H-pyrrolo[2,3-c]pyridin-3(2H)-ylidene)ethyl)amino)-1H-pyrazol-1-yl)propanenitrile;
(Z)-3-(1-((6-(Dimethylamino)pyridin-3-yl)amino)ethylidene)-5-(2-fluoro-6-methoxyphenyl)-1H-pyrrolo[2,3-c]pyridin-2(3H)-one;
(Z)-N,2-dimethyl-2-(3-((1-(5-(4-methylpyridin-3-yl)-2-oxo-1H-pyrrolo[2,3-c]pyridin-3(2H)-ylidene)ethyl)amino)-1H-pyrazol-1-yl)propanamide;
(Z)-5-(4-Methylpyridin-3-yl)-3-(1-((1,3,5-trimethyl-1H-pyrazol-4-yl)amino)ethylidene)-1H-pyrrolo[2,3-c]pyridin-2(3H)-one;
(Z)-3-(1-((1-(1-Acetylazetidin-3-yl)-1H-pyrazol-3-yl)amino)ethylidene)-5-(4-methylpyridin-3-yl)-1H-pyrrolo[2,3-c]pyridin-2(3H)-one;
(Z)-5-(2-Fluoro-6-methylphenyl)-3-(1-((1-methyl-1H-pyrazol-3-yl)amino)ethylidene)-1H-pyrrolo[2,3-c]pyridin-2(3H)-one;
(Z)-3-(1-((1H-Pyrazol-3-yl)amino)ethylidene)-5-(4-methylpyridin-3-yl)-1H-pyrrolo[2,3-c]pyridin-2(3H)-one;
(Z)-5-(4-Methylpyridin-3-yl)-3-(1-((6-methylpyridin-3-yl)amino)ethylidene)-1H-pyrrolo[2,3-c]pyridin-2(3H)-one;
(Z)-5-(2-Fluoro-6-methoxyphenyl)-3-(1-((5-(piperazin-1-yl)pyridin-2-yl)amino)ethylidene)-1H-pyrrolo[2,3-c]pyridin-2(3H)-one;
(Z)-5-(2-Fluoro-6-methoxyphenyl)-3-(1-((6-(piperazin-1-yl)pyridin-3-yl)amino)ethylidene)-1H-pyrrolo[2,3-c]pyridin-2(3H)-one;
(Z)-5-Methyl-2-((1-(5-(4-methylpyridin-3-yl)-2-oxo-1H-pyrrolo[2,3-c]pyridin-3(2H)-ylidene)ethyl)amino)-4,5-dihydropyrazolo[1,5-a]pyrazin-6(7H)-one;
(Z)-5-Methyl-2-((1-(5-(4-methylpyridin-3-yl)-2-oxo-1H-pyrrolo[2,3-c]pyridin-3(2H)-ylidene)ethyl)amino)-6,7-dihydropyrazolo[1,5-a]pyrazin-4(5H)-one;
(Z)-N,N-Dimethyl-2-(3-((1-(5-(4-methylpyridin-3-yl)-2-oxo-1H-pyrrolo[2,3-c]pyridin-3(2H)-ylidene)ethyl)amino)-1H-pyrazol-1-yl)acetamide;
(Z)-3-(1-((1,5-dimethyl-1H-pyrazol-3-yl)amino)ethylidene)-5-(4-methylpyridin-3-yl)-1H-pyrrolo[2,3-c]pyridin-2(3H)-one;
(Z)-3-(1-((1-(1-Acetylpiperidin-4-yl)-1H-pyrazol-3-yl)amino)ethylidene)-5-(4-methylpyridin-3-yl)-1H-pyrrolo[2,3-c]pyridin-2(3H)-one;
(Z)-5-(4-Methylpyridin-3-yl)-3-(1-((1-(2-morpholino-2-oxoethyl)-1H-pyrazol-3-yl)amino)ethylidene)-1H-pyrrolo[2,3-c]pyridin-2(3H)-one;
(Z)-3-(1-((1,3-Dimethyl-1H-pyrazol-5-yl)amino)ethylidene)-5-(4-methylpyridin-3-yl)-1H-pyrrolo[2,3-c]pyridin-2(3H)-one;
(S,Z)-3-(1-((1-(2-Hydroxypropyl)-1H-pyrazol-3-yl)amino)ethylidene)-5-(4-methylpyridin-3-yl)-1H-pyrrolo[2,3-c]pyridin-2(3H)-one;
(S,Z)-3-(1-((1-(1-Hydroxypropan-2-yl)-1H-pyrazol-3-yl)amino)ethylidene)-5-(4-methylpyridin-3-yl)-1H-pyrrolo[2,3-c]pyridin-2(3H)-one;
(Z)-3-(1-((1,3-Dimethyl-1H-pyrazol-4-yl)amino)ethylidene)-5-(4-methylpyridin-3-yl)-1H-pyrrolo[2,3-c]pyridin-2(3H)-one;
(R,Z)-3-(1-((1-(2-Hydroxypropyl)-1H-pyrazol-3-yl)amino)ethylidene)-5-(4-methylpyridin-3-yl)-1H-pyrrolo[2,3-c]pyridin-2(3H)-one;
(R,Z)-3-(1-((1-(1-Hydroxypropan-2-yl)-1H-pyrazol-3-yl)amino)ethylidene)-5-(4-methylpyridin-3-yl)-1H-pyrrolo[2,3-c]pyridin-2(3H)-one;
(Z)-8-(3-(1-((1-Methyl-1H-pyrazol-3-yl)amino)ethylidene)-2-oxo-2,3-dihydro-1H-pyrrolo[2,3-c]pyridin-5-yl)-2H-pyrido[4,3-b][1,4]oxazin-3(4H)-one;
(Z)-3-(1-((1-Methyl-1H-pyrazol-3-yl)amino)ethylidene)-5-(4-methylpyridin-3-yl)-2-oxoindoline-6-carbonitrile;
(Z)-5-(5-Amino-4-methylpyridin-3-yl)-3-(1-((1-methyl-1H-pyrazol-3-yl)amino)ethylidene)-1H-pyrrolo[2,3-c]pyridin-2(3H)-one;
(Z)-5-(2-Fluoro-6-methylphenyl)-3-(1-((1-(2-hydroxy-2-methylpropyl)-1H-pyrazol-3-yl)amino)ethylidene)-1H-pyrrolo[2,3-c]pyridin-2(3H)-one;
(Z)-2-(3-((1-(5-(2-Fluoro-6-methylphenyl)-2-oxo-1H-pyrrolo[2,3-c]pyridin-3(2H)-ylidene)ethyl)amino)-1H-pyrazol-1-yl)-2-methylpropanenitrile;
(Z)-3-(1-((1-(2-Methoxyethyl)-1H-pyrazol-3-yl)amino)ethylidene)-5-(4-methylpyridin-3-yl)-1H-pyrrolo[2,3-c]pyridin-2(3H)-one;
(Z)-5-(2-Fluoro-6-methoxyphenyl)-3-(1-((1-(2-hydroxy-2-methylpropyl)-1H-pyrazol-3-yl)amino)ethylidene)-1H-pyrrolo[2,3-c]pyridin-2(3H)-one;
(Z)-3-(1-((1-(1-Hydroxy-2-methylpropan-2-yl)-1H-pyrazol-3-yl)amino)ethylidene)-5-(4-methylpyridin-3-yl)-1H-pyrrolo[2,3-c]pyridin-2(3H)-one;
(Z)-3-(1-((6,7-Dihydro-4H-pyrazolo[5,1-c][1,4]oxazin-2-yl)amino)ethylidene)-5-(4-methylpyridin-3-yl)-1H-pyrrolo[2,3-c]pyridin-2(3H)-one;
(Z)-5-(4-Methylpyridin-3-yl)-3-(1-((1-(1-(oxetan-3-yl)piperidin-4-yl)-1H-pyrazol-3-yl)amino)ethylidene)-1H-pyrrolo[2,3-c]pyridin-2(3H)-one;
(Z)-5-(5-Fluoro-4-methylpyridin-3-yl)-3-(1-((1-methyl-1H-pyrazol-4-yl)amino)ethylidene)-1H-pyrrolo[2,3-c]pyridin-2(3H)-one;
(Z)-5-Isopropyl-2-((1-(5-(4-methylpyridin-3-yl)-2-oxo-1H-pyrrolo[2,3-c]pyridin-3(2H)-ylidene)ethyl)amino)-4,5-dihydropyrazolo[1,5-a]pyrazin-6(7H)-one;
(Z)-3-(1-((5-(2-Hydroxypropan-2-yl)-1-methyl-1H-pyrazol-3-yl)amino)ethylidene)-5-(4-methylpyridin-3-yl)-1H-pyrrolo[2,3-c]pyridin-2(3H)-one;
(Z)-3-(1-((1-Methyl-1H-pyrazol-4-yl)amino)ethylidene)-5-(4-methylpyridin-3-yl)-2-oxoindoline-6-carbonitrile;
(Z)-3-(1-((5-Cyclopropyl-1-methyl-1H-pyrazol-3-yl)amino)ethylidene)-5-(4-methylpyridin-3-yl)-1H-pyrrolo[2,3-c]pyridin-2(3H)-one;
(R,Z)-3-(1-((1-(2-Hydroxypropyl)-1H-pyrazol-4-yl)amino)ethylidene)-5-(4-methylpyridin-3-yl)-1H-pyrrolo[2,3-c]pyridin-2(3H)-one;
(Z)-5-(5-Fluoro-4-methylpyridin-3-yl)-3-(1-((1-methyl-1H-pyrazol-3-yl)amino)ethylidene)-1H-pyrrolo[2,3-c]pyridin-2(3H)-one;
(Z)-2,2-Difluoro-N-methyl-2-(3-((1-(5-(4-methylpyridin-3-yl)-2-oxo-1H-pyrrolo[2,3-c]pyridin-3(2H)-ylidene)ethyl)amino)-1H-pyrazol-1-yl)acetamide;
(S,Z)-3-(1-((1-(2-Hydroxypropyl)-1H-pyrazol-4-yl)amino)ethylidene)-5-(4-methylpyridin-3-yl)-1H-pyrrolo[2,3-c]pyridin-2(3H)-one;
(Z)-3-(1-((1-Methyl-1H-pyrazol-4-yl)amino)ethylidene)-5-(2-methylpyridin-3-yl)-1H-pyrrolo[2,3-c]pyridin-2(3H)-one;
(R,Z)-2-(4-((1-(5-(4-Methylpyridin-3-yl)-2-oxo-1H-pyrrolo[2,3-c]pyridin-3-(2H)-ylidene)ethyl)amino)-1H-pyrazol-1-yl)propanenitrile;
(Z)-3-(1-((1-Methyl-1H-pyrazol-4-yl)amino)ethylidene)-5-(4-methylpyridin-3-yl)indolin-2-one;
(Z)-3-(1-((1-Methyl-1H-pyrazol-3-yl)amino)ethylidene)-5-(2-methylpyridin-3-yl)-1H-pyrrolo[2,3-c]pyridin-2(3H)-one;
(Z)-3-(1-((1-Methyl-1H-1,2,4-triazol-3-yl)amino)ethylidene)-5-(4-methylpyridin-3-yl)-1H-pyrrolo[2,3-c]pyridin-2(3H)-one;
(Z)-5-(2-Fluoro-6-methylphenyl)-3-(1-((1-methyl-1H-pyrazol-4-yl)amino)ethylidene)-1H-pyrrolo[2,3-c]pyridin-2(3H)-one;
(R,Z)-3-(1-((1-(3-Hydroxybutyl)-1H-pyrazol-4-yl)amino)ethylidene)-5-(4-methylpyridin-3-yl)-1H-pyrrolo[2,3-c]pyridin-2(3H)-one;
(Z)-3-(1-((1-(3-Hydroxy-3-methylbutyl)-1H-pyrazol-4-yl)amino)ethylidene)-5-(4-methylpyridin-3-yl)-1H-pyrrolo[2,3-c]pyridin-2(3H)-one;
(S,Z)-2-(4-((1-(5-(4-Methylpyridin-3-yl)-2-oxo-1H-pyrrolo[2,3-c]pyridin-3(2H)-ylidene)ethyl)amino)-1H-pyrazol-1-yl)propanenitrile;
(Z)-3-(1-((1-((1-Hydroxycyclopropyl)methyl)-1H-pyrazol-3-yl)amino)ethylidene)-5-(4-methylpyridin-3-yl)-1H-pyrrolo[2,3-c]pyridin-2(3H)-one;
(R,Z)-3-(1-((1-(3-Hydroxybutyl)-1H-pyrazol-3-yl)amino)ethylidene)-5-(4-methylpyridin-3-yl)-1H-pyrrolo[2,3-c]pyridin-2(3H)-one;
(Z)-3-(1-((1-(2,2-Difluoroethyl)-5-methyl-1H-pyrazol-3-yl)amino)ethylidene)-5-(4-methylpyridin-3-yl)-1H-pyrrolo[2,3-c]pyridin-2(3H)-one;
(R,Z)-2-(4-((1-(5-(2-Fluoro-6-methoxyphenyl)-2-oxo-1H-pyrrolo[2,3-c]pyridin-3(2H)-ylidene)ethyl)amino)-1H-pyrazol-1-yl)propanenitrile;
(S,Z)-2-(4-((1-(5-(2-Fluoro-6-methoxyphenyl)-2-oxo-1H-pyrrolo[2,3-c]pyridin-3(2H)-ylidene)ethyl)amino)-1H-pyrazol-1-yl)propanenitrile;
(S,Z)-5-(2-Fluoro-6-methoxyphenyl)-3-(1-((1-(2-hydroxypropyl)-1H-pyrazol-4-yl)amino)ethylidene)-1H-pyrrolo[2,3-c]pyridin-2(3H)-one;
(Z)-3-(1-((1-((1-Hydroxycyclopropyl)methyl)-1H-pyrazol-4-yl)amino)ethylidene)-5-(4-methylpyridin-3-yl)-1H-pyrrolo[2,3-c]pyridin-2(3H)-one;
(Z)-3-(1-((1-Methyl-1H-indazol-3-yl)amino)ethylidene)-5-(4-methylpyridin-3-yl)-1H-pyrrolo[2,3-c]pyridin-2(3H)-one;
(S,Z)-3-(1-((1-3-Hydroxybutyl)-1H-pyrazol-4-yl)amino)ethylidene)-5-(4-methylpyridin-3-yl)-1H-pyrrolo[2,3-c]pyridin-2(3H)-one;
(Z)-3-(1-((6-((1-(Cyclopropanecarbonyl)piperidin-4-yl)oxy)pyridin-3-yl)amino)ethylidene)-5-(4-methylpyridin-3-yl)-1H-pyrrolo[2,3-c]pyridin-2(3H)-one;
(Z)-5-(4-Methylpyridin-3-yl)-3-(1-((5-methylthiazol-2-yl)amino)ethylidene)-1H-pyrrolo[2,3-c]pyridin-2(3H)-one;
(Z)-3-(1-((5-Methyl-1H-pyrazol-3-yl)amino)ethylidene)-5-(4-methylpyridin-3-yl)-1H-pyrrolo[2,3-c]pyridin-2(3H)-one;
(Z)-3-(1-((1-(Ethylsulfonyl)-5-methyl-1 H-pyrazol-3-yl)amino)ethylidene)-5-(4-methylpyridin-3-yl)-1H-pyrrolo[2,3-c]pyridin-2(3H)-one;
(Z)-3-(1-((1-(2-Hydroxy-2-methylpropyl)-5-methyl-1H-pyrazol-3-yl)amino)ethylidene)-5-(4-methylpyridin-3-yl)-1H-pyrrolo[2,3-c]pyridin-2(3H)-one;
(Z)-3-(1-((1-Ethyl-5-methyl-1H-pyrazol-3-yl)amino)ethylidene)-5-(4-methylpyridin-3-yl)-1H-pyrrolo[2,3-c]pyridin-2(3H)-one;
(Z)-3-(1-((1-(3-Hydroxy-3-methylbutyl)-5-methyl-1H-pyrazol-3-yl)amino)ethylidene)-5-(4-methylpyridin-3-yl)-1H-pyrrolo[2,3-c]pyridin-2(3H)-one;
(S,Z)-3-(1-((1-(2-Hydroxypropyl)-5-methyl-1H-pyrazol-3-yl)amino)ethylidene)-5-(4-methylpyridin-3-yl)-1H-pyrrolo[2,3-c]pyridin-2(3H)-one;
(Z)-3-(1-((1-(Difluoromethyl)-5-methyl-1H-pyrazol-3-yl)amino)ethylidene)-5-(4-methylpyridin-3-yl)-1H-pyrrolo[2,3-c]pyridin-2(3H)-one;
(Z)-3-(1-((1-Isobutyl-5-methyl-1H-pyrazol-3-yl)amino)ethylidene)-5-(4-methylpyridin-3-yl)-1H-pyrrolo[2,3-c]pyridin-2(3H)-one;
(Z)-4-Methyl-3-(3-(1-((1-methyl-1H-pyrazol-4-yl)amino)ethylidene)-2-oxo-2,3-dihydro-1H-pyrrolo[2,3-c]pyridin-5-yl)benzonitrile;
(R,Z)-3-(1-((1-(2-Hydroxypropyl)-5-methyl-1H-pyrazol-3-yl)amino)ethylidene)-5-(4-methylpyridin-3-yl)-1H-pyrrolo[2,3-c]pyridin-2(3H)-one;
(Z)-3-(1-((1-(cyclopropylmethyl)-5-methyl-1H-pyrazol-3-yl)amino)ethylidene)-5-(4-methylpyridin-3-yl)-1H-pyrrolo[2,3-c]pyridin-2(3H)-one;
(R,Z)-3-(1-((1-(3-Hydroxybutyl)-5-methyl-1H-pyrazol-3-yl)amino)ethylidene)-5-(4-methylpyridin-3-yl)-1H-pyrrolo[2,3-c]pyridin-2(3H)-one;
(Z)-3-(1-((1,5-dimethyl-1H-pyrazol-3-yl)amino)ethylidene)-5-(2-fluoro-6-methylphenyl)-1H-pyrrolo[2,3-c]pyridin-2(3H)-one;
(Z)-3-(1-((6,7-Dihydro-4H-pyrazolo[5,1-c][1,4]oxazin-2-yl)amino) ethylidene)-5-(2-fluoro-6-methylphenyl)-1H-pyrrolo[2,3-c]pyridin-2(3H)-one;
(Z)-3-(1-((6,7-Dihydro-4H-pyrazolo[5,1-c][1,4]oxazin-2-yl)amino) ethylidene)-5-(4-methylpyridin-3-yl)indolin-2-one;
(Z)-3-(1-((1,5-Dimethyl-1H-pyrazol-3-yl)amino)ethylidene)-5-(4-methylpyridin-3-yl)indolin-2-one;
(S,Z)-3-(1-((3-Hydroxybutyl)-5-methyl-1H-pyrazol-3-yl)amino)ethylidene)-5-(4-methylpyridin-3-yl)-1H-pyrrolo[2,3-c]pyridin-2(3H)-one;
(S,Z)-3-(1-((3-hydroxybutyl)-1H-pyrazol-3-yl)amino)ethylidene)-5-(4-methylpyridin-3-yl)-1H-pyrrolo[2,3-c]pyridin-2(3H)-one;
(Z)-2-Methyl-2-(5-methyl-3-((1-(5-(4-Methylpyridin-3-yl)-2-oxo-1H-pyrrolo[2,3-c]pyridin-3(2H)-ylidene)ethyl)amino)-1H-pyrazol-1-yl)propanenitrile;
(R,Z)-2-(5-Methyl-3-((1-(5-(4-methylpyridin-3-yl)-2-oxo-1H-pyrrolo[2,3-c]pyridin-3(2H)-ylidene)ethyl)amino)-1H-pyrazol-1-yl)propanenitrile;
(R,Z)-2-(3-((1-(5-(2-Fluoro-6-methylphenyl)-2-oxo-1H-pyrrolo[2,3-c]pyridin-3(2H)-ylidene)ethyl)amino)-5-methyl-1H-pyrazol-1-yl)propanenitrile;
(Z)-5-(2-Fluoro-6-methylphenyl)-3-(1-((1-(2-hydroxy-2-methylpropyl)-5-methyl-1H-pyrazol-3-yl)amino)ethylidene)-1H-pyrrolo[2,3-c]pyridin-2(3H)-one;
(Z)-3-(1-((1-(2,2-Difluoroethyl)-5-methyl-1H-pyrazol-3-yl)amino)ethylidene)-5-(2-fluoro-6-methylphenyl)-1H-pyrrolo[2,3-c]pyridin-2(3H)-one;
(Z)-5-(2-Fluoro-6-methylphenyl)-3-(1-((1-isobutyl-5-methyl-1H-pyrazol-3-yl)amino)ethylidene)-1H-pyrrolo[2,3-c]pyridin-2(3H)-one;
(Z)-5-(2-Fluoro-6-methylphenyl)-3-(1-((1-(3-hydroxy-3-methylbutyl)-5-methyl-1H-pyrazol-3-yl)amino)ethylidene)-1H-pyrrolo[2,3-c]pyridin-2(3H)-one;
(Z)-3-(1-((1-Isopentyl-5-methyl-1H-pyrazol-3-yl)amino)ethylidene)-5-(4-methylpyridin-3-yl)-1H-pyrrolo[2,3-c]pyridin-2(3H)-one;
(Z)-5-(2-Fluoro-6-methylphenyl)-3-(1-((5-methylthiazol-2-yl)amino)ethylidene)-1H-pyrrolo[2,3-c]pyridin-2(3H)-one;
(Z)-2-((1-(5-(2-Fluoro-6-methylphenyl)-2-oxo-1H-pyrrolo[2,3-c]pyridin-3(2H)-ylidene)ethyl)amino)-5-isopropyl-4,5-dihydropyrazolo[1,5-a]pyrazin-6(7H)-one;
(Z)-2-(3-((1-(5-(2-Fluoro-6-methylphenyl)-2-oxo-1H-pyrrolo[2,3-c]pyridin-3(2H)-ylidene)ethyl)amino)-5-methyl-1H-pyrazol-1-yl)-2-methylpropanenitrile;
(S,Z)-2-(3-((1-(5-(2-Fluoro-6-methylphenyl)-2-oxo-1H-pyrrolo[2,3-c]pyridin-3(2H)-ylidene)ethyl)amino)-5-methyl-1H-pyrazol-1-yl)propanenitrile;
(S,Z)-5-(2-Fluoro-6-methylphenyl)-3-(1-((1-(2-hydroxypropyl)-5-methyl-1H-pyrazol-3-yl)amino)ethylidene)-1H-pyrrolo[2,3-c]pyridin-2(3H)-one;
(Z)-3-(1-((1,5-Dimethyl-1H-pyrazol-3-yl)amino)ethylidene)-5-(5-fluoro-2-methylphenyl)-1H-pyrrolo[2,3-c]pyridin-2(3H)-one;
(Z)-3-(1-((1-Isopropyl-5-methyl-1H-pyrazol-3-yl)amino)ethylidene)-5-(4-methylpyridin-3-yl)-1H-pyrrolo[2,3-c]pyridin-2(3H)-one;
(Z)-3-(1-((1-Ethyl-5-methyl-1H-pyrazol-3-yl)amino)ethylidene)-5-(2-fluoro-6-methylphenyl)-1H-pyrrolo[2,3-c]pyridin-2(3H)-one;
(Z)-3-(1-((1-(2,2-Difluoroethyl)-5-methyl-1H-pyrazol-3-yl)amino)ethylidene)-5-(5-fluoro-2-methylphenyl)-1H-pyrrolo[2,3-c]pyridin-2(3H)-one;
(S,Z)-2-(5-Methyl-3-((1-(5-(4-methylpyridin-3-yl)-2-oxo-1H-pyrrolo[2,3-c]pyridin-3(2H)-ylidene)ethyl)amino)-1H-pyrazol-1-yl)propanenitrile;
(Z)-3-(1-((1-(Difluoromethyl)-5-methyl-1H-pyrazol-3-yl)amino)ethylidene)-5-(5-fluoro-2-methylphenyl)-1H-pyrrolo[2,3-c]pyridin-2(3H)-one;
(R,Z)-5-(2-Fluoro-6-methylphenyl)-3-(1-((1-(2-hydroxypropyl)-5-methyl-1H-pyrazol-3-yl)amino)ethylidene)-1H-pyrrolo[2,3-c]pyridin-2(3H)-one;
(Z)-3-(1-((4,5-Dimethylthiazol-2-yl)amino)ethylidene)-5-(4-methylpyridin-3-yl)-1H-pyrrolo[2,3-c]pyridin-2(3H)-one;
(Z)-3-(1-((6,7-Dihydro-4H-pyrazolo[5,1-c][1,4]oxazin-2-yl)amino)ethylidene)-5-(5-fluoro-2-methylphenyl)-1H-pyrrolo[2,3-c]pyridin-2(3H)-one and
(Z)-7-(1-((1-methyl-1H-pyrazol-3-yl)amino)ethylidene)-2-(4-methylpyridin-3-yl)-5H-pyrrolo[3,2-d]pyrimidin-6(7H)-one or pharmaceutically acceptable salt thereof.

14. The compound of formula according to claim 1 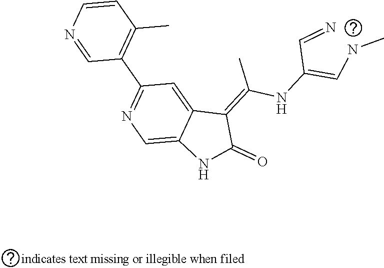 [SMILES: CN/C=C(\C=N)N/C(C)=C1\C(=O)Nc2cnc(-c3cnccc3C)cc21]
or a stereoisomer thereof, or a pharmaceutically acceptable salt thereof.

15. The compound of formula according to claim 1 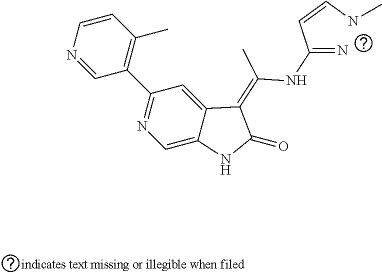 [SMILES: CN/C=C\C(=N)N/C(C)=C1\C(=O)Nc2cnc(-c3cnccc3C)cc21]
or a stereoisomer thereof, or a pharmaceutically acceptable salt thereof.

16. The compound of formula according to claim 1 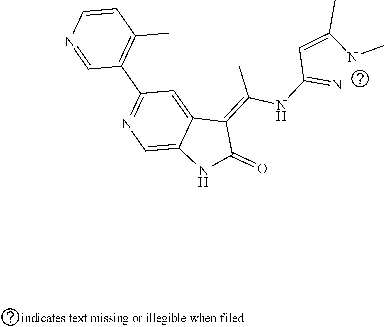 [SMILES: CN/C(C)=C\C(=N)N/C(C)=C1\C(=O)Nc2cnc(-c3cnccc3C)cc21]
or a stereoisomer thereof, or a pharmaceutically acceptable salt thereof.

17. The compound of formula according to claim 1 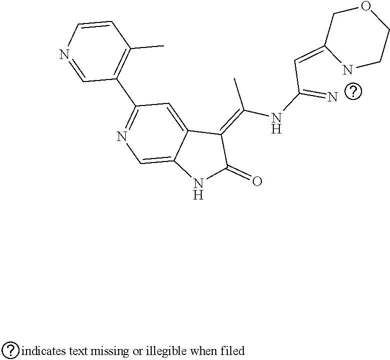 [SMILES: C/C(NC(=N)/C=C1/COCCN1)=C1/C(=O)Nc2cnc(-c3cnccc3C)cc21]
or a stereoisomer thereof, or a pharmaceutically acceptable salt thereof.

18. The compound of formula according to claim 1 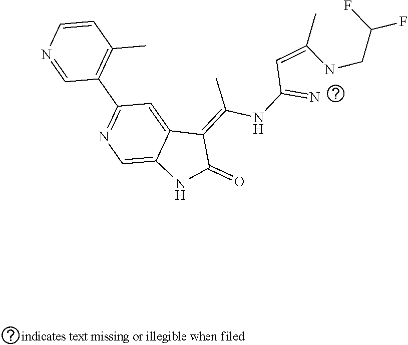 [SMILES: C/C(=C/C(=N)N/C(C)=C1\C(=O)Nc2cnc(-c3cnccc3C)cc21)NCC(F)F]
or a stereoisomer thereof, or a pharmaceutically acceptable salt thereof.

19. The compound of formula according to claim 1 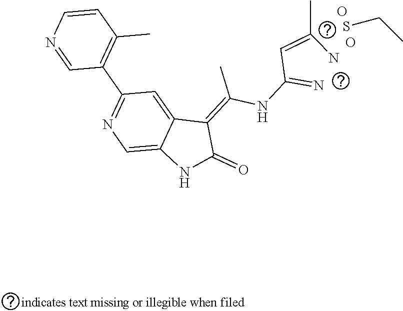
or a stereoisomer thereof, or a pharmaceutically acceptable salt thereof.

20. The compound of formula according to claim 1 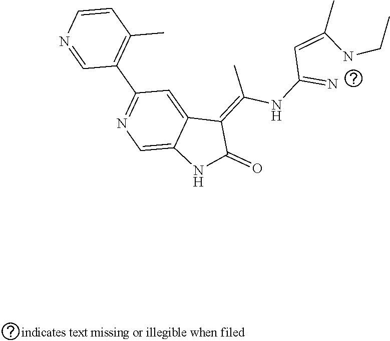 [SMILES: CCN/C(C)=C\C(=N)N/C(C)=C1\C(=O)Nc2cnc(-c3cnccc3C)cc21]
or a stereoisomer thereof, or a pharmaceutically acceptable salt thereof.

21. The compound of formula according to claim 1 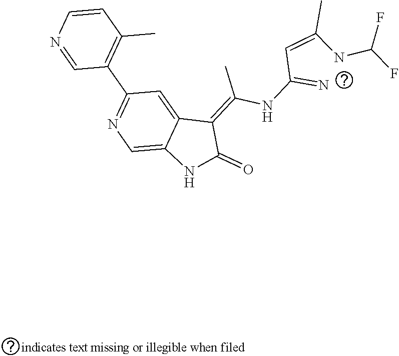 [SMILES: C/C(=C/C(=N)N/C(C)=C1\C(=O)Nc2cnc(-c3cnccc3C)cc21)NC(F)F]
or a stereoisomer thereof, or a pharmaceutically acceptable salt thereof.

22. A pharmaceutical composition comprising a compound according to claim 1 and a pharmaceutically acceptable excipient.

23. The pharmaceutical composition according to claim 22, wherein the pharmaceutically acceptable excipient is a carrier or diluent.

24. A method of treating a MAP4K1 (HPK1) mediated disease, disorder, syndrome, or condition in a subject comprising administering an effective amount of a compound according to claim 1.

25.-26. (canceled)

27. The method according to claim 24, wherein the disease, disorder, syndrome or condition is cancer.
